(19) United States
(12) Patent Application Publication
(10) Pub. No.: US 2022/0275043 A1
(43) Pub. Date: Sep. 1, 2022

(54) SOLUBLE MULTIMERIC IMMUNOGLOBULIN-SCAFFOLD BASED FUSION PROTEINS AND USES THEREOF

(71) Applicant: Massachusetts Institute of Technology, Cambridge, MA (US)

(72) Inventors: Darrell J. IRVINE, Arlington, MA (US); Leyuan MA, Brookline, MA (US)

(21) Appl. No.: 17/257,671

(22) PCT Filed: Jul. 17, 2019
(86) PCT No.: PCT/US19/42280

§ 371 Date: Jan. 4, 2021

Related U.S. Application Data
(60) Provisional application No. 62/699,422, filed on Jul. 17, 2018.

(51) Int. Cl. C07K 14/725; C07K 14/74

(52) U.S. Cl. CPC C07K 14/7051; C07K 14/70539; C07K 2317/52; C07K 2317/567; C07K 2319/30; C07K 2319/73

(57) ABSTRACT

The present disclosure provides soluble, multimeric fusion proteins that bind to a component of the MHC/TCR immune complex, wherein the fusion proteins comprise a soluble T cell receptor (TCR) or soluble Major Histocompatibility Complex (MHC) linked to an immunoglobulin framework by a multimerization domain. The disclosure also features compositions and methods of using the same for therapeutic or diagnostic use.

### CROSS-REFERENCE TO RELATED APPLICATIONS

This application is a 35 U.S.C. § 371 national stage filing of International Application No. PCT/US2019/042280 filed on Jul. 17, 2019, which claims the benefit of U.S. Patent Application No. 62/699,422, filed on Jul. 17, 2018. The entire contents of each of these applications are incorporated herein by reference in their entirety.

### SEQUENCE LISTING

The instant application contains a Sequence Listing which has been submitted electronically in ASCII format via EFS-Web and is hereby incorporated by reference in its entirety. Said ASCII copy, created on Jan. 4, 2021, is named MITN-045US_Sequence-Listing.txt and is 161634 bytes in size.

### BACKGROUND

The use of therapeutic antibodies have led to significant advances in the treatment of cancer and other diseases. However, most therapeutic antibodies only have access to antigens expressed on the cell surface and surface expressing disease-specific antigens (e.g., tumor antigens) are rare. In contrast, the immune response to intracellular antigens, as well as the stimulation and maintenance of efficient cytotoxic responses are controlled by the interaction of the T-cell receptor (TCR) and both intra- and extra-cellular peptides presented in the context of major histocompatibility complex (MHC) class I and II molecules.

Accordingly, alternative therapeutic strategies which focus on TCRs have been reported. One challenge with respect to TCRs as opposed to antibodies, however, is that the former are not secreted from the cells in which they are made. A variety of approaches for producing soluble TCRs have been reported, including the production of TCR multimers via biotin-streptavidin technology, isolation of α and β chains from bacterial inclusion bodies (WO 2013/057586), and hybrid, single-chain TCR-IgG molecules connected via a flexible linker (e.g., STAR™ technology, Altor Bioscience Corporation). However, all of these strategies have been hampered by difficulties associated with low stability, low expression yields, aggregation of purified proteins and mis-folding.

Efforts to improve production and stability include the generation of disulfide-bond linked TCRs (dsTCRs), which have a non-native bridge between the TCR constant domains. While demonstrating increased stability, TCRs generated by this method must still be isolated and refolded from inclusion bodies. TCRs fused to other soluble polypeptides, and high affinity TCR-mimic antibodies also have been developed which contain stabilizing mutations in an effort to improve production and secretion. However, success using this technology has been limited due to the difficulty of their production, low secretion levels and risk of off-target toxicity (reviewed om Trenevska et al., Front. Immunol. 8:1001, 2017). Thus, despite recent improvements in the technology for generating soluble TCRs and TCR-multimers, the methods are still laborious with expression levels that vary extensively between individual clones (reviewed in Loset et al., Front. Oncol. 4:378, 2015).

Strategies which utilize soluble peptide-loaded Major Histocompatibility Complex class I and II molecules (pMHC) also have been reported. For example, pMHC multimers have been used for detection of antigen-responsive T cells since the first report by Altman et al. (Science 274:94-96, 1996) in which pMHC class I avidin-biotin-based tetramers were used in flow cytometry to detect MHC multimer-binding T cells. However, since MHC molecules are largely unstable when they are not part of a complex with peptide, pMHC-based technologies were initially restricted by the tedious production of molecules, where each peptide required an individual folding and purification procedure (Bakker et al. Curr. Opin. Immunol. 17:428-433, 2005). More recently, a variety of MHC molecules with covalently linked peptides have also been reported (e.g., reviewed by Goldberg, et al. J. Cell. Mol. Med. 15:1822-1832, 2011). However, broad application of soluble MHC multimers has been hindered, for example, due to difficulties in producing soluble MHC class II molecules, as well as complications caused by low TCR-MHC avidity.

Accordingly, there is still a need for methods of routinely and efficiently producing high quantities of soluble TCR and pMHC based multimeric proteins which bind to antigenic peptides with sufficient affinity for use as both diagnostic and therapeutic agents for disorders involving regulation of the immune system.

### SUMMARY OF THE DISCLOSURE

The present disclosure is based, at least in part, on the discovery that soluble, multimeric proteins containing an immunoglobulin (Igg) framework operably linked to a component of a TCR/MHC complex (e.g., a binding portion of a TCR or MHC molecule) by a flexible multimerization domain demonstrate enhanced stabilization and can be produced in a short period of time with high protein yields. The propensity of Igg heavy chain and light chain constant regions to dimerize provides a framework to display multiple TCR or MHC receptors. However, directly linking TCR or MHC receptor variable regions to the Igg framework of an antibody (e.g., Igg heavy chain constant region and/or Igg light chain constant region) results in low protein yield. Without being bound by theory, exchange of TCR or MHC receptor variable regions with the variable regions of an antibody likely results in low protein stability, protein mis-folding or protein aggregation that are detrimental for protein production. However, as disclosed herein, linking multiple TCR or MHC receptors to an Igg framework using flexible multimerization domains greatly enhances protein yield. For example, fusion of two TCRs to an Igg framework by multimerization domains results in a sizeable increase in protein yield compared to the same multimeric TCR-Igg fusion protein lacking the flexible multimerization domains.

Accordingly, in one aspect, the disclosure provides a soluble fusion polypeptide comprising a component of a TCR/MHC complex operatively linked to an immunoglobulin framework via a multimerization domain.

In one aspect, the disclosure provides a soluble, multimeric protein comprising two or more soluble fusion polypeptides comprising a component of a TCR/MHC complex operatively linked to an immunoglobulin framework via a multimerization domain. As described herein, assembly of the fusion polypeptides through the multimerization domain and/or the immunoglobulin framework provides a multimeric display of TCRs or MHC receptors (e.g., a dimer, trimer, tetramer or hexamer of the TCR or MHC receptor).

In one example, assembly of fusion proteins comprising a TCR polypeptide operatively linked to an immunoglobulin heavy chain constant region or immunoglobulin light chain constant region via a multimerization domain, provides a soluble, multimeric TCR fusion protein that is a TCR dimer. As described herein, the resulting TCR-Igg dimer has higher affinity for antigen peptide presented by a cellular MHC I receptor as compared to a TCR monomer. Thus, without being bound by theory, stable display of multiple TCRs provides increased affinity for MHC-presented antigen peptide.

In another example, assembly of fusion proteins comprising a single-chain peptide-MHC class I polypeptide operatively linked to an immunoglobulin heavy chain constant region or immunoglobulin light chain constant region via a multimerization domain, provides a soluble, multimeric MHC class I-immunoglobulin fusion protein that is a pMHCI dimer or tetramer. As described herein, the resulting pMHCI-Igg dimer and tetramer efficiently bind to T cells expressing a TCR specific to the pMHCI.

In another aspect, the disclosure provides a nucleic acid encoding a fusion polypeptide of the disclosure. In certain embodiments, the nucleic acid comprises one or more recombinant expression vectors. In certain embodiments, the nucleic acid comprises a single recombinant vector. In one example, a nucleic acid encoding a multimeric TCR-immunoglobulin fusion protein comprising a single recombinant vector has a 3-fold increase in protein expression compared to a nucleic acid encoding a multimeric TCR-immunoglobulin fusion protein comprising two recombinant vectors.

In related embodiments, the disclosure provides a host cell comprising a nucleic acid encoding one or more fusion polypeptides of the disclosure.

In another aspect, the disclosure provides a method of producing a soluble, multimeric protein of the disclosure, comprising providing a host cell expressing the two or more fusion polypeptides of the disclosure under conditions sufficient to promote formation of a multimeric protein comprising two or more fusion polypeptides, and isolating the multimeric protein.

In another aspect, the disclosure provides compositions and kits comprising the soluble, multimeric protein of the disclosure.

In another aspect, the disclosure provides methods of using the soluble, multimeric protein of the disclosure for diagnostic and therapeutic applications.

Accordingly, in one aspect, the disclosure provides a soluble, multimeric fusion protein that binds to a component of the MHC/TCR immune complex, comprising: (a) a first fusion protein comprising a soluble T cell receptor (TCR) or a soluble Major Histocompatibility Complex (MHC) linked to an immunoglobulin framework by a first multimerization domain; and (b) a second fusion protein comprising a soluble TCR or a soluble MHC linked to an immunoglobulin framework by a second multimerization domain that binds to the first multimerization domain; wherein the first and second fusion protein form a soluble multimeric fusion protein.

In some embodiments, disclosure provides a soluble, multimeric fusion protein wherein the first fusion protein comprises a soluble TCR polypeptide comprising a variable alpha (Vα) domain, and optionally a constant alpha (Cα) domain, and the second fusion protein comprises a soluble TCR polypeptide comprising a variable β domain (Vβ), and optionally a constant β domain (Cβ).

In some embodiments, disclosure provides a soluble, multimeric fusion protein wherein the first fusion protein comprises a soluble TCR polypeptide comprising a Vα domain, a Vβ domain and a Cβ domain, and the second fusion protein comprises soluble TCR polypeptide comprising a Vα domain, a Vβ domain and a Cβ domain.

In some embodiments, disclosure provides a soluble, multimeric fusion protein wherein at least one fusion protein comprises a soluble TCR polypeptide operably linked to an immunoglobulin heavy chain constant region or fragment thereof. In some embodiments, disclosure provides a soluble, multimeric fusion protein wherein at least two fusion proteins comprise a soluble TCR polypeptide operably linked to an immunoglobulin heavy chain constant region or fragment thereof. In some embodiments, disclosure provides a soluble, multimeric fusion protein wherein at least three fusion proteins comprise a soluble TCR polypeptide operably linked to an immunoglobulin heavy chain constant region or fragment thereof.

In some embodiments, disclosure provides a soluble, multimeric fusion protein wherein the first fusion protein comprises a soluble TCR polypeptide operably linked to an immunoglobulin heavy chain constant region or fragment thereof, and the second fusion protein comprises a soluble TCR polypeptide operably linked to an immunoglobulin light chain constant region or fragment thereof. In some embodiments, disclosure provides a soluble, multimeric fusion protein wherein at least two first fusion proteins comprise a soluble TCR polypeptide operably linked to an immunoglobulin heavy chain constant region or fragment thereof, and at least two second fusion proteins comprise a soluble TCR polypeptide operably linked to an immunoglobulin light chain constant region or fragment thereof.

In any of the preceding embodiments, the disclosure provides a soluble, multimeric fusion protein wherein the multimeric protein fusion is a dimer, a trimer, a tetramer or a hexamer. In any of the preceding embodiments, the disclosure provides a soluble, multimeric fusion protein wherein the multimeric protein fusion is a dimer. In any of the preceding embodiments, the disclosure provides a soluble, multimeric fusion protein wherein the multimeric protein fusion is a tetramer.

In some embodiments, the disclosure provides a soluble, multimeric TCR-immunoglobulin fusion protein comprising (a) a first fusion protein comprising the structure VαCα-X¹-Ig(Fc), wherein Vα is a TCR α variable region, Cα is TCR α constant region, X¹ is a first multimerization domain, and Ig(Fc) is an immunoglobulin Fc domain or fragment thereof; and (b) a second fusion protein comprising the structure VβCβ-X²-Ig(C_{L}), wherein Vβ is a TCR β variable region, Cβ is a TCR β constant region, X¹ is a second multimerization domain, and Ig(C_{L}) is an immunoglobulin light chain constant region or fragment thereof; wherein the first and second fusion proteins form a soluble, multimeric TCR-immunoglobulin protein. In some embodiments, the soluble, multimeric TCR-immunoglobulin fusion protein is a dimer. In some embodiments, the soluble, multimeric TCR-immunoglobulin fusion protein is a tetramer.

In some embodiments, the disclosure provides a soluble, multimeric TCR-immunoglobulin fusion protein comprising (a) a first fusion protein comprising the structure VαVβCβ-X¹-Ig(Fc), wherein Vα is a TCR α variable region, Vβ is a TCR β variable region, Cβ is a TCR α variable region, X¹ is a first multimerization domain, and Ig(Fc) is an immunoglobulin Fc domain or fragment thereof; and (b) a second fusion protein comprising the structure VαVβCβ-X²-Ig(C_{L}), wherein Vα is a TCR α variable region, Vβ is a TCR β variable region, Cβ is a TCR α variable region, X¹ is a second multimerization domain, and Ig(C_{L}) is an immunoglobulin light chain constant region; and wherein the first and second fusion proteins form a soluble, multimeric TCR-immunoglobulin fusion protein. In some embodiments, the soluble, multimeric TCR-immunoglobulin fusion protein is a dimer. In some embodiments, the soluble, multimeric TCR-immunoglobulin fusion protein is a tetramer.

In some embodiments, the disclosure provides a soluble, multimeric TCR-immunoglobulin fusion protein comprising (a) a first fusion protein comprising the structure Vα-X¹-Ig(C_{H}), wherein Vα is a TCR α variable region, X¹ is a first multimerization domain, and Ig(C_{H}) is an immunoglobulin heavy chain constant region or fragment thereof; and (b) a second fusion protein comprising the structure Vβ-X²-Ig(C_{L}), wherein Vβ is a TCR β variable region, X¹ is a second multimerization domain, and Ig(C_{L}) is an immunoglobulin light chain constant region or fragment thereof; wherein the first fusion protein and the second fusion protein form a soluble, multimeric TCR-immunoglobulin fusion protein. In some embodiments, the multimeric fusion protein comprises two first fusion proteins and two second fusion proteins. In some embodiments, the immunoglobulin heavy chain constant region or fragment thereof of the two first fusion proteins and the immunoglobulin light chain constant region of the two second fusion proteins forms an immunoglobulin framework. In some embodiments, the soluble, multimeric TCR-immunoglobulin fusion protein is a dimer.

In some embodiments, the disclosure provides a soluble, multimeric TCR-immunoglobulin fusion protein comprising: (a) a first fusion protein comprising the structure VαCα-X¹-Ig(C_{H}), wherein Vα is a TCR α variable region, Cα is TCR α constant region, X¹ is a first multimerization domain, and Ig(C_{H}) is an immunoglobulin heavy chain constant region or fragment thereof; and (b) a second fusion protein comprising the structure VβCβ-X²-Ig(C_{L}), wherein Vβ is a TCR 13 variable region, Cβ is a TCR β constant region, X¹ is a second multimerization domain, and Ig(C_{L}) is an immunoglobulin light chain constant region or fragment thereof; wherein the at least one first fusion protein and the at least one second fusion protein form a soluble, multimeric TCR-immunoglobulin fusion protein. In some embodiments, the multimeric fusion protein comprises two first fusion proteins and two second fusion proteins. In some embodiments, the immunoglobulin heavy chain constant region or fragment thereof of the two first fusion proteins and the immunoglobulin light chain constant region of the two second fusion proteins forms an immunoglobulin framework. In some embodiments, the soluble, multimeric TCR-immunoglobulin fusion protein is a dimer.

In some embodiments, the disclosure provides a soluble, multimeric TCR-immunoglobulin fusion protein comprising (a) a first fusion protein comprising the structure VαVβCβ-X¹-Ig(C_{H}), wherein Vα is a TCR α variable region, Vβ is a TCR β variable region, Cβ is a TCR 13 constant region, X¹ is a first multimerization domain, and Ig(C_{H}) is an immunoglobulin heavy chain constant region or fragment thereof; and (b) a second fusion protein comprising the structure VαVβCβ-X²-Ig(C_{L}), wherein Vα is a TCR α variable region, Vβ is a TCR β variable region, Cβ is a TCR 13 constant region, X¹ is a second multimerization domain, and Ig(C_{L}) is an immunoglobulin light chain constant region; and wherein the first fusion protein and the second fusion protein form a soluble, multimeric TCR-immunoglobulin fusion protein. In some embodiments, the multimeric fusion protein comprises two first fusion proteins and two second fusion proteins. In some embodiments, the immunoglobulin heavy chain constant region or fragment thereof of the two first fusion proteins and the immunoglobulin light chain constant region of the two second fusion proteins forms an immunoglobulin framework. In some embodiments, the soluble, multimeric TCR-immunoglobulin fusion protein is a tetramer.

In some embodiments, the disclosure provides a soluble, multimeric TCR-immunoglobulin fusion protein that comprises one or more fusion proteins comprising the structure VαVβCβ-X-Ig(C_{H}), wherein Vα is a TCR α variable region, Vβ is a TCR β variable region, Cβ is a TCR β constant region, X is a multimerization domain, and Ig(C_{H}) is an immunoglobulin heavy chain constant region or fragment thereof; and wherein the one or more fusion proteins form a soluble, multimeric TCR-immunoglobulin fusion protein. In some embodiments, the multimeric TCR-immunoglobulin fusion protein comprises two fusion proteins. In some embodiments, the immunoglobulin heavy chain constant region or fragment thereof of the two fusion proteins forms an immunoglobulin framework. In some embodiments, the soluble, multimeric TCR-immunoglobulin protein is a dimer.

In some embodiments, the disclosure provides a soluble, multimeric TCR-immunoglobulin fusion protein that comprises one or more fusion proteins comprising the structure VαVβCβ-X-Ig(C_{H}), wherein Vα is a TCR α variable region, Vβ is a TCR β variable region, Cβ is a TCR β constant region, X is a multimerization domain, and Ig(C_{H}) is an immunoglobulin heavy chain constant region or fragment thereof; and wherein the one or more fusion proteins form a soluble, multimeric TCR-immunoglobulin fusion protein. In some embodiments, the multimeric TCR-immunoglobulin fusion protein comprises three fusion proteins. In some embodiments, the three fusion proteins of the soluble, multimeric TCR-immunoglobulin fusion protein are linked through the multimerization domains. In some embodiments, the soluble, multimeric TCR-immunoglobulin fusion protein is a trimer.

In any of the preceding embodiments, the disclosure provides a soluble, multimeric TCR-immunoglobulin fusion protein wherein each TCR-fusion protein in the multimeric protein binds to the same peptide antigen. In some embodiments, the disclosure provides a soluble, multimeric TCR-immuno globulin fusion protein wherein at least two of the TCR-fusion protein in the multimeric protein bind to different peptide antigens.

In one aspect, the disclosure provides a soluble, multimeric fusion protein that binds to a component of the MHC/TCR immune complex, comprising: (a) a first fusion protein comprising a soluble T cell receptor (TCR) or a soluble Major Histocompatibility Complex (MHC) linked to an immunoglobulin framework by a first multimerization domain; and (b) a second fusion protein comprising a soluble TCR or a soluble MHC linked to an immunoglobulin framework by a second multimerization domain that binds to the first multimerization domain; wherein the first and second fusion protein form a soluble multimeric fusion protein. In some embodiments, the first and second fusion proteins each comprise a soluble MHC class I polypeptide operatively linked to a β2-microglobulin polypeptide. In some embodiments, the multimeric protein fusion is a dimer, a trimer, a tetramer or a hexamer. In some embodiments, the multimeric fusion protein is a dimer. In some embodiments, the multimeric fusion protein is a tetramer.

In some embodiments, the disclosure provides a soluble, multimeric fusion protein wherein each fusion protein comprises an MHC class I α domain and a β2-microglobulin polypeptide. In some embodiments, the disclosure provides a soluble, multimeric fusion protein wherein each fusion protein comprises an MHC class I α1 domain, a MHC class I α2 domain, a MHC class I α3 domain and a β2 microglobulin polypeptide. In some embodiments, the disclosure provides a soluble, multimeric fusion protein wherein at least one fusion protein comprises a soluble MHC class I α domain and a β2-microglobulin polypeptide operably linked to an immunoglobulin heavy chain constant region or fragment thereof. In some embodiments, the disclosure provides a soluble, multimeric fusion protein wherein at least one fusion protein comprises a soluble MHC class I α domain and a β2-microglobulin polypeptide operably linked to an immunoglobulin light chain constant region or fragment thereof. In some embodiments, the disclosure provides a soluble, multimeric fusion protein wherein at least one fusion protein comprises a soluble MHC class I α domain and a β2-microglobulin polypeptide operably linked to an immunoglobulin heavy chain constant region, and at least one second fusion protein comprises a soluble MHC class I α domain and a β2-microglobulin polypeptide operably linked to an immunoglobulin light chain constant region or fragment thereof.

In some embodiments, the disclosure provides a soluble, multimeric MHCI-immunoglobulin fusion protein comprising (a) a first fusion protein comprising the structure β2M-MHCIα-X¹-Ig(Fc), wherein β2M is a soluble β2-microglobulin polypeptide, MHCIα is a soluble MHC class I α chain, X¹ is a first multimerization domain, and Ig(Fc) is an immunoglobulin Fc domain or fragment thereof, and (b) a second fusion protein comprising the structure β2M-MHCIα-X²-Ig(Fc), wherein β2M is a soluble β2-microglobulin polypeptide, MHCIα is a soluble MHC class I α chain, X² is a second multimerization domain, and Ig(Fc) is an immunoglobulin heavy chain constant domain or fragment thereof, wherein the first and second fusion proteins form a soluble, multimeric MHC Class I-immunoglobulin protein.

In some embodiments, the disclosure provides a soluble, multimeric MHCI-immunoglobulin fusion protein comprising (a) a first fusion protein comprising the structure: β2M-MHCIα-X¹-Ig(Fc), wherein β2M is a soluble β2-microglobulin polypeptide, MHCIα is a soluble MHC class I α chain, X¹ is a first multimerization domain, and Ig(Fc) is an immunoglobulin Fc domain or fragment thereof, and (b) a second fusion protein comprising the structure β2M-MHCIα-X²-Ig(C_{L}), wherein β2M is a soluble β2-microglobulin polypeptide, MHCIα is a soluble MHC class I α chain, X² is a second multimerization domain, and Ig(C_{L}) is an immunoglobulin light chain constant region or fragment thereof, wherein the first and second fusion proteins form a soluble, multimeric MHC Class I-immunoglobulin protein.

In some embodiments, the disclosure provides a soluble, multimeric MHC Class I-immunoglobulin fusion protein comprising (a) a first fusion protein comprising the structure β2M-MHCIα-X¹-Ig(C_{H}), wherein β2M is a soluble β2-microglobulin polypeptide, MHCIα is a soluble MHC class I α chain, X¹ is a first multimerization domain, and Ig(C_{H}) is an immunoglobulin heavy chain constant region or fragment thereof; and (b) a second fusion protein comprising the structure β2M-MHCIα-X²-Ig(C_{L}), wherein β2M is a soluble β2-microglobulin polypeptide, MHCIα is a soluble MHC class I α chain, X² is a second multimerization domain, and Ig(C_{L}) is an immunoglobulin light chain constant region or fragment thereof, wherein the first fusion protein and the second fusion protein form a soluble, multimeric MHC Class I-immunoglobulin fusion protein. In some embodiments, the multimeric MHCI-immunoglobulin fusion protein comprises two first fusion proteins and two second fusion proteins. In some embodiments, the immunoglobulin heavy chain constant region or fragment thereof of the two first fusion proteins and the immunoglobulin light chain constant region or fragment thereof of the two second fusion proteins forms an immunoglobulin framework. In some embodiments, the multimeric fusion protein is a tetramer.

In some embodiments, the disclosure provides a soluble, multimeric MHCI-immunoglobulin fusion protein comprising one or more fusion proteins comprising the structure β2M-MHCIα-X-Ig(C_{H}), wherein β2M is a soluble β2-microglobulin polypeptide, MHCIα is a soluble MHC class I α chain, X is a multimerization domain, and Ig(C_{H}) is an immunoglobulin heavy chain constant region or fragment thereof; and wherein the one or more fusion proteins form a soluble, multimeric MHC Class I-immunoglobulin fusion protein. In some embodiments, the soluble, multimeric MHCI-immunoglobulin fusion protein comprises two fusion proteins. In some embodiments, the immunoglobulin heavy chain constant region or fragment thereof of the two fusion proteins forms an immunoglobulin framework. In some embodiments, the multimeric MHCI-immunoglobulin fusion protein is a dimer.

In some embodiments, the disclosure provides a soluble, multimeric MHCI-immunoglobulin fusion protein comprising one or more fusion proteins comprising the structure β2M-MHCIα-X-Ig(C_{H}), wherein β2M is a soluble β2-microglobulin polypeptide, MHCIα is a soluble MHC class I α chain, X is a multimerization domain, and Ig(C_{H}) is an immunoglobulin heavy chain constant region or fragment thereof; and wherein the one or more fusion proteins form a soluble, multimeric MHC Class I-immunoglobulin fusion protein. In some embodiments, the soluble, multimeric MHCI-immunoglobulin fusion protein comprises three fusion proteins. In some embodiment, the three fusion proteins of the soluble, multimeric MHCI-immunoglobulin fusion protein are linked through the multimerization domain. In some embodiments, the soluble, multimeric MHCI-immunoglobulin fusion protein is a trimer.

In some embodiments, the disclosure provides a soluble, multimeric MHCI-immunoglobulin fusion protein wherein at least one fusion protein comprises a peptide loaded MHC (pMHC). In some embodiments, the disclosure provides a soluble, multimeric MHCI-immunoglobulin fusion protein wherein each fusion protein comprises a peptide loaded MHC (pMHC), and wherein the loaded peptides are the same or different.

In some embodiments, the disclosure provides a soluble multimeric MHC class I-immunoglobulin fusion protein comprising (a) a first fusion protein comprising the structure Ag-β2M-MHCIα-X¹-Ig(Fc), wherein Ag is an antigenic peptide, β2M is a soluble β2-microglobulin polypeptide, MHCIα is a soluble MHC class I α chain, X¹ is a first multimerization domain, and Ig(Fc) is an immunoglobulin Fc domain or fragment thereof, and (b) a second fusion protein comprising the structure Ag-β2M-MHCIα-X²-Ig(C_{L}), wherein Ag is an antigenic peptide, β2M is a soluble β2-microglobulin polypeptide, MHCIα is a soluble MHC class I α chain, X² is a second multimerization domain, and Ig(C_{L}) is an immunoglobulin light chain constant region or fragment thereof, wherein the first and second fusion proteins form a soluble, multimeric MHC Class I-immunoglobulin protein.

In some embodiments, the disclosure provides a soluble, multimeric MHCI-immunoglobulin fusion protein comprising (a) a first fusion protein comprising the structure Ag-β2M-MHCIα-X¹-Ig(C_{H}), wherein Ag is an antigenic peptide, β2M is a soluble β2-microglobulin polypeptide, MHCIα is a soluble MHC class I α chain, X¹ is a first multimerization domain, and Ig(C_{H}) is an immunoglobulin heavy chain constant region or fragment thereof; and (b) a second fusion protein comprising the structure Ag-β2M-MHCIα-X²-Ig(C_{L}), wherein Ag is an antigenic peptide, β2M is a soluble β2-microglobulin polypeptide, MHCIα is a soluble MHC class I α chain, X² is a second multimerization domain, and Ig(C_{L}) is an immunoglobulin light chain constant region or fragment thereof, wherein the first fusion protein and the second fusion protein form a soluble, multimeric MHC Class I-immunoglobulin protein. In some embodiments, the multimeric fusion protein comprises two first fusion proteins and two second fusion proteins. In some embodiments, the immunoglobulin heavy chain constant region or fragment thereof of the two first fusion proteins and the immunoglobulin light chain constant region of the two second fusion proteins forms an immunoglobulin framework. In some embodiments, the soluble, multimeric MHCI-immunoglobulin fusion protein is a tetramer.

In some embodiments, the disclosure provides a soluble, multimeric MHCI-immunoglobulin fusion protein comprising one or more fusion proteins comprising the structure Ag-β2M-MHCIα-X-Ig(C_{H}), wherein Ag is an antigenic peptide, β2M is a soluble β2-microglobulin polypeptide, MHCIα is a soluble MHC class I α chain, X is a multimerization domain, and Ig(C_{H}) is an immunoglobulin heavy chain constant region or fragment thereof, wherein the one or more fusion proteins form a soluble, multimeric MHC Class I-immunoglobulin protein. In some embodiments, the soluble, multimeric MHCI-immunoglobulin fusion protein comprises two fusion proteins. In some embodiments, the immunoglobulin heavy chain constant region or fragment thereof of the two fusion proteins forms an immunoglobulin framework. In some embodiments, the soluble, multimeric MHCI-immunoglobulin fusion protein is a dimer.

In some embodiments, the disclosure provides a soluble, multimeric MHCI-immunoglobulin fusion protein comprising one or more fusion proteins comprising the structure Ag-β2M-MHCIα-X-Ig(C_{H}), wherein Ag is an antigenic peptide, β2M is a soluble β2-microglobulin polypeptide, MHCIα is a soluble MHC class I α chain, X is a multimerization domain, and Ig(C_{H}) is an immunoglobulin heavy chain constant region or fragment thereof, wherein the one or more fusion proteins form a soluble, multimeric MHC Class I-immunoglobulin protein. In some embodiments, the soluble, multimeric MHCI-immunoglobulin fusion protein comprises three fusion proteins. In some embodiments, the three fusion proteins are linked through a multimerization domain. In some embodiments, the soluble, multimeric MHCI-immunoglobulin fusion protein is a trimer.

In one aspect, the disclosure provides a soluble, multimeric fusion protein that binds to a component of the MHC/TCR immune complex, comprising: (a) a first fusion protein comprising a soluble T cell receptor (TCR) or a soluble Major Histocompatibility Complex (MHC) linked to an immunoglobulin framework by a first multimerization domain; and (b) a second fusion protein comprising a soluble TCR or a soluble MHC linked to an immunoglobulin framework by a second multimerization domain that binds to the first multimerization domain; wherein the first and second fusion protein form a soluble multimeric fusion protein. In some embodiments, the first and second fusion proteins each comprise a soluble MHC class II polypeptide. In some embodiments, the disclosure provides a soluble, multimeric protein fusion comprising first and second fusion proteins comprising a soluble MHC class II polypeptide, wherein the multimeric protein fusion is a dimer, a trimer, a tetramer or a hexamer. In some embodiments, the multimeric protein fusion is a dimer. In some embodiments, the multimeric fusion protein is a tetramer.

In some embodiments, the disclosure provides a soluble, multimeric fusion protein wherein at least one fusion protein comprises an MHC II α domain and an MHC II β domain. In some embodiments, the disclosure provides a soluble, multimeric fusion protein wherein at least two fusion proteins comprises an MHC II α domain and a MHC II β domain. In some embodiments, the disclosure provides a soluble, multimeric fusion protein wherein at least one fusion protein comprises an MHC II α domain, and at least a second fusion protein comprises an MHC II β domain. In some embodiments, the MHC II α domain is an α1 domain. In some embodiments, the MHC II α domain is an α2 domain.

In some embodiments, the disclosure provides a soluble, multimeric fusion protein wherein the first and second fusion proteins each comprise a soluble MHC class II polypeptide, and wherein each fusion protein binds to the same MHC class II molecule. In some embodiments, at least two fusion proteins bind to different MHC class II molecules.

In some embodiments, the disclosure provides a soluble, multimeric fusion protein wherein at least one fusion protein comprises a soluble MHC class II polypeptide operably linked to an immunoglobulin heavy chain constant region or fragment thereof. In some embodiments, the disclosure provides a soluble, multimeric fusion protein wherein at least one fusion protein comprises a soluble MHC class II polypeptide operably linked to an immunoglobulin light chain constant region or fragment thereof. In some embodiments, the disclosure provides a soluble, multimeric fusion protein wherein at least one fusion protein comprises a soluble MHC class II polypeptide operably linked to an immunoglobulin heavy chain constant region, and at least a second fusion protein comprises a soluble MHC class II polypeptide operably linked to an immunoglobulin light chain constant region or fragment thereof.

In some embodiments, the disclosure provides a soluble, multimeric MHC class II-immunoglobulin fusion protein comprising (a) a first fusion protein comprising the structure MHCIIα-MHCIIβ-X¹-Ig(Fc), wherein MHCIIα is a soluble MHC class II α domain, MHCIIβ is a soluble MHC class II β domain, X¹ is a first multimerization domain, and Ig(Fc) is an immunoglobulin Fc domain or fragment thereof; and (b) a second fusion protein comprising the structure MHCIIα-MHCIIβ-X²-Ig(Fc), wherein MHCIIα is a soluble MHC class II α domain, MHCIIβ is a soluble MHC class II β domain, X² is a second multimerization domain, and Ig(Fc) is an immunoglobulin Fc domain or fragment thereof, wherein the first and second fusion proteins form a soluble, multimeric MHC Class II-immunoglobulin fusion protein.

In some embodiments, the disclosure provides a soluble, multimeric MHC class II-immunoglobulin fusion protein comprising (a) a first fusion protein comprising the structure MHCIIα-MHCIIβ-X¹-Ig(Fc), wherein MHCIIα is a soluble MHC class II α domain, MHCIIβ is a soluble MHC class II β domain, X¹ is a first multimerization domain, and Ig(Fc) is an immunoglobulin Fc domain or fragment thereof; and (b) a second fusion protein comprising the structure MHCIIα-MHCIIβ-X²-Ig(C_{L}), wherein MHCIIα is a soluble MHC class II α domain, MHCIIβ is a soluble MHC class 1113 domain, X² is a second multimerization domain, and Ig(C_{L}) is an immunoglobulin light chain constant region or fragment thereof, and wherein the first and second fusion proteins form a soluble, multimeric MHC Class II-immunoglobulin protein fusion.

In some embodiments, the disclosure provides a soluble, multimeric MHC class II-immunoglobulin fusion protein comprising (a) a first fusion protein comprising the structure MHCIIα-MHCIIβ-X¹-Ig(C_{H}), wherein MHCIIα is a soluble MHC class II α domain, MHCIIβ is a soluble MHC class II β domain, X¹ is a first multimerization domain, and Ig(C_{H}) is an immunoglobulin heavy chain constant region or fragment thereof; and (b) a second fusion protein comprising the structure MHCIIα-MHCIIβ-X²-Ig(C_{L}), wherein MHCIIα is a soluble MHC class II α domain, MHCIIβ is a soluble MHC class II β domain, X² is a second multimerization domain, and Ig(C_{L}) is an immunoglobulin light chain constant region or fragment thereof, wherein the first fusion protein and the second fusion protein form a soluble, multimeric MHC class II-immunoglobulin fusion protein. In some embodiments, the multimeric fusion protein comprises two first fusion proteins and two second fusion proteins. In some embodiments, the immunoglobulin heavy chain constant region or fragment thereof of the two first fusion proteins and the immunoglobulin light chain constant region of the two second fusion proteins forms an immunoglobulin framework. In some embodiments, the multimeric MHC class II-immunoglobulin fusion protein is a tetramer.

In some embodiments, the disclosure provides a soluble, multimeric MHC class II-immunoglobulin fusion protein comprising (a) a first fusion protein comprising the structure MHCIIα-X¹-Ig(C_{H}), wherein MHCIIα is a soluble MHC class II α domain, X¹ is a first multimerization domain, and Ig(C_{H}) is an immunoglobulin heavy chain constant region or fragment thereof; and (b) a second fusion protein comprising the structure MHCIIβ-X¹-Ig(C_{L}), wherein MHCIIβ is a soluble MHC class II β domain, X¹ is a second multimerization domain, and Ig(C_{L}) is an immunoglobulin light chain constant region or fragment thereof, wherein the first fusion protein and the second fusion protein form a soluble, multimeric MHC class II-immunoglobulin fusion protein. In some embodiments, the multimeric fusion protein comprises two first fusion proteins and two second fusion proteins. In some embodiments, the immunoglobulin heavy chain constant region or fragment thereof of the two first fusion proteins and the immunoglobulin light chain constant region of the two second fusion proteins forms an immunoglobulin framework. In some embodiments, the multimeric MHC class II-immunoglobulin fusion protein is a dimer.

In some embodiments, the disclosure provides a soluble, multimeric MHC class II-immunoglobulin fusion protein comprising one or more fusion proteins comprising the structure MHCIIα-MHCIIβ-X-Ig(C_{H}), wherein MHCIIα is a soluble MHC class II α domain, MHCIIβ is a soluble MHC class II β domain, X is a multimerization domain, and Ig(C_{H}) is an immunoglobulin heavy chain constant region or fragment thereof; wherein the one or more fusion proteins form a soluble, multimeric MHC Class II-immunoglobulin protein. In some embodiments, the multimeric fusion protein comprises two fusion proteins. In some embodiments, the immunoglobulin heavy chain constant region or fragment thereof of the two fusion proteins forms an immunoglobulin framework. In some embodiments, the multimeric MHC class II-immunoglobulin fusion protein is a dimer.

In some embodiments, the disclosure provides a soluble, multimeric MHC class II-immunoglobulin fusion protein comprising one or more fusion proteins comprising the structure MHCIIα-MHCIIβ-X-Ig(C_{H}), wherein MHCIIα is a soluble MHC class II α domain, MHCIIβ is a soluble MHC class II β domain, X is a multimerization domain, and Ig(C_{H}) is an immunoglobulin heavy chain constant region or fragment thereof; wherein the one or more fusion proteins form a soluble, multimeric MHC Class II-immunoglobulin protein. In some embodiments, the multimeric fusion protein comprises three fusion proteins. In some embodiments, the three fusion proteins are linked through the multimerization domain. In some embodiments, the multimeric MHC class II-immunoglobulin fusion protein is a trimer.

In some embodiments, the disclosure provides a soluble, multimeric MHC class II-immunoglobulin fusion protein wherein at least one fusion protein comprises a peptide loaded MHC (pMHC). In some embodiments, the disclosure provides a soluble, multimeric MHC class II-immunoglobulin fusion protein wherein each fusion protein comprises a pMHC. In some embodiments, the peptide is operably linked to the soluble MHC class II polypeptide of the at least one fusion protein, optionally via an amino acid linker. In some embodiments, the peptide is operably linked to the soluble MHC class II α domain of the at least one fusion protein.

In any of the foregoing embodiments, the disclosure provides a soluble, multimeric fusion protein wherein the first and second multimerization domains are leucine zipper dimerization domains. In some embodiments, the first multimerization domain and/or the second multimerization domain is LZR that comprises an amino acid sequence identified by SEQ ID NO: 8. In some embodiments, the first multimerization domain and/or the second multimerization domain is LZL that comprises an amino acid sequence identified by SEQ ID NO: 6. In some embodiments, the first multimerization domain is LZR that comprises an amino acid sequence identified by SEQ ID NO: 8, and the second multimerization domain is LZL that comprises an amino acid sequence identified by SEQ ID NO: 6. In some embodiments, the first multimerization domain is LZL that comprises an amino acid sequence identified by SEQ ID NO: 6, and the second multimerization domain is LZR that comprises an amino acid sequence identified by SEQ ID NO: 8.

In some embodiments, the disclosure provides a soluble, multimeric fusion protein wherein the multimerization domains are self-trimerization domains. In some embodiments, the self-trimerization domain comprises a collagen-like scaffold comprising (GX₁X₂)ₙ, wherein G is glycine, X₁ and X₂ are any amino acid residues, and n is at least 5. In some embodiments, X₁ and X₂ are proline. In some embodiments, the self-trimerization domain comprises (GPP)₁₀ (SEQ ID NO: 60).

In some embodiments, the disclosure provides a soluble, multimeric fusion protein wherein the first or second multimerization domain comprises a leucine zipper domain operatively linked to a self-trimerization domain.

In any of the foregoing embodiments, the disclosure provides a soluble, multimeric fusion protein wherein at least one fusion protein comprises a peptide linker positioned between the soluble TCR polypeptide or the soluble MHC polypeptide and the multimerization domain. In some embodiments, the peptide linker comprises a Gly-Ser linker. In some embodiments, the Gly-Ser linker is selected from the group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG.

In some embodiments, the disclosure provides a soluble, multimeric fusion protein wherein at least one fusion protein comprises a Gly-Ser linker positioned between the soluble TCR polypeptide or the soluble MHC polypeptide and the multimerization domain, and wherein the Gly-Ser linker is selected from the group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG.

In any one of the foregoing embodiments, the disclosure provides a soluble, multimeric fusion protein wherein at least one fusion protein comprises a peptide linker positioned between the multimerization domain and the immunoglobulin framework. In some embodiments, the peptide linker comprises a Gly-Ser linker. In some embodiments, the Gly-Ser linker comprises the amino acid sequence GGSGG (SEQ ID NO: 12).

In any one of the foregoing embodiments, the disclosure provides a soluble, multimeric fusion protein comprising a signal peptide.

In some embodiments, the disclosure provides a soluble, multimeric TCR-immunoglobulin fusion protein wherein the soluble TCR polypeptide in at least one fusion protein binds to an MHC peptide. In some embodiments, the MHC peptide is derived from an from a cancer antigen, a viral antigen, a bacterial antigen, a parasitic antigen or an allergen. In some embodiments, the MHC peptide is derived from a cancer antigen. In some embodiments, the MHC peptide is derived from the human endogenous retrovirus (HERV-K) envelope protein. In some embodiments, the MHC peptide is derived from a viral antigen. In some embodiments, the MHC peptide is derived from the human immunodeficiency virus (HIV) group antigens (Gag) protein. In some embodiments, the MHC peptide is the HLA-A02-restricted FLGKIWPSYK epitope (SEQ ID NO: 59).

In some embodiments, the disclosure provides a soluble, multimeric MHC-immunoglobulin fusion protein wherein at least one fusion protein comprises a soluble MHC that binds to a peptide antigen derived from a cancer antigen, a viral antigen, a bacterial antigen, a parasitic antigen or an allergen.

In some embodiments, the disclosure provides a soluble, multimeric TCR-immunoglobulin fusion protein comprising (a) a first fusion protein comprising the structure VαCα-X¹-Ig(C_{H}), wherein Vα is a TCR α variable region, Cα is TCR α constant region, X¹ is a multimerization domain comprising a first leucine zipper domain, and Ig(C_{H}) is an immunoglobulin heavy chain constant region or fragment thereof; and (b) a second fusion protein comprising the structure VβCβ-X²-Ig(C_{L}), wherein Vβ is a TCR β variable region, Cβ is a TCR β constant region, X² is a multimerization domain comprising a second leucine zipper domain, and Ig(C_{L}) is an immunoglobulin light chain constant region or fragment thereof; wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric TCR-immunoglobulin protein that is a TCR dimer. In some embodiments, the first fusion protein comprises a TCR α chain comprising an amino acid sequence set forth by SEQ ID NO: 64 (HERV-K TCRalpha) and the second fusion protein comprises a TCR β chain comprising an amino acid sequence set forth by SEQ ID NO: 66 (HERV-K TCRbeta). In some embodiments, the first fusion protein comprises a TCR α chain comprising an amino acid sequence set forth by SEQ ID NO: 76 (FK10 TCRalpha) and the second fusion protein comprises a TCR β chain comprising an amino acid sequence set forth by SEQ ID NO: 78 (FK10 TCRbeta). In some embodiments, the first leucine zipper domain is LZL that comprises an amino acid sequence identified by SEQ ID NO: 6. In some embodiments, the second leucine zipper domain is LZR that comprises an amino acid sequence identified by SEQ ID NO: 8. In some embodiments, the TCR polypeptide is linked to the multimerization domain by a Gly-Ser linker. In some embodiments, the multimerization domain is linked to the immunoglobulin domain by a Gly-Ser linker. In some embodiments, the Gly-Ser linker is selected from a group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG. In some embodiments, the Ig(C_{H}) is a human IgG immunoglobulin heavy chain constant region or fragment thereof. In some embodiments, the Ig(C_{L}) is a human IgG immunoglobulin light chain constant region or fragment thereof.

In some embodiments, the disclosure provides a soluble, multimeric MHC Class I-immunoglobulin fusion protein comprising (a) a first fusion protein comprising the structure β2M-MHCIα-X¹-Ig(C_{H}), wherein β2M is a soluble β2-microglobulin polypeptide, MHCIα is a soluble MHC class I α chain, X¹ is a multimerization domain comprising a first leucine zipper domain, and Ig(C_{H}) is an immunoglobulin heavy chain constant region or fragment thereof; and (b) a second fusion protein comprising the structure β2M-MHCIα-X²-Ig(C_{L}), wherein β2M is a soluble β2-microglobulin polypeptide, MHCIα is a soluble MHC class I α chain, X² is a multimerization domain comprising a second leucine zipper domain, and Ig(C_{L}) is an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric MHC Class I-immunoglobulin fusion protein that is an MHC Class I receptor tetramer. In some embodiments, the first leucine zipper domain is LZL that comprises an amino acid sequence identified by SEQ ID NO: 6. In some embodiments, the second leucine zipper domain is LZR that comprises an amino acid sequence identified by SEQ ID NO: 8. In some embodiments, the MHC polypeptide is linked to the multimerization domain by a Gly-Ser linker. In some embodiments, the multimerization domain is linked to the immunoglobulin domain by a Gly-Ser linker. In some embodiments, the Gly-Ser linker is selected from a group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG. In some embodiments, the Ig(C_{H}) is a human IgG immunoglobulin heavy chain constant region or fragment thereof. In some embodiments, the Ig(C_{L}) is a human IgG immunoglobulin light chain constant region or fragment thereof.

In some embodiments, the disclosure provides a soluble, multimeric MHC Class I-immunoglobulin fusion protein comprising a fusion protein comprising the structure β2M-MHCIα-X-Ig(C_{H}), wherein β2M is a soluble β2-microglobulin polypeptide, MHCIα is a soluble MHC class I α chain, X is a multimerization domain comprising a first leucine zipper domain, and Ig(C_{H}) is an immunoglobulin heavy chain constant region or fragment thereof, wherein the multimeric fusion protein comprises two fusion proteins, wherein the immunoglobulin heavy chain constant region or fragment thereof of the fusion proteins forms an immunoglobulin framework, and wherein the soluble, multimeric MHC Class I-immunoglobulin fusion protein is an MHC Class I receptor dimer. In some embodiments, the first leucine zipper domain is LZL that comprises an amino acid sequence identified by SEQ ID NO: 6. In some embodiments, the second leucine zipper domain is LZR that comprises an amino acid sequence identified by SEQ ID NO: 8. In some embodiments, the MHC polypeptide is linked to the multimerization domain by a Gly-Ser linker. In some embodiments, the multimerization domain is linked to the immunoglobulin domain by a Gly-Ser linker. In some embodiments, the Gly-Ser linker is selected from a group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG. In some embodiments, the Ig(C_{H}) is a human IgG immunoglobulin heavy chain constant region or fragment thereof. In some embodiments, the Ig(C_{L}) is a human IgG immunoglobulin light chain constant region or fragment thereof.

In some embodiments, the disclosure provides a soluble, multimeric MHC Class I-immunoglobulin fusion protein comprising (a) a first fusion protein comprising the structure Ag-β2M-MHCIα-X¹-Ig(C_{H}), wherein Ag is antigenic peptide, β2M is a soluble β2-microglobulin polypeptide, MHCIα is a soluble MHC class I α chain, X¹ is a multimerization domain comprising a first leucine zipper domain, and Ig(C_{H}) is an immunoglobulin heavy chain constant region or fragment thereof; and (b) a second fusion protein comprising the structure Ag-β2M-MHCIα-X²-Ig(C_{L}), wherein Ag is antigenic peptide, wherein β2M is a soluble β2-microglobulin polypeptide, MHCIα is a soluble MHC class I α chain, X² is a multimerization domain comprising a second leucine zipper domain, and Ig(C_{L}) is an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric MHC Class I-immunoglobulin fusion protein that is an MHC Class I receptor tetramer. In some embodiments, the first leucine zipper domain is LZL that comprises an amino acid sequence identified by SEQ ID NO: 6. In some embodiments, the second leucine zipper domain is LZR that comprises an amino acid sequence identified by SEQ ID NO: 8. In some embodiments, the MHC polypeptide is linked to the multimerization domain by a Gly-Ser linker. In some embodiments, the multimerization domain is linked to the immunoglobulin domain by a Gly-Ser linker. In some embodiments, the Gly-Ser linker is selected from a group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG. In some embodiments, the Ig(C_{H}) is a human IgG immunoglobulin heavy chain constant region or fragment thereof. In some embodiments, the Ig(C_{L}) is a human IgG immunoglobulin light chain constant region or fragment thereof.

In some embodiments, the disclosure provides a soluble, multimeric MHC Class I-immunoglobulin fusion protein comprising a fusion protein comprising the structure Ag-β2M-MHCIα-X-Ig(C_{H}), wherein Ag is antigenic peptide, β2M is a soluble β2-microglobulin polypeptide, MHCIα is a soluble MHC class I α chain, X is a multimerization domain comprising a first leucine zipper domain, and Ig(C_{H}) is an immunoglobulin heavy chain constant region or fragment thereof; wherein the multimeric fusion protein comprises two fusion proteins, wherein the immunoglobulin heavy chain constant region or fragment thereof of the two fusion proteins forms an immunoglobulin framework, and wherein the soluble, multimeric MHC Class I-immunoglobulin fusion protein is an MHC Class I receptor dimer. In some embodiments, the first leucine zipper domain is LZL that comprises an amino acid sequence identified by SEQ ID NO: 6. In some embodiments, the second leucine zipper domain is LZR that comprises an amino acid sequence identified by SEQ ID NO: 8. In some embodiments, the MHC polypeptide is linked to the multimerization domain by a Gly-Ser linker. In some embodiments, the multimerization domain is linked to the immunoglobulin domain by a Gly-Ser linker. In some embodiments, the Gly-Ser linker is selected from a group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG. In some embodiments, the Ig(C_{H}) is a human IgG immunoglobulin heavy chain constant region or fragment thereof. In some embodiments, the Ig(C_{L}) is a human IgG immunoglobulin light chain constant region or fragment thereof.

In some embodiments, the disclosure provides a composition comprising the soluble, multimeric protein fusion complex described herein.

In some embodiments, the disclosure provides a pharmaceutical composition comprising the soluble, multimeric protein fusion complex described herein, and a pharmaceutically acceptable carrier.

In some embodiments, the disclosure provides a nucleic acid encoding the first fusion protein of a soluble, multimeric protein fusion complex described herein. In some embodiments, the disclosure provides a nucleic acid encoding the second fusion protein of a soluble, multimeric protein fusion complex described herein. In some embodiments, the disclosure provides a nucleic acid encoding the first fusion protein and the second fusion protein of a soluble, multimeric protein fusion complex described herein.

In some embodiments, the disclosure provides a recombinant expression vector comprising a nucleic acid encoding the first fusion protein of a soluble, multimeric protein fusion complex described herein. In some embodiments, the disclosure provides a recombinant expression vector comprising a nucleic acid the second fusion protein of a soluble, multimeric protein fusion complex described herein. In some embodiments, the disclosure provides a recombinant expression vector comprising a nucleic acid the first fusion protein and the second fusion protein of a soluble, multimeric protein fusion complex described herein. In some embodiments, the recombinant expression vector comprises a nucleic acid encoding a self-cleaving amino acid sequence positioned between the nucleic acid encoding the first fusion protein and the nucleic acid encoding the second fusion protein. In some embodiments, the self-cleaving amino acid sequence is derived from a 2A peptide. In some embodiments, the self-cleaving amino acid sequence comprises a 2A peptide selected from porcine teschovirus-1 (P2A), equine rhinitis A virus (E2A), Thosea asigna virus (T2A), foot-and-mouth disease virus (F2A), or any combination thereof. In some embodiments, the nucleic acid encodes a furin recognition site upstream of the self-cleaving amino acid sequence, optionally linked via a Gly-Ser linker.

In some embodiments, the disclosure provides a host cell comprising a nucleic acid disclosed herein or an expression vector disclosed herein.

In some embodiments, the disclosure provides a method for treating or preventing an allergic reaction in a subject in need thereof by administering a multimeric protein fusion complex disclosed herein, a composition disclosed herein, or a pharmaceutical composition disclosed herein, in an amount sufficient to suppress or reduce a T cell response associated with the allergy.

In some embodiments, the disclosure provides a method for treating or preventing graft-versus-host disease (GvHD) in a subject who has received or will receive an organ transplant or tissue graft, by administering a multimeric protein fusion complex disclosed herein, a composition disclosed herein, or a pharmaceutical composition disclosed herein, in an amount sufficient to suppress or reduce an immune response to the transplant.

In some embodiments, the disclosure provides a method for treating an autoimmune disease in a subject by administering a multimeric protein fusion complex disclosed herein, a composition disclosed herein, or a pharmaceutical composition disclosed herein, in an amount sufficient to suppress or reduce the autoimmune response.

In some embodiments, the disclosure provides a method for treating cancer in a subject by administering a multimeric protein fusion complex disclosed herein, a composition disclosed herein, or a pharmaceutical composition disclosed herein in an amount sufficient to induce or enhance an immune response to the cancer.

In some embodiments, the disclosure provides a method for treating an infection caused by an infectious agent in a subject by administering a multimeric protein fusion complex disclosed herein, a composition disclosed herein, or a pharmaceutical composition disclosed herein, in an amount sufficient to induce or enhance an immune response to the infectious agent.

In some embodiments, the disclosure provides a kit comprising a container comprising a multimeric protein fusion complex disclosed herein, and an optional pharmaceutically acceptable carrier, a composition disclosed herein, and an optional pharmaceutically acceptable carrier, or a pharmaceutical composition disclosed herein, and a package insert, wherein the kit comprises instructions for administration of the protein fusion, composition or pharmaceutical composition for treating or preventing an allergic reaction by suppressing or reducing a T cell response associated with the allergy in a subject in need thereof.

In some embodiments, the disclosure provides a kit comprising a container comprising a multimeric protein fusion complex disclosed herein, and an optional pharmaceutically acceptable carrier, a composition disclosed herein, and an optional pharmaceutically acceptable carrier, or a pharmaceutical composition disclosed herein, and a package insert, wherein the kit comprises instructions for administration of the protein fusion, composition or pharmaceutical composition for treating or preventing (GvHD) by suppressing or reducing an immune response to a transplant in a subject who has received or will receive an organ transplant or a tissue graft.

In some embodiments, the disclosure provides a kit comprising a container comprising a multimeric protein fusion complex disclosed herein, and an optional pharmaceutically acceptable carrier, a composition disclosed herein, and an optional pharmaceutically acceptable carrier, or a pharmaceutical composition disclosed herein, and a package insert, wherein the kit comprises instructions for administration of the protein fusion, composition or pharmaceutical composition for treating or delaying progression of an autoimmune disease or suppressing or reducing an autoimmune response in a subject in need thereof.

In some embodiments, the disclosure provides a kit comprising a container comprising a multimeric protein fusion complex disclosed herein, and an optional pharmaceutically acceptable carrier, a composition disclosed herein, and an optional pharmaceutically acceptable carrier, or a pharmaceutical composition disclosed herein, and a package insert, wherein the kit comprises instructions for administration of the protein fusion, composition or pharmaceutical composition for treating or delaying progression of cancer or reducing or inhibiting tumor growth in a subject in need thereof.

In some embodiments, the disclosure provides a kit comprising a container comprising a multimeric protein fusion complex disclosed herein, and an optional pharmaceutically acceptable carrier, a composition disclosed herein, and an optional pharmaceutically acceptable carrier, or a pharmaceutical composition disclosed herein, and a package insert, wherein the kit comprises instructions for administration of the protein fusion, composition or pharmaceutical composition for treating an infection caused by an infectious agent by inducing or enhancing an immune response against the infectious agent in a subject in need thereof.

In some embodiments, the disclosure describes the use of a multimeric protein fusion complex described herein, a composition described herein, or a pharmaceutical composition described herein, for the manufacture of a medicament for treating or delaying progression of cancer or reducing or inhibiting tumor growth in a subject in need thereof.

In some embodiments, the disclosure describes the use of a multimeric protein fusion complex described herein, a composition described herein, or a pharmaceutical composition described herein for the manufacture of a medicament for treating an infection caused by an infectious agent by inducing or enhancing an immune response against the infectious agent in a subject in need thereof.

In some embodiments, the disclosure describes the use of a multimeric protein fusion complex described herein, a composition described herein, or a pharmaceutical composition described herein in the manufacture of a medicament for treating or delaying progression of cancer or reducing or inhibiting tumor growth in a subject in need thereof.

In some embodiments, the disclosure describes the use of a multimeric protein fusion complex described herein, a composition described herein, or a pharmaceutical composition described herein in the manufacture of a medicament for treating an infection caused by an infectious agent by inducing or enhancing an immune response against the infectious agent in a subject in need thereof.

In some embodiments, the disclosure describes the use of a multimeric protein fusion complex described herein, a composition described herein, or a pharmaceutical composition described herein for use as a medicament.

These and other aspects and embodiments will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and/or the arrangement of components set forth in the following description or illustrated in the drawings.

### BRIEF DESCRIPTION OF THE FIGURES

The accompanying drawings are not intended to be drawn to scale. For purposes of clarity, not every component may be labeled in every drawing.

FIG. 1 is a general schematic representation of multimeric TCR/pMHC multimers with an immunoglobulin framework.

FIG. 2A illustrates four different structural designs of the OT1-TCR-Igg. FIG. 2B is a bar graph depicting quantification of secreted OT1-Igg from the four designs via ELISA with the VRC01 antibody expression construct as a control.

FIG. 3 is a bar graph quantifying production of TCR-Igg constructs with different length glycine/serine linkers connecting the TCR chain and multimerization domain.

FIG. 4A illustrates the split design expression construct of the TCR-Igg fusion with heavy chain and light chains in separate expression constructs, a and b, respectively. FIG. 4B illustrates the integrated design expression construct of the TCR-Igg fusion with both heavy and light chain integrated into a single expression construct via a P2A peptide, ab. FIG. 4C is a bar graph depicting quantification of TCR-Igg production from cells expressing either the split design (a+b) or integrated design (ab) constructs.

FIG. 5 depicts FACS staining of SIINFEKL-H2Kb expressing K562 cells with raw supernatant collected from either cells expressing either the split design (a+b) or integrated design (ab) constructs. An anti-mouse SIINFEKL bound H-2Kb antibody, clone 25-D.16, was used as a positive control.

FIG. 6A is a graph depicting FACS staining intensity of single chain SIINFKEL-H2Kb expressing K562 cells with a titration of OT1-TCR-Igg. FIG. 6B is a graph depicting the staining of SIINKFEL peptide loaded B16F10 melanoma cells with a titration of OT1-TCR-Igg.

FIG. 7 is a graph depicting staining of Ova-expressing B16F10 melanoma cells with fluorescent OT1-TCR-Igg following treatment with or without IFN gamma to induce antigen presentation of Ova peptides (e.g., SIINFKEL).

FIG. 8A is a photograph of a regular PAGE-gel of raw cell culture supernatant. FIG. 8B is a photograph of a PAGE-gel analyzing the size of TCR-Igg components produced following exposure to denaturing and either non-reducing or reducing conditions. FIG. 8C-8D is a photograph of a PAGE-gel in which TCR-Igg was treated with denaturing and reducing conditions (FIG. 8C) or denaturing and non-reducing conditions (FIG. 8D) and assessed by Western Blot. Arrows indicate expected band.

FIG. 9A depicts a dimeric TCR-Igg design and FIG. 9B depicts a single chain dimeric TCR design. FIG. 9C is a bar graph depicting a comparison of protein secretion quantification from both dimeric TCR-Igg designs with or without stabilizing mutations.

FIG. 10A is a bar graph showing expression of mouse OT1-TCR-Igg measured as protein concentration in supernatant from transfected cells as compared to a negative control that was supernatant from untransfected cells. FIG. 10B is a bar graph showing expression of human HERV-K-TCR-hIgG1 and FK10-TCR-hIgG1 measured as protein concentration in supernatant from transfected cells as compared to a negative control that was supernatant from untransfected cells.

FIG. 11A illustrates a structure of tetrameric single chain TCR-Igg structure: single chain TCR(VαVβCβ)-LZL-CH1-CH2-CH3+single chain TCR(VαVβCβ)-LZR-CL. FIG. 11B illustrates the structure of a trimeric single chain TCR-Igg: TCR(VαVβCβ)-CPP-CH2-CH3. FIG. 11C illustrates a structure of a hexameric single chain TCR-Igg: single chain TCR(VαVβCβ)-LZL-CPP-CH2-CH3+single chain TCR(VαVβCβ)-LZR-CPP.

FIG. 12A illustrates the application of TCR-Igg to recruit innate immune cells. FIG. 12B illustrates the application of FITC conjugated TCR-Igg molecules as an adaptor to recruit adoptively transferred universal CAR-T cells to kill target cells. FIG. 12C illustrates TCR-Igg covalently linked to an anti-CD3 scFV as a Bispecific T cell Engager (BITE) to recruit endogenous T cells for therapy.

FIG. 13A illustrates the design of a single chain pMHC1 dimer or tetramer. FIG. 13B illustrates the design of a single chain MHC1 dimer or tetramer with empty groove for peptide loading. FIG. 13C is a bar graph quantifying the secretion of either pMHCI-Igg dimer/tetramer or empty MHCI-Igg dimer/tetramer as evaluated by ELISA. SIINFEKL-H2Kb was a used as a model.

FIG. 13D depicts FACS staining of OT1 T cells using raw cell supernatant containing either SIINFEKL-H2Kb-Igg dimer or tetramer.

FIG. 14A depicts the nucleotide sequence and FIG. 14B depicts the amino acid sequence of a fusion protein comprising murine OTI TCRα-LZL-IgG heavy chain.

FIG. 15A depicts the nucleotide sequence and FIG. 15B depicts the amino acid sequence of a fusion protein comprising murine OTI TCRβ-LZR-IgG light chain.

FIG. 16A depicts the nucleotide sequence and FIG. 16B depicts the amino acid sequence of a fusion protein comprising murine wild-type 2C TCRα-LZL-IgG heavy chain.

FIG. 17A depicts the nucleotide sequence and FIG. 17B depicts the amino acid sequence of a fusion protein comprising murine wild-type 2C TCRβ-LZR-IgG light chain.

FIG. 18A depicts the nucleotide sequence and FIG. 18B depicts the amino acid sequence of a fusion protein comprising a mutated murine mut6 2C TCRα-LZL-IgG heavy chain.

FIG. 19A depicts the nucleotide sequence and FIG. 19B depicts the amino acid sequence of a fusion protein comprising mutated murine mut6 2C TCRβ-LZR-IgG light chain.

FIG. 20A depicts the nucleotide sequence and FIG. 20B depicts the amino acid sequence of a peptide loaded β2-microglobulin-MHCIα-IgG heavy chain fusion protein, β2M signal peptide-SIINFEKL-β2M-H2Kb-LZL-IgG_{HC}.

FIG. 21A depicts the nucleotide sequence and FIG. 21B depicts the amino acid sequence of a peptide loaded β2-microglobulin-MHCIα-IgG light chain fusion protein, β2M signal peptide-SIINFEKL-β2M-H2Kb-LZR-IgG_{LC}.

FIG. 22A depicts the nucleotide sequence and FIG. 22B depicts the amino acid sequence of the single vector insert encoding two chains of the murine OTI TCR IgG fusion proteins, TCRα-LZL-IgG_{HC}-furin-GSG-HIS-GSG-P2A-OTI/TCRβ-LZR-IgC_{LC}.

FIG. 23A depicts the nucleotide sequence and FIG. 23B depicts the amino acid sequence of a fusion protein comprising human HERV-K TCRα-LZL-IgG1 heavy chain.

FIG. 24 depicts the nucleotide and amino acid sequences of a fusion protein comprising human HERV-K TCRβ-LZR-IgG1 light chain.

FIG. 25A depicts the nucleotide sequence and FIG. 25B depicts the amino acid sequence of a fusion protein comprising human FK10 TCRα-LZL-IgG1 heavy chain.

FIG. 26 depicts the nucleotide and amino acid sequences of a fusion protein comprising human FK10 TCRβ-LZR-IgG1 light chain.

### DETAILED DESCRIPTION

The disclosure provides soluble, multimeric fusion proteins containing an immunoglobulin-based framework in which each polypeptide chain of the framework is operably linked to a soluble TCR or a soluble pMHC by a flexible multimerization domain. The structure of each polypeptide chain results in efficient multimerization to produce stable soluble TCR and pMHC multimers (e.g., dimers, tetramers, hexamers, etc.) without the need for additional mutations or disulfide bond formation. In addition, the flexibility of the multimerization domain allows all TCR or pMHC monomers within the multimeric protein to be oriented in the same direction for maximal engagement of targets for optimal binding avidity for the targeted antigenic peptide.

The disclosure provides methods for producing soluble, multimeric proteins with high protein yields within a short time period, thus reducing the time and cost for providing sufficient amounts of the soluble, multimeric proteins. Accordingly, the compositions and methods described herein are suitable for routine laboratory research, as well as large scale industrial and clinical applications.

### Soluble Multimeric Fusion Proteins

The disclosure provides soluble multimeric fusion proteins which binds to a component of the MHC/TCR immune complex, wherein each fusion protein in the multimer comprises a soluble T cell receptor (TCR) or a soluble Major Histocompatibility Complex (MHC) linked to an immunoglobulin framework (e.g., immunoglobulin heavy chain constant region or immunoglobulin light chain constant region) via a multimerization domain.

### Soluble Multimeric TCR-Immunoglobulin Fusion Proteins

Accordingly, in one aspect, the disclosure provides a soluble multimeric fusion protein wherein each fusion protein in the multimer comprises a soluble TCR polypeptide which binds to a peptide antigen. In some embodiments, the multimeric TCR-fusion protein is a dimer, a trimer, a tetramer or a hexamer. In one embodiment, the multimeric TCR-fusion protein is a dimer. In another embodiment, the multimeric TCR-fusion protein is a tetramer.

In some embodiments, each TCR-fusion protein in the multimeric protein binds to the same peptide antigen. In other embodiments, at least two of the TCR-fusion protein in the multimeric protein bind to a different peptide antigens.

In some embodiments, at least one soluble TCR polypeptide in the multimer is operably linked to an immunoglobulin heavy chain constant region or fragment thereof. In some embodiments, the multimeric TCR-fusion protein comprises two soluble TCR polypeptides operably linked to an immunoglobulin heavy chain constant region or fragment thereof.

In some embodiments, the multimeric TCR-fusion protein comprises at least one fusion protein comprising a soluble TCR polypeptide operably linked to an immunoglobulin heavy chain constant region or fragment thereof. In some embodiments, the multimeric TCR-fusion protein comprises two fusion proteins comprising a soluble TCR polypeptide operably linked to an immunoglobulin heavy chain constant region or fragment thereof. In some embodiments, the multimeric TCR-fusion protein comprises three fusion proteins comprising a soluble TCR polypeptide operably linked to an immunoglobulin heavy chain constant region or fragment thereof.

In some embodiments, the multimeric TCR-immunoglobulin fusion protein comprises two fusion proteins, each comprising a soluble TCR polypeptide operably linked to an immunoglobulin heavy chain constant region or fragment thereof, wherein the two fusion proteins are the same, and wherein the immunoglobulin heavy chain constant region or fragment thereof of the first fusion protein and the immunoglobulin heavy chain constant region or fragment thereof of the second fusion protein forms an immunoglobulin framework, thereby forming a multimeric TCR-immunoglobulin fusion protein that is a TCR dimer, trimer, tetramer, or hexamer. In some embodiments, the multimeric TCR-immunoglobulin fusion protein is a TCR dimer. In some embodiments, the multimeric TCR-immunoglobulin fusion protein is a TCR tetramer.

In some embodiments, the multimeric TCR-immunoglobulin fusion protein comprises two fusion proteins, each comprising a soluble TCR polypeptide operably linked to an immunoglobulin heavy chain constant region or fragment thereof, wherein the two fusion proteins are different, and wherein the immunoglobulin heavy chain constant region or fragment thereof of the first fusion protein and the immunoglobulin heavy chain constant region or fragment thereof of the second fusion protein forms an immunoglobulin framework, thereby forming a multimeric TCR-immunoglobulin fusion protein that is a TCR dimer, trimer, tetramer, or hexamer. In some embodiments, the multimeric TCR-immunoglobulin fusion protein is a TCR dimer. In some embodiments, the multimeric TCR-immunoglobulin fusion protein is a TCR tetramer. In some embodiments, the two fusion proteins comprise an immunoglobulin heavy chain constant region or fragment thereof that is the same. In some embodiments, the two fusion proteins comprise an immunoglobulin heavy chain constant region or fragment thereof that is different. In some embodiments, the two fusion proteins comprise soluble TCR polypeptides that are the same, wherein the soluble TCRs bind to the same peptide antigen. In some embodiments, the two fusion proteins comprise soluble TCR polypeptides that are different, wherein the soluble TCRs bind to different peptide antigens.

In some embodiments, the multimeric TCR-immunoglobulin fusion protein comprises three fusion proteins, each comprising a soluble TCR-polypeptide operably linked to an immunoglobulin heavy chain constant region or fragment thereof, wherein the three fusion proteins are the same, and wherein the three fusion proteins form a multimeric TCR-immunoglobulin fusion protein that is a TCR dimer, trimer, tetramer, or hexamer. In some embodiments, the multimeric TCR-immunoglobulin fusion protein is a TCR trimer.

In some embodiments, the multimeric TCR-immunoglobulin fusion protein comprises three fusion proteins, each comprising a soluble TCR-polypeptide operably linked to an immunoglobulin heavy chain constant region or fragment thereof, wherein the three fusion proteins are different, and wherein the three fusion proteins form a multimeric TCR-immunoglobulin fusion protein that is a TCR dimer, trimer, tetramer, or hexamer. In some embodiments, the three fusion proteins comprise an immunoglobulin heavy chain constant region or fragment thereof that is the same. In some embodiments, the three fusion proteins comprise an immunoglobulin heavy chain constant region or fragment thereof that is different. In some embodiments, the three fusion proteins comprise soluble TCR polypeptides that are the same, wherein the soluble TCRs bind to the same peptide antigen. In some embodiments, the three fusion proteins comprise soluble TCR polypeptides that are different, wherein the soluble TCRs bind to different peptide antigens.

In some embodiments, at least one soluble TCR polypeptide in the multimer is operably linked to an immunoglobulin heavy chain constant region or fragment thereof, and at least one second soluble TCR polypeptide in the multimer is operably linked to an immunoglobulin light chain constant region or fragment thereof.

In some embodiments, the multimeric TCR-immunoglobulin fusion protein comprises a first fusion protein comprising a soluble TCR polypeptide operably linked to an immunoglobulin heavy chain constant region or fragment thereof and a second fusion protein comprising a soluble TCR polypeptide operably linked to an immunoglobulin light chain constant region or fragment thereof. In some embodiments, the multimeric TCR-immunoglobulin fusion protein comprises two first fusion proteins comprising a soluble TCR polypeptide operably linked to an immunoglobulin heavy chain constant region or fragment thereof and two second fusion protein comprising a soluble TCR polypeptide operably linked to an immunoglobulin light chain constant region or fragment thereof, wherein the immunoglobulin heavy chain constant region or fragment thereof of the first fusion proteins and the immunoglobulin light chain constant region of the second fusion proteins forms an immunoglobulin framework, thereby forming a multimeric TCR-immunoglobulin fusion protein that is a TCR dimer, trimer, tetramer or hexamer. In some embodiments, the multimeric TCR-immunoglobulin fusion protein is a TCR dimer. In some embodiments, the multimeric TCR-immunoglobulin fusion protein is a TCR tetramer.

In some embodiments, one fusion protein in the multimeric protein comprises a soluble TCR polypeptide comprising a variable alpha (Vα) domain, and a second fusion protein in the multimeric protein comprises a soluble TCR polypeptide comprising a variable beta (Vβ) domain.

In some embodiments, one fusion protein in the multimeric protein comprises a soluble TCR polypeptide comprising a variable alpha (Vα) domain and a constant alpha (Cα) domain, and a second fusion protein in the multimeric protein comprises a soluble TCR comprising a variable β domain (Vβ) and a constant β domain (Cβ). In some embodiments, one fusion protein in the multimeric protein comprises a soluble TCR polypeptide comprising a Vα domain, a Vβ domain and a Cβ domain, and a second fusion protein comprises a soluble TCR comprising a Vα domain, a Vβ domain and a Cβ domain.

In certain embodiments, the disclosure provides a soluble multimeric T-cell receptor (TCR)-immunoglobulin fusion protein comprising (a) a first fusion protein comprising the structure, VαCα-X¹-Ig(Fc), wherein Vα is a TCR α variable region, Cα is TCR α constant region, X¹ is a first multimerization domain, and Ig(Fc) is an immunoglobulin Fc domain or fragment thereof; and (b) a second fusion protein comprising the structure, VβCβ-X²-Ig(C_{L}), wherein Vβ is a TCR β variable region, Cβ is a TCR β constant region, X² is a second multimerization domain, and Ig(C_{L}) is an immunoglobulin light chain constant region or fragment thereof, wherein the first and second fusion proteins form a soluble, multimeric TCR-immunoglobulin protein complex.

In certain embodiments, the disclosure provides soluble T-cell receptor (TCR)-immunoglobulin protein complex comprising (a) a first fusion protein comprising the structure, VαVβCβ-X¹-Ig(Fc), wherein Vα is a TCR α variable region, Vβ is a TCR β variable region, Cβ is a TCR α variable region, X¹ is a first multimerization domain, and Ig(Fc) is an immunoglobulin Fc domain or fragment thereof; and (b) a second fusion protein comprising the structure, VαVβCβ-X²-Ig(C_{L}), wherein Vα is a TCR α variable region, Vβ is a TCR β variable region, Cβ is a TCR α variable region, X² is a second multimerization domain, and Ig(C_{L}) is an immunoglobulin light chain constant region, wherein the fusion proteins form a soluble, multimeric TCR-immunoglobulin protein complex.

In some embodiments, the disclosure provides a soluble, multimeric TCR-immunoglobulin fusion protein comprising (a) a first fusion protein comprising the structure, Vα-X¹-Ig(C_{H}), wherein Vα is a TCR α variable region, X¹ is a first multimerization domain, and Ig(C_{H}) is an immunoglobulin heavy chain constant region or fragment thereof; and (b) a second fusion protein comprising the structure Vβ-X²-Ig(C_{L}), wherein Vβ is a TCR β variable region, X² is a second multimerization domain, and Ig(C_{L}) is an immunoglobulin light chain constant region or fragment thereof, wherein the first fusion protein and the second fusion protein form a soluble, multimeric TCR-immunoglobulin protein. In some embodiments, the soluble, multimeric TCR-immunoglobulin fusion protein comprises two first fusion proteins comprising the structure Vα-X¹-Ig(C_{H}) and two second fusion proteins comprising the structure Vβ-X²-Ig(C_{L}). In some embodiments, the immunoglobulin heavy chain constant region or fragment thereof of the two first fusion proteins and the immunoglobulin light chain constant region of the two second fusion proteins forms an immunoglobulin framework, thereby forming a soluble, multimeric TCR-immunoglobulin fusion protein that is a TCR dimer.

In some embodiments, the disclosure provides a soluble, multimeric TCR-immunoglobulin fusion protein comprising (a) a first fusion protein comprising the structure VαCα-X¹-Ig(C_{H}), wherein Vα is a TCR α variable region, Cα is TCR α constant region, X¹ is a first multimerization domain, and Ig(C_{H}) is an immunoglobulin heavy chain constant region or fragment thereof; and (b) a second fusion protein comprising the structure, VβCβ-X²-Ig(C_{L}), wherein Vβ is a TCR β variable region, Cβ is a TCR β constant region, X² is a second multimerization domain, and Ig(C_{L}) is an immunoglobulin light chain constant region or fragment thereof, wherein the first fusion protein and the second fusion protein form a soluble, multimeric TCR-immunoglobulin protein. In some embodiments, the soluble, multimeric TCR-immunoglobulin fusion protein comprises two first fusion proteins comprising the structure VαCα-X¹-Ig(C_{H}) and two second fusion proteins comprising the structure VβCβ-X¹-Ig(C_{L}). In some embodiments, the immunoglobulin heavy chain constant region or fragment thereof of the two first fusion proteins and the immunoglobulin light chain constant region of the two second fusion proteins form an immunoglobulin framework, thereby forming a soluble, multimeric TCR-immunoglobulin fusion protein that is a TCR dimer.

In some embodiments, the disclosure provides a soluble, multimeric TCR-immunoglobulin fusion protein comprising, (a) a first fusion protein comprising the structure, VαVβCβ-X¹-Ig(C_{H}), wherein Vα is a TCR α variable region, Vβ is a TCR β variable region, Cβ is a TCR β constant region, X¹ is a first multimerization domain, and Ig(C_{H}) is an immunoglobulin heavy chain constant region or fragment thereof; and (b) a second fusion protein comprising the structure, VαVβCβ-X²-Ig(C_{L}), wherein Vα is a TCR α variable region, Vβ is a TCR β variable region, Cβ is a TCR β constant region, X¹ is a second multimerization domain, and Ig(C_{L}) is an immunoglobulin light chain constant region; wherein the first fusion protein and the second fusion protein form a soluble, multimeric TCR-immunoglobulin fusion protein. In some embodiments, the soluble, multimeric TCR-immunoglobulin fusion protein comprises two first fusion proteins comprising the structure VαVβCβ-X¹-Ig(C_{H}) and two second fusion proteins comprising the structure VαVβCβ-X²-Ig(C_{L}). In some embodiments, the immunoglobulin heavy chain constant region or fragment thereof of the two first fusion proteins and the immunoglobulin light chain constant region of the two second fusion proteins form an immunoglobulin framework, thereby forming a soluble, multimeric TCR-immunoglobulin fusion protein that is a TCR tetramer.

In some embodiments, the disclosure provides a soluble, multimeric TCR-immunoglobulin fusion protein comprising one or more fusion proteins comprising the structure VαVβCβ-X-Ig(C_{H}), wherein Vα is a TCR α variable region, Vβ is a TCR β variable region, Cβ is a TCR β constant region, X is a multimerization domain, and Ig(C_{H}) is an immunoglobulin heavy chain constant region or fragment thereof; and wherein the one or more fusion proteins form a soluble, multimeric TCR-immunoglobulin fusion protein.

In some embodiments, the disclosure provides a soluble, multimeric TCR-immunoglobulin fusion protein comprising two fusion proteins comprising the structure, VαVβCβ-X-Ig(C_{H}), wherein Vα is a TCR α variable region, Vβ is a TCR β variable region, Cβ is a TCR β constant region, X is a multimerization domain, and Ig(C_{H}) is an immunoglobulin heavy chain constant region or fragment thereof; and wherein the two fusion proteins form a soluble, multimeric TCR-immunoglobulin fusion protein. In some embodiments, the immunoglobulin heavy chain constant region or fragment thereof of the two fusion proteins forms an immunoglobulin framework, thereby forming a soluble, multimeric TCR-immunoglobulin fusion protein that is a TCR dimer.

In some embodiments, the disclosure provides a soluble, multimeric TCR-immunoglobulin fusion protein comprising three fusion proteins comprising the structure, VαVβCβ-X-Ig(C_{H}), wherein Vα is a TCR α variable region, Vβ is a TCR β variable region, Cβ is a TCR β constant region, X is a multimerization domain, and Ig(C_{H}) is an immunoglobulin heavy chain constant region or fragment thereof; and wherein the three fusion proteins form a soluble, multimeric TCR-immunoglobulin fusion protein. In some embodiments, the three fusion proteins are linked through the multimerization domain, thereby forming a soluble, multimeric TCR-immunoglobulin fusion protein that is a TCR trimer.

### Soluble Multimeric MHC Class I-Immunoglobulin Fusion Proteins

In another aspect, the disclosure provides a soluble multimeric fusion protein wherein each fusion protein in the multimeric protein comprises a soluble MHC polypeptide. In some embodiments, the multimeric MHC-fusion protein is a dimer, a trimer, a tetramer or a hexamer. In one embodiment, the multimeric MHC-fusion protein is a dimer. In another embodiment, the multimeric MHC-fusion protein is a tetramer.

In some embodiments, each fusion protein in the multimeric protein comprises a soluble MHC class I polypeptide. In some embodiments, each fusion protein in the MHC-multimeric protein comprises a soluble MHC class I α domain and a β2-microglobulin polypeptide. The soluble MHC class I molecule can comprise, e.g., the α1 domain of an MHC class I molecule, the α2 domain of an MHC class I molecule, or both the α1 domain and the α2 domain of an MHC class I molecule. In some embodiments, the soluble MHC class I molecule comprises a β2 microglobulin polypeptide. In some embodiments, the soluble MHC class I molecule comprises the α3 domain of an MHC class I molecule. In some embodiments, the soluble MHC class I molecule comprises the α1 domain of an MHC class I molecule, the α2 domain of an MHC class I molecule, and the α3 domain of an MHC class I molecule.

In some embodiments, the soluble MHC class I molecule comprises a β2 microglobulin polypeptide operably linked, optionally via a peptide linker, to the α1 domain of an MHC class I molecule. In some embodiments, the soluble MHC class I molecule comprises a β2 microglobulin polypeptide operably linked, optionally via a peptide linker, to the α1 domain of an MHC class I molecule, wherein the MHC class I molecule further comprises an α2 domain. In some embodiments, the soluble MHC class I molecule comprises a β2 microglobulin polypeptide operably linked, optionally via a peptide linker, to the α1 domain of an MHC class I molecule, wherein the MHC class I molecule further comprises an α2 domain and a α3 domain.

In some embodiments, the soluble MHC class I molecule comprising a β2 microglobulin and a MHC class I α domain (e.g., α1+α2, α1+α2+α3) further comprises a peptide antigen. In some embodiments, the peptide antigen is operably linked, optionally via a linker, to the β2 microglobulin domain.

In some embodiments, at least one fusion protein in the MHC-multimeric protein comprises a soluble MHC class I α domain and a β2-microglobulin polypeptide operably linked to an immunoglobulin heavy chain constant region or fragment thereof.

In some embodiments, the soluble, multimeric MHC class I-immunoglobulin fusion protein comprises two fusion proteins, each comprising a β2-microglobulin and a MHC class I α domain that is operably linked to an immunoglobulin heavy chain constant region or fragment thereof, wherein the two fusion proteins are the same, and wherein the immunoglobulin heavy chain of the first fusion protein and the immunoglobulin heavy chain of the second fusion protein form an immunoglobulin framework, thereby forming a multimeric MHC class I-immunoglobulin fusion protein that is a MHC class I receptor dimer, trimer, tetramer or hexamer. In some embodiments, the multimeric MHC class I-immunoglobulin fusion protein is a MHC class I receptor dimer. In some embodiments, the multimeric MHC class I-immunoglobulin fusion protein is a MHC class I receptor tetramer.

In some embodiments, the soluble, multimeric MHC class I-immunoglobulin fusion protein comprises two fusion proteins, each comprising a β2-microglobulin and a MHC class I α domain that is operably linked to an immunoglobulin heavy chain constant region or fragment thereof, wherein the two fusion proteins are different, and wherein the immunoglobulin heavy chain of the first fusion protein and the immunoglobulin heavy chain of the second fusion protein form an immunoglobulin framework, thereby forming a soluble, multimeric MHC class I-immunoglobulin fusion protein that is a MHC class I receptor dimer, trimer, tetramer or hexamer. In some embodiments, the multimeric MHC class I-immunoglobulin fusion protein is a MHC class I receptor dimer. In some embodiments, the multimeric MHC class I-immunoglobulin fusion protein is a MHC class I receptor tetramer. In some embodiments, the two fusion proteins comprise an immunoglobulin heavy chain constant region or fragment thereof that is the same. In some embodiments, the two fusion proteins comprise an immunoglobulin heavy chain constant region or fragment thereof that is different. In some embodiments, the two fusion proteins comprise soluble MHC class I polypeptides that are the same, wherein the soluble MHC class I receptors bind to the same peptide antigen. In some embodiments, the two fusion proteins comprise soluble MHC class I polypeptides that are different, wherein the soluble MHC class I receptors bind to different peptide antigens.

In some embodiments, the soluble, multimeric MHC class I-immunoglobulin fusion protein comprises three fusion proteins, each comprising a β2-microglobulin and a MHC class I α domain that is operably linked to an immunoglobulin heavy chain constant region or fragment thereof, wherein the three fusion proteins are the same, and wherein the three fusion proteins form a soluble, multimeric MHC class I-immunoglobulin fusion protein that is a MHC class I receptor dimer, trimer, tetramer or hexamer. In some embodiments, the multimeric MHC class I-immunoglobulin fusion protein is a MHC class I receptor trimer. In some embodiments, the soluble, multimeric MHC class I-immunoglobulin fusion protein comprises three fusion proteins, each comprising a β2-microglobulin and a MHC class I α domain that is operably linked to an immunoglobulin heavy chain constant region or fragment thereof, wherein the three fusion proteins are different, and wherein the three fusion proteins form a soluble, multimeric MHC class I-immunoglobulin fusion protein that is a MHC class I receptor dimer, trimer, tetramer or hexamer. In some embodiments, the multimeric MHC class I-immunoglobulin fusion protein is a MHC class I receptor trimer.

In some embodiments, at least one fusion protein in the MHC-multimeric protein comprises a soluble MHC class I α domain and a β2-microglobulin polypeptide operably linked to an immunoglobulin light chain constant region or fragment thereof. In some embodiments, one fusion protein in the MHC-multimer comprises a soluble MHC class I α domain and a β2-microglobulin polypeptide operably linked to an immunoglobulin heavy chain constant region, and a second fusion protein in the MHC multimer comprises a soluble MHC class I α domain and a β2-microglobulin polypeptide operably linked to an immunoglobulin light chain constant region or fragment thereof.

In some embodiments, the soluble, multimeric MHC class I-immunoglobulin fusion protein comprises a first fusion protein comprising a soluble MHC class I α domain and a β2-microglobulin polypeptide operably linked to an immunoglobulin heavy chain constant region and a second fusion protein comprising a soluble MHC class I α domain and a β2-microglobulin polypeptide operably linked to an immunoglobulin light chain constant region or fragment thereof, wherein the immunoglobulin heavy chain constant region or fragment thereof of the first fusion protein and the immunoglobulin light chain constant region or fragment thereof of the second fusion protein form an immunoglobulin framework, thereby forming a soluble, multimeric MHC class I-immunoglobulin fusion protein that is an MHC class I receptor dimer, trimer, tetramer or hexamer.

In some embodiments, the soluble, multimeric MHC class I-immunoglobulin fusion protein comprises two first fusion proteins, each comprising a soluble MHC class I α domain and a β2-microglobulin polypeptide operably linked to an immunoglobulin heavy chain constant region, and two second fusion proteins, each comprising a soluble MHC class I α domain and a β2-microglobulin polypeptide operably linked to an immunoglobulin light chain constant region or fragment thereof, wherein the immunoglobulin heavy chain constant region or fragment thereof of the first fusion proteins and the immunoglobulin light chain constant region or fragment thereof of the second fusion proteins form an immunoglobulin framework, thereby forming a soluble, multimeric MHC class I-immunoglobulin fusion protein that is an MHC class I receptor dimer, trimer, tetramer or hexamer. In some embodiments, the soluble, multimeric MHC class I-immunoglobulin fusion protein is an MHC class I receptor dimer. In some embodiments, the soluble, multimeric MHC class I-immunoglobulin fusion protein is an MHC class I receptor tetramer.

In certain embodiments, the disclosure provides a soluble multimeric MHC class I-immunoglobulin fusion protein comprising (a) a first fusion protein comprising the structure β2M-MHCIα-X¹-Ig(Fc), wherein β2M is a soluble β2-microglobulin polypeptide, MHCIα is a soluble MHC class I α chain, X¹ is a first multimerization domain, and Ig(Fc) is an immunoglobulin Fc domain or fragment thereof, and (b) a second fusion protein comprising the structure β2M-MHCIα-X²-Ig(Fc), wherein β2M is a soluble β2-microglobulin polypeptide, MHCIα is a soluble MHC class I α chain, X² is a second multimerization domain, and Ig(Fc) is an immunoglobulin heavy chain constant domain or fragment thereof, wherein the first and second fusion proteins form a soluble, multimeric MHC Class I-immunoglobulin protein.

In certain embodiments, the disclosure provides a soluble multimeric MHC class I-immunoglobulin fusion protein comprising (a) a first fusion protein comprising the structure β2M-MHCIα-X¹-Ig(Fc), wherein β2M is a soluble β2-microglobulin polypeptide, MHCIα is a soluble MHC class I α chain, X¹ is a first multimerization domain, and Ig(Fc) is an immunoglobulin Fc domain or fragment thereof, and (b) a second fusion protein comprising the structure β2M-MHCIα-X²-Ig(C_{L}), wherein β2M is a soluble β2-microglobulin polypeptide, MHCIα is a soluble MHC class I α chain, X² is a second multimerization domain, and Ig(C_{L}) is an immunoglobulin light chain constant region or fragment thereof, wherein the first and second fusion proteins form a soluble, multimeric MHC Class I-immunoglobulin protein.

In some embodiments, the disclosure provides a soluble, multimeric MHC Class I-immunoglobulin fusion protein comprising, (a) a first fusion protein comprising the structure, β2M-MHCIα-X¹-Ig(C_{H}), wherein β2M is a soluble β2-microglobulin polypeptide, MHCIα is a soluble MHC class I α chain, X¹ is a first multimerization domain, and Ig(C_{H}) is an immunoglobulin heavy chain constant region or fragment thereof; and (b) a second fusion protein comprising the structure, β2M-MHCIα-X²-Ig(C_{L}), wherein β2M is a soluble β2-microglobulin polypeptide, MHCIα is a soluble MHC class I α chain, X² is a second multimerization domain, and Ig(C_{L}) is an immunoglobulin light chain constant region or fragment thereof, wherein the first fusion protein and the second fusion protein form a soluble, multimeric MHC Class I-immunoglobulin fusion protein. In some embodiments, the soluble, multimeric MHC Class I-immunoglobulin fusion protein comprises two first fusion proteins comprising the structure β2M-MHCIα-X¹-Ig(C_{H}) and two second fusion proteins comprising the structure β2M-MHCIα-X²-Ig(C_{L}). In some embodiments, the immunoglobulin heavy chain constant region or fragment thereof of the two first fusion proteins and the immunoglobulin light chain constant region or fragment thereof of the two second fusion proteins form an immunoglobulin framework, thereby forming a soluble, multimeric MHC Class I-immunoglobulin fusion protein that is a MHC class I receptor tetramer.

In some embodiments, the disclosure provides a soluble, multimeric MHC Class I-immunoglobulin fusion protein comprising one or more fusion proteins comprising the structure, β2M-MHCIα-X-Ig(C_{H}), wherein β2M is a soluble β2-microglobulin polypeptide, MHCIα is a soluble MHC class I α chain, X is a multimerization domain, and Ig(C_{H}) is an immunoglobulin heavy chain constant region or fragment thereof; and wherein the one or more fusion proteins form a soluble, multimeric MHC Class I-immunoglobulin fusion protein.

In some embodiments, the disclosure provides a soluble, multimeric MHC Class I-immunoglobulin fusion protein comprising two fusion proteins comprising the structure, β2M-MHCIα-X-Ig(C_{H}), wherein β2M is a soluble β2-microglobulin polypeptide, MHCIα is a soluble MHC class I α chain, X is a multimerization domain, and Ig(C_{H}) is an immunoglobulin heavy chain constant region or fragment thereof; and wherein the two fusion proteins form a soluble, multimeric MHC Class I-immunoglobulin fusion protein. In some embodiments, the immunoglobulin heavy chain constant region or fragment thereof of the two fusion proteins forms an immunoglobulin framework, thereby forming a soluble, multimeric MHC Class I-immunoglobulin fusion protein that is a MHC class I receptor dimer.

In some embodiments, the disclosure provides a soluble, multimeric MHC Class I-immunoglobulin fusion protein comprising three fusion proteins comprising the structure, β2M-MHCIα-X-Ig(C_{H}), wherein β2M is a soluble β2-microglobulin polypeptide, MHCIα is a soluble MHC class I α chain, X is a multimerization domain, and Ig(C_{H}) is an immunoglobulin heavy chain constant region or fragment thereof; and wherein the three fusion proteins form a soluble, multimeric MHC Class I-immunoglobulin fusion protein. In some embodiments, the three fusion proteins are linked through the multimerization domain, thereby forming a multimeric MHC Class I-immunoglobulin fusion protein that is a MHC class I receptor trimer or hexamer. In some embodiments, the multimeric MHC Class I-immunoglobulin fusion protein is a MHC class I receptor trimer.

In various embodiments, at least one fusion protein in the MHC I-multimer comprises a peptide loaded MHC (pMHC). In some embodiments, two or more fusion proteins in the MHC I-multimer comprise a peptide loaded MHC (pMHC). In one embodiment, each fusion protein in the MHC I-multimer comprises a pMHC. In some embodiments, the antigen peptide is operably linked, optionally via a linker, to an MHC class I polypeptide comprising a MHC class I α domain and a β2-microglobulin domain. In some embodiments, the antigen peptide is operably linked to the β2-microglobulin domain, optionally via an amino acid linker.

In one embodiment, the disclosure provides a soluble multimeric MHC class I-immunoglobulin fusion protein comprising (a) a first fusion protein comprising the structure Ag-β2M-MHCIα-X¹-Ig(Fc), wherein Ag is an antigenic peptide, β2M is a soluble β2-microglobulin polypeptide, MHCIα is a soluble MHC class I α chain, X¹ is a first multimerization domain, and Ig(Fc) is an immunoglobulin Fc domain or fragment thereof, and (b) a second fusion protein comprising the structure Ag-β2M-MHCIα-X²-Ig(C_{L}), wherein Ag is an antigenic peptide, β2M is a soluble β2-microglobulin polypeptide, MHCIα is a soluble MHC class I α chain, X² is a second multimerization domain, and Ig(C_{L}) is an immunoglobulin light chain constant region or fragment thereof, wherein the first and second fusion proteins form a soluble, multimeric MHC Class I-immunoglobulin protein.

In some embodiments, the disclosure provides a soluble, multimeric MHC Class I-immunoglobulin fusion protein comprising, (a) a first fusion protein comprising the structure, Ag-β2M-MHCIα-X¹-Ig(C_{H}), wherein Ag is an antigenic peptide, wherein β2M is a soluble β2-microglobulin polypeptide, MHCIα is a soluble MHC class I α chain, X¹ is a first multimerization domain, and Ig(C_{H}) is an immunoglobulin heavy chain constant region or fragment thereof; and (b) a second fusion protein comprising the structure, Ag-β2M-MHCIα-X²-Ig(C_{L}), wherein Ag is an antigenic peptide, wherein β2M is a soluble β2-microglobulin polypeptide, MHCIα is a soluble MHC class I α chain, X² is a second multimerization domain, and Ig(C_{L}) is an immunoglobulin light chain constant region or fragment thereof, wherein the at least one first fusion protein and the at least one second fusion protein form a soluble, multimeric MHC Class I-immunoglobulin fusion protein. In some embodiments, the soluble, multimeric MHC Class I-immunoglobulin fusion protein comprises two first fusion proteins comprising the structure Ag-β2M-MHCIα-XI-Ig(C_{H}) and two second fusion proteins comprising the structure Ag-β2M-MHCIα-X²-Ig(C_{L}). In some embodiments, the immunoglobulin heavy chain constant region or fragment thereof of the two first fusion proteins and the immunoglobulin light chain constant region or fragment thereof of the two second fusion proteins forms an immunoglobulin framework, thereby forming a soluble, multimeric MHC Class I-immunoglobulin fusion protein that is a MHC class I receptor tetramer.

In some embodiments, the disclosure provides a soluble, multimeric MHC Class I-immunoglobulin fusion protein comprising one or more fusion proteins comprising the structure, Ag-β2M-MHCIα-X-Ig(C_{H}), wherein Ag is an antigenic peptide, wherein β2M is a soluble β2-microglobulin polypeptide, MHCIα is a soluble MHC class I α chain, X is a multimerization domain, and Ig(C_{H}) is an immunoglobulin heavy chain constant region or fragment thereof; and wherein the one or more fusion proteins form a soluble, multimeric MHC Class I-immunoglobulin fusion protein.

In some embodiments, the disclosure provides a soluble, multimeric MHC Class I-immunoglobulin fusion protein comprising two fusion proteins comprising the structure, Ag-β2M-MHCIα-X-Ig(C_{H}), wherein Ag is an antigenic peptide, wherein β2M is a soluble β2-microglobulin polypeptide, MHCIα is a soluble MHC class I α chain, X is a multimerization domain, and Ig(C_{H}) is an immunoglobulin heavy chain constant region or fragment thereof; and wherein the two fusion proteins form a soluble, multimeric MHC Class I-immunoglobulin fusion protein. In some embodiments, the immunoglobulin heavy chain constant region or fragment thereof of the two fusion proteins forms an immunoglobulin framework, thereby forming a soluble, multimeric MHC Class I-immunoglobulin fusion protein that is a MHC class I receptor dimer.

In some embodiments, the disclosure provides a soluble, multimeric MHC Class I-immunoglobulin fusion protein comprising three fusion proteins comprising the structure, Ag-β2M-MHCIα-X-Ig(C_{H}), wherein Ag is an antigenic peptide, wherein β2M is a soluble β2-microglobulin polypeptide, MHCIα is a soluble MHC class I α chain, X is a multimerization domain, and Ig(C_{H}) is an immunoglobulin heavy chain constant region or fragment thereof; and wherein the three fusion proteins form a soluble, multimeric MHC Class I-immunoglobulin fusion protein. In some embodiments, the three fusion proteins are linked through the multimerization domain, thus forming a multimeric MHC Class I-immunoglobulin fusion protein that is a MHC class I trimer or hexamer. In some embodiments, the multimeric MHC Class I-immunoglobulin fusion protein is a MHC class I receptor trimer.

In certain embodiments, MHCIα comprises an α1 domain of an MHC class I molecule, the α2 domain of an MHC class I molecule, or both the α1 domain and the α2 domain of an MHC class I molecule. In some embodiments, MHCIα comprises an α1 domain, an α2 domain, and an α3 domain of an MHC class I molecule.

### Soluble Multimeric MHC Class II-Immunoglobulin Fusion Proteins

In other embodiments, each fusion protein in the multimeric protein comprises a soluble MHC class II polypeptide. In some embodiments, at least one fusion protein in the multimeric protein comprises an MHC II α domain, and an MHC II β domain. In some embodiments, at least two fusion proteins in the multimeric protein comprises an MHC II α domain, and an MHC II β domain. In other embodiments, the MHC-multimer comprises at least one fusion protein comprising a soluble MHC II α domain, and at least one fusion protein comprising a soluble MHC II β domain. The soluble MHC class II molecule can comprise, e.g., the α1 domain of a first MHC class II molecule and the β1 domain of a second MHC class II molecule. In some embodiments, the soluble MHC class II polypeptide comprises the α1 domain and α2 domain of a first MHC class II molecule and the β1 domain and β2 domain of a second MHC class II molecule. The first and second MHC class II molecule can be the same or different MHC class II molecules. In some embodiments, the soluble MHC class II molecule comprises the α2 domain of an MHC class II molecule. In some embodiments, the soluble MHC class II molecule comprises the β2 domain of an MHC class II molecule.

In some embodiments, at least one fusion protein in the MHC-multimeric protein comprises a soluble MHC class II α domain and an MHC II β domain operably linked to an immunoglobulin heavy chain constant region or fragment thereof.

In some embodiments, the soluble, multimeric MHC class II-immunoglobulin fusion protein comprises a fusion protein comprising a soluble MHC class II α domain and a MHC II β domain that is operably linked to an immunoglobulin heavy chain constant region or fragment thereof. In some embodiments, the soluble, multimeric MHC class II-immunoglobulin fusion protein comprises two fusion proteins, each comprising a soluble MHC class II α domain and a MHC II β domain that is operably linked to an immunoglobulin heavy chain constant region or fragment thereof, wherein the two fusion proteins are the same, and wherein the immunoglobulin heavy chain of the first fusion protein and the immunoglobulin heavy chain of the second fusion protein form an immunoglobulin framework, thereby forming a multimeric MHC class II-immunoglobulin fusion protein that is a MHC class II receptor dimer, trimer, tetramer or hexamer. In some embodiments, the multimeric MHC class II-immunoglobulin fusion protein is a MHC class II receptor dimer. In some embodiments, the multimeric MHC class II-immunoglobulin fusion protein is a MHC class II receptor tetramer.

In some embodiments, the soluble, multimeric MHC class II-immunoglobulin fusion protein comprises two fusion proteins, each comprising a soluble MHC class II α domain and an MHC II β domain that is operably linked to an immunoglobulin heavy chain constant region or fragment thereof, wherein the two fusion proteins are different, and wherein the immunoglobulin heavy chain of the first fusion protein and the immunoglobulin heavy chain of the second fusion protein form an immunoglobulin framework, thereby forming a soluble, multimeric MHC class II-immunoglobulin fusion protein that is a MHC class II receptor dimer, trimer, tetramer or hexamer. In some embodiments, the multimeric MHC class II-immunoglobulin fusion protein is a MHC class II receptor dimer. In some embodiments, the multimeric MHC class II-immunoglobulin fusion protein is a MHC class II receptor tetramer. In some embodiments, the two fusion proteins comprise an immunoglobulin heavy chain constant region or fragment thereof that is the same. In some embodiments, the two fusion proteins comprise an immunoglobulin heavy chain constant region or fragment thereof that is different. In some embodiments, the two fusion proteins comprise soluble MHC class II polypeptides that are the same, wherein the soluble MHC class II receptors bind to the same peptide antigen. In some embodiments, the two fusion proteins comprise soluble MHC class II polypeptides that are different, wherein the soluble MHC class II receptors bind to different peptide antigens.

In some embodiments, the soluble, multimeric MHC class II-immunoglobulin fusion protein comprises three fusion proteins, each comprising a soluble MHC class II α domain and an MHC II β domain that is operably linked to an immunoglobulin heavy chain constant region or fragment thereof, wherein the three fusion proteins are the same, and wherein the three fusion proteins form a soluble, multimeric MHC class II-immunoglobulin fusion protein that is a MHC class II receptor dimer, trimer, tetramer or hexamer. In some embodiments, the soluble, multimeric MHC class II-immunoglobulin fusion protein comprises three fusion proteins, each comprising a soluble MHC class II α domain and an MHC II β domain that is operably linked to an immunoglobulin heavy chain constant region or fragment thereof, wherein the three fusion proteins are different, and wherein the three fusion proteins form a soluble, multimeric MHC class II-immunoglobulin fusion protein that is a MHC class II receptor dimer, trimer, tetramer or hexamer. In some embodiments, the multimeric MHC class II-immunoglobulin fusion protein is a MHC class II receptor trimer.

In some embodiments, at least one fusion protein in the MHC-multimeric protein comprises a soluble MHC class II α domain and a MHC II β domain operably linked to an immunoglobulin light chain constant region or fragment thereof. In some embodiments, one fusion protein in the MHC-multimer comprises a soluble MHC class II α domain operably linked to an immunoglobulin heavy chain constant region, and a second fusion protein in the MHC-multimer comprises a soluble MHC class II β domain operably linked to an immunoglobulin light chain constant region or fragment thereof.

In some embodiments, the soluble, multimeric MHC class II-immunoglobulin fusion protein comprises a first fusion protein comprising a soluble MHC class II α domain and a MHC II β domain operably linked to an immunoglobulin heavy chain constant region, and a second fusion protein comprising soluble MHC class II α domain and a MHC II β domain operably linked to an immunoglobulin light chain constant region or fragment thereof, wherein the immunoglobulin heavy chain constant region or fragment thereof of the first fusion protein and the immunoglobulin light chain constant region or fragment thereof of the second fusion protein form an immunoglobulin framework, thereby forming a soluble, multimeric MHC class II-immunoglobulin fusion protein that is a MHC class II receptor dimer, trimer, tetramer or hexamer. In some embodiments, the multimeric MHC class II-immunoglobulin fusion protein is a MHC class II receptor dimer. In some embodiments, the multimeric MHC class II-immunoglobulin fusion protein is a MHC class II receptor tetramer.

In some embodiments, the soluble, multimeric MHC class II-immunoglobulin fusion protein comprises two first fusion proteins, each comprising soluble MHC class II α domain operably linked to an immunoglobulin heavy chain constant region, and two second fusion proteins, each comprising a soluble MHC II β domain operably linked to an immunoglobulin light chain constant region or fragment thereof, wherein the immunoglobulin heavy chain constant region or fragment thereof of the first fusion proteins and the immunoglobulin light chain constant region or fragment thereof of the second fusion proteins form an immunoglobulin framework, thereby forming a soluble, multimeric MHC class II-immunoglobulin fusion protein to form a MHC class II receptor dimer, trimer, tetramer or hexamer. In some embodiments, the multimeric MHC class II-immunoglobulin fusion protein is a MHC class II receptor dimer. In certain embodiments, the disclosure provides a soluble multimeric MHC II-immunoglobulin fusion protein comprising (a) a first fusion protein comprising the structure, MHCIIα-MHCIIβ-X¹-Ig(Fc), wherein MHCIIα is a soluble MHC class II α domain, MHCIIβ is a soluble MHC class II β domain, X¹ is a first multimerization domain, and Ig(Fc) is an immunoglobulin Fc domain or fragment thereof; and (b) a second fusion protein comprising the structure, MHCIIα-MHCIIβ-X²-Ig(Fc), wherein MHCIIα is a soluble MHC class II α domain, MHCIIβ is a soluble MHC class II β domain, X¹ is a second multimerization domain, and Ig(Fc) is an immunoglobulin Fc domain or fragment thereof, wherein the first and second fusion proteins form a soluble, multimeric MHCII-immunoglobulin protein fusion.

In certain embodiments, the disclosure provides a soluble multimeric MHC II-immunoglobulin fusion protein comprising (a) a first fusion protein comprising the structure, MHCIIα-MHCIIβ-X¹-Ig(Fc), wherein MHCIIα is a soluble MHC class II α domain, MHCIIβ is a soluble MHC class II β domain, X¹ is a first multimerization domain, and Ig(Fc) is an immunoglobulin Fc domain or fragment thereof; and (b) a second fusion protein comprising the structure, MHCIIα-MHCIIβ-X²-Ig(C_{L}), wherein MHCIIα is a soluble MHC class II α domain, MHCIIβ is a soluble MHC class II β domain, X¹ is a second multimerization domain, and Ig(C_{L}) is an immunoglobulin light chain constant region or fragment thereof, wherein the first and second fusion proteins form a soluble, multimeric MHCII-immunoglobulin protein fusion.

In some embodiments, the disclosure provides a soluble, multimeric MHC class II-immunoglobulin fusion protein comprising, (a) a first fusion protein comprising the structure, MHCIIα-MHCIIβ-X¹-Ig(C_{H}), wherein MHCIIα is a soluble MHC class II α domain, MHCIIβ is a soluble MHC class II β domain, X¹ is a first multimerization domain, and Ig(C_{H}) is an immunoglobulin heavy chain constant region or fragment thereof; and (b) a second fusion protein comprising the structure, MHCIIα-MHCIIβ-X¹-Ig(C_{L}), wherein MHCIIα is a soluble MHC class II α domain, MHCIIβ is a soluble MHC class II β domain, X¹ is a second multimerization domain, and Ig(C_{L}) is an immunoglobulin light chain constant region or fragment thereof, wherein the at least one first fusion protein and the at least one second fusion protein form a soluble, multimeric MHC class II-immunoglobulin fusion protein. In some embodiments, the soluble, multimeric MHC Class II-immunoglobulin fusion protein comprises two first fusion proteins comprising the structure MHCIIα-MHCIIβ-X¹-Ig(C_{H}) and two second fusion proteins comprising the structure MHCIIα-MHCIIβ-V-Ig(C_{L}). In some embodiments, the immunoglobulin heavy chain constant region or fragment thereof of the two first fusion proteins and the immunoglobulin light chain constant region or fragment thereof of the two second fusion proteins form an immunoglobulin framework, thereby forming a soluble, multimeric MHC Class II-immunoglobulin fusion protein that is a MHC class II receptor tetramer.

In some embodiments, the disclosure provides a soluble, multimeric MHC class II-immunoglobulin fusion protein comprising, (a) a first fusion protein comprising the structure, MHCIIα-X¹-Ig(C_{H}), wherein MHCIIα is a soluble MHC class II α domain, X¹ is a first multimerization domain, and Ig(C_{H}) is an immunoglobulin heavy chain constant region or fragment thereof; and (b) a second fusion protein comprising the structure, MHCIIβ-X¹-Ig(C_{L}), wherein MHCIIβ is a soluble MHC class II β domain, X¹ is a second multimerization domain, and Ig(C_{L}) is an immunoglobulin light chain constant region or fragment thereof, wherein the first fusion protein and the second fusion protein form a soluble, multimeric MHC class II-immunoglobulin fusion protein. In some embodiments, the soluble, multimeric MHC Class II-immunoglobulin fusion protein comprises two first fusion proteins comprising the structure MHCIIα-X¹-Ig(C_{H}) and two second fusion proteins comprising the structure MHCIIβ-X²-Ig(C_{L}). In some embodiments, the immunoglobulin heavy chain constant region or fragment thereof of the two first fusion proteins and the immunoglobulin light chain constant region or fragment thereof of the two second fusion proteins form an immunoglobulin framework, thereby forming a soluble, multimeric MHC Class II-immunoglobulin fusion protein that is a MHC class II receptor dimer.

In some embodiments, the disclosure provides a soluble, multimeric MHC class II-immunoglobulin fusion protein comprising one or more fusion proteins comprising the structure, MHCIIα-MHCIIβ-X-Ig(C_{H}), wherein MHCIIα is a soluble MHC class II α domain, MHCIIβ is a soluble MHC class II β domain, X is a multimerization domain, and Ig(C_{H}) is an immunoglobulin heavy chain constant region or fragment thereof; and wherein the one or more fusion proteins form a soluble, multimeric MHC Class II-immunoglobulin protein.

In some embodiments, the disclosure provides a soluble, multimeric MHC class II-immunoglobulin fusion protein comprising two fusion proteins comprising the structure, MHCIIα-MHCIIβ-X-Ig(C_{H}), wherein MHCIIα is a soluble MHC class II α domain, MHCIIβ is a soluble MHC class II β domain, X is a multimerization domain, and Ig(C_{H}) is an immunoglobulin heavy chain constant region or fragment thereof; and wherein the two fusion proteins form a soluble, multimeric MHC Class II-immunoglobulin protein. In some embodiments, the immunoglobulin heavy chain constant region or fragment thereof of the two fusion proteins forms an immunoglobulin framework, thereby forming a soluble, multimeric MHC Class II-immunoglobulin fusion protein that is a MHC class II receptor dimer.

In some embodiments, the disclosure provides a soluble, multimeric MHC class II-immunoglobulin fusion protein comprising three fusion proteins comprising the structure, MHCIIα-MHCIIβ-X-Ig(C_{H}), wherein MHCIIα is a soluble MHC class II α domain, MHCIIβ is a soluble MHC class II β domain, X is a multimerization domain, and Ig(C_{H}) is an immunoglobulin heavy chain constant region or fragment thereof; and wherein the three fusion proteins form a soluble, multimeric MHC Class II-immunoglobulin protein. In some embodiments, the three fusion proteins are linked through the multimerization domain, thus forming a soluble, multimeric MHC Class II-immunoglobulin fusion protein that is a MHC class II receptor trimer or hexamer. In some embodiments, the soluble, multimeric MHC Class II-immunoglobulin fusion protein is a MHC class II receptor trimer. In some embodiments, MHCIIα comprises an MHC class II α1 domain. In some embodiments, MHCIIα is comprises an MHC class II α2 domain

In various embodiments, at least one fusion protein in the MHC II-multimer comprises a peptide loaded MHC (pMHC). In some embodiments, one fusion protein in the MHC-II multimer comprises a pMHC. In some embodiments, two or more fusion proteins in the MHC II-multimer comprise a pMHC. In one embodiment, each fusion protein in the MHC II-multimer comprises a pMHC. In some embodiments, the antigen peptide is operably linked, optionally via a linker, to an MHC class II polypeptide comprising a MHC class II α domain. In some embodiments, the antigen peptide is operably linked, optionally via a linker, to an MHC class II polypeptide comprising a MHC class II α domain and a MHC class II β domain. In some embodiments, the antigen peptide is operably linked to the MHC class II α domain, optionally via an amino acid linker.

In some embodiments, the disclosure provides a soluble, multimeric MHC class II-immunoglobulin fusion protein comprising, (a) a first fusion protein comprising the structure, Ag-MHCIIα-MHCIIβ-X¹-Ig(C_{H}), Ag is an antigenic peptide, wherein MHCIIα is a soluble MHC class II α domain, MHCIIβ is a soluble MHC class II β domain, X¹ is a first multimerization domain, and Ig(C_{H}) is an immunoglobulin heavy chain constant region or fragment thereof; and (b) a second fusion protein comprising the structure, Ag-MHCIIα-MHCIIβ-X¹-Ig(C_{L}), Ag is an antigenic peptide, wherein MHCIIα is a soluble MHC class II α domain, MHCIIβ is a soluble MHC class II β domain, X¹ is a second multimerization domain, and Ig(C_{L}) is an immunoglobulin light chain constant region or fragment thereof, wherein the first fusion protein and the second fusion protein form a soluble, multimeric MHC class II-immunoglobulin fusion protein. In some embodiments, the soluble, multimeric MHC Class II-immunoglobulin fusion protein comprises two first fusion proteins comprising the structure Ag-MHCIIα-MHCIIβ-X¹-Ig(C_{H}) and two second fusion proteins comprising the structure Ag-MHCIIα-MHCIIβ-X²-Ig(C_{L}). In some embodiments, the immunoglobulin heavy chain constant region or fragment thereof of the two first fusion proteins and the immunoglobulin light chain constant region or fragment thereof of the two second fusion proteins form an immunoglobulin framework, thereby forming a soluble, multimeric MHC Class II-immunoglobulin fusion protein that is a MHC class II receptor tetramer.

In some embodiments, the disclosure provides a soluble, multimeric MHC class II-immunoglobulin fusion protein comprising, (a) a first fusion protein comprising the structure, Ag-MHCIIα-X¹-Ig(C_{H}), Ag is an antigenic peptide, wherein MHCIIα is a soluble MHC class II α domain, X¹ is a first multimerization domain, and Ig(C_{H}) is an immunoglobulin heavy chain constant region or fragment thereof; and (b) a second fusion protein comprising the structure, MHCIIβ-X¹-Ig(C_{L}), wherein MHCIIβ is a soluble MHC class II β domain, X¹ is a second multimerization domain, and Ig(C_{L}) is an immunoglobulin light chain constant region or fragment thereof, wherein the first fusion protein and the second fusion protein form a soluble, multimeric MHC class II-immunoglobulin fusion protein. In some embodiments, the soluble, multimeric MHC Class II-immunoglobulin fusion protein comprises two first fusion proteins comprising the structure Ag-MHCIIα-X¹-Ig(C_{H}) and two second fusion proteins comprising the structure MHCIIβ-X²-Ig(C_{L}). In some embodiments, the immunoglobulin heavy chain constant region or fragment thereof of the two first fusion proteins and the immunoglobulin light chain constant region or fragment thereof of the two second fusion proteins form an immunoglobulin framework, thereby forming a soluble, multimeric MHC Class II-immunoglobulin fusion protein that is a MHC class II receptor dimer.

In some embodiments, the disclosure provides a soluble, multimeric MHC class II-immunoglobulin fusion protein comprising one or more fusion proteins comprising the structure, Ag-MHCIIα-MHCIIβ-X-Ig(C_{H}), Ag is an antigenic peptide, wherein MHCIIα is a soluble MHC class II α domain, MHCIIβ is a soluble MHC class II β domain, X is a multimerization domain, and Ig(C_{H}) is an immunoglobulin heavy chain constant region or fragment thereof; and wherein the one or more fusion proteins form a soluble, multimeric MHC Class II-immunoglobulin protein.

In some embodiments, the disclosure provides a soluble, multimeric MHC class II-immunoglobulin fusion protein comprising two fusion proteins comprising the structure, Ag-MHCIIα-MHCIIβ-X-Ig(C_{H}), Ag is an antigenic peptide, wherein MHCIIα is a soluble MHC class II α domain, MHCIIβ is a soluble MHC class II β domain, X is a multimerization domain, and Ig(C_{H}) is an immunoglobulin heavy chain constant region or fragment thereof; and wherein the two fusion proteins form a soluble, multimeric MHC Class II-immunoglobulin protein. In some embodiments, the immunoglobulin heavy chain constant region or fragment thereof of the two fusion proteins forms an immunoglobulin framework, thereby forming a soluble, multimeric MHC Class II-immunoglobulin fusion protein that is a MHC class II receptor dimer.

In some embodiments, the disclosure provides a soluble, multimeric MHC class II-immunoglobulin fusion protein comprising three fusion proteins comprising the structure, Ag-MHCIIα-MHCIIβ-X-Ig(C_{H}), Ag is an antigenic peptide, wherein MHCIIα is a soluble MHC class II α domain, MHCIIβ is a soluble MHC class II β domain, X is a multimerization domain, and Ig(C_{H}) is an immunoglobulin heavy chain constant region or fragment thereof; and wherein the three fusion proteins form a soluble, multimeric MHC Class II-immunoglobulin protein. In some embodiments, the three fusion proteins are linked through the multimerization domain, thus forming a soluble, multimeric MHC Class II-immunoglobulin fusion protein that is a MHC class II receptor trimer or hexamer. In some embodiments, the soluble, multimeric MHC Class II-immunoglobulin fusion protein is a MHC class II receptor trimer.

The immunoglobulin framework of the multimeric fusion proteins of the disclosure comprise one or more Fc domains (e.g., 2, 3, 4, 5, or 6 Fc domains). In certain embodiments, the Fc domains may be of different types. In certain embodiments, at least one Fc domain present in the multimeric fusion protein comprises a hinge domain or portion thereof. In certain embodiments, the multimeric fusion protein disclosed herein comprises at least one Fc domain which comprises at least one CH2 domain or portion thereof. In certain embodiments, the multimeric fusion protein disclosed herein comprises at least one Fc domain which comprises at least one CH3 domain or portion thereof. In certain embodiments, the multimeric fusion protein disclosed herein comprises at least one Fc domain which comprises at least one CH4 domain or portion thereof. In certain embodiments, the multimeric fusion protein disclosed herein comprises at least one Fc domain which comprises at least one hinge domain or portion thereof and at least one CH2 domain or portion thereof (e.g., in the hinge-CH2 orientation). In certain embodiments, the multimeric fusion protein disclosed herein comprises at least one Fc domain which comprises at least one CH2 domain or portion thereof and at least one CH3 domain or portion thereof (e.g., in the CH2-CH3 orientation). In certain embodiments, the multimeric fusion protein disclosed herein comprises at least one Fc domain comprising at least one hinge domain or portion thereof, at least one CH2 domain or portion thereof, and least one CH3 domain or portion thereof, for example in the orientation hinge-CH2-CH3, hinge-CH3-CH2, or CH2-CH3-hinge.

In some embodiments, the fusion protein comprises at least one complete Fc region derived from one or more immunoglobulin heavy chains (e.g., an Fc domain including hinge, CH2, and CH3 domains, although these need not be derived from the same antibody). In certain embodiments, the fusion protein comprises at least two complete Fc domains derived from one or more immunoglobulin heavy chains. In certain embodiments, the complete Fc domain is derived from a human IgG immunoglobulin heavy chain (e.g., human IgG1). It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species. Moreover, the Fc domain or portion thereof may be derived from any immunoglobulin class, including IgM, IgG, IgD, IgA, and IgE, and any immunoglobulin isotype, including IgG1, IgG2, IgG3, and IgG4.

In some embodiments, the immunoglobulin framework of the multimeric fusion proteins provided herein comprises an immunoglobulin light chain constant region (CL or C_{L}), or a fragment thereof. The light chain region can be a naturally occurring CL, or a naturally occurring CL in which one or more amino acids have been substituted, added or deleted, provided that the CL has a desired biological property. In some embodiments, the immunoglobulin framework comprises a CL which is a kappa or lambda constant region. In some embodiments, the CL is a human kappa or lambda light chain constant region or fragment thereof. In some embodiments, the CL may comprise a C-terminal lysine.

In various embodiments of these aspects of the disclosure, the multimerization domains of the soluble multimeric fusion protein comprise leucine zipper or leucine zipper-like dimerization domains. In some embodiments, the leucine zipper domains are homodimeric. In some embodiments, the leucine zipper or leucine zipper-like domains are heterodimeric. In some embodiments, the leucine zipper or leucine zipper-like domains are selected from SYNZIP 1 to SYNZIP 48, and BATF, FOS, ATF4, ATF3, BACH1, JUND, NFE2L3, and HEPTAD. In certain embodiments, one multimerization domain comprises the leucine zipper domain BZip (RR or LZR) and a second multimerization domain comprises the leucine zipper domain AZip (EE or LZL).

In some embodiments, the soluble, multimeric fusion protein comprises a multimerization domain is LZR that comprises an amino acid sequence identified by SEQ ID NO: 8. In some embodiments, the soluble, multimeric fusion protein comprises a multimerization domain is LZL that comprises an amino acid sequence identified by SEQ ID NO: 6. In some embodiments, the soluble, multimeric fusion protein comprises a first multimerization domain that is LZR that comprises an amino acid sequence identified by SEQ ID NO: 8 and a second multimerization domain that is LZL that comprises an amino acid sequence identified by SEQ ID NO: 6. In some embodiments, the soluble, multimeric fusion protein comprises a first multimerization domain that is LZL that comprises an amino acid sequence identified by SEQ ID NO: 6 and a second multimerization domain that is LZR that comprises an amino acid sequence identified by SEQ ID NO: 8.

In some embodiments, the multimerization domains of the soluble multimeric fusion protein comprise self-trimerization domains. In some embodiments, each self-trimerization domain comprises a collagen-like scaffold. In some embodiments, the collagen-like scaffold comprises (GX₁X₂)ₙ, wherein G is glycine, X₁ and X₂ are any amino acid residues, and n is at least 5. In some embodiments, X₁ and X₂ are proline. In one embodiment, the self-trimerization domain comprises (GPP)₁₀ that comprises an amino acid sequence set forth by SEQ ID NO: 60.

In other embodiments of these aspects of the disclosure, the soluble multimeric fusion protein comprises a peptide linker. The term “peptide linker” denotes a linear amino acid chain of natural and/or synthetic origin. The linker has the function to ensure that polypeptides conjugated to each other can perform their biological activity by allowing the polypeptides to fold correctly and to be presented properly. The peptide linker may contain repetitive amino acid sequences or sequences of naturally occurring polypeptides. In some embodiments, the peptide linker has a length of from 2 to 50 amino acids. In some embodiments, the peptide linker is between 3 and 30 amino acids, between 5 to 25 amino acids, between 5 to 20 amino acids, or between 10 and 20 amino acids.

In some embodiments, the peptide linker is rich in glycine, glutamine, and/or serine residues. These residues are arranged e.g. in small repetitive units of up to five amino acids. This small repetitive unit may be repeated for one to five times. At the amino- and/or carboxy-terminal ends of the multimeric unit up to six additional arbitrary, naturally occurring amino acids may be added. Other synthetic peptidic linkers are composed of a single amino acid, which is repeated between 10 to 20 times and may comprise at the amino- and/or carboxy-terminal end up to six additional arbitrary, naturally occurring amino acids. All peptidic linkers can be encoded by a nucleic acid molecule and therefore can be recombinantly expressed. As the linkers are themselves peptides, the polypeptide connected by the linker are connected to the linker via a peptide bond that is formed between two amino acids.

In some embodiments, a soluble multimeric TCR-fusion protein of the disclosure comprises a peptide linker positioned between the soluble TCR polypeptide and the multimerization domain. In some embodiments, a soluble multimeric MHC-fusion protein of the disclosure comprises a peptide linker positioned between the MHC polypeptide and the multimerization domain. In some embodiments, the peptide linker is a Gly-Ser linker. In certain embodiments, the Gly-Ser linker is selected from the group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG. In some embodiments, the Gly-Ser linker is (G₄S)₄ (SEQ ID NO: 9).

In some embodiments, a soluble multimeric TCR-fusion protein of the disclosure comprises a peptide linker positioned between the multimerization domain and the immunoglobulin framework (e.g., immunoglobulin heavy chain constant region, immunoglobulin light chain constant region). In some embodiments, a soluble multimeric MHC-fusion protein of the disclosure comprises a peptide linker positioned between the multimerization domain and the immunoglobulin framework (e.g., immunoglobulin heavy chain constant region, immunoglobulin light chain constant region). In certain embodiments, the Gly-Ser linker is selected from the group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG. In some embodiments, the Gly-Ser linker is G₂SG₂ (SEQ ID NO: 12).

In other embodiments of these aspects of the disclosure, the soluble multimeric fusion protein comprises signal peptide. Any suitable signal peptide which directs the protein to the cell membrane and facilitates secretion of the fusion protein may be used. Typically, the signal peptide is about 16-30 amino acids in length, and is located at the N-terminus of the fusion protein. In some embodiments, the signal peptide is a TCR signal peptide, a CD8 signal peptide, a β2M signal peptide, an IgG_{κ} light chain signal peptide, or an IL-2 signal peptide.

For example, in some embodiments, a pMHCI fusion protein of the soluble multimeric MHCI-fusion protein of the disclosure comprises a β2M signal peptide, a cognate peptide, a soluble β2M protein and a soluble MHC/HLA protein.

### Soluble TCR Polypeptides

Soluble TCR polypeptides for use in the compositions and methods of the disclosure can be obtained according to routine methods. Cloning and expression of soluble and T-cell receptors in various formats has been demonstrated (e.g. Moysey et al. (2004) Anal Biochem. 326:284-286; Wulfing & Plueckthun (1994) J Mol Biol. 242:655-669; Boulter et al. (2003) Protein Eng. 16:707-711; Schodin et al. (1996) Mol Immunol 33:819 829; Chung et al. (1994) Proc Natl Acad Sci USA 91:12654 12658; Plaksin et al. (1997) J Immunol 158:2218 2227; Willcox et al. (1999) Protein Sci 8:2418 2423; Weber et al. (2005) Proc Natl Acad Sci USA. 102:19033-19038; WO04050705A2; WO9618105A1; WO04033685A1; WO02066636A2; US 2005/0214284). WO02059263C2 describes transgenic animals comprising a humanized immune system to develop human TCR molecules. Soluble TCRs and portions thereof which bind to a peptide antigen of interest can also be produced by screening a phage library, for example, as disclosed in WO 2001/062908.

Additionally, once a soluble TCR, or portion thereof, that binds to a peptide antigen of interest has been identified, the amino acid sequence of the same (referred to herein as a “reference sequence”) can be modified to increase its binding affinity for the peptide antigen. The generation of high affinity binding soluble TCRs may be obtained using methods similar to antibody affinity maturation technologies (e.g. Boulter et al. Nat Biotechnol. (2005) 23:349-354; Chlewicki et al. (2005) J Mol Biol. 346:223-239; Shusta et al. (2000) 18:754-759; Holler et al. (2000) Proc Natl Acad Sci USA 97:5387 92). WO04044004A2, WO05116646A1 and WO9839482A1 describe ribosome and phage display of TCR chains and methods to select for TCR molecules against specific antigens. WO0148145A2 describes high affinity TCRs Manipulation of the extracellular variable domains of T-cell receptors has been performed for the purpose of specificity engineering via modification of the CDR-regions (WO05114215A2; WO0155366C2).

Soluble TCR polypeptides for use in the compositions and methods of the disclosure may be isolated and tested in a variety of ways known to those skilled in the art. Standard purification methods include chromatographic techniques, electrophoretic, immunological, precipitation, dialysis, and filtration, concentration, and chromatofocusing techniques. Purification can often be enabled by a particular fusion partner. For example, TCRs may be purified using glutathione resin if a GST fusion is employed, Ni²⁺-affinity chromatography if a His-tag is employed or immobilized anti-flag antibody if a flag-tag is used. For general guidance in suitable purification techniques, see e.g. Scopes, “Protein Purification: Principles and Practice”, 1994, 3rd ed., Springer-Science and Business Media Inc., NY or Roe, “Protein Purification Techniques: A Practical Approach”, 2001, Oxford University Press.

Exemplary soluble TCR polypeptides for use with the multimeric fusion proteins of the disclosure are fully functional and soluble. By the term “fully functional” or similar term is meant that the soluble TCR specifically binds ligand (e.g., a peptide antigen). Assays for detecting such specific binding include, but are not limited to standard immunoblot techniques such as Western blotting. In some embodiments, functional soluble TCRs are able to bind antigen with at least 70% of the affinity of the corresponding full-length TCR, in some embodiments at least about 80% of the affinity of the corresponding full-length TCR, in some embodiments at least about 90% of the affinity of the corresponding full-length TCR, in some embodiments at least about 95% of the affinity of the corresponding full-length TCR, for example, as determined by Western blot or Surface Plasma Resonance analysis.

### MHC Polypeptides

In some embodiments of any of the multimeric MHC-fusion proteins described herein, the soluble MHC molecule is a HLA-A, HLA-B, HLA-C, DP, DO, or DR MHC molecule. The sequences of exemplary MHC class I and class II molecules are known in the art and publicly accessible. For example, exemplary MHC class I alpha chains include, e.g., the sequences depicted in UniProt Id. Nos. P30511, P01891, P30493, and P13747. In some embodiments, the compound described herein comprises the α1 and α2 domains of an MHC class I molecule. In some embodiments, the compound described herein comprises the α1, α2, and α3 domains of an MHC class I molecule.

In some embodiments, the compound comprises a β2-microglobulin polypeptide, e.g., a human β2-microglobulin. In some embodiments, the β-2 microglobulin is wild-type human β-2 microglobulin. In some embodiments, the β-2 microglobulin comprises an amino acid sequence that is at least 80, 85, 90, 95, or 99% identical to the amino acid sequence of the human β-2 microglobulin (UniProt Id. No. P61769).

### Immunoglobulin Framework

Fc domains and light chain constant regions useful for producing the multimeric fusion proteins disclosed herein may be obtained from a number of different sources. For example, the sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. Similarly, a variety of Fc domain gene sequences (e.g., mouse and human constant region gene sequences) are available in the form of publicly accessible deposits.

Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or portions thereof) can be derived from these sequences using art recognized techniques. The genetic material obtained using any of the foregoing methods may then be altered or synthesized to obtain polypeptides suitable for use in the methods disclosed herein. It will further be appreciated that the scope of this invention encompasses alleles, variants and mutations of constant region DNA sequences.

Fc domain sequences can be cloned, e.g., using the polymerase chain reaction and primers which are selected to amplify the domain of interest. To clone an Fc domain sequence from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270). PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7: 1509); rapid amplification of cDNA ends (Ruberti, F. et al. 1994. J. Immunol. Methods 173:33); antibody leader sequences (Larrick et al. Biochem Biophys Res Commun 1989; 160: 1250). The cloning of antibody sequences is further described in Newman et al., U.S. Pat. No. 5,658,570, filed Jan. 25, 1995, which is herein incorporated by reference.

The constant region domains or portions thereof making up an Fc domain of the multimeric fusion protein disclosed herein may be derived from different immunoglobulin molecules. For example, a multimeric fusion protein disclosed herein may comprise a CH2 domain or portion thereof derived from an IgG1 molecule and a CH3 region or portion thereof derived from an IgG3 molecule. In another example, the multimeric fusion protein comprises an Fc domain comprising a hinge domain derived, in part, from an IgG1 molecule and, in part, from an IgG3 molecule. As set forth herein, it will be understood by one of ordinary skill in the art that an Fc domain may be altered such that it varies in amino acid sequence from a naturally occurring antibody molecule.

In certain embodiments, the multimeric fusion protein disclosed herein lacks one or more constant region domains of a complete Fc region, i.e., they are partially or entirely deleted. In certain embodiments, the multimeric fusion protein disclosed herein will lack an entire CH2 domain. In certain embodiments, the multimeric fusion protein disclosed herein comprise CH2 domain-deleted Fc regions derived from a vector (e.g., from IDEC Pharmaceuticals, San Diego) encoding an IgG1 human constant region domain (see, e.g., WO02/060955A2 and WO02/096948A2). This exemplary vector is engineered to delete the C_{H}2 domain and provide a synthetic vector expressing a domain-deleted IgG1 constant region. It will be noted that these exemplary constructs are preferably engineered to fuse a binding CH3 domain directly to a hinge region of the respective Fc domain.

In other constructs it may be desirable to provide a peptide spacer between one or more constituent Fc domains. For example, a peptide spacer may be placed between a hinge region and a CH2 domain and/or between a CH2 and a CH3 domain. For example, compatible constructs could be expressed wherein the CH2 domain has been deleted and the remaining CH3 domain (synthetic or non-synthetic) is joined to the hinge region with a 1-20, 1-10, or 1-5 amino acid peptide spacer. Such a peptide spacer may be added, for instance, to ensure that the regulatory elements of the constant region domain remain free and accessible or that the hinge region remains flexible. Preferably, any linker peptide compatible used in the instant invention will be relatively non-immunogenic and not prevent proper folding of the Fc.

In certain embodiments, an Fc domain employed in the multimeric fusion protein disclosed herein is altered or modified, e.g., by amino acid mutation (e.g., addition, deletion, or substitution). As used herein, the term “Fc domain variant” refers to an Fc domain having at least one amino acid modification, such as an amino acid substitution, as compared to the wild-type Fc from which the Fc domain is derived. For example, wherein the Fc domain is derived from a human IgG1 antibody, a variant comprises at least one amino acid mutation (e.g., substitution) as compared to a wild type amino acid at the corresponding position of the human IgG1 Fc region.

In some embodiments, an Fc variant has altered antigen-dependent effector functions of the polypeptide, in particular ADCC or complement activation, e.g., as compared to a wild type Fc region. Such multimeric fusion proteins exhibit decreased binding to FcR gamma when compared to wild-type polypeptides and, therefore, mediate reduced effector function. Fc variants with decreased FcR gamma binding affinity are expected to reduce effector function, and such molecules are also useful, for example, for treatment of conditions in which target cell destruction is undesirable, e.g., where normal cells may express target molecules, or where chronic administration of the polypeptide might result in unwanted immune system activation. Amino acid mutations in the Fc domain which exhibit reduced binding to the Fc gamma receptor and Fc gamma receptor subtypes, reduced antibody dependent cell-mediated cytotoxicity, or reduced complement dependent cytotoxicity, have been described (e.g., U.S. Pat. Nos. 6,737,056; 5,624,821; U.S. 2006/0235208; 2003/0108548, each incorporated herein by reference in their entirety). In certain embodiments, the multimeric fusion protein disclosed herein comprises an amino acid substitution to an Fc domain which alters antigen-independent effector functions of the polypeptide, in particular the circulating half-life of the polypeptide.

The multimeric fusion protein disclosed herein may also comprise an amino acid substitution which alters the glycosylation of the multimeric fusion protein. For example, the Fc domain of the multimeric fusion protein may comprise an Fc domain having a mutation leading to reduced glycosylation (e.g., N- or O-linked glycosylation) or may comprise an altered glycoform of the wild-type Fc domain (e.g., a low fucose or fucose-free glycan). In certain embodiments, the multimeric fusion protein has an amino acid substitution near or within a glycosylation motif, for example, an N-linked glycosylation motif that contains the amino acid sequence NXT or NXS. Exemplary amino acid substitutions which reduce or alter glycosylation are disclosed in WO05/018572 and US2007/0111281, the contents of which are incorporated by reference herein. In certain embodiments, the multimeric fusion protein disclosed herein comprises at least one Fc domain having engineered cysteine residue or analog thereof which is located at the solvent-exposed surface. In certain embodiments, the multimeric fusion protein disclosed herein comprise an Fc domain comprising at least one engineered free cysteine residue or analog thereof that is substantially free of disulfide bonding with a second cysteine residue. Any of the above engineered cysteine residues or analogs thereof may subsequently be conjugated to a functional domain using art-recognized techniques (e.g., conjugated with a thiol-reactive heterobifunctional linker).

### Multimerization Domains

The multimeric proteins of the disclosure contain multimerization domains to promote self-assembly of individual multimeric fusion polypeptides into a dimeric, trimeric, tetrameric or hexameric protein. In some embodiments, the multimerization domain within each multimeric fusion polypeptide of a multimeric protein complex of the disclosure can bind specifically, e.g., one of the protein multimerization domains can bind specifically to a second multimerization domain. In some embodiments, specific binding between two multimeric fusion polypeptides can occur when two separate multimerization domains are present. In some embodiments, specific binding between two multimeric fusion polypeptides can occur when three or more separate multimerization domains are present. Exemplary multimerization domains are known in the art.

### Dimerization Domains

In some embodiments of any of the aspects described herein, the protein interaction domains can be leucine zipper domains or leucine zipper-like domains. Leucine zipper domains are a type of protein-protein interaction domain commonly found in transcription factors characterized by leucine residues evenly spaced through an α-helix. Leucine zippers may form heterodimers or homodimers. A number of leucine zipper domains, as well as their ability to bind each other, are known in the art and discussed further, e.g., in Reinke et al. JACS 2010 132:6025-31 and Thomposon et al. ACS Synth Biol 2012 1: 118-129; each of which is incorporated by reference herein in its entirety. Variants of leucine zipper domains have also been described (e.g., U.S. Pat. No. 9,865,833)

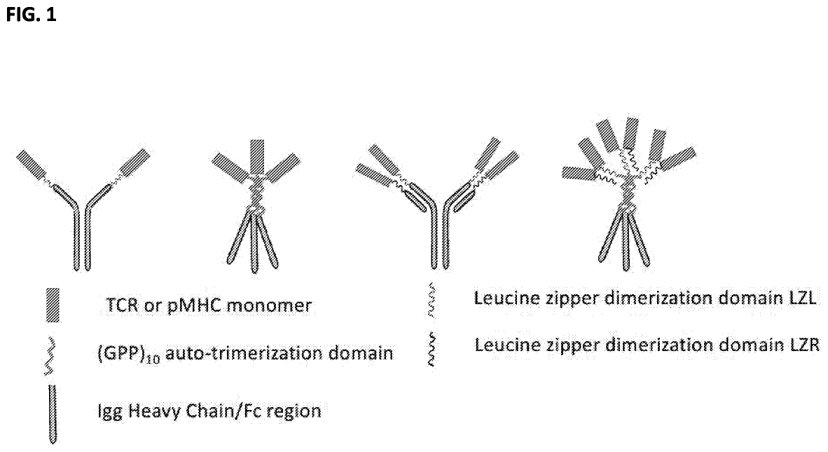

In some embodiments, one leucine zipper domain is BZip (RR) and the second leucine zipper domain is AZip (EE). In some embodiments, the amino acid sequence of a BZip (RR) leucine zipper domain is MDPDLEIRAAFLRQRNTALRTEVAELEQEVQRLE EVSQYETRYGPLGGGK (SEQ ID NO: 61). In some embodiments, the amino acid sequence of a AZip (EE) leucine zipper domain is MDPDLEIEAAFLERENTALETRVAELRQRVQRLRNRVSQYRTRYGPLGGGK (SEQ ID NO: 62). In some embodiments, one leucine zipper domain is an LZR leucine zipper domain comprising the amino acid sequence LEIEAAFLERENTALETRVAELRQRVQRLRNRVSQYRTRYGPL (SEQ ID NO: 8), and the second leucine zipper domain is an LZL leucine zipper domain comprising the amino acid sequence LEIRAAFLRQRNTALRTEVAELEQEVQRLENEVSQYETRYGPL (SEQ ID NO: 6). Further exemplary leucine zipper domains are described in Reinke et al. (JACS 2010 132:6025-31) which is incorporated by reference herein in its entirety. For example, suitable leucine zipper domains can include SYNZIP 1 to SYNZIP 48, and BATF, FOS, ATF4, ATF3, BACH1, JUND, NFE2L3, and HEPTAD. Binding affinities of various combinations of these domains have been described (e.g., at FIG. 1 of Reinke et al., supra).

In some embodiments, a suitable pair of leucine zipper domains has a dissociation constant (Kd) of 10 nM (10⁻⁸ M) or less. In some embodiments, a suitable pair of leucine zipper domains has a dissociation constant (Kd) of 1 nM or less. In some embodiments, a suitable pair of leucine zipper domains has a dissociation constant (Kd) of 10⁻¹⁰ M, 10⁻¹¹ M, 10⁻¹² M, 10⁻¹³ M, 10⁻¹⁴ M, 10⁻¹⁵ M, or less.

### Trimerization Domains

In some embodiments, the multimeric fusion protein complex provided herein comprises a trimerization positioned between the TCR or MHC and the immunoglobulin framework. In some embodiments, each trimerization domain is be directly linked to the TCR or MHC portion of the protein multimeric fusion and the Igg-framework. In some embodiments, the multimeric fusion protein comprises a peptide linker positioned between the trimerization domain and the TCR or MHC portion. In some embodiments, the multimeric fusion protein comprises a peptide linker positioned between the trimerization domain and the Igg-framework. In some embodiments, the multimeric fusion protein contains a peptide linker positioned between the TCR or MHC portion and the trimerization domain, and between the trimerization domain and the Igg-framework.

Trimerization domains are well known in the art. Non-limiting examples of trimerization domains suitable as a heterologous trimerization domain in the multimeric fusion protein of the invention include: the GCN4 leucine zipper (Harbury et al., 1993, “A switch between two-, three-, and four-stranded coiled coils in GCN4 leucine zipper mutants,” Science 262(5138):1401-7); a 35 amino-acid sequence from lung surfactant protein (Hoppe et al., 1994, “A parallel three stranded alpha-helical bundle at the nucleation site of collagen triple-helix formation,” FEBS Lett. 344(2-3):191-5); short, repeating heptad sequences from collagen (McAlinden et al., 2003, “Alpha-helical coiled-coil oligomerization domains are almost ubiquitous in the collagen superfamily,” J. Biol Chem. 278(43):42200-7. Epub 2003 Aug. 14); and the bacteriophage T4 fibritin “foldon” (see, e.g., Miroshnikov et al., 1998, “Engineering trimeric fibrous proteins based on bacteriophage T4 adhesins,” Protein Eng. 11(4):329-32). Other suitable trimerization domains are also disclosed in U.S. Pat. Nos. 6,911,205 and 8,147,843, and U.S. Pat. Appln. Pub. 2010/0136032.

In some embodiments, the trimerization domain comprises an alpha-helical coiled coil domain. Useful alpha-helical coiled coil domains include, but are not limited to those derived from Matrilin 1, Coronin 1a, dystrophia myotonica kinase (DMPK), Langerin, and combinations thereof. Such derivatives include, but are not limited to, coiled coil domains with wild type sequences as well as variants comprising one or more amino acid substitutions in the coiled coil domain wild type sequence. Coronin 1a proteins containing Coronin 1a trimerization domains are also sometimes synonymously referred to as any of Coronin-like protein A, Clipin-A, Coronin-like protein p57, Tryptophan aspartate-containing coat protein and the HUGO name CORO1A. multimeric fusion protein by using a trimerization domain, including a C-propeptide of procollagens, a coiled-coil neck domain of collectin family proteins, a C-terminal portion of FasL and a bacteriophage T4 fibritin foldon domain (Hoppe, H. J., P. N. Barlow, et al. (1994).

Exemplary trimerization domains comprise collagen-like triple-helical regions. Collagen is the most abundant protein in mammals. It is an extracellular matrix protein that contains one or more triple-helical regions (collagenous domains or collagen “scaffolds”) with a repeating triplet sequence of Gly-X-Y, where X and Y are any amino acid residues, with proline (amino acid code, P or Pro) as the residue most frequently incorporated. In the Y position, Pro is generally enzymatically modified to 4-hydroxyproline (amino acid code, 0 or Hyp), making Gly-Pro-Hyp the most common, as well as the most stabilizing, triplet in collagen. The presence of such triplets allows three polypeptide chains to fold into a triple-helical conformation. Descriptions of collagen-like peptides can be found in the description of the collagen-like domains of U.S. Pat. No. 8,669,350, which is hereby expressly incorporated by reference in its entirety.

In some embodiments, each multimeric fusion polypeptide in the multimeric protein comprises a collagen-like trimerization peptides comprising at least one stretch of at least 5, at least 10, consecutive repeats of Gly-Pro-Pro or Gly-Pro-Hyp triplets. In one embodiment, the self-trimerization domain comprises (GPP)₁₀ with an amino acid sequence set forth by SEQ ID NO: 60. In some embodiments, the collagen-like trimerization peptides can also include a perfect repeating Gly-Xaa-Yaa triplet, interrupted by a short imperfection, in which the first position of Gly or the third position of Yaa residue is missing. The stability of multimeric structures containing collagen like trimerization peptides can be determined by measuring the melting temperature of the trimers. Many studies have examined the melting temperatures/stability of G-P-X1 repeats. Frank et al., (2001); Persikov et al., (2000) Biochemistry 39, 14960-14967; Persikov et al., (2004) Protein Sci. 13: 893-902; and Mohs et al., (2007) J. Biol. Chem. 282: 29757-29765. Based on these studies, the stability of various repeat structures can be predicted.

### Linkers

In some embodiments, the one or more of the multimeric fusion polypeptides in the multimeric protein employ a linker to join any two or more domains in frame in a single polypeptide chain. In some embodiments, the soluble TCR or MHC portion of a multimeric fusion polypeptide is operably coupled to the multimerization domain (e.g., leucine zipper domain, auto-trimerization domain) via a linker. In some embodiments, the multimerization domain is operably coupled to the Igg-framework via a linker. In some embodiments, the multimeric fusion polypeptides in the multimeric protein comprise linkers between the TCR or MHC portion and the multimerization domain, and between the multimerization domain and the Igg-Framework.

In some embodiments, the linker is a polypeptide linker. Polypeptide linkers are at least one amino acid in length and can be of varying lengths. In some embodiments, a polypeptide linker is from about 1 to about 50 amino acids in length. As used in this context, the term “about” indicates +/−two amino acid residues. Since linker length must be a positive integer, the length of from about 1 to about 50 amino acids in length, means a length of from 1 to 48-52 amino acids in length. In some embodiments, a polypeptide linker is from about 10-20 amino acids in length. In some embodiments, a polypeptide linker is from about 15 to about 50 amino acids in length. In some embodiments, a polypeptide linker is from about 20 to about 45 amino acids in length. In some embodiments, a polypeptide linker is from about 15 to about 25 amino acids in length. In some embodiments, a polypeptide linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 or more amino acids in length.

Peptide linkers suitable for fusing each portion of a multimeric fusion polypeptide disclosed herein are well known in the art, and are disclosed in, e.g., US2010/0210511 US2010/0179094, and US2012/0094909, which are herein incorporated by reference in its entirety. Other linkers are provided, for example, in U.S. Pat. No. 5,525,491; Alfthan et al., Protein Eng., 1995, 8:725-731; Shan et al., J. Immunol., 1999, 162:6589-6595; Newton et al., Biochemistry, 1996, 35:545-553; Megeed et al.; Biomacromolecules, 2006, 7:999-1004; and Perisic et al., Structure, 1994, 12:1217-1226; each of which is incorporated by reference in its entirety.

In some embodiments, the polypeptide linker is synthetic. As used herein, the term “synthetic” with respect to a polypeptide linker includes peptides (or polypeptides) which comprise an amino acid sequence (which may or may not be naturally occurring) that is linked in a linear sequence of amino acids to a sequence (which may or may not be naturally occurring) to which it is not naturally linked in nature. For example, the polypeptide linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). Polypeptide linkers may be employed, for instance, to ensure that the binding portion (TCR or MHC), the multimerization domain and the Igg-Framework of each multimeric fusion polypeptide is juxtaposed to ensure proper folding and formation of a functional multimeric protein complex. Preferably, a polypeptide linker will be relatively non-immunogenic and not inhibit any non-covalent association among monomer subunits of a binding protein.

In certain embodiments, the linker is a Gly-Ser polypeptide linker, i.e., a peptide that consists of glycine and serine residues. One exemplary Gly-Ser polypeptide linker comprises the amino acid sequence (Gly₄Ser)n. In certain embodiments, n=1. In certain embodiments, n=2. In certain embodiments, n=3. In certain embodiments, n=4. In certain embodiments, n=5. In certain embodiments, n=6. Another exemplary Gly-Ser polypeptide linker comprises the amino acid sequence Ser(Gly₄Ser)n. In certain embodiments, n=1. In certain embodiments, n=2. In certain embodiments, n=3, i.e., Ser(Gly₄Ser)₃. In certain embodiments, n=4, i.e., Ser(Gly₄Ser)₄. In certain embodiments, n=5. In certain embodiments, n=6. In certain embodiments, n=7. In certain embodiments, n=8. In certain embodiments, n=9. In certain embodiments, n=10.

Other exemplary linkers include GS linkers (i.e., (GS)n), GGSG linkers (i.e., (GGSG)n), GSAT linkers, SEG linkers, and GGS linkers (i.e., (GGSGGS)n), wherein n is a positive integer (e.g., 1, 2, 3, 4, or 5). Other suitable linkers for use in multimeric fusion proteins can be found using publicly available databases, such as the Linker Database (ibi.vu.nl/programs/linkerdbwww). The Linker Database is a database of inter-domain linkers in multi-functional enzymes which serve as potential linkers in novel multimeric fusion proteins (see, e.g., George et al., Protein Engineering 2002; 15:871-9).

In some embodiments, a Gly-Ser linker is selected from the group consisting of: (G₄S)₄ (SEQ ID NO: 9), (G₄S)₃ (SEQ ID NO: 56), (G₄S)₂ (SEQ ID NO: 58), G₂SG₂ (SEQ ID NO: 12), or GSG.

Polypeptide linkers can be introduced into polypeptide sequences using techniques known in the art. Modifications can be confirmed by DNA sequence analysis. Plasmid DNA can be used to transform host cells for stable production of the polypeptides produced.

### Peptide Antigens

The peptides which associate with the MHC molecules and TCRs can either be derived from proteins made within the cell, in which case they typically associate with class I MHC molecules (Rock & Goldberg, 1999); or they can be derived from proteins which are acquired from outside of the cell, in which case they typically associate with class II MHC molecules (Watts, 1997). The peptides that evoke a cancer-specific CTL response most typically associate with class I MHC molecules. The peptides themselves are typically nine amino acids in length, but can vary from a minimum length of eight amino acids to a maximum of fourteen amino acids in length. Tumor antigens may also bind to class II MHC molecules on antigen presenting cells and provoke a T helper cell response. The peptides that bind to class II MHC molecules are generally twelve to nineteen amino acids in length, but can be as short as ten amino acids and as long as thirty amino acids.

Suitable antigens are known in the art and are available from commercial government and scientific sources. In one embodiment, the antigens are cancer antigen peptides or molecular adjuvants. The antigens may be purified or partially purified peptides or polypeptides derived from tumors. In some embodiments, the antigens are recombinant peptide or polypeptides produced by expressing DNA encoding the peptide antigen or polypeptide antigen in a heterologous expression system. The antigens can be DNA encoding all or part of an antigenic protein, polypeptide or peptide. The DNA may be in the form of vector DNA such as plasmid DNA.

In certain embodiments, candidate epitopes can be identified using a computer-implemented algorithm for candidate epitope identification. Such computer programs include, for example, the TEPITOPE program (see, e.g., Hammer et al., Adv. Immunol 66:67 100 (1997); Sturniolo et al., Nat. Biotechnol. 17:555 61 (1999); Manici et al., J Exp. Med. 189:871 76 (1999); de Lalla et al., J. Immunol. 163:1725 29 (1999); Cochlovius et al., J. Immunol. 165:4731 41 (2000); the disclosures of which are incorporated by reference herein), as well as other computer implemented algorithms (infra).

The computer-implemented algorithm for candidate epitope identification can identify candidate epitopes in, for example, a single protein, in a very large protein, in a group of related proteins (e.g., homologs, orthologs, or polymorphic variants), in a mixtures of unrelated proteins, in proteins of a tissue or organ, or in a proteome of an organism. Using this approach, it can be possible to interrogate complex tissues or organisms based on sequence information for expressed proteins (e.g., from deduced open reading frame or a cDNA library), in addition to analysis of known candidate molecular targets, as an efficient, sensitive and specific approach to identification of T cell epitopes.

Following identification of candidate epitopes, peptides or pools of peptides can be formed that correspond to the candidate epitope(s). For example, once a candidate epitope is identified, overlapping peptides can be prepared that span the candidate epitope, or portions thereof, to confirm binding of the epitope by the MHC class II molecule, and, as necessary, to refine the identification of that epitope. Alternatively, pools of peptides can be prepared including a plurality of candidate epitopes identified using a computer-implemented algorithm for candidate epitope identification.

In an exemplary embodiment, the NetMHC 4.0 program can be used. This program is based on a quantitative matrix algorithm for predicting peptide binding to MHC molecules. The program utilizes data from peptide-binding studies in which it was found that polymorphisms in MHC binding pockets dictate specificity. For example, the topography of pocket 9 of HLA-DR molecules has been found to be dependent on the DRB1 polymorphic residues 9, 37, 57, 60 and 61. The topography of a specific pocket can be generally independent of neighboring pockets, so that the constraints of pocket 9 for binding amino acid residues can be similar for different MHC alleles as long as they have identical DRB1 9, 37, 57, 60 and 61 residues.

The TEPITOPE program can be used to define pocket profiles and to minimize the number of peptide binding assays required to predict peptide binding properties. In the TEPITOPE program, results from peptide binding assays for small numbers of HLA molecules can be used to generate pocket profiles for a large number of HLA molecules. The combinations of the different modular pocket profiles can then be used to predict the overall peptide binding properties of a particular HLA molecule. The combinations of the different modular pocket profiles can be used to predict the overall peptide binding properties of antigens that contain promiscuous epitopes. The stringency of predicting peptide binding to a particular FIC can be set at different threshold values. For example, a setting of a 1% threshold implies that the peptides selected are the top 1% best binders. Similarly, a 10% threshold implies that the peptides selected are the top 10% best binders.

The identification of candidate peptide binding motifs can also be facilitated using both quantitative matrices (see, e.g., Marshall et al., J. Immunol. 154:5927 33 (1995); Hammer et al., Adv. Immunol. 66:67 100 (1997); Sturniolo et al., Nat. Biotechnol. 17:555 61 (1999); Rammensee et al., Immunogenet. 50:213 19 (1999); Brusic et al., Bioinformatics 14:121 30 (1998); Rammensee et al., Immunogenet. 41:178 228 (199); Southwood et al., J. Immunol. 160:3363 73 (1998); Brusic et al., Nucleic Acids Res. 26:368 71 (1998); Hammer et al., J. Exp. Med. 180:2353 58 (1994); the disclosures of which are incorporated by reference herein) and neural network approaches (see, e.g., Brusic et al., Bioinformatics 14:121 130 (1998); Honeyman et al., Nat. Biotechnol. 16:966 69 (1998); the disclosures of which are incorporated by reference herein).

### Cancer Antigens

In some embodiments, an MHC peptide antigen may be generated from a cancer antigen. A cancer antigen is an antigen that is typically expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell.

For example, the MHC peptide antigen may be generated from any of the following cancer antigens: MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A 10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21ras, RCAS1, a-fetoprotein, E-cadherin, a-catenin, β-catenin, γ-catenin, p120ctn, gp100Pmell 17, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, PI A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2. Additional cancer antigens include the tumor antigens described herein.

In some embodiments, the MHC peptide antigen is derived from the human endogenous retrovirus (HERV-K) envelope protein.

In certain embodiments, a tumor-associated antigen is determined by sequencing a patient's tumor cells and identifying mutated proteins that are only found in the tumor. In some embodiments, a tumor-associated antigen is determined by analyzing a patient's tumor cells and identifying modified proteins (e.g., glycosylation, phosphorylation) that are only found in the tumor. These antigens are referred to as “neoantigens.” Once a neoantigen has been identified, it can be used as the antigen for the albumin binding peptide conjugate or to derive a peptide antigen for an albumin binding peptide conjugate. In some embodiments, the multimeric proteins described herein include an MHC portion operably coupled with or without a linker domain to a peptide antigen derived from a neoantigen.

### Viral Antigens

In some embodiments, a peptide antigen may be generated from a viral antigen. A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

In some embodiments, the MHC peptide is derived from the human immunodeficiency virus (HIV) group antigens (Gag) protein. In some embodiments, the MHC peptide is the HLA-A02-restricted FLGKIWPSYK epitope (SEQ ID NO: 59).

### Bacterial Antigens

In some embodiments, a peptide antigen may be generated from a bacterial antigen. Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Haemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In some embodiments, a peptide antigen may be derived from a bacterial superantigen. Bacterial superantigens (SAGs) comprise a large family of disease-associated proteins that are produced predominantly by Staphylococcus aureus and Streptococcus pyogenes. SAGs function by simultaneously interacting with class II MHC and TCR molecules on antigen presenting cells and T lymphocytes, respectively (Sundberg E J, et al. Curr Opin Immunol. 2002; 14(1):36-44). Contrary to the processed antigenic peptides discussed above, SAGS bind to MHC molecules outside of their peptide binding grooves and interact predominantly with only the Vβ domains of TCRs, resulting in the stimulation of up to 20 percent of the entire T cell population. In this way, SAGs initiate a systemic release of inflammatory cytokines that results in various immune-mediated diseases including a condition known as toxic shock syndrome (TSS) that can ultimately lead to multi-organ failure and death. SAGs have also been implicated in the pathogeneses of arthritis, asthma and inflammatory bowel syndrome, and are classified as Category B Select Agents by the U.S. Centers for Disease Control and Prevention.

### Parasite Antigens

In other embodiments, a peptide antigen may be generated from a parasite antigen. Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

### Allergens and Environmental Antigens

In some embodiments, a peptide antigen can be generated from an allergen or environmental antigen. An allergen or environmental antigen, may be, for example, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula\ alder (Alnus), hazel (Corylus), hornbeam (Carpinus) and olive (Olea), cedar {Cryptomeria and Juniperus), Plane tree (Platanus), the order of Poales including e.g., grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

### Exemplary Soluble Multimeric Fusion Proteins

### Exemplary Multimeric TCR-Immunoglobulin Fusion Proteins

In some embodiments, the disclosure provides soluble multimeric fusion proteins, wherein each fusion protein in the multimer comprises a soluble TCR polypeptide linked to an immunoglobulin framework (e.g., immunoglobulin heavy chain constant region or immunoglobulin light chain constant region) via a multimerization domain.

In some embodiments, the soluble, multimeric TCR-immunoglobulin fusion protein comprises a first fusion protein comprising a TCR α variable domain operably linked to a first leucine zipper domain that is operably linked to an immunoglobulin heavy chain constant region or fragment thereof, and a second fusion protein comprising a TCR β variable domain operably linked to a second leucine zipper domain that is operably linked to an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric TCR-immunoglobulin protein that is a TCR dimer. In some embodiments, the first leucine zipper domain is a LZL leucine zipper domain (SEQ ID NO: 6). In some embodiments, the second leucine zipper domain is LZR leucine zipper domain (SEQ ID NO: 8).

In some embodiments, the soluble, multimeric TCR-immunoglobulin fusion protein comprises a first fusion protein comprising a TCR α domain (e.g., TCR α variable region and a constant region) operably linked to a first leucine zipper domain that is further operably linked to an immunoglobulin heavy chain constant region or fragment thereof and a second fusion protein comprising a TCR β domain (e.g., TCR β variable region and β constant region) operably linked to a second leucine zipper domain that is operably linked to an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric TCR-immunoglobulin protein that is a TCR dimer. In some embodiments, the first leucine zipper domain is a LZL leucine zipper domain (SEQ ID NO: 6). In some embodiments, the second leucine zipper domain is LZR leucine zipper domain (SEQ ID NO: 8). In some embodiments, the first fusion protein comprises a TCR α domain comprising an amino acid sequence set forth by SEQ ID NO: 64 (HERV-K TCRalpha) and the second fusion protein comprises a TCR β domain comprising an amino acid sequence set forth by SEQ ID NO: 66 (HERV-K TCRbeta). In some embodiments, the first fusion protein comprises a TCR α domain comprising an amino acid sequence set forth by SEQ ID NO: 76 (FK10 TCRalpha) and the second fusion protein comprises a TCR β domain comprising an amino acid sequence set forth by SEQ ID NO: 78 (FK10 TCRbeta).

In some embodiments, the soluble, multimeric TCR-immunoglobulin fusion protein comprises a first fusion protein comprising a TCR α variable region operably linked to a TCR β domain (e.g., TCR β variable region and β constant region) that is further operably linked to a first leucine zipper domain that is further operably linked to an immunoglobulin heavy chain constant region or fragment thereof and a second fusion protein comprising a TCR α variable region operably linked to a TCR β domain (e.g., TCR β variable region and β constant region) that is further operably linked to a second leucine zipper domain that is operably linked to an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric TCR-immunoglobulin protein that is a TCR tetramer. In some embodiments, the first leucine zipper domain is a LZL leucine zipper domain (SEQ ID NO: 6). In some embodiments, the second leucine zipper domain is LZR leucine zipper domain (SEQ ID NO: 8).

In some embodiments, the soluble, multimeric TCR-immunoglobulin fusion protein comprises at least one fusion protein comprising a TCR α variable region operably linked to a TCR β domain (e.g., TCR β variable region and β constant region) that is further operably linked to a leucine zipper domain that is further operably linked to an immunoglobulin heavy chain constant region or fragment thereof. In some embodiments, the leucine zipper domain is a LZL leucine zipper domain (SEQ ID NO: 6). In some embodiments, the leucine zipper domain is a LZR leucine zipper domain (SEQ ID NO: 8). In some embodiments, the multimeric TCR-immunoglobulin fusion protein comprises two fusion proteins, wherein the immunoglobulin heavy chain of the first fusion protein and the immunoglobulin heavy chain of the second fusion protein form an immunoglobulin framework, thereby forming a soluble, multimeric TCR-immunoglobulin fusion protein that is a TCR receptor dimer.

In some embodiments, the soluble, multimeric TCR-immunoglobulin fusion protein comprises at least one fusion protein comprising a TCR α variable region operably linked to a TCR β domain (e.g., TCR β variable region and β constant region) that is further operably linked to a collagen-like trimerization domain that is further operably linked to an immunoglobulin heavy chain constant region or fragment thereof. In some embodiments, the collagen-like trimerization domain comprises an amino acid sequence set forth by SEQ ID NO: 60 (GPP10). In some embodiments, the soluble, multimeric TCR-immunoglobulin fusion protein comprises three fusion proteins, wherein the fusion proteins are multimerized via the collagen-like trimerization domain, thereby forming a multimeric TCR-immunoglobulin fusion protein that is a TCR trimer.

### Exemplary Multimeric MHC Class I-Immunoglobulin Fusion Proteins

In some embodiments, the disclosure provides soluble multimeric fusion proteins, wherein each fusion protein in the multimer comprises a soluble MHC class I polypeptide linked to an immunoglobulin framework (e.g., immunoglobulin heavy chain constant region or immunoglobulin light chain constant region) via a multimerization domain.

In some embodiments, the soluble, multimeric MHC class I-immunoglobulin fusion protein comprises a fusion protein comprising a β2-microglobulin operably linked to a MHC class I α chain that is further operably linked to a leucine zipper domain that is further operably linked to an immunoglobulin heavy chain constant region or fragment thereof. In some embodiments, the leucine zipper domain is a LZL leucine zipper domain (SEQ ID NO: 6). In some embodiments, the leucine zipper domain is a LZR leucine zipper domain (SEQ ID NO: 8). In some embodiments, the multimeric MHC class I-immunoglobulin fusion protein comprises two fusion proteins, wherein the immunoglobulin heavy chain of the first fusion protein and the immunoglobulin heavy chain of the second fusion protein form an immunoglobulin framework, thereby forming a soluble, multimeric MHC class I-immunoglobulin fusion protein that is a MHC class I receptor dimer.

In some embodiments, the soluble, multimeric MHC class I-immunoglobulin fusion protein comprises a first fusion protein comprising a β2-microglobulin operably linked to a MHC class I α chain that is further operably linked to a first leucine zipper domain that is further operably linked to an immunoglobulin heavy chain constant region or fragment thereof, and a second fusion protein comprising a β2-microglobulin operably linked to a MHC class I α chain that is further operably linked to a second leucine zipper domain that is further operably linked to an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric MHC Class I-immunoglobulin fusion protein that is an MHC Class I receptor tetramer. In some embodiments, the first leucine zipper domain is a LZL leucine zipper domain (SEQ ID NO: 6). In some embodiments, the second leucine zipper domain is a LZR leucine zipper domain (SEQ ID NO: 8).

In some embodiments, the soluble, multimeric MHC class I-immunoglobulin fusion protein comprises at least one fusion protein comprising a β2-microglobulin operably linked to a MHC class I α chain that is further operably linked to a collagen-like trimerization domain that is further operably linked to an immunoglobulin heavy chain constant region or fragment thereof. In some embodiments, the collagen-like trimerization domain comprises an amino acid sequence set forth by SEQ ID NO: 60 (GPP10). In some embodiments, the soluble, multimeric MHC class I-immunoglobulin fusion protein comprises three fusion proteins, wherein the fusion proteins are multimerized via the collagen-like trimerization domain, thereby forming a multimeric MHC class I-immunoglobulin fusion protein that is a MHC class I receptor trimer.

In some embodiments, the soluble, multimeric MHC class I-immunoglobulin fusion protein comprises at least one fusion protein comprising a peptide antigen operably linked to a β2-microglobulin operably linked to a MHC class I α chain that is further operably linked to a leucine zipper domain that is further operably linked to an immunoglobulin heavy chain constant region or fragment thereof. In some embodiments, the leucine zipper domain is a LZL leucine zipper domain (SEQ ID NO: 6). In some embodiments, the leucine zipper domain is a LZR leucine zipper domain (SEQ ID NO: 8). In some embodiments, the multimeric MHC class I-immunoglobulin fusion protein comprises two fusion proteins, wherein the immunoglobulin heavy chain of the first fusion protein and the immunoglobulin heavy chain of the second fusion protein form an immunoglobulin framework, thereby forming a soluble, multimeric MHC class I-immunoglobulin fusion protein that is a MHC class I receptor dimer.

In some embodiments, the soluble, multimeric MHC class I-immunoglobulin fusion protein comprises a first fusion protein comprising a peptide antigen operably linked to a β2-microglobulin that is further operably linked to a MHC class I α chain that is further operably linked to a first leucine zipper domain that is further operably linked to an immunoglobulin heavy chain constant region or fragment thereof, and a second fusion protein comprising a peptide antigen operably linked to a β2-microglobulin that is further operably linked to a MHC class I α chain that is further operably linked to a second leucine zipper domain that is further operably linked to an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric MHC Class I-immunoglobulin fusion protein that is an MHC Class I receptor tetramer. In some embodiments, the first leucine zipper domain is a LZL leucine zipper domain (SEQ ID NO: 6). In some embodiments, the second leucine zipper domain is a LZR leucine zipper domain (SEQ ID NO: 8).

In some embodiments, the soluble, multimeric MHC class I-immunoglobulin fusion protein comprises at least one fusion protein comprising a peptide antigen operably linked to a β2-microglobulin that is further operably linked to a MHC class I α chain that is further operably linked to a collagen-like trimerization domain that is further operably linked to an immunoglobulin heavy chain constant region or fragment thereof. In some embodiments, the collagen-like trimerization domain comprises an amino acid sequence set forth by SEQ ID NO: 60 (GPP10). In some embodiments, the soluble, multimeric MHC class I-immunoglobulin fusion protein comprises three fusion proteins, wherein the fusion proteins are multimerized via the collagen-like trimerization domain, thereby forming a multimeric MHC class I-immunoglobulin fusion protein that is a MHC class I receptor trimer.

### Exemplary Multimeric MHC Class II-Immunoglobulin Fusion Proteins

In some embodiments, the disclosure provides soluble multimeric fusion proteins, wherein each fusion protein in the multimer comprises a soluble MHC class II polypeptide linked to an immunoglobulin framework (e.g., immunoglobulin heavy chain constant region or immunoglobulin light chain constant region) via a multimerization domain.

In some embodiments, the soluble, multimeric MHC class II-immunoglobulin fusion protein comprises at least one fusion protein comprising a MHC class II α domain operably linked to a MHC class II β domain that is further operably linked to a leucine zipper domain that is further operably linked to an immunoglobulin heavy chain constant region or fragment thereof. In some embodiments, the leucine zipper domain is a LZL leucine zipper domain (SEQ ID NO: 6). In some embodiments, the leucine zipper domain is a LZR leucine zipper domain (SEQ ID NO: 8). In some embodiments, the multimeric MHC class II-immunoglobulin fusion protein comprises two fusion proteins, wherein the immunoglobulin heavy chain of the first fusion protein and the immunoglobulin heavy chain of the second fusion protein form an immunoglobulin framework, thereby forming a soluble, multimeric MHC class II-immunoglobulin fusion protein that is a MHC class II receptor dimer.

In some embodiments, the soluble, multimeric MHC class II-immunoglobulin fusion protein comprises a first fusion protein comprising a MHC class II α domain operably linked to a MHC class II β domain that is further operably linked to a first leucine zipper domain that is further operably linked to an immunoglobulin heavy chain constant region or fragment thereof, and a second fusion protein comprising a MHC class II α domain operably linked to a MHC class II β domain that is further operably linked to a second leucine zipper domain that is further operably linked to an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric MHC Class II-immunoglobulin fusion protein that is an MHC Class II receptor tetramer. In some embodiments, the first leucine zipper domain is a LZL leucine zipper domain (SEQ ID NO: 6). In some embodiments, the second leucine zipper domain is a LZR leucine zipper domain (SEQ ID NO: 8).

In some embodiments, the soluble, multimeric MHC class II-immunoglobulin fusion protein comprises a first fusion protein comprising a MHC class II α domain that is operably linked to a first leucine zipper domain that is further operably linked to an immunoglobulin heavy chain constant region or fragment thereof, and a fusion protein comprising a MHC class II β domain that is operably linked to a second leucine zipper domain that is further operably linked to an immunoglobulin light chain constant region or fragment thereof, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric MHC Class II-immunoglobulin fusion protein that is an MHC Class II receptor dimer. In some embodiments, the first leucine zipper domain is a LZL leucine zipper domain (SEQ ID NO: 6). In some embodiments, the second leucine zipper domain is a LZR leucine zipper domain (SEQ ID NO: 8).

In some embodiments, the soluble, multimeric MHC class II-immunoglobulin fusion protein comprises at least one fusion protein comprising a MHC class II α domain operably linked to a MHC class II β domain that is further operably linked to a collagen-like trimerization domain that is further operably linked to an immunoglobulin Fc domain. In some embodiments, the collagen-like trimerization domain comprises an amino acid sequence set forth by SEQ ID NO: 60 (GPP10). In some embodiments, the soluble, multimeric MHC class II-immunoglobulin fusion protein comprises three fusion proteins, wherein the fusion proteins are multimerized via the collagen-like trimerization domain, thereby forming a multimeric MHC class II-immunoglobulin fusion protein that is a trimer.

In some embodiments, the soluble, multimeric MHC class II-immunoglobulin fusion protein comprises a fusion protein comprising a peptide antigen operably linked to a MHC class II α domain that is further operably linked to a MHC class II β domain that is further operably linked to a leucine zipper domain that is further operably linked to an immunoglobulin heavy chain constant region or fragment thereof. In some embodiments, the leucine zipper domain is a LZL leucine zipper domain (SEQ ID NO: 6). In some embodiments, the leucine zipper domain is a LZR leucine zipper domain (SEQ ID NO: 8). In some embodiments, the multimeric MHC class II-immunoglobulin fusion protein comprises two fusion proteins, wherein the immunoglobulin heavy chain of the first fusion protein and the immunoglobulin heavy chain of the second fusion protein form an immunoglobulin framework, thereby forming a soluble, multimeric MHC class II-immunoglobulin fusion protein that is a MHC class II receptor dimer.

In some embodiments, the soluble, multimeric MHC class II-immunoglobulin fusion protein comprises a first fusion protein comprising a peptide antigen operably linked to a MHC class II α domain that is further operably linked to a MHC class II β domain that is further operably linked to a first leucine zipper domain that is further operably linked to an immunoglobulin heavy chain constant region or fragment thereof, and a second fusion protein comprising a peptide antigen operably linked to a MHC class II α domain that is further operably linked to a MHC class II β domain that is further operably linked to a second leucine zipper domain that is further operably linked to an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric MHC Class II-immunoglobulin fusion protein that is an MHC Class II receptor tetramer. In some embodiments, the first leucine zipper domain is a LZL leucine zipper domain (SEQ ID NO: 6). In some embodiments, the second leucine zipper domain is a LZR leucine zipper domain (SEQ ID NO: 8).

In some embodiments, the soluble, multimeric MHC class II-immunoglobulin fusion protein comprises a first fusion protein comprising a peptide antigen operably linked to a MHC class II α domain that is operably linked to a first leucine zipper domain that is further operably linked to an immunoglobulin heavy chain constant region or fragment thereof, and a second fusion protein comprising a MHC class II β domain that is operably linked to a second leucine zipper domain that is further operably linked to an immunoglobulin light chain constant region or fragment thereof, wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric MHC Class II-immunoglobulin fusion protein that is an MHC Class II receptor dimer. In some embodiments, the first leucine zipper domain is a LZL leucine zipper domain (SEQ ID NO: 6). In some embodiments, the second leucine zipper domain is a LZR leucine zipper domain (SEQ ID NO: 8).

In some embodiments, the soluble, multimeric MHC class II-immunoglobulin fusion protein comprises at least one fusion protein comprising a peptide antigen operably linked to a MHC class II α domain operably linked to a MHC class II β domain that is further operably linked to a collagen-like trimerization domain that is further operably linked to an immunoglobulin heavy chain constant region or fragment thereof. In some embodiments, the collagen-like trimerization domain comprises an amino acid sequence set forth by SEQ ID NO: 60 (GPP10). In some embodiments, the soluble, multimeric MHC class II-immunoglobulin fusion protein comprises three fusion proteins, wherein the fusion proteins are multimerized via the collagen-like trimerization domain, thereby forming a multimeric MHC class II-immunoglobulin fusion protein that is a MHC class II receptor trimer.

### Nucleic Acids, Vectors and Host Cells

The disclosure also provides isolated nucleic acids encoding the soluble multimeric fusion proteins and portions thereof disclosed here. In some embodiments, the nucleic acids are present in vectors, optionally expression vectors. In some embodiments, the nucleic acids are present in expression vectors under transcriptional and optionally translational control of regulatory sequences sufficient to express the nucleic acids in cells, optionally prokaryotic cells and optionally eukaryotic cells. In some embodiments, the cells are mammalian cells, and in some embodiments the mammalian cells are human cells.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The nucleic acids can be constructed based on chemical synthesis and/or enzymatic ligation reactions using procedures known in the art (see e.g., Sambrook & and Russell, 2001; and Ausubel et al., 1989). For example, a nucleic acid can be chemically synthesized using naturally-occurring nucleotides and/or variously modified nucleotides designed to increase the biological stability of the molecules and/or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N.sup.6-isopentenyladenine, 1-methylguanine, 1-methylinosine, 2,2-dimethylguanine, 2-methyladenine, 2-methylguanine, 3-methylcytosine, 5-methylcytosine, N.sup.6-substituted adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N.sup.6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl)uracil, and 2,6-diaminopurine. Alternatively or in addition, one or more of the nucleic acids of the presently disclosed subject matter can be purchased from a commercial source such as, but not limited to Macromolecular Resources of Fort Collins, Colo. and Synthegen of Houston, Tex.

In another aspect, the nucleic acids of the presently disclosed subject matter can in some embodiments be incorporated into a vector, optionally an expression vector, further optionally a recombinant expression vector. The presently disclosed subject matter thus provides in some embodiments recombinant expression vectors comprising any of the nucleic acids disclosed herein. As used herein, the phrases “expression vector” and “recombinant expression vector” refer to genetically-modified oligonucleotide and/or polynucleotide constructs that permit the expression of an mRNA, protein, polypeptide, and/or peptide by a host cell, when the construct comprises a nucleotide sequence encoding the mRNA, protein, polypeptide, and/or peptide, and the vector is contacted with the cell under conditions sufficient to have the mRNA, protein, polypeptide, and/or peptide expressed within the cell. Expression vectors can comprise any type of nucleotides, including, but not limited to DNA and RNA, which can be single-stranded or double-stranded, synthesized or obtained in part from natural sources, and which can contain natural, non-natural, and/or altered nucleotides.

Suitable vectors include those designed for propagation and expansion or for expression or both, such as plasmids and viruses. In some embodiments, an expression vector comprises regulatory sequences, including but not limited to transcription, translation, initiation, and termination codons, which are specific to the type of host (e.g., bacterium, fungus, plant, or animal) into which the vector is to be introduced, as appropriate and taking into consideration whether the vector is DNA- or RNA-based. The expression vectors of the presently disclosed subject matter can be prepared using standard recombinant DNA techniques described in, for example, Sambrook & Russell, 2001; Ausubel et al., 1989. Constructs of expression vectors, which can be circular or linear, can be prepared to contain a replication system functional in a prokaryotic or eukaryotic host cell. Replication systems can be derived, e.g., from ColE1, 2μ plasmid, γ, SV40, bovine papilloma virus, and the like.

In some embodiments, the vector can be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as γG10, .lamda.GT11, γZapII (Stratagene), γEMBL4, and γNM1149, also can be used. Examples of plant expression vectors include pBI01, pBI101.2, pBI101.3, pBI121, and pBIN19 (Clontech). Examples of animal expression vectors include pEUK-C1, pMAM, and pMAMneo (Clontech).

In some embodiments, the recombinant expression vector is a viral vector, including but not limited both integrating and non-integrating viral vectors. Exemplary viral vectors include, but are not limited to adenoviral vectors, lentiviral vectors, retroviral vectors, episomal vectors, and non-episomal vectors, and are disclosed for example, in U.S. Pat. Nos. 8,119,772; 8,552,150; 6,277,633 and 6,521,457; and U.S. Patent Application Publication Nos. 2012/0135034 and 2008/0254008. Lentiviral vector systems are also commercially available from Cell Biolabs, Inc. of San Diego, Calif., United States of America and OriGene Technologies, Inc. of Rockville, Md., United States of America. In some embodiments, a vector is a viral episomal vector, optionally based on adenovirus and/or adeno-associated virus (AAV), for example, as described in WO 2002/085287. One example of a suitable non-viral episomal vector is disclosed in WO 1998/007876.

In some embodiments, the expression vector is a bicistronic or multicistronic expression vectors. In some embodiments, the bicistronic or multicistronic vector, comprises an internal ribosomal entry site (IRES). In some embodiments, the bicistronic or mulicistronic vector comprises an amino acid cleavage sequence amino-terminal to one or more of the encoded polypeptide components of the multimeric fusion protein. In some embodiments, the cleavage sequence comprises from about 2 to about 20 amino acids. In certain embodiments, the cleavage sequence comprises from about 2 to about 15, about 2 to about 10, or about 2 to about 5 amino acids.

In some embodiments, the self-cleaving amino acid sequence is derived from a 2A peptide. In certain embodiments, the self-cleaving amino acid sequence comprises a 2A peptide from porcine teschovirus-1 (P2A), equine rhinitis A virus (E2A), Thosea asigna virus (T2A), foot-and-mouth disease virus (F2A), or any combination thereof (see, e.g., Kim et al., PLOS One 6:e18556, 2011, which 2A nucleic acid and amino acid sequences are incorporated herein by reference in their entirety). In one embodiment, the bicistronic or multicistronic victor comprises a nucleic acid encoding a 2A peptide derived from porcine teschovirus-1 (P2A).

In some embodiments, a furin recognition site is inserted upstream of the 2A peptides. Insertion of a furin recognition sequences between a first encoded polypeptide and an encoded 2A peptide is useful for removal of 2A residues from the first encoded polypeptide as described by Chng, et al (2015) mAbs 7:403-4121; Fang, et al (2005) Nat. Biotech 23:584-590; and Fang, et al. (2007) Mol. Ther. 15:1153-1159 which are incorporated by reference herein. The furin recognition sequence comprises a minimal cleavage site of Arg-X-X-Arg. The preferred cleavage sequence is Arg-X-Lys/Arg-Arg.

In some embodiments, an expression vector can comprise a native or non-native promoter operably linked to a nucleotide sequence encoding one or more of the polypeptide components of the multimeric fusion protein provided herein. The selection of promoters, in some embodiments strong, weak, inducible, tissue-specific, and/or developmental-specific, is within the ordinary skill of the artisan. Similarly, the combining of a nucleotide sequence with a promoter is also within the skill of the artisan. The promoter can be in some embodiments a non-viral promoter or a viral promoter including, but not limited to a cytomegalovirus (CMV) promoter, an SV40 promoter, an RSV promoter, a promoter found in the long-terminal repeat of a retrovirus, etc.

In some embodiments, an expression vector of the disclosure can also include one or more marker genes, which allow for selection of transformed or transfected hosts. Marker genes can include biocide resistance, e.g., resistance to antibiotics, heavy metals, etc., complementation in an auxotrophic host to provide prototrophy, and the like. Suitable marker genes for an expression vectors can include, for example, neomycin/G418 resistance genes, hygromycin resistance genes, histidinol resistance genes, tetracycline resistance genes, and ampicillin resistance genes.

Further, expression vectors can in some embodiments be made to include a suicide gene. As used herein, the phrase “suicide gene” refers to a nucleotide sequence that causes a cell expressing the nucleotide sequence to die. A suicide gene can in some embodiments be a nucleotide sequence that confers sensitivity upon a cell expressing the nucleotide sequence as a transcription product and/or as a translation product to an agent (such as but not limited to a drug) such that when the cell is contacted with and/or exposed to the agent, the agent directly or indirectly causes the cell to die. Suicide genes are known in the art and include, for example, the Herpes Simplex Virus (HSV) thymidine kinase (TK) gene, cytosine daminase, purine nucleoside phosphorylase, and nitroreductase (see e.g., Springer, 2004).

In certain embodiments, the vector is a single expression vector which co-expresses the components of the multimeric fusion protein. In some embodiments, the vector comprises a nucleic acid encoding a first fusion protein comprising VαCα-X¹-Ig(Fc) and a second fusion protein comprising VβCβ-X²-Ig(C_{L}) separated by a P2A peptide. In some embodiments, the Ig(Fc) comprises C_{H}1-C_{H}2-C_{H}3. In some embodiments, the vector comprises a nucleic acid encoding a first fusion protein comprising VαVβCβ-X¹-Ig(Fc) and a second fusion protein comprising VβCβ-X²-Ig(C_{L}) separated by a P2A peptide. In some embodiments, the vector comprises a nucleic acid encoding a first fusion protein comprising β2M-MHCIα-X¹-Ig(Fc) and a second fusion protein comprising MHCIα-X²-Ig(Fc) separated by a P2A peptide. In some embodiments, the vector comprises a nucleic acid encoding a first fusion protein comprising β2M-MHCIα-X¹-Ig(Fc) and a second fusion protein comprising β2M-MHCIα-X²-Ig(C_{L}) separated by a P2A peptide. In some embodiments, the vector comprises a nucleic acid encoding a first fusion protein comprising Ag-β2M-MHCIα-X¹-Ig(Fc) and a second fusion protein comprising Ag-β2M-MHCIα-X²-Ig(C_{L}) separated by a P2A peptide. In some embodiments, the vector comprises a nucleic acid encoding a first fusion protein comprising MHCIIα-MHCIIβ-X¹-Ig(Fc) and a second fusion protein comprising MHCIIα-MHCIIβ-X²-Ig(Fc) separated by a P2A peptide. In some embodiments, the vector comprises a nucleic acid encoding a first fusion protein comprising MHCIIα-MHCIIβ-X¹-Ig(Fc) and a second fusion protein comprising Ag-MHCIIα-MHCIIβ-X²-Ig(C_{L}) separated by a P2A peptide. In some embodiments, the nucleic acid further comprises a furin recognition sequence downstream of the encoded first fusion protein and upstream of the encoded P2A peptide.

The disclosure further provides host cells which express a soluble, multimeric fusion proteins disclosed herein. Expression constructs provided herein can be introduced into host cells using any technique known in the art. These techniques include, but are not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, and calcium phosphate-mediated transfection.

Any of a large number of available and well-known host cells may be used. The selection of a particular host is dependent upon a number of factors recognized by the art. These include, for example, compatibility with the chosen expression vector, toxicity of the peptides encoded by the nucleic acid, rate of transformation or transfection, ease of recovery of the peptides, expression characteristics, bio-safety and costs. A balance of these factors must be struck with the understanding that not all hosts may be equally effective for the expression of a particular nucleic acid sequence. Within these general guidelines, useful microbial hosts include bacteria (such as E. coli), yeast (such as Saccharomyces) and other fungi, insects, plants, mammalian (including human) cells in culture, or other hosts known in the art.

Thus a host cell can include a prokaryotic or eukaryotic cell in which production of the fusion protein is specifically intended. Thus host cells specifically include yeast, fly, worm, plant, fungal, frog, mammalian cells and organs that are capable of propagating nucleic acid encoding the fusion. Non-limiting examples of mammalian cell lines which can be used include CHO dhfr-cells (Urlaub and Chasm, Proc. Natl. Acad. Sci. USA, 77:4216 (1980)), 293 cells (Graham et al., J. Gen. Virol., 36:59 (1977)) or myeloma cells like SP2 or NSO (Galfre and Milstein, Meth. Enzymol., 73(B):3 (1981)).

### Methods of Production

Methods of producing soluble, multimeric fusion proteins of the disclosure, either recombinantly or by covalently linking two protein segments, are well known. Preferably, fusion proteins are expressed recombinantly, as products of expression constructs. In some embodiments, the disclosure provides expression constructs which comprise a polynucleotide which encodes one or more fusion proteins in which an immunoglobulin framework is C-terminal to the soluble TCR or MHC polypeptide.

In some embodiments, polynucleotides in expression constructs provided by the disclosure can comprise nucleotide sequences coding for a signal sequence. Expression of these constructs results in secretion of a soluble multimeric fusion protein of the disclosure. The multimeric fusion proteins described herein largely may be made in transformed or transfected host cells using recombinant DNA techniques. Next, the transformed or transfected host is cultured and purified. Host cells may be cultured under conventional fermentation or culture conditions so that the desired compounds are expressed. Such fermentation and culture conditions are well known in the art. The expressed polypeptides can be purified from the expression system using routine biochemical procedures, and can be used, e.g., as therapeutic agents, as described herein.

The multimeric fusion proteins may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al., Biochem Intl 1985; 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods for molecule expression/synthesis are generally known in the art to one of ordinary skill.

### Additional Modifications

In some embodiments, the soluble, multimeric fusion protein is conjugated to an active agent. In some embodiments, the active agent is selected from the group consisting of a detectable label, an immunostimulatory molecule, and a therapeutic agent. In some embodiments, the detectable label is selected from the group consisting of biotin, streptavidin, an enzyme or catalytically active fragment thereof, a radionuclide, a nanoparticle, a paramagnetic metal ion, or a fluorescent, phosphorescent, or chemiluminescent molecule. In some embodiments, the therapeutic agent is selected from the group consisting of an alkylating agent, an antimetabolite, a natural product having pharmacological activity, a mitotic inhibitor, an antibiotic, a cytotoxic agent, and a chemotherapeutic agent.

In some embodiments, suitable labels include biotin, streptavidin, a cell toxin of, e.g., plant or bacterial origin such as, e.g., diphtheria toxin (DT), shiga toxin, abrin, cholera toxin, ricin, saporin, pseudomonas exotoxin (PE), pokeweed antiviral protein, or gelonin. Biologically active fragments of such toxins are well known in the art and include, e.g., DT A chain and ricin A chain. Additionally, the toxin can be an agent active at the cell surface such as, e.g., phospholipase enzymes (e.g., phospholipase C). See e.g., Moskaug et al., 1989; Pastan et al., 1986; Pastan et al., 1992; Olsnes & Pihl, 1981; PCT International Patent Application Publication No. WO 1994/29350; PCT International Patent Application Publication No. WO 1994/04689; and U.S. Pat. No. 5,620,939 for disclosure relating to making and using proteins comprising effectors or tags. An example of a tag that performs a biotin acceptor function is a BirA tag, as described in Beckett et al., 1999. As further described in Examples below, a BirA tag sequence can be included in a TCR, TCR-like molecule, and/or a portion thereof to promote biotinylation of the protein. Further, a tag can be a chemotherapeutic drug such as, e.g., vindesine, vincristine, vinblastin, methotrexate, adriamycin, bleomycin, or cisplatin.

In some embodiments, a radioactive label can be directly conjugated to the amino acid backbone of the polypeptide. Alternatively, the radioactive label can be included as part of a larger molecule (e.g., ¹²⁵I in meta-[¹²⁵I]iodophenyl-N-hydroxysuccinimide ([¹²⁵I]mIPNHS) which binds to free amino groups to form meta-iodophenyl (mIP) derivatives of relevant proteins (see, e.g., Rogers et al. (1997) J Nucl Med 38:1221-1229) or chelate (e.g., to DOTA or DTPA) which is in turn bound to the protein backbone. Methods of conjugating the radioactive labels or larger molecules/chelates containing them to the polypeptides described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Other suitable detectable labels include polyhistidine, fluorescent label, chemiluminescent label, nuclear magnetic resonance active label, chromophore label, positron emitting isotope detectable by a positron emission tomography (“PET”) scanner, enzymatic markers such as beta-galactosidase and peroxidase including horse radish peroxidase, a nanoparticle, a paramagnetic metal ion, a contrast agent or an antigenic tag. For example, suitable fluorescent labels include, but are not limited to, a ¹⁵²Eu label, a fluorescein label, an isothiocyanate label, a rhodamine label, a phycoerythrin label, a phycocyanin label, an allophycocyanin label, an o-phthaldehyde label, a Texas Red label, a fluorescamine label, a lanthanide phosphor label, a fluorescent protein label, for example a green fluorescent protein (GFP) label, or a quantum dot label. Examples of chemiluminescent labels include a luminal label, an isoluminal label, an aromatic acridinium ester label, an imidazole label, an acridinium salt label, an oxalate ester label, a luciferin label, a luciferase label, an aequorin label, etc.

Methods for conjugating a fluorescent label (sometimes referred to as a “fluorophore”) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating a polypeptide, with the fluorophore under conditions that facilitate binding of the fluorophore to the protein. See, e.g., Welch and Redvanly (2003) “Handbook of Radiopharmaceuticals: Radiochemistry and Applications,” John Wiley and Sons (ISBN 0471495603).

In some embodiments, the polypeptides described herein can be glycosylated. In some embodiments, a polypeptide described herein can be subjected to enzymatic or chemical treatment, or produced from a cell, such that the polypeptide has reduced or absent glycosylation. Methods for producing polypeptides with reduced glycosylation are known in the art (e.g., U.S. Pat. No. 6,933,368; Wright et al. (1991) EMBO J 10(10):2717-2723; and Co et al. (1993) Mol Immunol 30:1361).

Enzyme markers that may be used include any readily detectable enzyme activity or enzyme substrate. Such enzymes include malate dehydrogenase, staphylococcal nuclease, delta-5-steroid isomerase, alcohol dehydrogenase, glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, acetylcholine esterase, luciferase, and DNA polymerase.

### Assays for Characterization

The soluble multimeric proteins of the disclosure can be characterized for their specificity and binding affinity for particular antigens using any immunological or biochemical based method known in the art. For example, specific binding of a soluble TCR or MHC, may be determined for example using immunological or biochemical based methods such as, but not limited to, an ELISA assay, SPR assays, immunoprecipitation assay, affinity chromatography, and equilibrium dialysis as described above Immunoassays which can be used to analyze immunospecific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. Such assays are routine and well known in the art.

Methods of testing a TCR-Igg of the disclosure for an ability to recognize a target and/or a cell and for antigen specificity are known in the art. For example, methods of measuring the release of cytokines (e.g., interferon-γ (IFNγ), granulocyte/monocyte colony stimulating factor (GM-CSF), tumor necrosis factor α (TNF-α), or interleukin 2 (IL-2). In addition, immune function can be evaluated by measurement of cellular cytoxicity. Binding of TCR-Igg can also be characterized by flow cytometry in which target cells loaded with cognate peptide are co-incubated with a serial titration of TCR-Igg that is either directly conjugated to a fluorophore or that can be further stained with a secondary fluorophore-conjugated anti-Igg antibody.

### Compositions

In one aspect, the disclosure provides for a pharmaceutical composition comprising a soluble, multimeric protein (e.g., TCR-Igg, or pMHC-Igg) described herein, with a pharmaceutically acceptable diluent, carrier, solubilizer, emulsifier, preservative and/or adjuvant.

In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the multimeric fusion protein, or isolated monoclonal antibody, or antigen binding fragment, described herein.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising a multimeric fusion protein, or isolated monoclonal antibody, or antigen binding fragment, described herein, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising a multimeric fusion protein, or isolated monoclonal antibody, or antigen binding fragment, described herein, can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a multimeric fusion protein, described herein, in a pharmaceutically acceptable vehicle. In certain embodiments, a vehicle for parenteral injection is sterile distilled water in which a multimeric fusion protein, described herein, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, a multimeric fusion protein can be formulated as a dry powder for inhalation. In certain embodiments, an inhalation solution comprising a multimeric fusion protein can be formulated with a propellant for aerosol delivery. In certain embodiments, solutions can be nebulized. Pulmonary administration is further described in PCT application No. PCT/US94/001875, which describes pulmonary delivery of chemically modified proteins.

In certain embodiments, it is contemplated that formulations can be administered orally. In certain embodiments, a multimeric fusion protein that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of the multimeric fusion protein, or isolated monoclonal antibody, or antigen binding fragment. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of the multimeric fusion protein in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving a multimeric fusion protein in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising a multimeric fusion protein of the disclosure to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which a multimeric fusion protein are being used, the route of administration, and the size (body weight, body surface or organ size) and/or condition (the age and general health) of the patient. In certain embodiments, the clinician can titer the dosage and modify the route of administration to obtain the optimal therapeutic effect.

In certain embodiments, the frequency of dosing will take into account the pharmacokinetic parameters of a multimeric fusion protein in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intraocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration. In certain embodiments, it can be desirable to use a pharmaceutical composition comprising a multimeric fusion protein, in an ex vivo manner. In such instances, cells, tissues and/or organs that have been removed from the patient are exposed to a pharmaceutical composition comprising a multimeric fusion protein, after which the cells, tissues and/or organs are subsequently implanted back into the patient.

In certain embodiments, a multimeric fusion protein, can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

Pharmaceutical compositions described herein also can be administered in combination therapy, i.e., combined with other agents. For example, the combination therapy can include a composition described herein with at least one or more additional therapeutic agents. Co-administration with other multimeric fusion proteins is also encompassed by the disclosure.

### Administration

The compositions described herein are useful in, inter alia, methods for treating or preventing a variety of autoimmune and related disorders, allergy, inflammation, and/or graft or transplant rejection in a subject. The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), or intrathecal injection (IT). The injection can be in a bolus or a continuous infusion.

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some embodiments, a multimeric fusion protein of the present disclosure is therapeutically delivered to a subject by way of local administration.

A suitable dose of a multimeric fusion protein of the present disclosure, which dose is capable of treating or preventing immunological disorders in a subject, can depend on a variety of factors including, e.g., the age, sex, and weight of a subject to be treated and the particular inducer compound used. Other factors affecting the dose administered to the subject include, e.g., the type or severity of the immunological disorder. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will also depend upon the judgment of the treating medical practitioner (e.g., doctor or nurse). Suitable dosages are described herein.

A pharmaceutical composition can include a therapeutically effective amount of a multimeric fusion protein of the present disclosure described herein. Such effective amounts can be readily determined by one of ordinary skill in the art based, in part, on the effect of the administered antibody, or the combinatorial effect of the antibody and one or more additional active agents, if more than one agent is used. A therapeutically effective amount of a multimeric fusion protein described herein can also vary according to factors such as the disease state, age, sex, and weight of the individual, and the ability of the antibody (and one or more additional active agents) to elicit a desired response in the individual, e.g., reduction in tumor growth. For example, a therapeutically effective amount of a fusion protein can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the a multimeric fusion proteins of the present disclosure can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

In some embodiments, the composition contains any of the multimeric fusion protein of the present disclosure and one or more (e.g., two, three, four, five, six, seven, eight, nine, 10, or 11 or more) additional therapeutic agents such that the composition as a whole is therapeutically effective. For example, a composition can contain a multimeric fusion protein of the present disclosure and an anti-inflammatory agent, wherein the a multimeric fusion protein and agent are each at a concentration that when combined are therapeutically effective for treating or preventing an immunological disorder in a subject.

Toxicity and therapeutic efficacy of such compositions can be determined by known pharmaceutical procedures in cell cultures or experimental animals (e.g., animal models of any of the cancers described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. A multimeric fusion protein that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of a multimeric fusion protein of the present disclosure lies generally within a range of circulating concentrations that include an ED₅₀ with little or no toxicity. The dosage may vary within this range depending upon the dosage form employed and the route of administration utilized. For a multimeric fusion protein of the present disclosure, the therapeutically effective dose can be estimated initially from cell culture assays. A dose can be formulated in animal models to achieve a circulating plasma concentration range that includes the IC₅₀ (i.e., the concentration of the fusion protein which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

In some embodiments, the methods can be performed in conjunction with other therapies for autoimmune and related diseases. For example, the composition can be administered to a subject at the same time, prior to, or after, radiation, surgery, targeted or cytotoxic chemotherapy, anti-inflammatory therapy, steroid therapy, chemoradiotherapy, hormone therapy, immunotherapy, immunosuppressive therapy, antithyroid therapy, antibiotic therapy, gene therapy, cell transplant therapy, precision medicine, genome editing therapy, or other pharmacotherapy.

In some embodiments, a fusion protein, or an antibody or an antigen-binding fragment thereof described herein can be administered to a subject as a monotherapy. Alternatively, as described above, the fusion protein, or the antibody or fragment thereof can be administered to a subject as a combination therapy with another treatment, e.g., another treatment for an autoimmune or related disease. For example, the combination therapy can include administering to the subject (e.g., a human patient) one or more additional agents that provide a therapeutic benefit to a subject who has, or is at risk of developing, an autoimmune or related diseases. In some embodiments, a fusion protein, or an antibody and the one or more additional active agents are administered at the same time. In other embodiments, the fusion protein, or antibody or antigen binding fragment thereof is administered first in time and the one or more additional active agents are administered second in time. In some embodiments, the one or more additional active agents are administered first in time and the fusion protein, or antibody or antigen binding fragment thereof is administered second in time.

A multimeric fusion protein described herein can replace or augment a previously or currently administered therapy. For example, upon treating with a multimeric fusion protein, administration of the one or more additional active agents can cease or diminish, e.g., be administered at lower levels. In some embodiments, administration of the previous therapy can be maintained. In some embodiments, a previous therapy will be maintained until the level of the multimeric fusion protein reaches a level sufficient to provide a therapeutic effect. The two therapies can be administered in combination.

Monitoring a subject (e.g., a human patient) for an improvement in an immunological disorder or disease means evaluating the subject for a change in a disease parameter, e.g., a reduction in inflammation. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for an immunological disorder or related disease described herein.

### Use

The compositions described herein can be used in a number of in vitro, ex vivo, and in vivo applications. For example, the multimeric fusion proteins described herein can be contacted to cultured cells in vitro or in vivo, or administered to a subject (e.g., a mammal, such as a human) to modulate the activation of an immune cell (e.g., a T cell) and/or modulate an immune response to an antigen of interest. For example, a T cell or a plurality of immune cells comprising T cells can be contacted with one or more of the multimeric fusion proteins described herein in an amount effective to enhance activation of the immune cell by the antigen. The effective amount of the agent is the amount required to modulate the activation of the immune cell by the antigen, that is, to produce an enhanced or reduced activation level to the antigen as compared to the level of activation produced by the immune cell in the absence of the agent

### Methods of Detection

In some embodiments, the presently disclosed soluble, multimeric fusion proteins, and portions thereof can be employed as diagnostic agents. By way of example and not limitation, the presently disclosed multimeric fusion proteins, and portions thereof can be employed in a detection and/or diagnostic assay such as but not limited to immunohistochemistry to localize their cognate MHC antigens in samples from subjects. For example, a tumor biopsy could be contacted with a multimeric fusion protein and/or a portion thereof that has been conjugated with a detectable label under conditions sufficient for the presently disclosed multimeric fusion protein, and portions thereof to bind to its antigen/epitope, and this binding can be detected using standard techniques. Such an approach can be used, for example, for assaying tumor biopsies to determine whether the cells present in the biopsy express a given antigen and, in some embodiments, to what extent the antigen is expressed in the tumor cells. For those antigens that are expressed specifically by tumor cells, such an approach can also be used to assess tumor margins by determining whether or not the cells at the periphery of a tumor biopsy express or do not express a given antigen.

In some embodiments, the soluble, multimeric fusion protein is labeled with a detectable agent as described herein. In some embodiments, the soluble, multimeric fusion protein is labeled with an agent suitable for PET for detection of a cancer specific antigen expression in patients in a non-invasive manner Such methods are suitable for early cancer diagnosis, monitoring of cancer progression, and monitoring of patient response to cancer therapies.

### Therapeutic Applications

In another aspect, the disclosure provides methods of preventing and/or treating a variety of immunological disorders using the multimeric fusion proteins provided herein.

In some embodiments, provided is a method of activating antigen-specific T cells by administering a soluble, multimeric fusion protein of the disclosure in an amount sufficient to induce a T cell response. In some embodiments, the soluble multimeric fusion protein is a multimeric TCR-fusion protein. In some embodiments, the soluble multimeric fusion protein is a multimeric MHC-fusion protein.

In some embodiments, the disclosure provides a method for treating or preventing an allergic reaction in subject in need thereof by administering a soluble, multimeric fusion protein of the disclosure in an amount sufficient to suppress or reduce a T cell response associated with the allergy. In some embodiments, the soluble multimeric fusion protein is a multimeric TCR-fusion protein. In some embodiments, the soluble multimeric fusion protein is a multimeric MHC-fusion protein.

In some embodiments, the disclosure provides a method for treating or preventing graft-versus-host disease a subject who has received or will receive an organ transplant or tissue graft, by administering a soluble, multimeric fusion protein of the disclosure in an amount sufficient to suppress or reduce an immune response to the transplant. In some embodiments, the soluble multimeric fusion protein is a multimeric TCR-fusion protein. In some embodiments, the soluble multimeric fusion protein is a multimeric MHC-fusion protein.

In some embodiments, the disclosure provides a method for treating an autoimmune disease in a subject by administering a soluble, multimeric fusion protein of the disclosure in an amount sufficient to suppress or reduce the autoimmune response. In some embodiments, the soluble multimeric fusion protein is a multimeric TCR-fusion protein. In some embodiments, the soluble multimeric fusion protein is a multimeric MHC-fusion protein. Autoimmune disorders which may be treated according to the methods of the invention include, but are not limited to, Crohn's disease, multiple sclerosis, myasthenia gravis, rheumatoid arthritis, Goodpasture's syndrome, T-cell mediated hepatitis, graft vs. host disease, autoimmune uveitis, and/or autoimmune diabetes.

In some embodiments, the disclosure provides a method for treating cancer in a subject by administering a soluble, multimeric fusion protein of the disclosure in an amount sufficient to induce or enhance an immune response to the cancer. In some embodiments, the soluble, multimeric protein binds to a cancer antigen. In some embodiments, the soluble multimeric fusion protein is a multimeric TCR-fusion protein. In some embodiments, the soluble multimeric fusion protein is a multimeric MHC-fusion protein.

In some embodiments, the disclosure provides a method for treating an infection caused by an infectious agent in a subject by administering a soluble, multimeric protein of the disclosure in an amount sufficient to induce or enhance an immune response to the infectious agent. In some embodiments, the soluble multimeric fusion protein is a multimeric TCR-fusion protein. In some embodiments, the soluble multimeric fusion protein is a multimeric MHC-fusion protein.

In some embodiments, the subject is afflicted with a persistent infectious disease (e.g., viral infectious diseases including HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. In addition, bacterial, fungal and other pathogenic infections are included, such as Aspergillus, Brugia, Candida, Chlamydia, Coccidia, Cryptococcus, Dirofilaria, Gonococcus, Histoplasma, Leishmania, Mycobacterium, Mycoplasma, Paramecium, pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum. Also included are National Institute of Allergy and Infectious Diseases (NIAID) priority pathogens. These include Category A agents, such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever) and related viruses); Category B agents, such as Coxiella burnetti (Q fever), Brucella species (brucellosis), Burkholderia mallei (glanders), alphaviruses (Venezuelan encephalomyelitis, eastern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain O157:H7, Vibrio cholerae, Cryptosporidium parvum; Category C agents, such as nipah virus, hantaviruses, tickborne hemorrhagic fever viruses, tickborne encephalitis viruses, yellow fever, and multidrug-resistant tuberculosis; helminths, such as Schistosoma and Taenia; and protozoa, such as Leishmania (e.g., L. mexicana) and Plasmodium.

### Kits

A kit can include a soluble, multimeric fusion protein as disclosed herein, and instructions for use. The kits may comprise, in a suitable container, one or more controls, and various buffers, reagents, enzymes and other standard ingredients well known in the art.

In some embodiments, the container can include at least one vial, well, test tube, flask, bottle, syringe, or other container means, into which a soluble, multimeric fusion protein may be placed, and in some instances, suitably aliquoted. Where an additional component is provided, the kit can contain additional containers into which this component may be placed. The kits can also include a means for containing a soluble, multimeric fusion protein, and any other reagent containers in close confinement for commercial sale. Such containers may include injection or blow-molded plastic containers into which the desired vials are retained. Containers and/or kits can include labeling with instructions for use and/or warnings.

In some embodiments, a kit comprises a multimeric protein fusion complex of the disclosure, and an optional pharmaceutically acceptable carrier, a composition of the disclosure, and an optional pharmaceutically acceptable carrier, or a pharmaceutical composition of the disclosure, and a package insert, wherein the kit comprises instructions for administration of the protein fusion, composition or pharmaceutical composition for treating or preventing an allergic reaction by suppressing or reducing a T cell response associated with the allergy in a subject in need thereof.

In some embodiments, a kit comprises a multimeric protein fusion complex of the disclosure, and an optional pharmaceutically acceptable carrier, a composition of the disclosure, and an optional pharmaceutically acceptable carrier, or a pharmaceutical composition of the disclosure, and a package insert, wherein the kit comprises instructions for administration of the protein fusion, composition or pharmaceutical composition for treating or preventing (GvHD) by suppressing or reducing an immune response to a transplant in a subject who has received or will receive an organ transplant or a tissue graft.

In some embodiments, a kit comprises a multimeric protein fusion complex of the disclosure, and an optional pharmaceutically acceptable carrier, a composition of the disclosure, and an optional pharmaceutically acceptable carrier, or a pharmaceutical composition of the disclosure, and a package insert, wherein the kit comprises instructions for administration of the protein fusion, composition or pharmaceutical composition for treating or delaying progression of an autoimmune disease or suppressing or reducing an autoimmune response in a subject in need thereof.

In some embodiments, a kit comprises a multimeric protein fusion complex of the disclosure, and an optional pharmaceutically acceptable carrier, a composition of the disclosure, and an optional pharmaceutically acceptable carrier, or a pharmaceutical composition of the disclosure, and a package insert, wherein the kit comprises instructions for administration of the protein fusion, composition or pharmaceutical composition for treating or delaying progression of cancer or reducing or inhibiting tumor growth in a subject in need thereof.

In some embodiments, a kit comprises a multimeric protein fusion complex of the disclosure, and an optional pharmaceutically acceptable carrier, a composition of the disclosure, and an optional pharmaceutically acceptable carrier, or a pharmaceutical composition of the disclosure, and a package insert, wherein the kit comprises instructions for administration of the protein fusion, composition or pharmaceutical composition for treating an infection caused by an infectious agent by inducing or enhancing an immune response against the infectious agent in a subject in need thereof.

### Definitions

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “and/or” when used in the context of a list of entities, refers to the entities being present singly or in any possible combination or subcombination.

The term “ameliorating” refers to any therapeutically beneficial result in the treatment of a disease state, e.g., immune disorder, including prophylaxis, lessening in the severity or progression, remission, or cure thereof.

“Amino acid” “Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

The term “antigen-binding portion”, as used herein, refers to one or more fragments of a soluble T cell receptor (TCR) that retains the ability to specifically bind to an antigen.

The term “antigenic determinant” or “epitope” refers to a site on an antigen to which the variable domain of a T-cell receptor, immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given TCR or antibody (i.e., epitope mapping) are well known in the art and include, for example, immunoblotting and immunoprecipitation assays, wherein overlapping or contiguous peptides from the antigen are tested for reactivity with the given TCR or immunoglobulin. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “avidity” as used herein, refers to the binding strength of as a function of the cooperative interactivity of multiple binding sites of a multivalent molecule (e.g., a soluble multimeric TCR- or pMHC-immunoglobulin protein) with a target molecule. A number of technologies exist to characterize the avidity of molecular interactions including switchSENSE and surface plasmon resonance (Gjelstrup et al., J. Immunol. 188:1292-1306, 2012); Vorup-Jensen, Adv. Drug. Deliv. Rev. 64:1759-1781, 2012).

“Binding affinity” generally refers to the strength of the sum total of noncovalent interactions between a single binding site of a molecule (e.g., a TCR, pMHC) and its binding partner. Unless indicated otherwise, as used herein, “binding affinity” refers to intrinsic binding affinity which reflects a 1:1 interaction between members of a binding pair (e.g., TCR and antigen). The affinity of a molecule X for its partner Y can generally be represented by the dissociation constant (Kd). For example, the Kd can be about 200 nM, 150 nM, 100 nM, 60 nM, 50 nM, 40 nM, 30 nM, 20 nM, 10 nM, 8 nM, 6 nM, 4 nM, 2 nM, 1 nM, or stronger. Affinity can be measured by common methods known in the art, including those described herein. Low-affinity TCRs generally bind antigen slowly and tend to dissociate readily, whereas high-affinity TCRs generally bind antigen faster and tend to remain bound longer. A variety of methods of measuring binding affinity are known in the art, any of which can be used for purposes of the present disclosure.

As used herein, the terms “carrier” and “pharmaceutically acceptable carrier” includes any and all solvents, dispersion media, coatings, antibacterial and antifungal agents, isotonic and absorption delaying agents, and the like that are physiologically compatible.

The term “EC50,” as used herein, refers to the concentration of a TCR or an antigen-binding portion thereof, which induces a response, either in an in vitro or an in vivo assay, which is 50% of the maximal response, i.e., halfway between the maximal response and the baseline.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, the term “Fc region” refers to the portion of a native immunoglobulin formed by the respective Fc domains (or Fc moieties) of its two heavy chains. As used herein, the term “Fc domain” refers to a portion of a single immunoglobulin (Ig) heavy chain wherein the Fc domain does not comprise an Fv domain. As such, an Fc domain can also be referred to as “Ig” or “IgG.” In some embodiments, an Fc domain begins in the hinge region just upstream of the papain cleavage site and ends at the C-terminus of the antibody.

A “fusion protein” or “fusion polypeptide” as used interchangeably herein refers to a recombinant protein prepared by linking or fusing two or more polypeptides into a single protein molecule, optionally via amino acid linkers. In some embodiments, a fusion protein of the disclosure comprises a polypeptide comprising a component of the MHC/TCR immune complex (e.g., a binding portion of a TCR or MHC molecule), a multimerization domain, and an immunoglobulin domain. In some embodiments, the component of the MHC/TCR immune complex comprises a soluble TCR polypeptide (e.g., a Vα domain, a Cα domain, a Vβ, a Cβ domain or any combination thereof). In some embodiments, the component of the MHC/TCR immune complex comprises a soluble MHC class I polypeptide (e.g., a soluble MHC class I α domain and a β2-microglobulin domain). In some embodiments, the soluble MHC class I polypeptide further comprises an operably linked antigenic peptide that binds to the MHC class I receptor peptide binding groove. In some embodiments, the component of the MHC/TCR immune complex comprises a soluble MHC class II polypeptide (e.g., a MHCII α1 domain, MHCII α2 domain, MHCII β1 domain, MHCII β2 domain or any combination thereof). In some embodiments, the soluble MHC class II polypeptide further comprises an operably linked antigenic peptide that binds to the MHC class II receptor peptide binding groove. In some embodiments, the multimerization domain comprises a leucine zipper domain disclosed herein. In some embodiments, the multimerization domain comprises a collagen-like trimerization domain disclosed herein. In some embodiments, the immunoglobulin domain comprises an immunoglobulin heavy chain constant region or fragment thereof. In some embodiments, the immunoglobulin domain comprises an immunoglobulin light chain constant region or fragment thereof.

As used herein, “half-life” refers to the time taken for the serum or plasma concentration of a polypeptide to reduce by 50%, in vivo, for example due to degradation and/or clearance or sequestration by natural mechanisms.

As used herein, “immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

The term “immunoglobulin-framework” or “Igg-framework”, as used herein refers to a multimeric protein comprising an immunoglobulin heavy constant region and/or light chain constant regions, or fragments thereof. The immunoglobulin heavy constant region comprises the constant domains (e.g., CH1, CH2, CH3, CH4 domains) of an immunoglobulin heavy chain (e.g., γ, α, δ, μ or ε heavy chains). The immunoglobulin light chain constant region comprises the CL domain of an immunoglobulin light chain (e.g., λ or κ light chains) For example, in some embodiments the Igg-framework can contain two or more immunoglobulin heavy constant regions, or fragments thereof. In some embodiments, the Igg-framework comprises two immunoglobulin heavy constant chain and two light chain constant chains. The multimerization of an Igg-framework is promoted by covalent bonds (e.g., disulfide bonds) and non-covalent interactions (e.g., electrostatic interactions, hydrogen bonding, hydrophobic interactions).

As used herein, the terms “linked,” “conjugated,” “fused,” or “fusion,” are used interchangeably when referring to the joining together of two more elements or components or domains, by whatever means including recombinant or chemical means. In some embodiments, two or more polypeptides are linked, conjugated, or fused by an amino acid linker by recombinant or chemical means. In some embodiments, two or more polypeptides are linked, conjugated, or fused by glycine-serine linker of the disclosure by recombinant or chemical means.

As used herein, “local administration” or “local delivery,” refers to delivery that does not rely upon transport of the composition or agent to its intended target tissue or site via the vascular system. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site.

The term “Major Histocompatibility Complex” or “MHC” refers to genomic locus containing a group of genes that encode the polymorphic cell-membrane-bound glycoproteins known as MHC classical class I and class II molecules that regulate the immune response by presenting peptides of fragmented proteins to circulating cytotoxic and helper T lymphocytes, respectively. In humans this group of genes is also called the “human leukocyte antigen” or “HLA” system. Human MHC class I genes encode, for example, HLA-A, HL-B and HLA-C molecules. HLA-A is one of three major types of human MHC class I cell surface receptors. The others are HLA-B and HLA-C. The HLA-A protein is a heterodimer, and is composed of a heavy α chain and smaller β chain. The α chain is encoded by a variant HLA-A gene, and the β chain (β2-microglobulin) is an invariant β2 microglobulin molecule. The β2 microglobulin protein is coded for by a separate region of the human genome. HLA-A*02 (A*02) is a human leukocyte antigen serotype within the HLA-A serotype group. The serotype is determined by the antibody recognition of the α2 domain of the HLA-A α-chain. For A*02, the α chain is encoded by the HLA-A*02 gene and the β chain is encoded by the B2M locus. Human MHC class II genes encode, for example, HLA-DPA1, HLA-DPB1, HLA-DQA1, HLA-DQB1, HLA-DRA and HLA-DRB1. The complete nucleotide sequence and gene map of the human major histocompatibility complex is publicly available (e.g., The MHC sequencing consortium, Nature 401:921-923, 1999).

As used herein, the terms “MHC molecule” and “MHC protein” are used herein to refer to the polymorphic glycoproteins encoded by the MHC class I and MHC class II genes, which are involved in the presentation of peptide antigens to T cells. The terms “MHC class I” or “MHC I” are used interchangeably to refer to protein molecules comprising an α chain composed of three domains (α1, α2 and α3), and a second, invariant β2-microglobulin. The α 3 domain is transmembrane, anchoring the MHC class I molecule to the cell membrane. Antigen-derived peptide antigens, which are located in the peptide-binding groove, in the central region of the α 1/α 2 heterodimer. MHC Class I molecules such as HLA-A are part of a process that presents short polypeptides to the immune system. These polypeptides are typically 9-11 amino acids in length and originate from proteins being expressed by the cell. MHC class I molecules present antigen to CD8+ cytotoxic T cells. The terms “MHC class II” and “MHC II” are used interchangeably to refer to protein molecules containing an α chain with two domains (α1 and α2) and a β chain with two domains β1 and β2). The peptide-binding groove is formed by the α1/β1 heterodimer. MHC class II molecules present antigen to specific CD4+ T cells. Antigens delivered endogenously to APCs are processed primarily for association with MHC class I. Antigens delivered exogenously to APCs are processed primarily for association with MHC class II.

The term “multimeric protein”, “multimeric fusion protein” or “multimeric protein complex” are used interchangeably herein and refer to a protein comprising two or more of the same or different, polypeptide chains, wherein the protein comprises at least two receptor binding sites for interaction in an MHC/TCR immune complex (e.g., a TCR, MHC class I receptor, or MHC class II receptor binding site). For example, a multimeric protein or protein complex that is a dimer comprises 2 binding sites for interaction in a MHC/TCR immune complex, a multimeric protein or protein complex that is a trimer comprises 3 binding sites for interaction in a MHC/TCR immune complex, multimeric protein or protein complex that is a tetramer comprises 4 binding sites for interaction in a MHC/TCR immune complex, a multimeric protein or protein complex that is a hexamer comprises 6 binding sites for interaction in a MHC/TCR immune complex

In some embodiments, a multimeric fusion protein is assembled from two or more fusion proteins, each comprising a soluble TCR polypeptide, wherein the multimeric fusion protein provides two or more TCR binding sites that can form an MHC/TCR immune complex (e.g., a TCR dimer, trimer, tetramer, or hexamer). In some embodiments, a multimeric fusion protein is assembled from two or more fusion proteins, each comprising a soluble MHC class I polypeptide, wherein the multimeric fusion protein provides two or more MHC class I binding sites that can bind to antigenic peptide and form an MHC class I/TCR immune complex (e.g., a MHC class I receptor dimer, trimer, tetramer, or hexamer). In some embodiments, a multimeric fusion protein is assembled from two or more fusion proteins, each comprising a soluble MHC class II polypeptide, wherein the multimeric fusion protein provides two or more MHC class II binding sites that can bind to antigenic peptide and form an MHC class II/TCR immune complex (e.g., a MHC class II receptor dimer, trimer, tetramer, or hexamer). In some embodiments, the two or more fusion proteins are assembled by binding interactions of an immunoglobulin framework, binding interactions of multimerization domains, or a combination thereof. “Multimerization” as used herein refers to the assembly of two or more polypeptide chains (e.g., fusion polypeptides containing a soluble TCR or pMHC operably linked to a immunoglobulin constant domain). A “multimerization domain” as used herein refers to an amino acid sequence within a polypeptide which promotes assembly of two or more polypeptides into a protein multimer (e.g., homomultimer or heteromultimer). A “dimerization domain” refers to an amino acid sequence within a polypeptide that promotes assembly of the polypeptide into dimers, and a “trimerization domain” refers to an amino acid sequence within a polypeptide that promotes assembly of the polypeptide into trimers with the same or different polypeptide chains. For example, a dimerization domain or a trimerization domain can promote assembly of a protein into dimers or trimers via associations with other dimerization or trimerization domains (of additional polypeptides with the same or a different amino acid sequence). The term is also used to refer to a polynucleotide that encodes the amino acid sequence of multimerization domain.

“Nucleic acid” refers to deoxyribonucleotides or ribonucleotides and polymers thereof in either single- or double-stranded form. Unless specifically limited, the term encompasses synthetic or isolated nucleic acids, and chemically, enzymatically, or metabolically modified forms thereof. For example, a nucleic acid can contain known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. The term “isolated nucleic acid” is intended to refer to nucleic acids encoding a polypeptide which are substantially free of other sequences which naturally flank the nucleic acid in human genomic DNA.

As used herein, the terms “operably linked” or “operably coupled” refer to a juxtaposition wherein the components described (e.g., polypeptides, nucleic acids) are in a relationship permitting them to function in their intended manner.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion).

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The terms “isolated protein” and “isolated polypeptide” are used interchangeably to refer to a protein (e.g., a soluble, multimeric protein) which has been separated or purified from other components (e.g., proteins, cellular material) and/or chemicals. Typically, a polypeptide is purified when it constitutes at least 60 (e.g., at least 65, 70, 75, 80, 85, 90, 92, 95, 97, or 99) % by weight of the total protein in the sample.

As used herein, the term “preventing” when used in relation to a condition, refers to administration of a composition which reduces the frequency of, or delays the onset of, symptoms of a medical condition in a subject relative to a subject which does not receive the composition.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the terms “specific binding,” “selective binding,” “selectively binds,” and “specifically binds,” refer to TCR binding to an antigen and/or an epitope thereof (including but not limited to a peptide, optionally in complex with an MHC molecule). As such, a TCR or portion thereof is said to “specifically” bind an antigen and/or an epitope thereof when the dissociation constant (Kd) is less than about 1 μM, less that about 100 nM, less than about 10 nM, or even lower. The term “K_{D},” as used herein, is intended to refer to the dissociation equilibrium constant of a particular TCR-antigen interaction. The term “Ka” as used herein, is intended to refer to the on rate constant for the association of a TCR with the antigen. Specific binding affinity may be determined according to routine methods, for example, by surface plasmon resonance (SPR) technology in which the TCR or TCR-fusion protein complex binds to the antigen with an affinity that is at least two-fold greater than its affinity for binding to a non-specific antigen (e.g., BSA, casein) other than the predetermined antigen or a closely-related antigen. The phrases “a TCR recognizing an antigen” and “TCR specific for an antigen” are used interchangeably herein with the term “a TCR which binds specifically to an antigen.”

As used herein, the term “subject” or “patient” includes any human or non-human animal that receive treatment. For example, the methods and compositions of the present disclosure can be used to treat a subject with an immune disorder. The term “non-human animal” includes all vertebrates, e.g., mammals and non-mammals, such as non-human primates, sheep, dog, cow, chickens, amphibians, reptiles, etc.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising a fusion protein described herein).

The term “T cell” refers to a type of white blood cell that can be distinguished from other white blood cells by the presence of a T cell receptor on the cell surface. There are several subsets of T cells, including, but not limited to, T helper cells (a.k.a. T_{H} cells or CD4⁺ T cells) and subtypes, including T_{H}1, T_{H}2, T_{H}3, T_{H}17, T_{H}9, and T_{FH} cells, cytotoxic T cells (a.k.a T_{C} cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺ FOXP3⁺ T_{reg} cells, CD4⁺FOXP3⁻ T_{reg} cells, Tr1 cells, Th3 cells, and T_{reg}17 cells, natural killer T cells (a.k.a. NKT cells), mucosal associated invariant T cells (MAITs), and gamma delta T cells (γδ T cells), including Vγ9/Vδ2 T cells. The term “T cell cytotoxicity” includes any immune response that is mediated by CD8+ T cell activation.

As used herein, the phrase “T cell receptor” and the term “TCR” refer to a surface protein of a T cell that allows the T cell to recognize an antigen and/or an epitope thereof, typically bound to one or more major histocompatibility complex (MHC) molecules. A TCR functions to recognize an antigenic determinant and to initiate an immune response. Typically, TCRs are heterodimers comprising two different protein chains. In the vast majority of T cells, the TCR comprises an alpha (α) chain and a beta (β) chain Each chain comprises two extracellular domains: a variable (V) region and a constant (C) region, the latter of which is membrane-proximal. The variable domains of α-chains and of β-chains consist of three hypervariable regions that are also referred to as the complementarity determining regions (CDRs). The CDRs, in particular CDR3, are primarily responsible for contacting antigens and thus define the specificity of the TCR, although CDR1 of the α-chain can interact with the N-terminal part of the antigen, and CDR1 of the α-chain interacts with the C-terminal part of the antigen. Approximately 5% of T cells have TCRs made up of gamma and delta (γ/δ) chains. All numbering of the amino acid sequences and designation of protein loops and sheets of the TCRs is according to the IMGT numbering scheme (IMGT, the international ImMunoGeneTics information system@imgt.cines.fr; http://imgt.cines.fr; Lefranc et al., (2003) Dev Comp Immunol 27:55 77; Lefranc et al. (2005) Dev Comp Immunol 29:185-203).

As used herein, the terms “soluble T-cell receptor” and “sTCR” refer to single chain or heterodimeric truncated variants of TCRs, which comprise extracellular portions of the TCR α-chain and β-chain (e.g., linked by a disulfide bond), but which lack the transmembrane and cytosolic domains of the full-length protein. The sequence (amino acid or nucleic acid) of the soluble TCR α-chain and β-chains may be identical to the corresponding sequences in a native TCR or may comprise variant soluble TCR α-chain and β-chain sequences, as compared to the corresponding native TCR sequences. The term “soluble T-cell receptor” as used herein encompasses soluble TCRs with variant or non-variant soluble TCR α-chain and β-chain sequences. The variations may be in the variable or constant regions of the soluble TCR α-chain and β-chain sequences and can include, but are not limited to, amino acid deletion, insertion, substitution mutations as well as changes to the nucleic acid sequence, which do not alter the amino acid sequence. Variants retain the binding functionality of their parent molecules. In some embodiments, a soluble TCR comprises a single-chain TCR polypeptide comprising a TCR α variable region, a TCR β variable region, and a TCR β constant region operably linked, optionally via an amino acid linker to form a soluble, single chain TCR receptor.

As used herein, the term “soluble MHC class I receptor” refers to single chain or heterodimeric truncated variants of MHC class I receptors, comprising the extracellular portions of the MHC class I α domain and β2-microglobulin polypeptide, but which lack the transmembrane and cytosolic domains of the full-length protein. In some embodiments, the soluble MHC class I receptor comprises MHC domains that enable binding of antigenic peptide. The sequence (amino acid or nucleic acid) of the soluble MHC class I α domain and β2-microglobulin polypeptide are in some embodiments, identical to the corresponding sequences in a native MHC class I receptor, while in other embodiments, comprise variant MHC class I receptor sequences as compared to the native corresponding MHC class I receptor sequences. In some embodiments, the soluble MHC class I α domain and β2-microglobulin polypeptide domain are operably linked, optionally via an amino acid linker.

As used herein, the term “soluble MHC class II receptor” refers to single chain or heterodimeric truncated of variants of MHC class II receptors, comprising the extracellular portions of the MHC class II α domain (e.g., α1 or α1+α2) and β domain (e.g., β1 or β1+β2), but which lack the transmembrane and cytosolic domains of the full-length protein. In some embodiments, the soluble MHC class II receptor comprises MHC domains that enable binding of antigenic peptide (e.g., α1+β1 domains). The sequence (amino acid or nucleic acid) of the soluble MHC class I α domain β domain are in some embodiments, identical to the corresponding sequences in a native MHC class II receptor, while in other embodiments, comprise variant MHC class II receptor sequences as compared to the native corresponding MHC class II receptor sequences. In some embodiments, the soluble MHC class II α domain and β domain are operably linked, optionally via an amino acid linker (e.g., single chain α1+β1 domains).

As used herein, a “TCR/pMHC complex” refers to a protein complex formed by binding between T cell receptor (TCR), or soluble portion thereof, and a peptide-loaded MHC molecule. Accordingly, a “component of a TCR/pMHC complex” refers to one or more subunits of a TCR (e.g., Vα, Vβ, Cα, Cβ), or to one or more subunits of a MHC or pMHC class I or II molecule.

The term “therapeutically effective amount” is an amount of a composition that is effective to ameliorate a symptom of a disease. A therapeutically effective amount can be a “prophylactically effective amount” as prophylaxis can be considered therapy.

The terms “treat,” “treating,” and “treatment,” as used herein, refer to therapeutic or preventative measures described herein. The methods of “treatment” employ administration to a subject, in need of such treatment, a soluble multimeric protein fusion complex of the present disclosure, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

Various aspects of the disclosure are described in further detail in the following subsections.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al, Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B (1992).

### Example 1—Production of Soluble TCR-Igg Multimers

To achieve decent yield, current soluble TCR production protocol often relies on stabilization mutations or simply using high-affinity TCRs which seems to have higher intrinsic stability but may rarely be found. Although helpful, stabilizing mutations are likely not applicable to all TCRs, and in certain situations, introducing mutations may alter TCR interaction specificity leading to off-target toxicity. The novel design described herein harnesses a generic stabilization strategy that potentially works on any TCR and often results in very good protein yield, 10-30 mg/L.

Most current soluble TCRs are in conventional biotin-streptavidin tetrameric form that produces enough avidity for TCR-target interaction. Yet, the tetrahedral nature of streptavidin does not allow full engagement of its tetravalency, mostly allowing for bivalent or trivalent interactions only. The Igg-based flexible design disclosed herein allows all monomers to face the same direction, thus dramatically enhancing binding to target cells.

The natural dimerization potential of Igg molecules offers a unique feature to multimerize low affinity TCRs or pMHCs for increased avidity. Additionally, the innate immune cell engaging Fc domain combined with the antigen-targeting capability of the TCR or MHC domain offers potential for new therapeutic modalities. However, unlike a fusion of a variable region of typical Igg, fusing the variable region of a TCR to an Igg constant region yields only low amount of secreted protein. Without being bound by theory, this is likely due to the incompatible modular structures of antibody variable region and TCR variable region. This incompatibility results in low protein production.

A number of strategies were evaluated in order to develop a framework for producing soluble TCR and pMHC multimers fused to an Igg with optimal yield and therapeutic potential. These strategies included (1) an Igg framework with natural dimerization and therapeutic potential; (2) a pair of preferential dimerization leucine zipper domains (LZL and LZR; SEQ ID NOS: 6 and 8) with super-high affinity (10⁻¹⁵M); (3) an experimentally derived collagen-like trimerization domain (GPP₁₀; SEQ ID NO: 60). A combination of Igg and leucine zipper/GPP yields a variety of multimeric fusion frameworks carrying innate immune cell engaging FC region for both TCR and pMHC (FIG. 1).

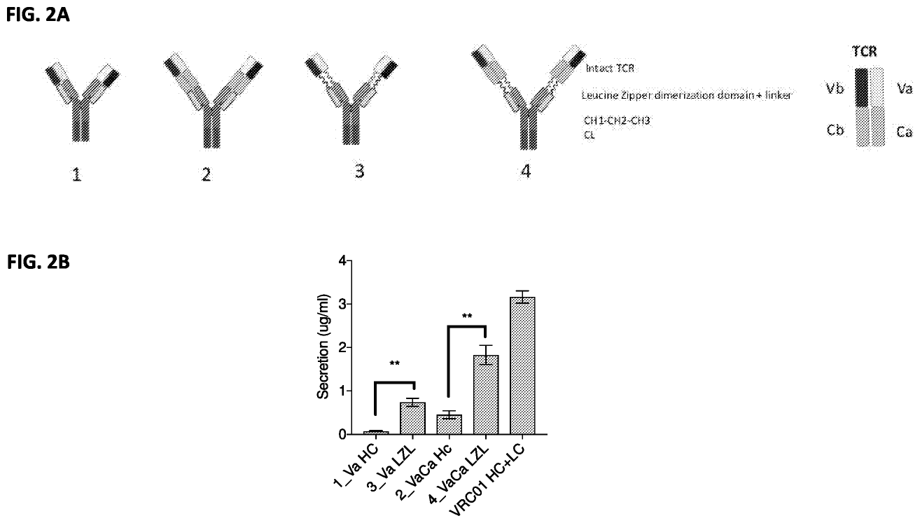

TCR-Igg fusion protein requires sufficient stability to be assembled properly and secreted at decent level. Multiple TCR-Igg fusion designs were tested for expression by fusing different portion of TCR into Igg (e.g., mouse IgG2c) as depicted in FIG. 2A. These included direct fusion of the TCR variable domains (with or without TCR constant domains) to the Igg constant domains and fusion of TCR variable domains (with or without TCR constant domains) to Igg constant domains by a leucine zipper dimerization domain.

| | |
|---|---|
| (1) | TCR-Va-CH1-CH2-CH3; TCR-Vb-CL. |
| (2) | TCR-VaCa-CH1-CH2-CH3; TCR-VbCb-CL. |
| (3) | TCR-Va-LZL-CH1-CH2-CH3; TCR-Vb-LZR-CL. |
| (4) | TCR-VaCa-LZL-CH1-CH2-CH3; TCR-VbCb-LZR-CL |

To evaluate these constructs, the murine OTI TCR (monomer KD=˜7 uM), which recognizes the ovalbumin₂₅₇₋₂₆₄ peptide SIINFEKL, was selected for proof-of principle and for optimization. Construct 4 as shown in FIG. 2A comprised 1) an OT1 TCRα variable and constant domains that were operably linked via a glycine-serine linker (e.g., (G₄S)₄) to a leucine zipper domain (e.g., LZL) that was operably linked via a glycine-serine linker to the CH1-CH2-CH3 domains of murine IgG2c and 2) an OT1 TCRβ variable region that was operably linked via a glycine-serine linker (e.g., (G₄S)₄) to a leucine zipper domain (e.g., LZL) that was operably linked via a glycine-serine linker to the CL domain of murine IgG2c. The construct 3 as shown in FIG. 2A comprised the same components, but with the OT1 TCRα and TCRβ chains comprising a variable region only.

As shown in FIG. 2A, constructs 1 and 2 lacked the leucine zipper multimerization domains. Instead, construct 1 comprised OT1 TCR Vα and TCR Vβ chains fused directly to the CH1-CH2-CH3 domains and CL domain of murine IgG2c via short Gly-Ser linkers. Construct 2 comprised the same fusion as construct 1, but included TCR Cα and TCR Cβ domains.

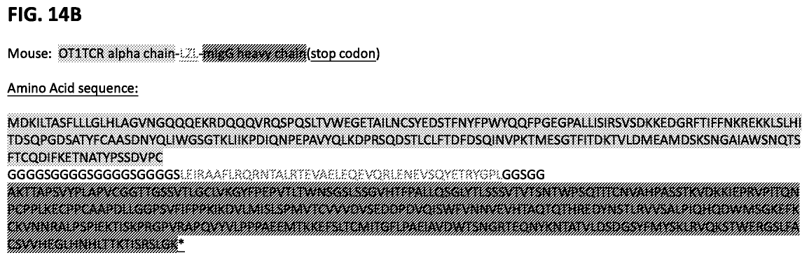

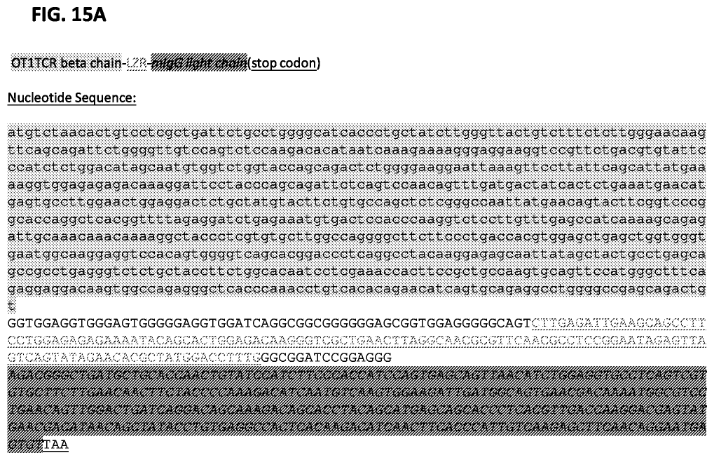

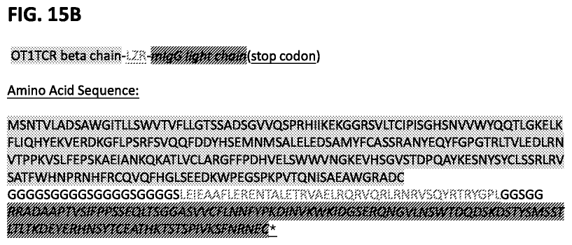

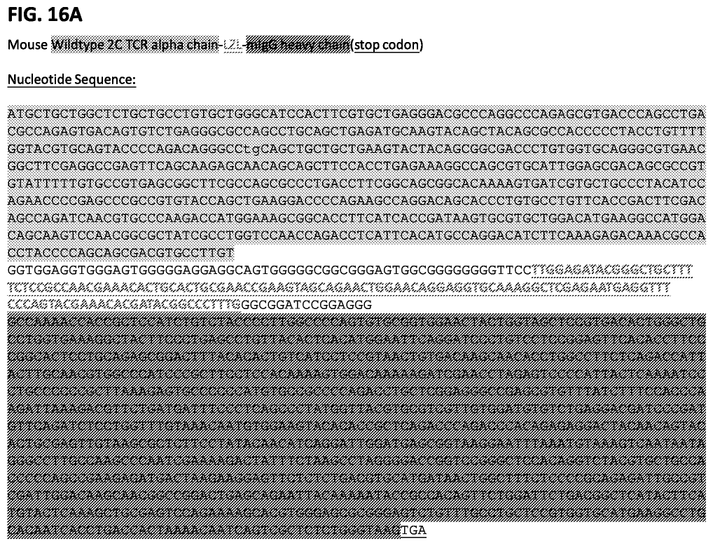

Components of the OT1 TCR-Igg are identified in Table 1, and the nucleotide and amino acid sequences of construct 4 are further shown in FIGS. 14A-14B and FIGS. 15A-15B.

TABLE 1
components of TCR-Igg fusion proteins comprising a mouse
OT1 TCR and mouse IgG2c framework

| | SEQ ID NO | SEQ ID NO |
|---|---|---|
| Component | (nucleic acid) | (amino acid) |
| OTI TCRα | 1 | 2 |
| OTI TCRβ | 3 | 4 |
| LZL | 5 | 6 |
| LZR | 7 | 8 |
| IgG2c heavy chain (CH1-CH2-CH3) | 13 | 14 |
| IgG2c light chain (CL) | 15 | 16 |
| Linker (GGGGS)₄ | 9 | 10 |
| Linker GGSGG | 11 | 12 |
| OTI TCRα-LZL-mIgG HC | 17 | 18 |
| OTI TCRβ-LZR-mIgG CL | 19 | 20 |

To express the fusion proteins shown in FIG. 2A, 180 μg of mixed plasmids (e.g., 1:1 molarity ratio of OTI TCRα-LZL-mIgG HC and OTI TCRβ-LZR-mIgG LC) were transfected into Expi293F cells using ExpiFectamine method in 180 ml total volume. After 5 days of culturing with constant shaking, cells were pelleted at 6500 RPM for 20 minutes and the supernatant was sterilized with 0.22 μm filter unit.

TCR-Igg were then purified using HiTrap Protein G HP column (GE) on AKTA pure FPLC system. The column was equilibrated with PBS (with 5 column volumes (CV)) before loading the supernatant. After all the supernatant passed through, the column was washed with 1×PBS (15 CV), and TCR-Igg was eluted with 0.1M Glycine-HCL (with 5 CV). A small aliquot was analyzed by SDS-PAGE to confirm protein size and purity.

It was determined that incorporation of high affinity leucine zipper dramatically increased fusion protein secretion, up to 2 μg/ml with design (4) in a pilot experiment (FIG. 2B). The secretion level was found to be almost half of that for a control antibody (e.g., the VRC01 antibody). With the optimal protein production protocol described above, purification of 1.5 mg protein was obtained with less than 150 ml culture.

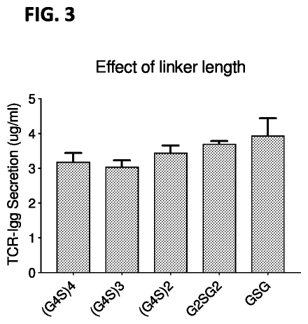

The TCR-Igg fusions comprising a leucine zipper fused to the TCR via a glycine/serine linker. The effect of increasing the length of the glycine/serine linkers connecting the TCR and Igg chains was assessed. As shown in FIG. 3, short glycine/serine linkers (e.g., a GSG linker) resulted in increased fusion protein secretion, up to approximately 4 μg/mL with a short GSG linker compared to approximately 3 μg/mL with a longer linker (e.g., a (G₄S)₄ or (G₄S)₃ linker). Thus, shorter linker lengths may contribute to optimal pairing and secretion of the TCR-Igg fusion protein.

### Example 2—Single Expression Vector Construction

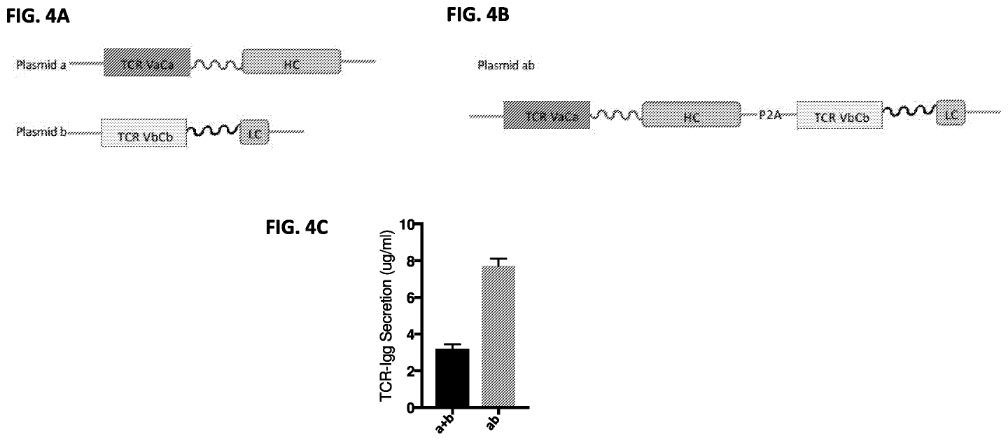

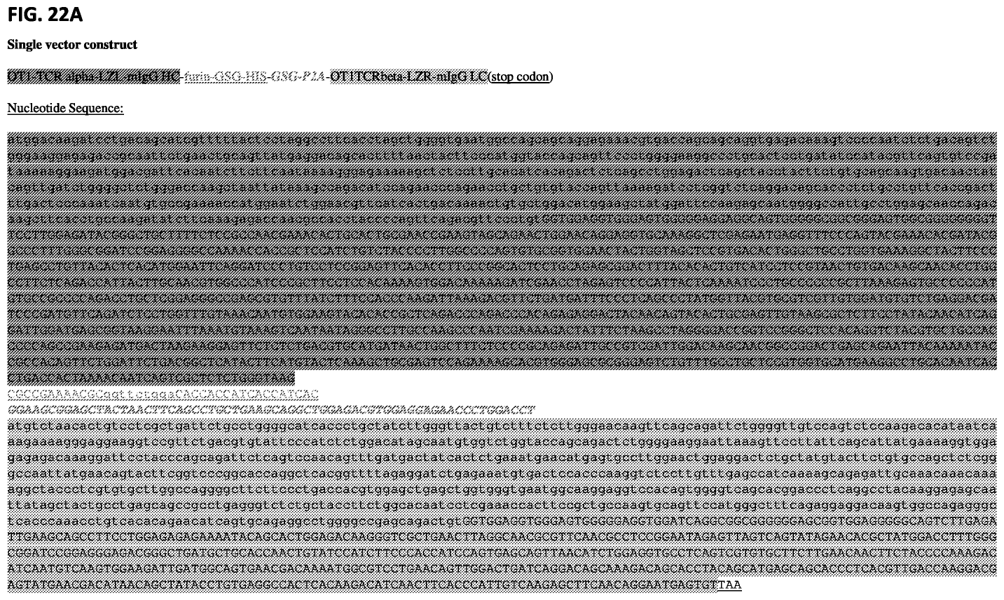

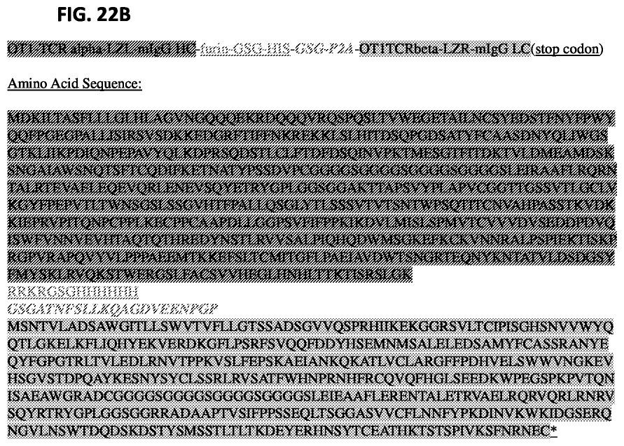

Given that the two halves of the fusion molecules were coded by two separate plasmids (FIG. 4A), simultaneous co-transfection of equivalent amount of both heavy chain and light chain into the same cell is required for correct assembly. To maximize fusion protein assembly and production, a fusion protein with the structure TCR-VαCα-LZL-CH1-CH2-CH3 and TCR-VbCb-LZR-CL was combined into a single co-expression construct separated by a self-cleaving P2A peptide (FIG. 4B). The plasmid further comprised TCR-VαCα-LZL-CH1-CH2-CH3 operably linked to a furin-GSG-His sequence such that the expressed protein comprised a His tag (e.g., HHHHHH) for purposes of manipulation and purification, and a cleavage substrate for furin to allow enzymatic removal of the His tag and residues of the 2A cleavage sequence. The plasmid encoded furin-GSG-His sequence operably linked to self-cleaving P2A peptide that was further operably linked to the TCR-VbCb-LZR-C_{L} domain. The nucleotide and amino acid sequences of the single vector are identified by SEQ ID NO: 53 and SEQ ID NO: 54 respectively, and shown in FIGS. 22A-22B. The components are further identified by sequence in Table 2.

TABLE 2
components of single vector plasmid encoding TCR-Igg fusion
protein comprising a mouse OT1 TCR and mouse IgG2c framework

| | SEQ ID NO | SEQ ID NO |
|---|---|---|
| Component | (nucleic acid) | (amino acid) |
| OTI TCRα | 1 | 2 |
| OTI TCRβ | 3 | 4 |
| LZL | 5 | 6 |
| LZR | 7 | 8 |
| IgG2c heavy chain (CH1-CH2-CH3) | 13 | 14 |
| IgG2c light chain (CL) | 15 | 16 |
| Linker (GGGGS)₄ | 9 | 10 |
| Linker GGSGG | 11 | 12 |
| OTI TCRα-LZL-mIgG HC | 17 | 18 |
| OTI TCRβ-LZR-mIgG LC | 19 | 20 |
| Furin-GSG-His | 49 | 50 |
| GSG-P2A | 51 | 52 |
| OTI TCRα-LZL-IgG_{HC}-furin-GSG- | 53 | 54 |
| His-GSG-P2A-OTI TCRβ-LZR-IgG_{LC} | | |

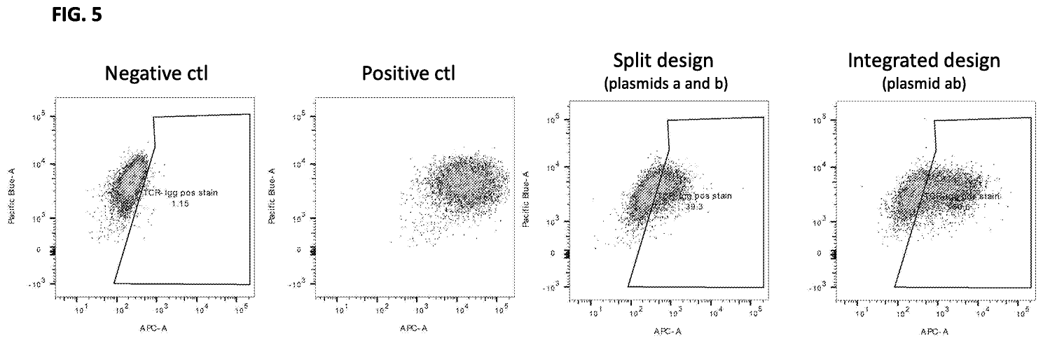

Expression of the multimeric fusion protein from the single construct yielded a further increase in protein yield by almost 3 fold (FIG. 4C). Such production increase was further confirmed by staining K562 cells that express single-chain SIINFEKL-H2Kb with raw supernatant from cells transfected with single or dual plasmids encoding TCR-Igg. Surface staining with supernatant was analyzed by flow cytometry Staining with an anti-mouse SIINFEKL-H2Kb antibody (clone 25-D.16) indicated that the cells were positive for MHCI (e.g., H2Kb) loaded with SIINFEKL peptide. Moreover, supernatant from cells transfected with a single construct gave stronger staining than supernatant from cells co-transfected with dual plasmids encoding TCR-Igg (FIG. 5).

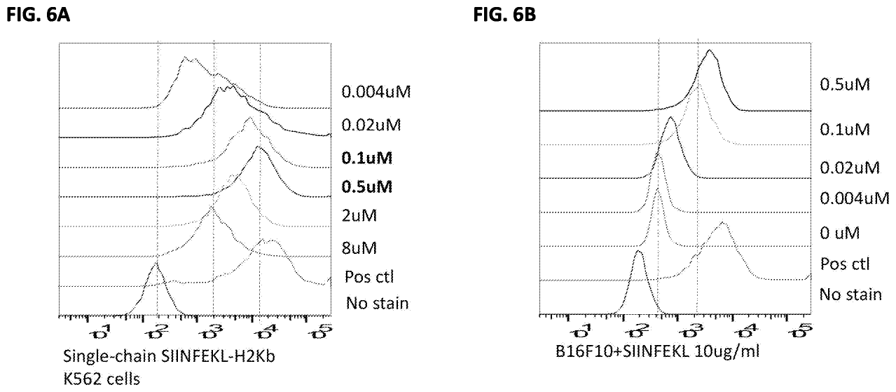

The reported affinity of OTI TCR monomer is about 7 μM. To further characterize the avidity of OT1-TCR-Igg dimer, purified OTI-TCR-Igg was titrated from 8 μM down to 4 nM and used to stain K562 cells expressing single chain SIINFEKL-H2Kb and B16F10 cells pulsed with SIINFEKL peptide. B16F10 cells were prepared before peptide loading by treating overnight with 50 U recombinant mouse IFN gamma to induce increased surface expression of MHCI (e.g., H2Kb). Cells were then collected as a single cell suspension and loaded with 10 μg/mL of SIINFEKL peptide at 37° C. for 1 hour. Anti-mouse SIINFEKL-bound H2Kb antibody, (clone 25-D1.16) was used as a positive control to measure surface levels of SIINFEKL-H2Kb. Surface staining with OT1-TCR-Igg was measured by flow cytometry. Although high concentration of TCR-Igg fusion has adverse effect on staining, at a moderate concentration of 0.5 μM, staining with TCR-Igg yielded almost equivalent staining to the positive control antibody (anti-mouse SIINFEKL bound H-2Kb, clone 25-D1.16). As low as 20 nM could clearly stain almost 100% of both SIINFEKL presenting K562 cells (FIG. 6A) and B16F10 cells (FIG. 6B).

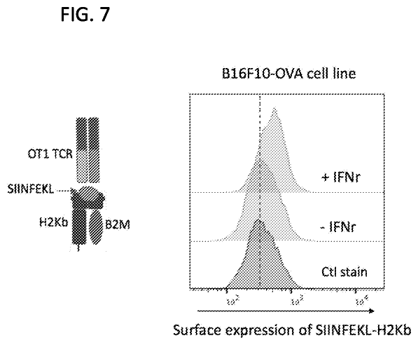

TCR-Igg fusion proteins were further evaluated for detection of endogenous antigen peptide presented by target cells. To do so, B16F10 cells that express ovalbumin protein were treated with recombinant mouse IFNgamma (IFNr) overnight. Treatment with IFNr results in increased MHCI (H-2Kb) expression and increased presentation of Ova antigenic peptides (e.g., SIINFEKL) by MHCI (e.g., H2Kb). The cells were then treated with fluorophore-labeled OT1-TCR-Igg. As shown in FIG. 7, cells that were not treated with IFNr showed no labeling by OT1-TCR-Igg as compared to unlabeled cells. However, cells treated with IFNr to induce antigen presentation demonstrated increased labeling by OT1-TCR-Igg, together indicating that the fusion protein selectively labels cells with surface presentation of endogenous antigen.

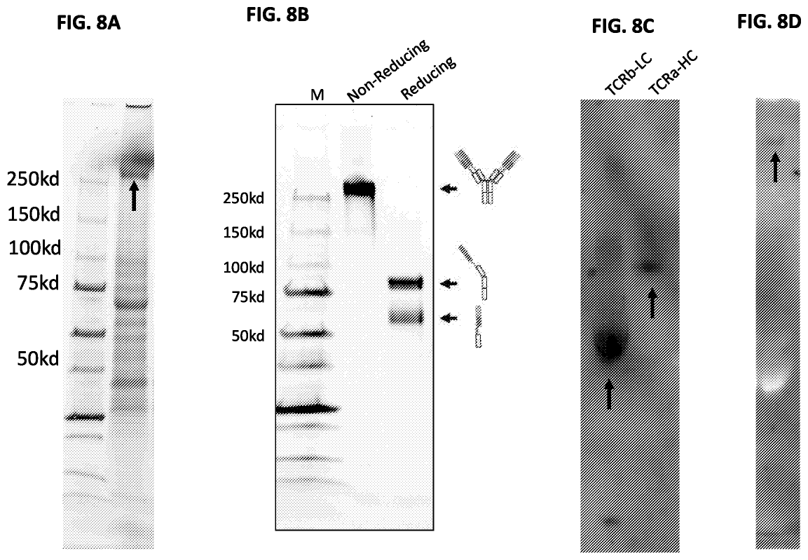

The OT1-TCR-Igg fusion protein was characterized by gel electrophoresis. To do so, 1 mL of raw cell culture supernatant was concentrated using a spin column and loaded in a stain-free PAGE-gel. Naïve PAGE gel showed that the multimeric TCR-Igg fusion was of expected size, ˜250 kd (FIG. 8A). Additionally, the structure of the multimeric TCR-Igg fusion protein was characterized by evaluating protein molecular weight following exposure to denaturing conditions with or without reducing agent. To do so, purified OT1-TCR-Igg was boiled in SDS buffer with or without the reducing agent DTT, then analyzed by stain-free PAGE gel. Under non-reducing conditions (without DTT), the dimer remained as a single molecule, while under reducing conditions (with DTT), two chains corresponding to the fusion protein light and heavy chains were detected as shown in FIG. 8B. Thus, the TCR-Igg fusion protein is composed of two chains linked via a disulfide bond that only dissociates under reducing conditions.

Furthermore, the TCR-Igg fusion protein was assessed by western blot following treatment with denaturing and reducing or non-reducing conditions. To do so, TCR-Igg was boiled in the presence of SDS with or without DTT, separated by gel electrophoresis and blotted with an anti-Igg antibody that could recognize both heavy and light chains Treatment with denaturing and reducing conditions yielded two fragments with sizes indicative of the heavy chain (˜70 kd) and light chain (˜50 kd) (FIG. 8C). While treatment with denaturing and non-reducing conditions yielded intact TCR-Igg with the expected molecular weight of ˜250 kd (FIG. 8D). These data indicate that multimeric TCR-Igg assembled correctly as expected, with disulfide bonds formed between CL and CH1, and the TCR alpha chain and beta chain successfully paired with each other.

### Example 3—High Protein Yields of Multimeric TCR Fusion Protein

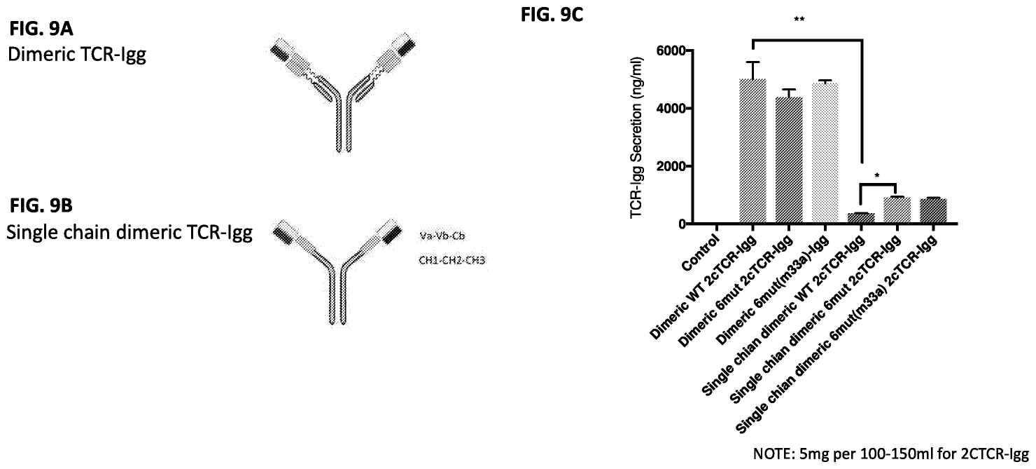

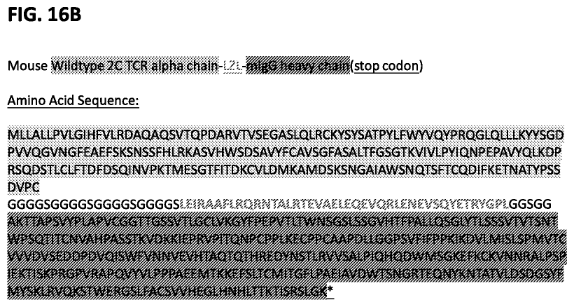

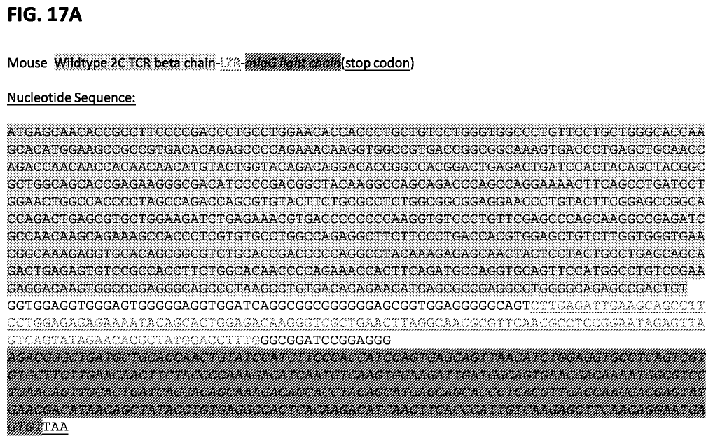

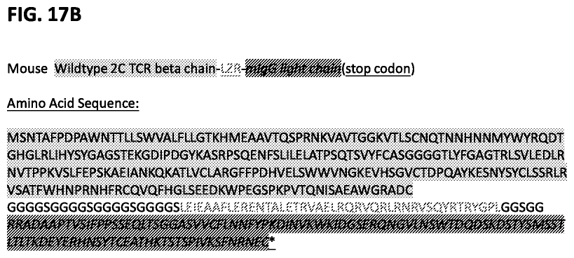

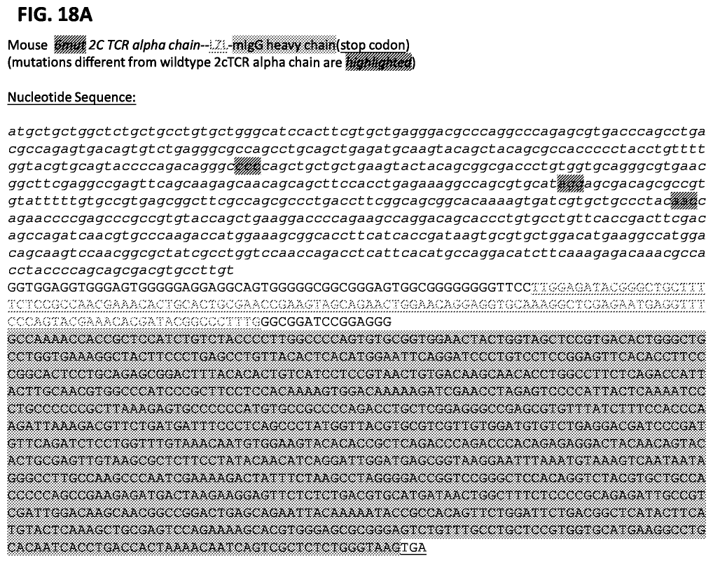

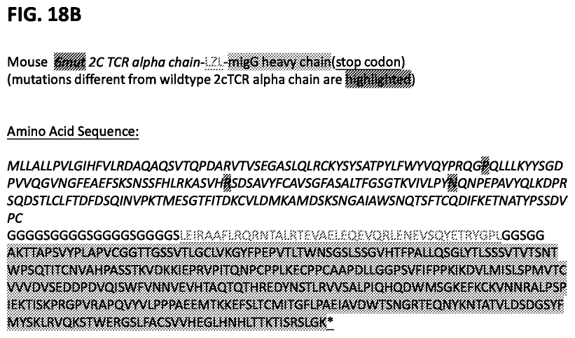

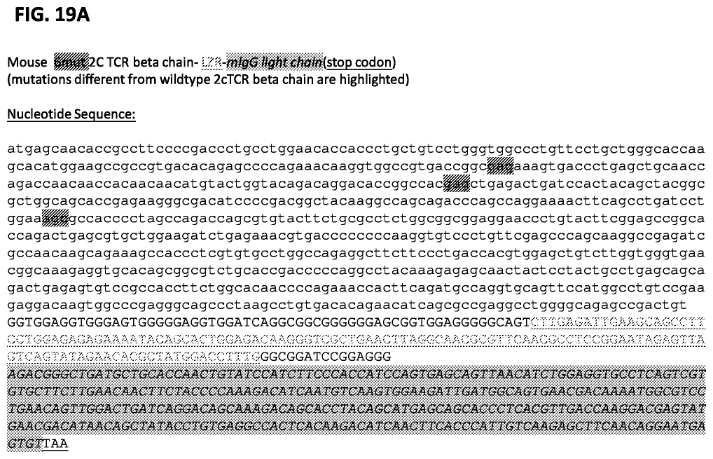

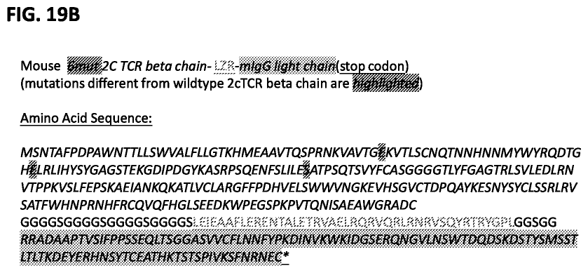

Using another model TCR, 2C-TCR, it was shown that the TCR-Igg dimer (FIG. 9A) has a much higher protein yield compared to previously reported TCR dimers formed by fusing a single chain TCR onto an Igg heavy chain (FIG. 9B). Expression levels were compared for the wild type 2C-TCR and for two different clones of the 2C-TCR comprising different stabilizing mutations: the 6mut clone and the 6mut(m33a) clone. The 6mut clone comprising six stabilizing mutations in the 2C-TCR as shown in FIG. 18 for the 2C-TCR alpha chain and in FIG. 19 for the 2C-TCR beta chain. The 6mut(m33a) clone comprising the 6mut stabilizing mutations with an additional stabilizing mutation to the 2C-TCR. The TCR was fused to a Igg comprising a heavy chain and light chain via a leucine zipper (e.g., dimeric TCR-Igg, FIG. 9A) or a single-chain TCR was fused to the Igg heavy chain (e.g., single chain dimeric TCR-Igg, FIG. 9B). The single-chain TCR comprised a TCR Vα domain operably linked to the TCR β chain (e.g., VβCβ) by a Gly-Ser linker. Components of the 2C-TCR fusion proteins are identified in Table 3. The nucleotide and amino acid sequence are further illustrated for 2C-TCRα-LZL-IgG_{HC} (FIGS. 16A-16B), 2C-TCRβ-LZR-IgG_{LC} (FIGS. 17A-17B), 6mut 2C-TCRα-LZL-IgG_{HC} (FIGS. 18A-18B), and 6mut 2C-TCRβ-LZR-IgG_{LC} (FIGS. 19A-19B).

TABLE 3
components of TCR-Igg fusion proteins comprising a mouse
2C-TCR or variant thereof and a mouse IgG2c framework

| | SEQ ID NO | SEQ ID NO |
|---|---|---|
| Component | (nucleic acid) | (amino acid) |
| Wild type 2C-TCRα | 21 | 22 |
| Wild type 2C-TCRβ | 23 | 24 |
| 6mut 2C-TCRα | 29 | 30 |
| 6mut 2C-TCRβ | 31 | 32 |
| LZL | 5 | 6 |
| LZR | 7 | 8 |
| IgG2c heavy chain | 13 | 14 |
| (CH1-CH2-CH3) | | |
| IgG2c light chain (CL) | 15 | 16 |
| Linker (GGGGS)₄ | 9 | 10 |
| Linker GGSGG | 11 | 12 |
| 2C-TCRα-LZL-IgG_{HC} | 25 | 26 |
| 2C-TCRβ-LZR-IgG_{LC} | 27 | 28 |
| 6mut 2C-TCRα-LZL-IgG_{HC} | 33 | 34 |
| 6mut 2C-TCRβ-LZR-IgG_{LC} | 35 | 36 |

The stabilizing mutations did provided increased stability of the 2C-TCR dimer that yielded increased secretion levels for the single chain dimeric TCR-Igg. However, the TCR-Igg fusion containing the leucine zipper stabilized the TCR-Igg dimer to a greater extent and resulted in much higher levels of secretion (>10 fold increase) regardless of whether the stabilizing mutations were incorporated or not (FIG. 9C). Thus, a TCR-Igg dimer comprising a leucine zipper demonstrated optimal stabilization and a superior dimerization strategy for natural TCRs.

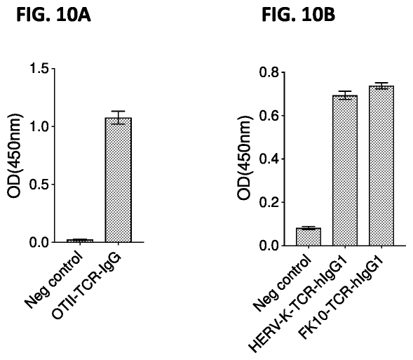

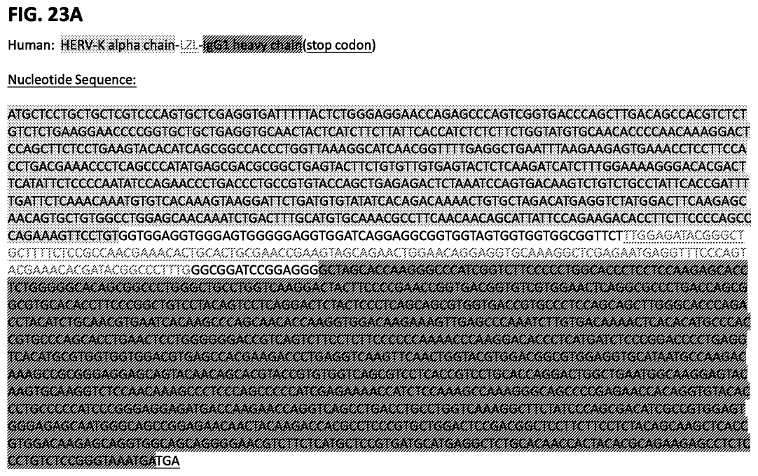

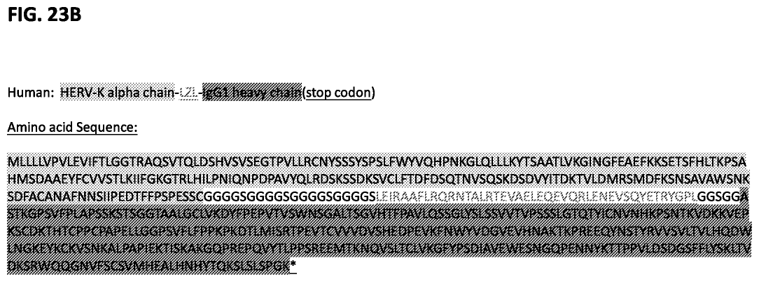

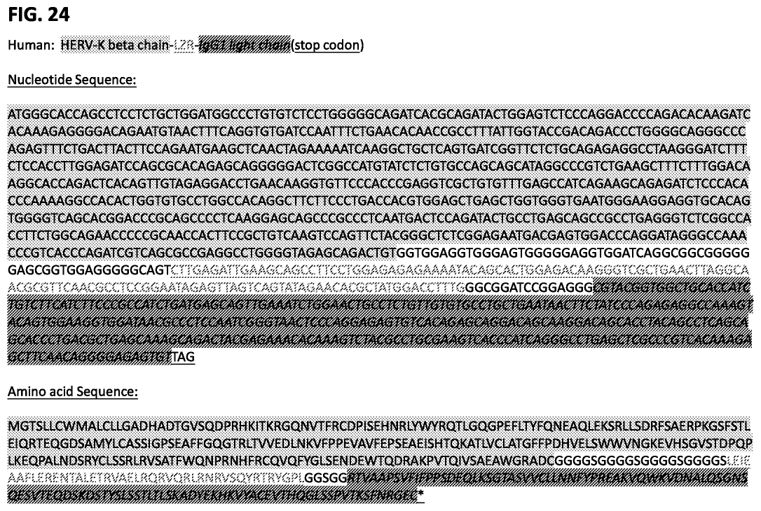

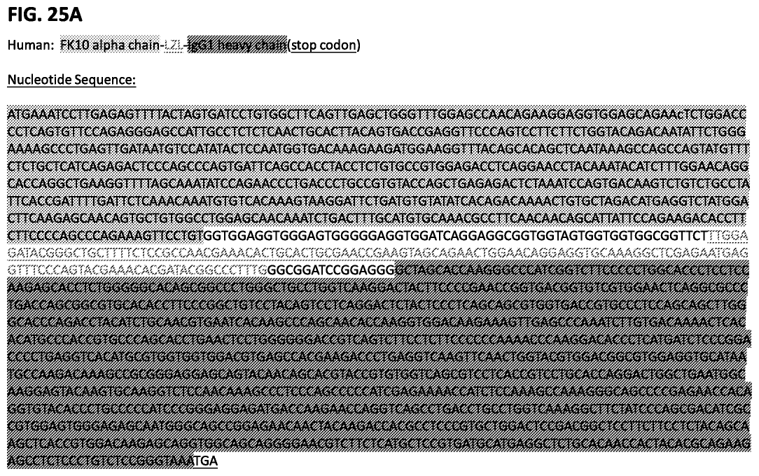

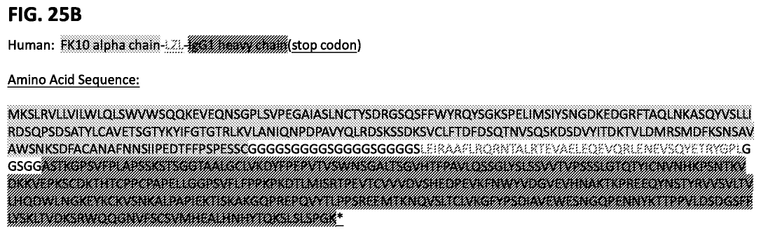

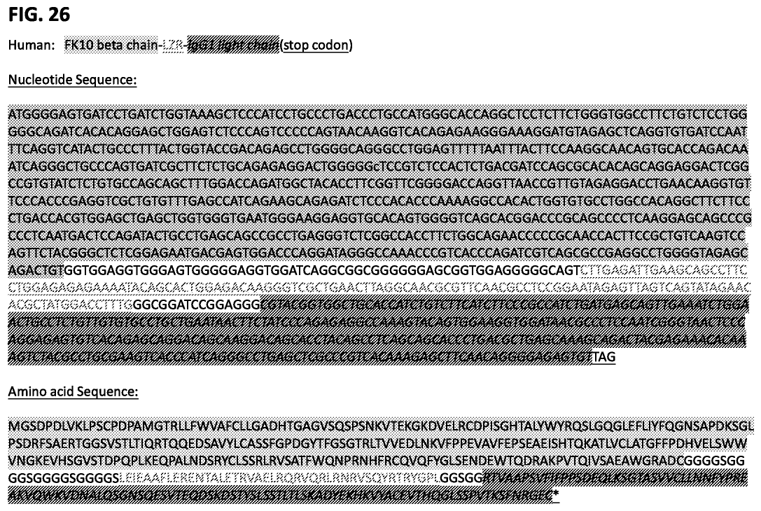

Additionally, it was further evaluated if this strategy is applicable for generating additional TCR-Igg fusion proteins. A mouse OTII-TCR-Igg fusion comprising a mouse OTII TCR specific to the chicken ovalbumin₃₂₃₋₃₃₉ antigen peptide and fused to a mouse IgG2c via a leucine zipper gave high expression. Cells were transfected as described in Example 1 with dual plasmids encoding OTII-TCR-VαCα-LZL-IgG2c(HC) and OTII-TCR-VbCb-LZR-IgG2c(LC). As shown in FIG. 10A, cells transfected to express mouse OTII-TCR-Igg had higher levels of protein in the supernatant than untransfected cells, indicating high expression of the OTII-TCR-Igg fusion. Substitution of the mouse IgG2c with human IgG1 and the mouse TCR with a human TCR (e.g., HERV-K-TCR and FK10-TCR) also resulted in high expression when comparing protein concentration in supernatant in transfected cells compared to untransfected cells as shown in FIG. 10B. The HERV-K-TCR is a TCR that recognizes a human endogenous retrovirus (HERV-K) envelope antigenic peptide that is a tumor associated antigen expressed by melanoma cells, but not healthy skin cells. The FK10-TCR is a TCR that recognizes the HLA-A02-restricted FLGKIWPSYK epitope derived from HIV Gag protein (Jones, et al. (2017) Biomaterials 117:44-53). Thus, either a mouse or human Igg scaffold supports robust production of both mouse and human soluble TCRs. Components of the HERV-K-TCR-IgG are identified in Table 4 and shown in FIGS. 23A-23B and FIG. 24. Components of the FK10-TCR-IgG are identified in Table 4 and shown in FIGS. 25A-25B and FIG. 26.

TABLE 4
components of TCR-Igg fusion proteins comprising a human
TCRs and a human IgG1 framework

| | SEQ ID NO | SEQ ID NO |
|---|---|---|
| Component | (nucleic acid) | (amino acid) |
| HERV-K TCR alpha | 63 | 64 |
| HERV-K TCR beta | 65 | 66 |
| FK10 TCR alpha | 75 | 76 |
| FK10 TCR beta | 77 | 78 |
| LZL | 5 | 6 |
| LZR | 7 | 8 |
| Linker (GGGGS)₄ | 9 | 10 |
| Linker GGSGG | 11 | 12 |
| IgG1 heavy chain | 67 | 68 |
| IgG1 light chain | 69 | 70 |
| HERV-K TCRα-LZL-IgG1 HC | 71 | 72 |
| HERV-K TCRβ-LZR-IgG1 CL | 73 | 74 |
| FK10 TCRα-LZL-IgG1 HC | 79 | 80 |
| FK10 TCRβ-LZR-IgG1 CL | 81 | 82 |

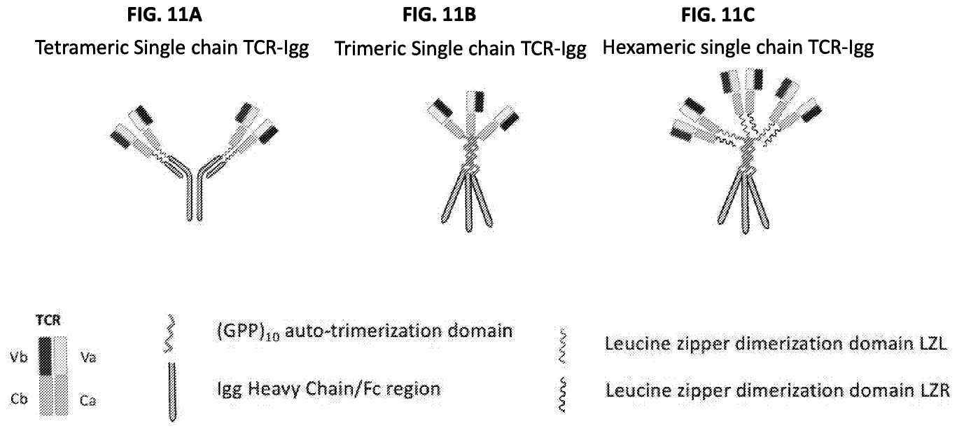

In addition to pairing natural TCRs to produce TCR-Igg dimer, this Igg framework may be used to generate soluble TCRs as trimers, tetramers or hexamers with similar high protein yields. (FIG. 11A-11C)

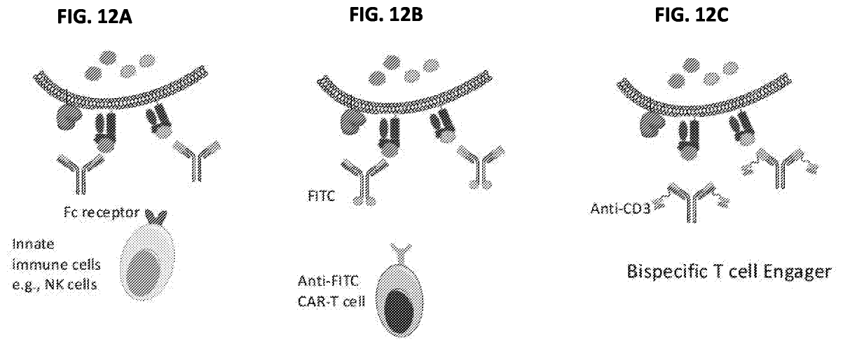

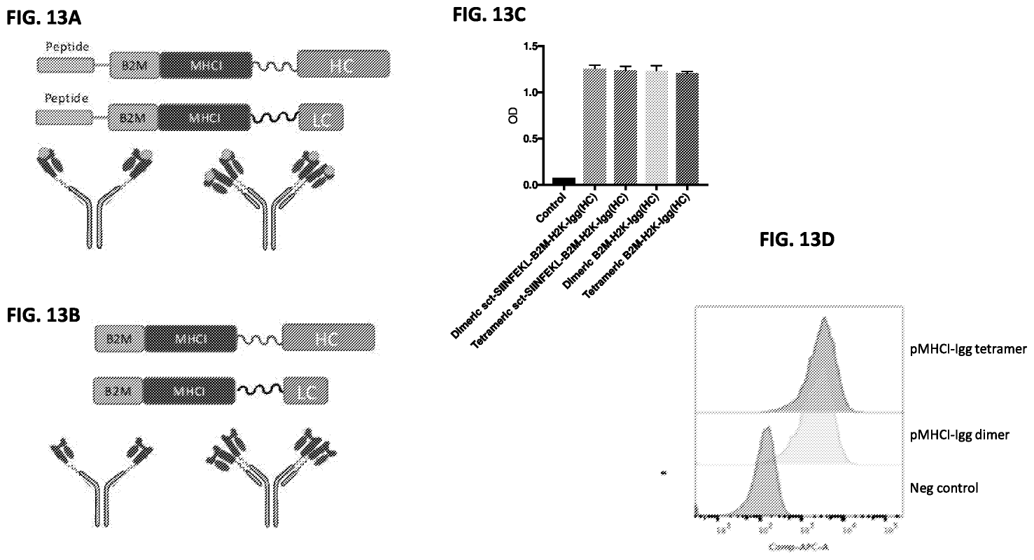

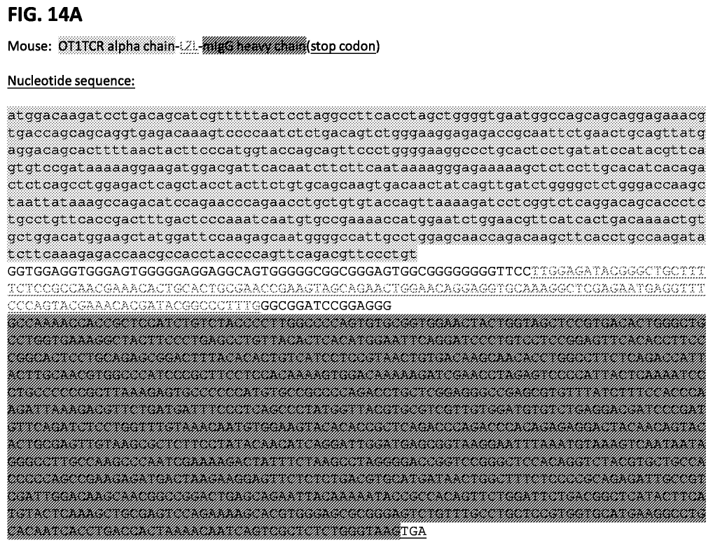

Moreover, this strategy may be used to generate a Bispecific T cell engager, for example, by fusing anti-CD3 scFV to TCR-Igg fusion (FIG. 12C), or conjugating FITC molecules onto TCR-Igg and direct universal CAR-T cells (anti-FITC CAR-T) for cancer therapy (FIG. 12B), or recruiting innate immune cells to target cells expressing a pMHCI by Fc receptor engagement (FIG. 12A).

### Example 4—Soluble Multimeric MCH Fusion Proteins

The current pMHC tetramers are mostly streptavidin or polymer based, which are often produced in bacteria and largely immunogenic. The Igg-framework design of Examples 1-3, enables high-yield production of pMHC multimers in mammalian cells. Custom based pMHC is generated by one-step cloning of single-chain MHC-peptide into the expression construct or cloning MHC-only into the expression construct then simply loading free peptide into the empty MHC-Igg multimer. The multimerization potential of leucine zipper Igg offers another possibility of generating soluble pMHC tetramer, which conventionally has been generated based on biotin-streptavidin interaction. A pMHC dimer can be readily constructed by fusing a single-chain peptide-MHCI onto the Igg heavy chain constant region via a leucine zipper. While, a pMHC tetramer is constructed by fusing a single-chain peptide-MHC onto both the Igg heavy chain and light chain constant region via a leucine zipper (FIG. 13A). Additionally, pMHC dimers or tetramers can be constructed by a fusing single-chain B2M-MHCI without covalently linked cognate peptide onto Igg heavy chain and light chain constant region via a leucine zipper (FIG. 13B), allowing for subsequent loading of cognate peptide onto the empty MHCI-Igg multimer.

As a proof of concept study, a single-chain SIINFEKL-B2M-H2Kb comprising Ova₂₅₇₋₂₆₅ peptide antigen operably linked to β2-microglobulin (B2M) that was further operably linked to the H2Kb a domain (e.g., α1+α2+α3 domains) was used as a model. Plasmid constructs encoding single chain SIINFEKL-B2M-H2Kb linked via a leucine zipper domain to a mouse IgG2c heavy chain only (e.g., dimeric sct-SIINFEKL-B2M-H2Kb-Igg(HC)) or to both the IgG2c heavy chain and light chains (e.g., tetrameric sct-SIINFEKL-B2M-H2Kb-Igg) were expressed as described in Example 1. Likewise, plasmids encoding empty MHCI (e.g., B2M-H2Kb) linked via a leucine zipper domain to a mouse IgG2c heavy chain only (e.g., dimeric B2M-H2K-Igg(HC)) or to both the IgG2c heavy chain and light chain (e.g., dimeric B2M-H2K-Igg) were expressed.

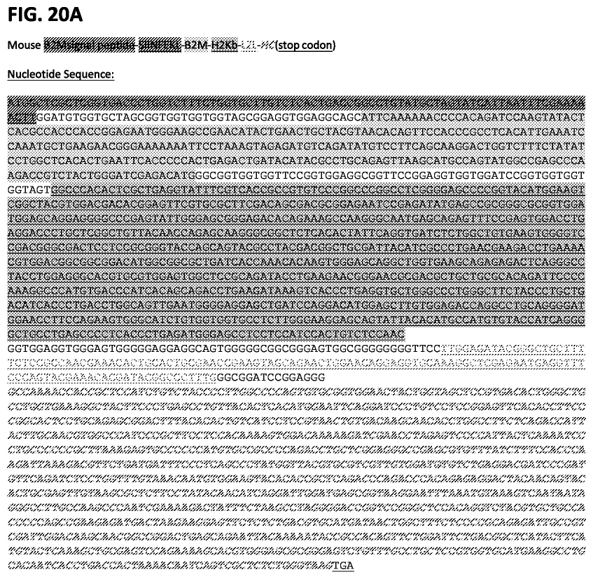

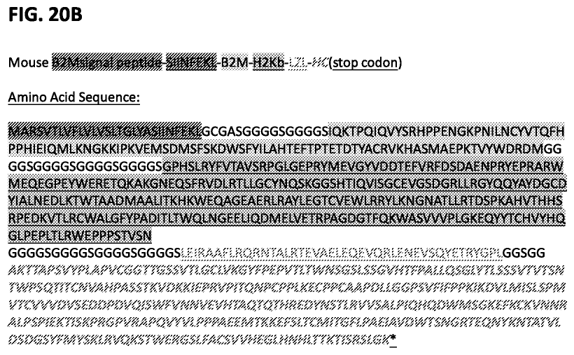

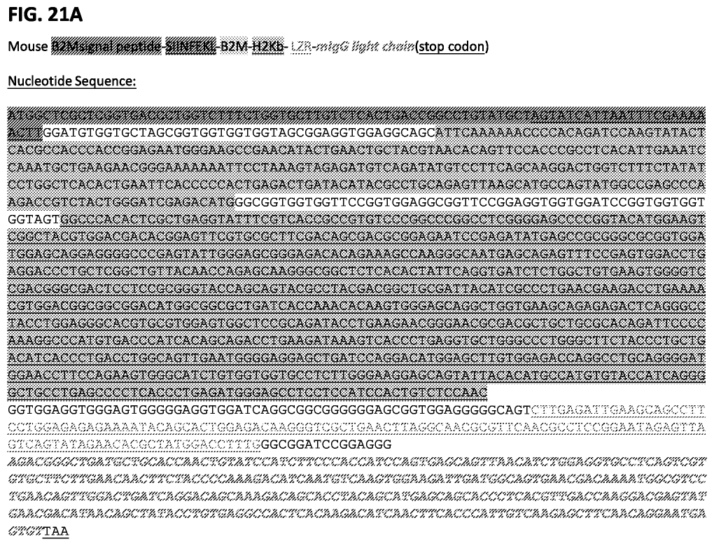

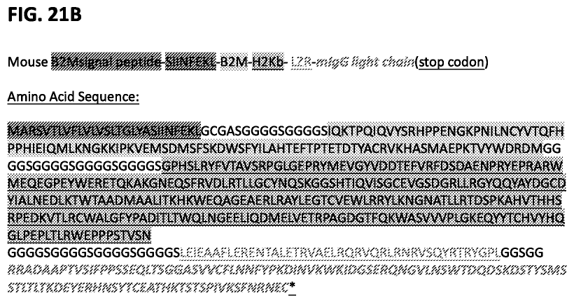

The B2M-H2Kb-Igg constructs evaluated comprised a B2M signal peptide operably linked to B2M-H2Kb (either with or without Ova₂₅₇₋₂₆₅ (e.g., SIINFEKL) peptide). The B2M-H2Kb was operably linked via a glycine serine linker (e.g., (GGGS)₄) to a leucine zipper domain. The leucine zipper was operably linked via a glycine serine linker to a IgG2c heavy chain or light chain. The construct components are indicated below and identified by sequence in Table 4.
1) Dimeric MHCI-Igg (with peptide)
   B2M signal peptide-Ova₂₅₇₋₂₆₅-B2M-H2Kb-LZL-CH1-CH2-CH3 (FIGS. 20A-20B)
2) Tetrameric MHCI-Igg (with peptide):
   B2M signal peptide-Ova₂₅₇₋₂₆₅-B2M-H2Kb-LZL-CH1-CH2-CH3 (FIGS. 20A-20B)
   B2M signal peptide-Ova₂₅₇₋₂₆₅-B2M-H2Kb-LZR-LC (FIGS. 21A-21B)
3) Dimeric MHCI-Igg (without peptide):
   B2M signal peptide-B2M-H2Kb-LZL-CH1-CH2-CH3
4) Tetrameric MHCI-Igg (without peptide):
   B2M signal peptide-B2M-H2Kb-LZL-CH1-CH2-CH3
   B2M signal peptide-B2M-H2Kb-LZR-LC

TABLE 5
components of MHCI-Igg fusion proteins comprising a
mouse B2M-H2Kb and a mouse IgG2c framework

| | SEQ ID NO | SEQ ID NO |
|---|---|---|
| Component | (nucleic acid) | (amino acid) |
| B2M signal peptide | 37 | 38 |
| Ova₂₅₇₋₂₆₅ | 39 | 40 |
| B2M | 41 | 42 |
| H2Kb | 43 | 44 |
| LZL | 5 | 6 |
| LZR | 7 | 8 |
| IgG2c heavy chain (CH1-CH2-CH3) | 13 | 14 |
| IgG2c light chain (CL) | 15 | 16 |
| Linker (GGGGS)₄ | 9 | 10 |
| Linker GGSGG | 11 | 12 |
| B2M signal peptide-Ova₂₅₇₋₂₆₅-B2M-H2Kb- | 45 | 46 |
| LZL-CH1-CH2-CH3 | | |
| B2M signal peptide-Ova₂₅₇₋₂₆₅-B2M- | 47 | 48 |
| H2Kb-LZR-CL | | |

The level of expression was measured. High amounts of secreted SIINFEKL-B2M-H2Kb dimer or tetramer or B2M-H2Kb dimer or tetramer were detected in cell supernatant as measured by ELISA (FIG. 13C).

The functionality of the secreted molecules was further evaluated. OT1 T cells expressing a TCR specific to SIINFEKL-H-2Kb were stained with raw cell culture supernatant from cells induced to express dimeric or tetrameric sct-SIINFEKL-B2M-H2K-Igg (e.g., single-chain peptide/MHCI linked via a leucine zipper domain to mouse Igg). The level of surface staining was assessed by flow cytometry. It was determined that 100% of OT1 T cells were successfully stained by either the dimer or tetramer (FIG. 13D).

Similar to pMHCI multimer, constructs containing leucine zipper Igg are being used for producing either an pMHCII tetramer by fusing single-chain MHCII or pMHCII dimer (see FIG. 13A-B) To further simplify the production of a tetramer, empty/peptide-null version of MHCI/II multimer (FIG. 13B-D) can be generated so that desired multimer can be directly produced by pulsing empty multimers with desired peptide.

### EQUIVALENTS

Those skilled in the art will recognize or be able to ascertain, using no more than routine experimentation, many equivalents of the specific embodiments described herein. Such equivalents are intended to be encompassed by the following claims.

SUMMARY OF SEQUENCES

| Description | Nucleotide Sequence | SEQ ID NO: | Amino Acid Sequence | SEQ ID NO: |
|---|---|---|---|---|
| Murine | ATGGACAAGATCCTGACAGCA | 1 | MDKILTASFLLLGLHLAGTI | 2 |
| OTI TCRα | TCGTTTTTACTCCTAGGCCTT | | NGQQQEKRDQQQVRQSPQSL | |
| | CACCTAGCTGGGGTGAATGGC | | TVWEGETAILNCSYEDSTFN | |
| | CAGCAGCAGGAGAAACGTGAC | | YFPWYQQFPGEGPALLISIR | |
| | CAGCAGCAGGTGAGACAAAGT | | SVSDKKEDGRFTIFFNKREK | |
| | CCCCAATCTCTGACAGTCTGG | | KLSLHITDSQPGDSATYFCA | |
| | GAAGGAGAGACCGCAATTCTG | | ASDNYQLIWGSGTKLIIKPD | |
| | AACTGCAGTTATGAGGACAGC | | IQNPEPAVYQLKDPRSQDST | |
| | ACTTTTAACTACTTCCCATGG | | LCLFTDFDSQINVPKTMESG | |
| | TACCAGCAGTTCCCTGGGGAA | | TFITDKTVLDMEAMDSKSNG | |
| | GGCCCTGCACTCCTGATATCC | | AIAWSNQTSFTCQDIFKETN | |
| | ATACGTTCAGTGTCCGATAAA | | ATYPSSDVPC | |
| | AAGGAAGATGGACGATTCACA | | | |
| | ATCTTCTTCAATAAAAGGGAG | | | |
| | AAAAAGCTCTCCTTGCACATC | | | |
| | ACAGACTCTCAGCCTGGAGAC | | | |
| | TCAGCTACCTACTTCTGTGCA | | | |
| | GCAAGTGACAACTATCAGTTG | | | |
| | ATCTGGGGCTCTGGGACCAAG | | | |
| | CTAATTATAAAGCCAGACATC | | | |
| | CAGAACCCAGAACCTGCTGTG | | | |
| | TACCAGTTAAAAGATCCTCGG | | | |
| | TCTCAGGACAGCACCCTCTGC | | | |
| | CTGTTCACCGACTTTGACTCC | | | |
| | CAAATCAATGTGCCGAAAACC | | | |
| | ATGGAATCTGGAACGTTCATC | | | |
| | ACTGACAAAACTGTGCTGGAC | | | |
| | ATGGAAGCTATGGATTCCAAG | | | |
| | AGCAATGGGGCCATTGCCTGG | | | |
| | AGCAACCAGACAAGCTTCACC | | | |
| | TGCCAAGATATCTTCAAAGAG | | | |
| | ACCAACGCCACCTACCCCAGT | | | |
| | TCAGACGTTCCCTGT | | | |
| Murine | | | | |
| OTI TCRβ | ATGTCTAACACTGTCCTCGCT | 3 | MSNTVLADSAWGITLLSWVT | 4 |
| | GATTCTGCCTGGGGCATCACC | | VFLLGTSSADSGVVQSPRHI | |
| | CTGCTATCTTGGGTTACTGTC | | IKEKGGRSVLTCIPISGHSN | |
| | TTTCTCTTGGGAACAAGTTCA | | VVWYQQTLGKELKFLIQHYE | |
| | GCAGATTCTGGGGTTGTCCAG | | KVERDKGFLPSRFSVQQFDD | |
| | TCTCCAAGACACATAATCAAA | | YHSEMNMSALELEDSAMYFC | |
| | GAAAAGGGAGGAAGGTCCGTT | | ASSRANYEQYFGPGTRLTVL | |
| | CTGACGTGTATTCCCATCTCT | | EDLRNVTPPKVSLFEPSKAE | |
| | GGACATAGCAATGTGGTCTGG | | IANKQKATLVCLARGFFPDH | |
| | TACCAGCAGACTCTGGGGAAG | | VELSWWVNGKEVHSGVSTDP | |
| | GAATTAAAGTTCCTTATTCAG | | QAYKESNYSYCLSSRLRVSA | |
| | CATTATGAAAAGGTGGAGAGA | | TFWHNPRNHFRCQVQFHGLS | |
| | GACAAAGGATTCCTACCCAGC | | EEDKWPEGSPKPVTQNISAE | |
| | AGATTCTCAGTCCAACAGTTT | | AWGRADC | |
| | GATGACTATCACTCTGAAATG | | | |
| | AACATGAGTGCCTTGGAACTG | | | |
| | GAGGACTCTGCTATGTACTTC | | | |
| | TGTGCCAGCTCTCGGGCCAAT | | | |
| | TATGAACAGTACTTCGGTCCC | | | |
| | GGCACCAGGCTCACGGTTTTA | | | |
| | GAGGATCTGAGAAATGTGACT | | | |
| | CCACCCAAGGTCTCCTTGTTT | | | |
| | GAGCCATCAAAAGCAGAGATT | | | |
| | GCAAACAAACAAAAGGCTACC | | | |
| | CTCGTGTGCTTGGCCAGGGGC | | | |
| | TTCTTCCCTGACCACGTGGAG | | | |
| | CTGAGCTGGTGGGTGAATGGC | | | |
| | AAGGAGGTCCACAGTGGGGTC | | | |
| | AGCACGGACCCTCAGGCCTAC | | | |
| | AAGGAGAGCAATTATAGCTAC | | | |
| | TGCCTGAGCAGCCGCCTGAGG | | | |
| | GTCTCTGCTACCTTCTGGCAC | | | |
| | AATCCTCGAAACCACTTCCGC | | | |
| | TGCCAAGTGCAGTTCCATGGG | | | |
| | CTTTCAGAGGAGGACAAGTGG | | | |
| | CCAGAGGGCTCACCCAAACCT | | | |
| | GTCACACAGAACATCAGTGCA | | | |
| | GAGGCCTGGGGCCGAGCAGAC | | | |
| | TGT | | | |
| LZL | TTGGAGATACGGGCTGCTTTT | 5 | LEIRAAFLRQRNTALRTEVA | 6 |
| | CTCCGCCAACGAAACACTGCA | | ELEQEVQRLENEVSQYETRY | |
| | CTGCGAACCGAAGTAGCAGAA | | GPL | |
| | CTGGAACAGGAGGTGCAAAGG | | | |
| | CTCGAGAATGAGGTTTCCCAG | | | |
| | TACGAAACACGATACGGCCCT | | | |
| | TTG | | | |
| LZR | CTTGAGATTGAAGCAGCCTTC | 7 | LEIEAAFLERENTALETRVA | 8 |
| | CTGGAGAGAGAAAATACAGCA | | ELRQRVQRLRNRVSQYRTRY | |
| | CTGGAGACAAGGGTCGCTGAA | | GPL | |
| | CTTAGGCAACGCGTTCAACGC | | | |
| | CTCCGGAATAGAGTTAGTCAG | | | |
| | TATAGAACACGCTATGGACCT | | | |
| | TTG | | | |
| Linker | GGTGGAGGTGGGAGTGGGGGA | 9 | GGGGSGGGGSGGGGSGGGGS | 10 |
| (GGGGS)₄ | GGAGGCAGTGGGGGCGGCGGG | | | |
| | AGTGGCGGGGGGGGTTCC | | | |
| Linker | GGCGGATCCGGAGGG | 11 | GGSGG | 12 |
| Murine | GCCAAAACCACCGCTCCATCT | 13 | AKTTAPSVYPLAPVCGGTTG | 14 |
| IgG heavy | GTCTACCCCTTGGCCCCAGTG | | SSVTLGCLVKGYFPEPVTLT | |
| chain | TGCGGTGGAACTACTGGTAGC | | WNSGSLSSGVHTFPALLQSG | |
| | TCCGTGACACTGGGCTGCCTG | | LYTLSSSVTVTSNTWPSQTI | |
| | GTGAAAGGCTACTTCCCTGAG | | TCNVAHPASSTKVDKKIEPR | |
| | CCTGTTACACTCACATGGAAT | | VPITQNPCPPLKECPPCAAP | |
| | TCAGGATCCCTGTCCTCCGGA | | DLLGGPSVFIFPPKIKDVLM | |
| | GTTCACACCTTCCCGGCACTC | | ISLSPMVTCVVVDVSEDDPD | |
| | CTGCAGAGCGGACTTTACACA | | VQISWFVNNVEVHTAQTQTH | |
| | CTGTCATCCTCCGTAACTGTG | | REDYNSTLRVVSALPIQHQD | |
| | ACAAGCAACACCTGGCCTTCT | | WMSGKEFKCKVNNRALPSPI | |
| | CAGACCATTACTTGCAACGTG | | EKTISKPRGPVRAPQVYVLP | |
| | GCCCATCCCGCTTCCTCCACA | | PPAEEMTKKEFSLTCMITGF | |
| | AAAGTGGACAAAAAGATCGAA | | LPAEIAVDWTSNGRTEQNYK | |
| | CCTAGAGTCCCCATTACTCAA | | NTATVLDSDGSYFMYSKLRV | |
| | AATCCCTGCCCCCCGCTTAAA | | QKSTWERGSLFACSVVHEGL | |
| | GAGTGCCCCCCATGTGCCGCC | | HNHLTTKTISRSLGK | |
| | CCAGACCTGCTCGGAGGGCCG | | | |
| | AGCGTGTTTATCTTTCCACCC | | | |
| | AAGATTAAAGACGTTCTGATG | | | |
| | ATTTCCCTCAGCCCTATGGTT | | | |
| | ACGTGCGTCGTTGTGGATGTG | | | |
| | TCTGAGGACGATCCCGATGTT | | | |
| | CAGATCTCCTGGTTTGTAAAC | | | |
| | AATGTGGAAGTACACACCGCT | | | |
| | CAGACCCAGACCCACAGAGAG | | | |
| | GACTACAACAGTACACTGCGA | | | |
| | GTTGTAAGCGCTCTTCCTATA | | | |
| | CAACATCAGGATTGGATGAGC | | | |
| | GGTAAGGAATTTAAATGTAAA | | | |
| | GTCAATAATAGGGCCTTGCCA | | | |
| | AGCCCAATCGAAAAGACTATT | | | |
| | TCTAAGCCTAGGGGACCGGTC | | | |
| | CGGGCTCCACAGGTCTACGTG | | | |
| | CTGCCACCCCCAGCCGAAGAG | | | |
| | ATGACTAAGAAGGAGTTCTCT | | | |
| | CTGACGTGCATGATAACTGGC | | | |
| | TTTCTCCCCGCAGAGATTGCC | | | |
| | GTCGATTGGACAAGCAACGGC | | | |
| | CGGACTGAGCAGAATTACAAA | | | |
| | AATACCGCCACAGTTCTGGAT | | | |
| | TCTGACGGCTCATACTTCATG | | | |
| | TACTCAAAGCTGCGAGTCCAG | | | |
| | AAAAGCACGTGGGAGCGCGGG | | | |
| | AGTCTGTTTGCCTGCTCCGTG | | | |
| | GTGCATGAAGGCCTGCACAAT | | | |
| | CACCTGACCACTAAAACAATC | | | |
| | AGTCGCTCTCTGGGTAAGTGA | | | |
| Murine | AGACGGGCTGATGCTGCACCA | 15 | RRADAAPTVSIFPPSSEQLT | 16 |
| IgG light | ACTGTATCCATCTTCCCACCA | | SGGASVVCFLNNFYPKDINV | |
| chain | TCCAGTGAGCAGTTAACATCT | | KWKIDGSERQNGVLNSWTDQ | |
| | GGAGGTGCCTCAGTCGTGTGC | | DSKDSTYSMSSTLTLTKDEY | |
| | TTCTTGAACAACTTCTACCCC | | ERHNSYTCEATHKTSTSPIV | |
| | AAAGACATCAATGTCAAGTGG | | KSFNRNEC | |
| | AAGATTGATGGCAGTGAACGA | | | |
| | CAAAATGGCGTCCTGAACAGT | | | |
| | TGGACTGATCAGGACAGCAAA | | | |
| | GACAGCACCTACAGCATGAGC | | | |
| | AGCACCCTCACGTTGACCAAG | | | |
| | GACGAGTATGAACGACATAAC | | | |
| | AGCTATACCTGTGAGGCCACT | | | |
| | CACAAGACATCAACTTCACCC | | | |
| | ATTGTCAAGAGCTTCAACAGG | | | |
| | AATGAGTGTTAA | | | |
| OTT fusion | ATGGACAAGATCCTGACAGCA | 17 | MDKILTASFLLLGLHLAGVN | 18 |
| TCRα- | TCGTTTTTACTCCTAGGCCTT | | GQQQEKRDQQQVRQSPQSLT | |
| LZL-IgG_{HC} | CACCTAGCTGGGGTGAATGGC | | VWEGETAILNCSYEDSTFNY | |
| | CAGCAGCAGGAGAAACGTGAC | | FPWYQQFPGEGPALLISIRS | |
| | CAGCAGCAGGTGAGACAAAGT | | VSDKKEDGRFTIFFNKREKK | |
| | CCCCAATCTCTGACAGTCTGG | | LSLHITDSQPGDSATYFCAA | |
| | GAAGGAGAGACCGCAATTCTG | | SDNYQLIWGSGTKLIIKPDI | |
| | AACTGCAGTTATGAGGACAGC | | QNPEPAVYQLKDPRSQDSTL | |
| | ACTTTTAACTACTTCCCATGG | | CLFTDFDSQINVPKTMESGT | |
| | TACCAGCAGTTCCCTGGGGAA | | FITDKTVLDMEAMDSKSNGA | |
| | GGCCCTGCACTCCTGATATCC | | IAWSNQTSFTCQDIFKETNA | |
| | ATACGTTCAGTGTCCGATAAA | | TYPSSDVPCGGGGSGGGGSG | |
| | AAGGAAGATGGACGATTCACA | | GGGSGGGGSLEIRAAFLRQR | |
| | ATCTTCTTCAATAAAAGGGAG | | NTALRTEVAELEQEVQRLEN | |
| | AAAAAGCTCTCCTTGCACATC | | EVSQYETRYGPLGGSGGAKT | |
| | ACAGACTCTCAGCCTGGAGAC | | TAPSVYPLAPVCGGTTGSSV | |
| | TCAGCTACCTACTTCTGTGCA | | TLGCLVKGYFPEPVTLTWNS | |
| | GCAAGTGACAACTATCAGTTG | | GSLSSGVHTFPALLQSGLYT | |
| | ATCTGGGGCTCTGGGACCAAG | | LSSSVTVTSNTWPSQTITCN | |
| | CTAATTATAAAGCCAGACATC | | VAHPASSTKVDKKIEPRVPI | |
| | CAGAACCCAGAACCTGCTGTG | | TQNPCPPLKECPPCAAPDLL | |
| | TACCAGTTAAAAGATCCTCGG | | GGPSVFIFPPKIKDVLMISL | |
| | TCTCAGGACAGCACCCTCTGC | | SPMVTCVVVDVSEDDPDVQI | |
| | CTGTTCACCGACTTTGACTCC | | SWFVNNVEVHTAQTQTHRED | |
| | CAAATCAATGTGCCGAAAACC | | YNSTLRVVSALPIQHQDWMS | |
| | ATGGAATCTGGAACGTTCATC | | GKEFKCKVNNRALPSPIEKT | |
| | ACTGACAAAACTGTGCTGGAC | | ISKPRGPVRAPQVYVLPPPA | |
| | ATGGAAGCTATGGATTCCAAG | | EEMTKKEFSLTCMITGFLPA | |
| | AGCAATGGGGCCATTGCCTGG | | EIAVDWTSNGRTEQNYKNTA | |
| | AGCAACCAGACAAGCTTCACC | | TVLDSDGSYFMYSKLRVQKS | |
| | TGCCAAGATATCTTCAAAGAG | | TWERGSLFACSVVHEGLHNH | |
| | ACCAACGCCACCTACCCCAGT | | LTTKTISRSLGK | |
| | TCAGACGTTCCCTGTGGTGGA | | | |
| | GGTGGGAGTGGGGGAGGAGGC | | | |
| | AGTGGGGGCGGCGGGAGTGGC | | | |
| | GGGGGGGGTTCCTTGGAGATA | | | |
| | CGGGCTGCTTTTCTCCGCCAA | | | |
| | CGAAACACTGCACTGCGAACC | | | |
| | GAAGTAGCAGAACTGGAACAG | | | |
| | GAGGTGCAAAGGCTCGAGAAT | | | |
| | GAGGTTTCCCAGTACGAAACA | | | |
| | CGATACGGCCCTTTGGGCGGA | | | |
| | TCCGGAGGGGCCAAAACCACC | | | |
| | GCTCCATCTGTCTACCCCTTG | | | |
| | GCCCCAGTGTGCGGTGGAACT | | | |
| | ACTGGTAGCTCCGTGACACTG | | | |
| | GGCTGCCTGGTGAAAGGCTAC | | | |
| | TTCCCTGAGCCTGTTACACTC | | | |
| | ACATGGAATTCAGGATCCCTG | | | |
| | TCCTCCGGAGTTCACACCTTC | | | |
| | CCGGCACTCCTGCAGAGCGGA | | | |
| | CTTTACACACTGTCATCCTCC | | | |
| | GTAACTGTGACAAGCAACACC | | | |
| | TGGCCTTCTCAGACCATTACT | | | |
| | TGCAACGTGGCCCATCCCGCT | | | |
| | TCCTCCACAAAAGTGGACAAA | | | |
| | AAGATCGAACCTAGAGTCCCC | | | |
| | ATTACTCAAAATCCCTGCCCC | | | |
| | CCGCTTAAAGAGTGCCCCCCA | | | |
| | TGTGCCGCCCCAGACCTGCTC | | | |
| | GGAGGGCCGAGCGTGTTTATC | | | |
| | TTTCCACCCAAGATTAAAGAC | | | |
| | GTTCTGATGATTTCCCTCAGC | | | |
| | CCTATGGTTACGTGCGTCGTT | | | |
| | GTGGATGTGTCTGAGGACGAT | | | |
| | CCCGATGTTCAGATCTCCTGG | | | |
| | TTTGTAAACAATGTGGAAGTA | | | |
| | CACACCGCTCAGACCCAGACC | | | |
| | CACAGAGAGGACTACAACAGT | | | |
| | ACACTGCGAGTTGTAAGCGCT | | | |
| | CTTCCTATACAACATCAGGAT | | | |
| | TGGATGAGCGGTAAGGAATTT | | | |
| | AAATGTAAAGTCAATAATAGG | | | |
| | GCCTTGCCAAGCCCAATCGAA | | | |
| | AAGACTATTTCTAAGCCTAGG | | | |
| | GGACCGGTCCGGGCTCCACAG | | | |
| | GTCTACGTGCTGCCACCCCCA | | | |
| | GCCGAAGAGATGACTAAGAAG | | | |
| | GAGTTCTCTCTGACGTGCATG | | | |
| | ATAACTGGCTTTCTCCCCGCA | | | |
| | GAGATTGCCGTCGATTGGACA | | | |
| | AGCAACGGCCGGACTGAGCAG | | | |
| | AATTACAAAAATACCGCCACA | | | |
| | GTTCTGGATTCTGACGGCTCA | | | |
| | TACTTCATGTACTCAAAGCTG | | | |
| | CGAGTCCAGAAAAGCACGTGG | | | |
| | GAGCGCGGGAGTCTGTTTGCC | | | |
| | TGCTCCGTGGTGCATGAAGGC | | | |
| | CTGCACAATCACCTGACCACT | | | |
| | AAAACAATCAGTCGCTCTCTG | | | |
| | GGTAAGTGA | | | |
| OTI fusion | ATGTCTAACACTGTCCTCGCT | 19 | MSNTVLADSAWGITLLSWVT | 20 |
| TCRβ- | GATTCTGCCTGGGGCATCACC | | VFLLGTSSADSGVVQSPRHI | |
| LZR-IgG_{LC} | CTGCTATCTTGGGTTACTGTC | | IKEKGGRSVLTCIPISGHSN | |
| | TTTCTCTTGGGAACAAGTTCA | | VVWYQQTLGKELKFLIQHYE | |
| | GCAGATTCTGGGGTTGTCCAG | | KVERDKGFLPSRFSVQQFDD | |
| | TCTCCAAGACACATAATCAAA | | YHSEMNMSALELEDSAMYFC | |
| | GAAAAGGGAGGAAGGTCCGTT | | ASSRANYEQYFGPGTRLTVL | |
| | CTGACGTGTATTCCCATCTCT | | EDLRNVTPPKVSLFEPSKAE | |
| | GGACATAGCAATGTGGTCTGG | | IANKQKATLVCLARGFFPDH | |
| | TACCAGCAGACTCTGGGGAAG | | VELSWWVNGKEVHSGVSTDP | |
| | GAATTAAAGTTCCTTATTCAG | | QAYKESNYSYCLSSRLRVSA | |
| | CATTATGAAAAGGTGGAGAGA | | TFWHNPRNHFRCQVQFHGLS | |
| | GACAAAGGATTCCTACCCAGC | | EEDKWPEGSPKPVTQNISAE | |
| | AGATTCTCAGTCCAACAGTTT | | AWGRADCGGGGSGGGGSGGG | |
| | GATGACTATCACTCTGAAATG | | GSGGGGSLEIEAAFLERENT | |
| | AACATGAGTGCCTTGGAACTG | | ALETRVAELRQRVQRLRNRV | |
| | GAGGACTCTGCTATGTACTTC | | SQYRTRYGPLGGSGGRRADA | |
| | TGTGCCAGCTCTCGGGCCAAT | | APTVSIFPPSSEQLTSGGAS | |
| | TATGAACAGTACTTCGGTCCC | | VVCFLNNFYPKDINVKWKID | |
| | GGCACCAGGCTCACGGTTTTA | | GSERQNGVLNSWTDQDSKDS | |
| | GAGGATCTGAGAAATGTGACT | | TYSMSSTLTLTKDEYERHNS | |
| | CCACCCAAGGTCTCCTTGTTT | | YTCEATHKTSTSPIVKSFNR | |
| | GAGCCATCAAAAGCAGAGATT | | NEC | |
| | GCAAACAAACAAAAGGCTACC | | | |
| | CTCGTGTGCTTGGCCAGGGGC | | | |
| | TTCTTCCCTGACCACGTGGAG | | | |
| | CTGAGCTGGTGGGTGAATGGC | | | |
| | AAGGAGGTCCACAGTGGGGTC | | | |
| | AGCACGGACCCTCAGGCCTAC | | | |
| | AAGGAGAGCAATTATAGCTAC | | | |
| | TGCCTGAGCAGCCGCCTGAGG | | | |
| | GTCTCTGCTACCTTCTGGCAC | | | |
| | AATCCTCGAAACCACTTCCGC | | | |
| | TGCCAAGTGCAGTTCCATGGG | | | |
| | CTTTCAGAGGAGGACAAGTGG | | | |
| | CCAGAGGGCTCACCCAAACCT | | | |
| | GTCACACAGAACATCAGTGCA | | | |
| | GAGGCCTGGGGCCGAGCAGAC | | | |
| | TGTGGTGGAGGTGGGAGTGGG | | | |
| | GGAGGTGGATCAGGCGGCGGG | | | |
| | GGGAGCGGTGGAGGGGGCAGT | | | |
| | CTTGAGATTGAAGCAGCCTTC | | | |
| | CTGGAGAGAGAAAATACAGCA | | | |
| | CTGGAGACAAGGGTCGCTGAA | | | |
| | CTTAGGCAACGCGTTCAACGC | | | |
| | CTCCGGAATAGAGTTAGTCAG | | | |
| | TATAGAACACGCTATGGACCT | | | |
| | TTGGGCGGATCCGGAGGGAGA | | | |
| | CGGGCTGATGCTGCACCAACT | | | |
| | GTATCCATCTTCCCACCATCC | | | |
| | AGTGAGCAGTTAACATCTGGA | | | |
| | GGTGCCTCAGTCGTGTGCTTC | | | |
| | TTGAACAACTTCTACCCCAAA | | | |
| | GACATCAATGTCAAGTGGAAG | | | |
| | ATTGATGGCAGTGAACGACAA | | | |
| | AATGGCGTCCTGAACAGTTGG | | | |
| | ACTGATCAGGACAGCAAAGAC | | | |
| | AGCACCTACAGCATGAGCAGC | | | |
| | ACCCTCACGTTGACCAAGGAC | | | |
| | GAGTATGAACGACATAACAGC | | | |
| | TATACCTGTGAGGCCACTCAC | | | |
| | AAGACATCAACTTCACCCATT | | | |
| | GTCAAGAGCTTCAACAGGAAT | | | |
| | GAGTGTTAA | | | |
| Murine 2C | | 21 | MLLALLPVLGIHFVLRDAQA | 22 |
| TCRα | ATGCTGCTGGCTCTGCTGCCT | | QSVTQPDARVTVSEGASLQL | |
| | GTGCTGGGCATCCACTTCGTG | | RCKYSYSATPYLFWYVQYPR | |
| | CTGAGGGACGCCCAGGCCCAG | | QGLQLLLKYYSGDPVVQGVN | |
| | AGCGTGACCCAGCCTGACGCC | | GFEAEFSKSNSSFHLRKASV | |
| | AGAGTGACAGTGTCTGAGGGC | | HWSDSAVYFCAVSGFASALT | |
| | GCCAGCCTGCAGCTGAGATGC | | FGSGTKVIVLPYIQNPEPAV | |
| | AAGTACAGCTACAGCGCCACC | | YQLKDPRSQDSTLCLFTDFD | |
| | CCCTACCTGTTTTGGTACGTG | | SQINVPKTMESGTFITDKCV | |
| | CAGTACCCCAGACAGGGCCtg | | LDMKAMDSKSNGAIAWSNQT | |
| | CAGCTGCTGCTGAAGTACTAC | | SFTCQDIFKETNATYPSSDV | |
| | AGCGGCGACCCTGTGGTGCAG | | PC | |
| | GGCGTGAACGGCTTCGAGGCC | | | |
| | GAGTTCAGCAAGAGCAACAGC | | | |
| | AGCTTCCACCTGAGAAAGGCC | | | |
| | AGCGTGCATTGGAGCGACAGC | | | |
| | GCCGTGTATTTTTGTGCCGTG | | | |
| | AGCGGCTTCGCCAGCGCCCTG | | | |
| | ACCTTCGGCAGCGGCACAAAA | | | |
| | GTGATCGTGCTGCCCTACATC | | | |
| | CAGAACCCCGAGCCCGCCGTG | | | |
| | TACCAGCTGAAGGACCCCAGA | | | |
| | AGCCAGGACAGCACCCTGTGC | | | |
| | CTGTTCACCGACTTCGACAGC | | | |
| | CAGATCAACGTGCCCAAGACC | | | |
| | ATGGAAAGCGGCACCTTCATC | | | |
| | ACCGATAAGTGCGTGCTGGAC | | | |
| | ATGAAGGCCATGGACAGCAAG | | | |
| | TCCAACGGCGCTATCGCCTGG | | | |
| | TCCAACCAGACCTCATTCACA | | | |
| | TGCCAGGACATCTTCAAAGAG | | | |
| | ACAAACGCCACCTACCCCAGC | | | |
| | AGCGACGTGCCTTGT | | | |
| Murine 2C | ATGAGCAACACCGCCTTCCCC | 23 | MSNTAFPDPAWNTTLLSWVA | 24 |
| TCRβ | GACCCTGCCTGGAACACCACC | | LFLLGTKHMEAAVTQSPRNK | |
| | CTGCTGTCCTGGGTGGCCCTG | | VAVTGGKVTLSCNQTNNHNN | |
| | TTCCTGCTGGGCACCAAGCAC | | MYWYRQDTGHGLRLIHYSYG | |
| | ATGGAAGCCGCCGTGACACAG | | AGSTEKGDIPDGYKASRPSQ | |
| | AGCCCCAGAAACAAGGTGGCC | | ENFSLILELATPSQTSVYFC | |
| | GTGACCGGCGGCAAAGTGACC | | ASGGGGTLYFGAGTRLSVLE | |
| | CTGAGCTGCAACCAGACCAAC | | DLRNVTPPKVSLFEPSKAEI | |
| | AACCACAACAACATGTACTGG | | ANKQKATLVCLARGFFPDHV | |
| | TACAGACAGGACACCGGCCAC | | ELSWWVNGKEVHSGVCTDPQ | |
| | GGACTGAGACTGATCCACTAC | | AYKESNYSYCLSSRLRVSAT | |
| | AGCTACGGCGCTGGCAGCACC | | FWHNPRNHFRCQVQFHGLSE | |
| | GAGAAGGGCGACATCCCCGAC | | EDKWPEGSPKPVTQNISAEA | |
| | GGCTACAAGGCCAGCAGACCC | | WGRADC | |
| | AGCCAGGAAAACTTCAGCCTG | | | |
| | ATCCTGGAACTGGCCACCCCT | | | |
| | AGCCAGACCAGCGTGTACTTC | | | |
| | TGCGCCTCTGGCGGCGGAGGA | | | |
| | ACCCTGTACTTCGGAGCCGGC | | | |
| | ACCAGACTGAGCGTGCTGGAA | | | |
| | GATCTGAGAAACGTGACCCCC | | | |
| | CCCAAGGTGTCCCTGTTCGAG | | | |
| | CCCAGCAAGGCCGAGATCGCC | | | |
| | AACAAGCAGAAAGCCACCCTC | | | |
| | GTGTGCCTGGCCAGAGGCTTC | | | |
| | TTCCCTGACCACGTGGAGCTG | | | |
| | TCTTGGTGGGTGAACGGCAAA | | | |
| | GAGGTGCACAGCGGCGTCTGC | | | |
| | ACCGACCCCCAGGCCTACAAA | | | |
| | GAGAGCAACTACTCCTACTGC | | | |
| | CTGAGCAGCAGACTGAGAGTG | | | |
| | TCCGCCACCTTCTGGCACAAC | | | |
| | CCCAGAAACCACTTCAGATGC | | | |
| | CAGGTGCAGTTCCATGGCCTG | | | |
| | TCCGAAGAGGACAAGTGGCCC | | | |
| | GAGGGCAGCCCTAAGCCTGTG | | | |
| | ACACAGAACATCAGCGCCGAG | | | |
| | GCCTGGGGCAGAGCCGACTGT | | | |
| 2C fusion | ATGCTGCTGGCTCTGCTGCCT | 25 | MLLALLPVLGIHFVLRDAQA | 26 |
| TCRα- | GTGCTGGGCATCCACTTCGTG | | QSVTQPDARVTVSEGASLQL | |
| LZL-IgG_{HC} | CTGAGGGACGCCCAGGCCCAG | | RCKYSYSATPYLFWYVQYPR | |
| | AGCGTGACCCAGCCTGACGCC | | QGLQLLLKYYSGDPVVQGVN | |
| | AGAGTGACAGTGTCTGAGGGC | | GFEAEFSKSNSSFHLRKASV | |
| | GCCAGCCTGCAGCTGAGATGC | | HWSDSAVYFCAVSGFASALT | |
| | AAGTACAGCTACAGCGCCACC | | FGSGTKVIVLPYIQNPEPAV | |
| | CCCTACCTGTTTTGGTACGTG | | YQLKDPRSQDSTLCLFTDFD | |
| | CAGTACCCCAGACAGGGCCtg | | SQINVPKTMESGTFITDKCV | |
| | CAGCTGCTGCTGAAGTACTAC | | LDMKAMDSKSNGAIAWSNQT | |
| | AGCGGCGACCCTGTGGTGCAG | | SFTCQDIFKETNATYPSSDV | |
| | GGCGTGAACGGCTTCGAGGCC | | PCGGGGSGGGGSGGGGSGGG | |
| | GAGTTCAGCAAGAGCAACAGC | | GSLEIRAAFLRQRNTALRTE | |
| | AGCTTCCACCTGAGAAAGGCC | | VAELEQEVQRLENEVSQYET | |
| | AGCGTGCATTGGAGCGACAGC | | RYGPLGGSGGAKTTAPSVYP | |
| | GCCGTGTATTTTTGTGCCGTG | | LAPVCGGTTGSSVTLGCLVK | |
| | AGCGGCTTCGCCAGCGCCCTG | | GYFPEPVTLTWNSGSLSSGV | |
| | ACCTTCGGCAGCGGCACAAAA | | HTFPALLQSGLYTLSSSVTV | |
| | GTGATCGTGCTGCCCTACATC | | TSNTWPSQTITCNVAHPASS | |
| | CAGAACCCCGAGCCCGCCGTG | | TKVDKKIEPRVPITQNPCPP | |
| | TACCAGCTGAAGGACCCCAGA | | LKECPPCAAPDLLGGPSVFI | |
| | AGCCAGGACAGCACCCTGTGC | | FPPKIKDVLMISLSPMVTCV | |
| | CTGTTCACCGACTTCGACAGC | | VVDVSEDDPDVQISWFVNNV | |
| | CAGATCAACGTGCCCAAGACC | | EVHTAQTQTHREDYNSTLRV | |
| | ATGGAAAGCGGCACCTTCATC | | VSALPIQHQDWMSGKEFKCK | |
| | ACCGATAAGTGCGTGCTGGAC | | VNNRALPSPIEKTISKPRGP | |
| | ATGAAGGCCATGGACAGCAAG | | VRAPQVYVLPPPAEEMTKKE | |
| | TCCAACGGCGCTATCGCCTGG | | FSLTCMITGFLPAEIAVDWT | |
| | TCCAACCAGACCTCATTCACA | | SNGRTEQNYKNTATVLDSDG | |
| | TGCCAGGACATCTTCAAAGAG | | SYFMYSKLRVQKSTWERGSL | |
| | ACAAACGCCACCTACCCCAGC | | FACSVVHEGLHNHLTTKTIS | |
| | AGCGACGTGCCTTGTGGTGGA | | RSLGK | |
| | GGTGGGAGTGGGGGAGGAGGC | | | |
| | AGTGGGGGCGGCGGGAGTGGC | | | |
| | GGGGGGGGTTCCTTGGAGATA | | | |
| | CGGGCTGCTTTTCTCCGCCAA | | | |
| | CGAAACACTGCACTGCGAACC | | | |
| | GAAGTAGCAGAACTGGAACAG | | | |
| | GAGGTGCAAAGGCTCGAGAAT | | | |
| | GAGGTTTCCCAGTACGAAACA | | | |
| | CGATACGGCCCTTTGGGCGGA | | | |
| | TCCGGAGGGGCCAAAACCACC | | | |
| | GCTCCATCTGTCTACCCCTTG | | | |
| | GCCCCAGTGTGCGGTGGAACT | | | |
| | ACTGGTAGCTCCGTGACACTG | | | |
| | GGCTGCCTGGTGAAAGGCTAC | | | |
| | TTCCCTGAGCCTGTTACACTC | | | |
| | ACATGGAATTCAGGATCCCTG | | | |
| | TCCTCCGGAGTTCACACCTTC | | | |
| | CCGGCACTCCTGCAGAGCGGA | | | |
| | CTTTACACACTGTCATCCTCC | | | |
| | GTAACTGTGACAAGCAACACC | | | |
| | TGGCCTTCTCAGACCATTACT | | | |
| | TGCAACGTGGCCCATCCCGCT | | | |
| | TCCTCCACAAAAGTGGACAAA | | | |
| | AAGATCGAACCTAGAGTCCCC | | | |
| | ATTACTCAAAATCCCTGCCCC | | | |
| | CCGCTTAAAGAGTGCCCCCCA | | | |
| | TGTGCCGCCCCAGACCTGCTC | | | |
| | GGAGGGCCGAGCGTGTTTATC | | | |
| | TTTCCACCCAAGATTAAAGAC | | | |
| | GTTCTGATGATTTCCCTCAGC | | | |
| | CCTATGGTTACGTGCGTCGTT | | | |
| | GTGGATGTGTCTGAGGACGAT | | | |
| | CCCGATGTTCAGATCTCCTGG | | | |
| | TTTGTAAACAATGTGGAAGTA | | | |
| | CACACCGCTCAGACCCAGACC | | | |
| | CACAGAGAGGACTACAACAGT | | | |
| | ACACTGCGAGTTGTAAGCGCT | | | |
| | CTTCCTATACAACATCAGGAT | | | |
| | TGGATGAGCGGTAAGGAATTT | | | |
| | AAATGTAAAGTCAATAATAGG | | | |
| | GCCTTGCCAAGCCCAATCGAA | | | |
| | AAGACTATTTCTAAGCCTAGG | | | |
| | GGACCGGTCCGGGCTCCACAG | | | |
| | GTCTACGTGCTGCCACCCCCA | | | |
| | GCCGAAGAGATGACTAAGAAG | | | |
| | GAGTTCTCTCTGACGTGCATG | | | |
| | ATAACTGGCTTTCTCCCCGCA | | | |
| | GAGATTGCCGTCGATTGGACA | | | |
| | AGCAACGGCCGGACTGAGCAG | | | |
| | AATTACAAAAATACCGCCACA | | | |
| | GTTCTGGATTCTGACGGCTCA | | | |
| | TACTTCATGTACTCAAAGCTG | | | |
| | CGAGTCCAGAAAAGCACGTGG | | | |
| | GAGCGCGGGAGTCTGTTTGCC | | | |
| | TGCTCCGTGGTGCATGAAGGC | | | |
| | CTGCACAATCACCTGACCACT | | | |
| | AAAACAATCAGTCGCTCTCTG | | | |
| | GGTAAGTGA | | | |
| 2C fusion | | 27 | MSNTAFPDPAWNTTLLSWVA | 28 |
| TCRβ- | ATGAGCAACACCGCCTTCCCC | | LFLLGTKHMEAAVTQSPRNK | |
| LZR-IgG_{LC} | GACCCTGCCTGGAACACCACC | | VAVTGGKVTLSCNQTNNHNN | |
| | CTGCTGTCCTGGGTGGCCCTG | | MYWYRQDTGHGLRLIHYSYG | |
| | TTCCTGCTGGGCACCAAGCAC | | AGSTEKGDIPDGYKASRPSQ | |
| | ATGGAAGCCGCCGTGACACAG | | ENFSLILELATPSQTSVYFC | |
| | AGCCCCAGAAACAAGGTGGCC | | ASGGGGTLYFGAGTRLSVLE | |
| | GTGACCGGCGGCAAAGTGACC | | DLRNVTPPKVSLFEPSKAEI | |
| | CTGAGCTGCAACCAGACCAAC | | ANKQKATLVCLARGFFPDHV | |
| | AACCACAACAACATGTACTGG | | ELSWWVNGKEVHSGVCTDPQ | |
| | TACAGACAGGACACCGGCCAC | | AYKESNYSYCLSSRLRVSAT | |
| | GGACTGAGACTGATCCACTAC | | FWHNPRNHFRCQVQFHGLSE | |
| | AGCTACGGCGCTGGCAGCACC | | EDKWPEGSPKPVTQNISAEA | |
| | GAGAAGGGCGACATCCCCGAC | | WGRADCGGGGSGGGGSGGGG | |
| | GGCTACAAGGCCAGCAGACCC | | SGGGGSLEIEAAFLERENTA | |
| | AGCCAGGAAAACTTCAGCCTG | | LETRVAELRQRVQRLRNRVS | |
| | ATCCTGGAACTGGCCACCCCT | | QYRTRYGPLGGSGGRRADAA | |
| | AGCCAGACCAGCGTGTACTTC | | PTVSIFPPSSEQLTSGGASV | |
| | TGCGCCTCTGGCGGCGGAGGA | | VCFLNNFYPKDINVKWKIDG | |
| | ACCCTGTACTTCGGAGCCGGC | | SERQNGVLNSWTDQDSKDST | |
| | ACCAGACTGAGCGTGCTGGAA | | YSMSSTLTLTKDEYERHNSY | |
| | GATCTGAGAAACGTGACCCCC | | TCEATHKTSTSPIVKSFNRN | |
| | CCCAAGGTGTCCCTGTTCGAG | | EC | |
| | CCCAGCAAGGCCGAGATCGCC | | | |
| | AACAAGCAGAAAGCCACCCTC | | | |
| | GTGTGCCTGGCCAGAGGCTTC | | | |
| | TTCCCTGACCACGTGGAGCTG | | | |
| | TCTTGGTGGGTGAACGGCAAA | | | |
| | GAGGTGCACAGCGGCGTCTGC | | | |
| | ACCGACCCCCAGGCCTACAAA | | | |
| | GAGAGCAACTACTCCTACTGC | | | |
| | CTGAGCAGCAGACTGAGAGTG | | | |
| | TCCGCCACCTTCTGGCACAAC | | | |
| | CCCAGAAACCACTTCAGATGC | | | |
| | CAGGTGCAGTTCCATGGCCTG | | | |
| | TCCGAAGAGGACAAGTGGCCC | | | |
| | GAGGGCAGCCCTAAGCCTGTG | | | |
| | ACACAGAACATCAGCGCCGAG | | | |
| | GCCTGGGGCAGAGCCGACTGT | | | |
| | GGTGGAGGTGGGAGTGGGGGA | | | |
| | GGTGGATCAGGCGGCGGGGGG | | | |
| | AGCGGTGGAGGGGGCAGTCTT | | | |
| | GAGATTGAAGCAGCCTTCCTG | | | |
| | GAGAGAGAAAATACAGCACTG | | | |
| | GAGACAAGGGTCGCTGAACTT | | | |
| | AGGCAACGCGTTCAACGCCTC | | | |
| | CGGAATAGAGTTAGTCAGTAT | | | |
| | AGAACACGCTATGGACCTTTG | | | |
| | GGCGGATCCGGAGGGAGACGG | | | |
| | GCTGATGCTGCACCAACTGTA | | | |
| | TCCATCTTCCCACCATCCAGT | | | |
| | GAGCAGTTAACATCTGGAGGT | | | |
| | GCCTCAGTCGTGTGCTTCTTG | | | |
| | AACAACTTCTACCCCAAAGAC | | | |
| | ATCAATGTCAAGTGGAAGATT | | | |
| | GATGGCAGTGAACGACAAAAT | | | |
| | GGCGTCCTGAACAGTTGGACT | | | |
| | GATCAGGACAGCAAAGACAGC | | | |
| | ACCTACAGCATGAGCAGCACC | | | |
| | CTCACGTTGACCAAGGACGAG | | | |
| | TATGAACGACATAACAGCTAT | | | |
| | ACCTGTGAGGCCACTCACAAG | | | |
| | ACATCAACTTCACCCATTGTC | | | |
| | AAGAGCTTCAACAGGAATGAG | | | |
| | TGTTAA | | | |
| Murine | ATGCTGCTGGCTCTGCTGCCT | 29 | MLLALLPVLGIHFVLRDAQA | 30 |
| 6mut 2C | GTGCTGGGCATCCACTTCGTG | | QSVTQPDARVTVSEGASLQL | |
| TCRα | CTGAGGGACGCCCAGGCCCAG | | RCKYSYSATPYLFWYVQYPR | |
| | AGCGTGACCCAGCCTGACGCC | | QGPQLLLKYYSGDPVVQGVN | |
| | AGAGTGACAGTGTCTGAGGGC | | GFEAEFSKSNSSFHLRKASV | |
| | GCCAGCCTGCAGCTGAGATGC | | HRSDSAVYFCAVSGFASALT | |
| | AAGTACAGCTACAGCGCCACC | | FGSGTKVIVLPYNQNPEPAV | |
| | CCCTACCTGTTTTGGTACGTG | | YQLKDPRSQDSTLCLFTDFD | |
| | CAGTACCCCAGACAGGGCCCC | | SQINVPKTMESGTFITDKCV | |
| | CAGCTGCTGCTGAAGTACTAC | | LDMKAMDSKSNGAIAWSNQT | |
| | AGCGGCGACCCTGTGGTGCAG | | SFTCQDIFKETNATYPSSDV | |
| | GGCGTGAACGGCTTCGAGGCC | | PC | |
| | GAGTTCAGCAAGAGCAACAGC | | | |
| | AGCTTCCACCTGAGAAAGGCC | | | |
| | AGCGTGCATAGGAGCGACAGC | | | |
| | GCCGTGTATTTTTGTGCCGTG | | | |
| | AGCGGCTTCGCCAGCGCCCTG | | | |
| | ACCTTCGGCAGCGGCACAAAA | | | |
| | GTGATCGTGCTGCCCTACAAC | | | |
| | CAGAACCCCGAGCCCGCCGTG | | | |
| | TACCAGCTGAAGGACCCCAGA | | | |
| | AGCCAGGACAGCACCCTGTGC | | | |
| | CTGTTCACCGACTTCGACAGC | | | |
| | CAGATCAACGTGCCCAAGACC | | | |
| | ATGGAAAGCGGCACCTTCATC | | | |
| | ACCGATAAGTGCGTGCTGGAC | | | |
| | ATGAAGGCCATGGACAGCAAG | | | |
| | TCCAACGGCGCTATCGCCTGG | | | |
| | TCCAACCAGACCTCATTCACA | | | |
| | TGCCAGGACATCTTCAAAGAG | | | |
| | ACAAACGCCACCTACCCCAGC | | | |
| | AGCGACGTGCCTTGT | | | |
| Murine | ATGAGCAACACCGCCTTCCCC | 31 | MSNTAFPDPAWNTTLLSWVA | 32 |
| 6mut 2C | GACCCTGCCTGGAACACCACC | | LFLLGTKHMEAAVTQSPRNK | |
| TCRβ | CTGCTGTCCTGGGTGGCCCTG | | VAVTGEKVTLSCNQTNNHNN | |
| | TTCCTGCTGGGCACCAAGCAC | | MYWYRQDTGHELRLIHYSYG | |
| | ATGGAAGCCGCCGTGACACAG | | AGSTEKGDIPDGYKASRPSQ | |
| | AGCCCCAGAAACAAGGTGGCC | | ENFSLILESATPSQTSVYFC | |
| | GTGACCGGCGAGAAAGTGACC | | ASGGGGTLYFGAGTRLSVLE | |
| | CTGAGCTGCAACCAGACCAAC | | DLRNVTPPKVSLFEPSKAEI | |
| | AACCACAACAACATGTACTGG | | ANKQKATLVCLARGFFPDHV | |
| | TACAGACAGGACACCGGCCAC | | ELSWWVNGKEVHSGVCTDPQ | |
| | GAGCTGAGACTGATCCACTAC | | AYKESNYSYCLSSRLRVSAT | |
| | AGCTACGGCGCTGGCAGCACC | | FWHNPRNHFRCQVQFHGLSE | |
| | GAGAAGGGCGACATCCCCGAC | | EDKWPEGSPKPVTQNISAEA | |
| | GGCTACAAGGCCAGCAGACCC | | WGRADC | |
| | AGCCAGGAAAACTTCAGCCTG | | | |
| | ATCCTGGAAAGCGCCACCCCT | | | |
| | AGCCAGACCAGCGTGTACTTC | | | |
| | TGCGCCTCTGGCGGCGGAGGA | | | |
| | ACCCTGTACTTCGGAGCCGGC | | | |
| | ACCAGACTGAGCGTGCTGGAA | | | |
| | GATCTGAGAAACGTGACCCCC | | | |
| | CCCAAGGTGTCCCTGTTCGAG | | | |
| | CCCAGCAAGGCCGAGATCGCC | | | |
| | AACAAGCAGAAAGCCACCCTC | | | |
| | GTGTGCCTGGCCAGAGGCTTC | | | |
| | TTCCCTGACCACGTGGAGCTG | | | |
| | TCTTGGTGGGTGAACGGCAAA | | | |
| | GAGGTGCACAGCGGCGTCTGC | | | |
| | ACCGACCCCCAGGCCTACAAA | | | |
| | GAGAGCAACTACTCCTACTGC | | | |
| | CTGAGCAGCAGACTGAGAGTG | | | |
| | TCCGCCACCTTCTGGCACAAC | | | |
| | CCCAGAAACCACTTCAGATGC | | | |
| | CAGGTGCAGTTCCATGGCCTG | | | |
| | TCCGAAGAGGACAAGTGGCCC | | | |
| | GAGGGCAGCCCTAAGCCTGTG | | | |
| | ACACAGAACATCAGCGCCGAG | | | |
| | GCCTGGGGCAGAGCCGACTGT | | | |
| Murine | ATGCTGCTGGCTCTGCTGCCT | 33 | MLLALLPVLGIHFVLRDAQA | 34 |
| 6mut 2C | GTGCTGGGCATCCACTTCGTG | | QSVTQPDARVTVSEGASLQL | |
| fusion | CTGAGGGACGCCCAGGCCCAG | | RCKYSYSATPYLFWYVQYPR | |
| TCRα- | AGCGTGACCCAGCCTGACGCC | | QGPQLLLKYYSGDPVVQGVN | |
| LZL-IgG_{HC} | AGAGTGACAGTGTCTGAGGGC | | GFEAEFSKSNSSFHLRKASV | |
| | GCCAGCCTGCAGCTGAGATGC | | HRSDSAVYFCAVSGFASALT | |
| | AAGTACAGCTACAGCGCCACC | | FGSGTKVIVLPYNQNPEPAV | |
| | CCCTACCTGTTTTGGTACGTG | | YQLKDPRSQDSTLCLFTDFD | |
| | CAGTACCCCAGACAGGGCCCC | | SQINVPKTMESGTFITDKCV | |
| | CAGCTGCTGCTGAAGTACTAC | | LDMKAMDSKSNGAIAWSNQT | |
| | AGCGGCGACCCTGTGGTGCAG | | SFTCQDIFKETNATYPSSDV | |
| | GGCGTGAACGGCTTCGAGGCC | | PCGGGGSGGGGSGGGGSGGG | |
| | GAGTTCAGCAAGAGCAACAGC | | GSLEIRAAFLRQRNTALRTE | |
| | AGCTTCCACCTGAGAAAGGCC | | VAELEQEVQRLENEVSQYET | |
| | AGCGTGCATAGGAGCGACAGC | | RYGPLGGSGGAKTTAPSVYP | |
| | GCCGTGTATTTTTGTGCCGTG | | LAPVCGGTTGSSVTLGCLVK | |
| | AGCGGCTTCGCCAGCGCCCTG | | GYFPEPVTLTWNSGSLSSGV | |
| | ACCTTCGGCAGCGGCACAAAA | | HTFPALLQSGLYTLSSSVTV | |
| | GTGATCGTGCTGCCCTACAAC | | TSNTWPSQTITCNVAHPASS | |
| | CAGAACCCCGAGCCCGCCGTG | | TKVDKKIEPRVPITQNPCPP | |
| | TACCAGCTGAAGGACCCCAGA | | LKECPPCAAPDLLGGPSVFI | |
| | AGCCAGGACAGCACCCTGTGC | | FPPKIKDVLMISLSPMVTCV | |
| | CTGTTCACCGACTTCGACAGC | | VVDVSEDDPDVQISWFVNNV | |
| | CAGATCAACGTGCCCAAGACC | | EVHTAQTQTHREDYNSTLRV | |
| | ATGGAAAGCGGCACCTTCATC | | VSALPIQHQDWMSGKEFKCK | |
| | ACCGATAAGTGCGTGCTGGAC | | VNNRALPSPIEKTISKPRGP | |
| | ATGAACGGCGCTATCGCCTGG | | VRAPQVYVLPPPAEEMTKKE | |
| | TCCAACCAGACCTCATTCACA | | FSLTCMITGFLPAEIAVDWT | |
| | TGCCAGGACATCTTCAAAGAG | | SNGRTEQNYKNTATVLDSDG | |
| | ACAAACGCCACCTACCCCAGC | | SYFMYSKLRVQKSTWERGSL | |
| | AGCGACGTGCCTTGTGGTGGA | | FACSVVHEGLHNHLTTKTIS | |
| | GGTGGGAGTGGGGGAGGAGGC | | RSLGK | |
| | AGTGGGGGCGGCGGGAGTGGC | | | |
| | GGGGGGGGTTCCTTGGAGATA | | | |
| | CGGGCTGCTTTTCTCCGCCAA | | | |
| | CGAAACACTGCACTGCGAACC | | | |
| | GAAGTAGCAGAACTGGAACAG | | | |
| | GAGGTGCAAAGGCTCGAGAAT | | | |
| | GAGGTTTCCCAGTACGAAACA | | | |
| | CGATACGGCCCTTTGGGCGGA | | | |
| | TCCGGAGGGGCCAAAACCACC | | | |
| | GCTCCATCTGTCTACCCCTTG | | | |
| | GCCCCCAGTGTGCGGTGGAAC | | | |
| | TACTGGTAGCTCCGTGACACT | | | |
| | GGGCTGCCTGGTGAAAGGCTA | | | |
| | CTTCCCTGAGCCTGTTACACT | | | |
| | CACATGGAATTCAGGATCCCT | | | |
| | GTCCTCCGGAGTTCACACCTT | | | |
| | CCCGGCACTCCTGCAGAGCGG | | | |
| | ACTTTACACACTGTCATCCTC | | | |
| | CGTAACTGTGACAAGCAACAC | | | |
| | CTGGCCTTCTCAGACCATTAC | | | |
| | TTGCAACGTGGCCCATCCCGC | | | |
| | TTCCTCCACAAAAGTGGACAA | | | |
| | AAAGATCGAACCTAGAGTCCC | | | |
| | CATTACTCAAAATCCCTGCCC | | | |
| | CCCGCTTAAAGAGTGCCCCCC | | | |
| | ATGTGCCGCCCCAGACCTGCT | | | |
| | CGGAGGGCCGAGCGTGTTTAT | | | |
| | CTTTCCACCCAAGATTAAAGA | | | |
| | CGTTCTGATGATTTCCCTCAG | | | |
| | CCCTATGGTTACGTGCGTCGT | | | |
| | TGTGGATGTGTCTGAGGACGA | | | |
| | TCCCGATGTTCAGATCTCCTG | | | |
| | GTTTGTAAACAATGTGGAAGT | | | |
| | ACACACCGCTCAGACCCAGAC | | | |
| | CCACAGAGAGGACTACAACAG | | | |
| | TACACTGCGAGTTGTAAGCGC | | | |
| | TCTTCCTATACAACATCAGGA | | | |
| | TTGGATGAGCGGTAAGGAATT | | | |
| | TAAATGTAAAGTCAATAATAG | | | |
| | GGCCTTGCCAAGCCCAATCGA | | | |
| | AAAGACTATTTCTAAGCCTAG | | | |
| | GGGACCGGTCCGGGCTCCACA | | | |
| | GGTCTACGTGCTGCCACCCCC | | | |
| | AGCCGAAGAGATGACTAAGAA | | | |
| | GGAGTTCTCTCTGACGTGCAT | | | |
| | GATAACTGGCTTTCTCCCCGC | | | |
| | AGAGATTGCCGTCGATTGGAC | | | |
| | AAGCAACGGCCGGACTGAGCA | | | |
| | GAATTACAAAAATACCGCCAC | | | |
| | AGTTCTGGATTCTGACGGCTC | | | |
| | ATACTTCATGTACTCAAAGCT | | | |
| | GCGAGTCCAGAAAAGCACGTG | | | |
| | GGAGCGCGGGAGTCTGTTTGC | | | |
| | CTGCTCCGTGGTGCATGAAGG | | | |
| | CCTGCACAATCACCTGACCAC | | | |
| | TAAAACAATCAGTCGCTCTCT | | | |
| | GGGTAAGTGA | | | |
| Murine | ATGAGCAACACCGCCTTCCCC | 35 | MSNTAFPDPAWNTTLLSWVA | 36 |
| 6mut 2C | GACCCTGCCTGGAACACCACC | | LFLLGTKHMEAAVTQSPRNK | |
| fusion | CTGCTGTCCTGGGTGGCCCTG | | VAVTGEKVTLSCNQTNNHNN | |
| TCRβ- | TTCCTGCTGGGCACCAAGCAC | | MYWYRQDTGHELRLIHYSYG | |
| LZR-IgG_{LC} | ATGGAAGCCGCCGTGACACAG | | AGSTEKGDIPDGYKASRPSQ | |
| | AGCCCCAGAAACAAGGTGGCC | | ENFSLILESATPSQTSVYFC | |
| | GTGACCGGCGAGAAAGTGACC | | ASGGGGTLYFGAGTRLSVLE | |
| | CTGAGCTGCAACCAGACCAAC | | DLRNVTPPKVSLFEPSKAEI | |
| | AACCACAACAACATGTACTGG | | ANKQKATLVCLARGFFPDHV | |
| | TACAGACAGGACACCGGCCAC | | ELSWWVNGKEVHSGVCTDPQ | |
| | GAGCTGAGACTGATCCACTAC | | AYKESNYSYCLSSRLRVSAT | |
| | AGCTACGGCGCTGGCAGCACC | | FWHNPRNHFRCQVQFHGLSE | |
| | GAGAAGGGCGACATCCCCGAC | | EDKWPEGSPKPVTQNISAEA | |
| | GGCTACAAGGCCAGCAGACCC | | WGRADCGGGGSGGGGSGGGG | |
| | AGCCAGGAAAACTTCAGCCTG | | SGGGGSLEIEAAFLERENTA | |
| | ATCCTGGAAAGCGCCACCCCT | | LETRVAELRQRVQRLRNRVS | |
| | AGCCAGACCAGCGTGTACTTC | | QYRTRYGPLGGSGGRRADAA | |
| | TGCGCCTCTGGCGGCGGAGGA | | PTVSIFPPSSEQLTSGGASV | |
| | ACCCTGTACTTCGGAGCCGGC | | VCFLNNFYPKDINVKWKIDG | |
| | ACCAGACTGAGCGTGCTGGAA | | SERQNGVLNSWTDQDSKDST | |
| | GATCTGAGAAACGTGACCCCC | | YSMSSTLTLTKDEYERHNSY | |
| | CCCAAGGTGTCCCTGTTCGAG | | TCEATHKTSTSPIVKSFNRN | |
| | CCCAGCAAGGCCGAGATCGCC | | EC | |
| | AACAAGCAGAAAGCCACCCTC | | | |
| | GTGTGCCTGGCCAGAGGCTTC | | | |
| | TTCCCTGACCACGTGGAGCTG | | | |
| | TCTTGGTGGGTGAACGGCAAA | | | |
| | GAGGTGCACAGCGGCGTCTGC | | | |
| | ACCGACCCCCAGGCCTACAAA | | | |
| | GAGAGCAACTACTCCTACTGC | | | |
| | CTGAGCAGCAGACTGAGAGTG | | | |
| | TCCGCCACCTTCTGGCACAAC | | | |
| | CCCAGAAACCACTTCAGATGC | | | |
| | CAGGTGCAGTTCCATGGCCTG | | | |
| | TCCGAAGAGGACAAGTGGCCC | | | |
| | GAGGGCAGCCCTAAGCCTGTG | | | |
| | ACACAGAACATCAGCGCCGAG | | | |
| | GCCTGGGGCAGAGCCGACTGT | | | |
| | GGTGGAGGTGGGAGTGGGGGA | | | |
| | GGTGGATCAGGCGGCGGGGGG | | | |
| | AGCGGTGGAGGGGGCAGTCTT | | | |
| | GAGATTGAAGCAGCCTTCCTG | | | |
| | GAGAGAGAAAATACAGCACTG | | | |
| | GAGACAAGGGTCGCTGAACTT | | | |
| | AGGCAACGCGTTCAACGCCTC | | | |
| | CGGAATAGAGTTAGTCAGTAT | | | |
| | AGAACACGCTATGGACCTTTG | | | |
| | GGCGGATCCGGAGGGAGACGG | | | |
| | GCTGATGCTGCACCAACTGTA | | | |
| | TCCATCTTCCCACCATCCAGT | | | |
| | GAGCAGTTAACATCTGGAGGT | | | |
| | GCCTCAGTCGTGTGCTTCTTG | | | |
| | AACAACTTCTACCCCAAAGAC | | | |
| | ATCAATGTCAAGTGGAAGATT | | | |
| | GATGGCAGTGAACGACAAAAT | | | |
| | GGCGTCCTGAACAGTTGGACT | | | |
| | GATCAGGACAGCAAAGACAGC | | | |
| | ACCTACAGCATGAGCAGCACC | | | |
| | CTCACGTTGACCAAGGACGAG | | | |
| | TATGAACGACATAACAGCTAT | | | |
| | ACCTGTGAGGCCACTCACAAG | | | |
| | ACATCAACTTCACCCATTGTC | | | |
| | AAGAGCTTCAACAGGAATGAG | | | |
| | TGTTAA | | | |
| Murine | ATGGCTCGCTCGGTGACCCTG | 37 | MARSVTLVFLVLVSLTGLYA | 38 |
| B2M | GTCTTTCTGGTGCTTGTCTCA | | | |
| signal | CTGACCGGCCTGTATGCT | | | |
| peptide | | | | |
| OVa₂₅₇₋₂₆₅ | | | | |
| | AGTATCATTAATTTCGAAAAA | 39 | SIINFEKL | 40 |
| | CTT | | | |
| Murine | ATTCAAAAAACCCCACAGATC | 41 | IQKTPQIQVYSRHPPENGKP | 42 |
| B2M | CAAGTATACTCACGCCACCCA | | NILNCYVTQFHPPHIEIQML | |
| | CCGGAGAATGGGAAGCCGAAC | | KNGKKIPKVEMSDMSFSKDW | |
| | ATACTGAACTGCTACGTAACA | | SFYILAHTEFTPTETDTYAC | |
| | CAGTTCCACCCGCCTCACATT | | RVKHASMAEPKTVYWDRDM | |
| | GAAATCCAAATGCTGAAGAAC | | | |
| | GGGAAAAAAATTCCTAAAGTA | | | |
| | GAGATGTCAGATATGTCCTTC | | | |
| | AGCAAGGACTGGTCTTTCTAT | | | |
| | ATCCTGGCTCACACTGAATTC | | | |
| | ACCCCCACTGAGACTGATACA | | | |
| | TACGCCTGCAGAGTTAAGCAT | | | |
| | GCCAGTATGGCCGAGCCCAAG | | | |
| | ACCGTCTACTGGGATCGAGAC | | | |
| | ATG | | | |
| Murine | GGCCCACACTCGCTGAGGTAT | 43 | GPHSLRYFVTAVSRPGLGEP | 44 |
| H2Kb | TTCGTCACCGCCGTGTCCCGG | | RYMEVGYVDDTEFVRFDSDA | |
| | CCCGGCCTCGGGGAGCCCCGG | | ENPRYEPRARWMEQEGPEYW | |
| | TACATGGAAGTCGGCTACGTG | | ERETQKAKGNEQSFRVDLRT | |
| | GACGACACGGAGTTCGTGCGC | | LLGCYNQSKGGSHTIQVISG | |
| | TTCGACAGCGACGCGGAGAAT | | CEVGSDGRLLRGYQQYAYDG | |
| | CCGAGATATGAGCCGCGGGCG | | CDYIALNEDLKTWTAADMAA | |
| | CGGTGGATGGAGCAGGAGGGG | | LITKHKWEQAGEAERLRAYL | |
| | CCCGAGTATTGGGAGCGGGAG | | EGTCVEWLRRYLKNGNATLL | |
| | ACACAGAAAGCCAAGGGCAAT | | RTDSPKAHVTHHSRPEDKVT | |
| | GAGCAGAGTTTCCGAGTGGAC | | LRCWALGFYPADITLTWQLN | |
| | CTGAGGACCCTGCTCGGCTGT | | GEELIQDMELVETRPAGDGT | |
| | TACAACCAGAGCAAGGGCGGC | | FQKWASVVVPLGKEQYYTCH | |
| | TCTCACACTATTCAGGTGATC | | VYHQGLPEPLTLRWEPPPST | |
| | TCTGGCTGTGAAGTGGGGTCC | | VSN | |
| | GACGGGCGACTCCTCCGCGGG | | | |
| | TACCAGCAGTACGCCTACGAC | | | |
| | GGCTGCGATTACATCGCCCTG | | | |
| | AACGAAGACCTGAAAACGTGG | | | |
| | ACGGCGGCGGACATGGCGGCG | | | |
| | CTGATCACCAAACACAAGTGG | | | |
| | GAGCAGGCTGGTGAAGCAGAG | | | |
| | AGACTCAGGGCCTACCTGGAG | | | |
| | GGCACGTGCGTGGAGTGGCTC | | | |
| | CGCAGATACCTGAAGAACGGG | | | |
| | AACGCGACGCTGCTGCGCACA | | | |
| | GATTCCCCAAAGGCCCATGTG | | | |
| | ACCCATCACAGCAGACCTGAA | | | |
| | GATAAAGTCACCCTGAGGTGC | | | |
| | TGGGCCCTGGGCTTCTACCCT | | | |
| | GCTGACATCACCCTGACCTGG | | | |
| | CAGTTGAATGGGGAGGAGCTG | | | |
| | ATCCAGGACATGGAGCTTGTG | | | |
| | GAGACCAGGCCTGCAGGGGAT | | | |
| | GGAACCTTCCAGAAGTGGGCA | | | |
| | TCTGTGGTGGTGCCTCTTGGG | | | |
| | AAGGAGCAGTATTACACATGC | | | |
| | CATGTGTACCATCAGGGGCTG | | | |
| | CCTGAGCCCCTCACCCTGAGA | | | |
| | TGGGAGCCTCCTCCATCCACT | | | |
| | GTCTCCAAC | | | |
| Murine | ATGGCTCGCTCGGTGACCCTG | 45 | MARSVTLVFLVLVSLTGLYA | 46 |
| fusion B2M | GTCTTTCTGGTGCTTGTCTCA | | SIINFEKLGCGASGGGGSGG | |
| signal | CTGACCGGCCTGTATGCTAGT | | GGSIQKTPQIQVYSRHPPEN | |
| peptide- | ATCATTAATTTCGAAAAACTT | | GKPNILNCYVTQFHPPHIEI | |
| OVa₂₅₇₋₂₆₅- | GGATGTGGTGCTAGCGGTGGT | | QMLKNGKKIPKVEMSDMSFS | |
| B2M-H2Kb- | GGTGGTAGCGGAGGTGGAGGC | | KDWSFYILAHTEFTPTETDT | |
| LZL-IgG_{HC} | AGCATTCAAAAAACCCCACAG | | YACRVKHASMAEPKTVYWDR | |
| | ATCCAAGTATACTCACGCCAC | | DMGGGGSGGGGSGGGGSGGG | |
| | CCACCGGAGAATGGGAAGCCG | | GSGPHSLRYFVTAVSRPGLG | |
| | AACATACTGAACTGCTACGTA | | EPRYMEVGYVDDTEFVRFDS | |
| | ACACAGTTCCACCCGCCTCAC | | DAENPRYEPRARWMEQEGPE | |
| | ATTGAAATCCAAATGCTGAAG | | YWERETQKAKGNEQSFRVDL | |
| | AACGGGAAAAAAATTCCTAAA | | RTLLGCYNQSKGGSHTIQVI | |
| | GTAGAGATGTCAGATATGTCC | | SGCEVGSDGRLLRGYQQYAY | |
| | TTCAGCAAGGACTGGTCTTTC | | DGCDYIALNEDLKTWTAADM | |
| | TATATCCTGGCTCACACTGAA | | AALITKHKWEQAGEAERLRA | |
| | TTCACCCCCACTGAGACTGAT | | YLEGTCVEWLRRYLKNGNAT | |
| | ACATACGCCTGCAGAGTTAAG | | LLRTDSPKAHVTHHSRPEDK | |
| | CATGCCAGTATGGCCGAGCCC | | VTLRCWALGFYPADITLTWQ | |
| | AAGACCGTCTACTGGGATCGA | | LNGEELIQDMELVETRPAGD | |
| | GACATGGGCGGTGGTGGTTCC | | GTFQKWASVVVPLGKEQYYT | |
| | GGTGGAGGCGGTTCCGGAGGT | | CHVYHQGLPEPLTLRWEPPP | |
| | GGTGGATCCGGTGGTGGTGGT | | STVSNGGGGSGGGGSGGGGS | |
| | AGTGGCCCACACTCGCTGAGG | | GGGGSLEIRAAFLRQRNTAL | |
| | TATTTCGTCACCGCCGTGTCC | | RTEVAELEQEVQRLENEVSQ | |
| | CGGCCCGGCCTCGGGGAGCCC | | YETRYGPLGGSGGAKTTAPS | |
| | CGGTACATGGAAGTCGGCTAC | | VYPLAPVCGGTTGSSVTLGC | |
| | GTGGACGACACGGAGTTCGTG | | LVKGYFPEPVTLTWNSGSLS | |
| | CGCTTCGACAGCGACGCGGAG | | SGVHTFPALLQSGLYTLSSS | |
| | AATCCGAGATATGAGCCGCGG | | VTVTSNTWPSQTITCNVAHP | |
| | GCGCGGTGGATGGAGCAGGAG | | ASSTKVDKKIEPRVPITQNP | |
| | GGGCCCGAGTATTGGGAGCGG | | CPPLKECPPCAAPDLLGGPS | |
| | GAGACACAGAAAGCCAAGGGC | | VFIFPPKIKDVLMISLSPMV | |
| | AATGAGCAGAGTTTCCGAGTG | | TCVVVDVSEDDPDVQISWFV | |
| | GACCTGAGGACCCTGCTCGGC | | NNVEVHTAQTQTHREDYNST | |
| | TGTTACAACCAGAGCAAGGGC | | LRVVSALPIQHQDWMSGKEF | |
| | GGCTCTCACACTATTCAGGTG | | KCKVNNRALPSPIEKTISKP | |
| | ATCTCTGGCTGTGAAGTGGGG | | RGPVRAPQVYVLPPPAEEMT | |
| | TCCGACGGGCGACTCCTCCGC | | KKEFSLTCMITGFLPAEIAV | |
| | GGGTACCAGCAGTACGCCTAC | | DWTSNGRTEQNYKNTATVLD | |
| | GACGGCTGCGATTACATCGCC | | SDGSYFMYSKLRVQKSTWER | |
| | CTGAACGAAGACCTGAAAACG | | GSLFACSVVHEGLHNHLTTK | |
| | TGGACGGCGGCGGACATGGCG | | TISRSLGK | |
| | GCGCTGATCACCAAACACAAG | | | |
| | TGGGAGCAGGCTGGTGAAGCA | | | |
| | GAGAGACTCAGGGCCTACCTG | | | |
| | GAGGGCACGTGCGTGGAGTGG | | | |
| | CTCCGCAGATACCTGAAGAAC | | | |
| | GGGAACGCGACGCTGCTGCGC | | | |
| | ACAGATTCCCCAAAGGCCCAT | | | |
| | GTGACCCATCACAGCAGACCT | | | |
| | GAAGATAAAGTCACCCTGAGG | | | |
| | TGCTGGGCCCTGGGCTTCTAC | | | |
| | CCTGCTGACATCACCCTGACC | | | |
| | TGGCAGTTGAATGGGGAGGAG | | | |
| | CTGATCCAGGACATGGAGCTT | | | |
| | GTGGAGACCAGGCCTGCAGGG | | | |
| | GATGGAACCTTCCAGAAGTGG | | | |
| | GCATCTGTGGTGGTGCCTCTT | | | |
| | GGGAAGGAGCAGTATTACACA | | | |
| | TGCCATGTGTACCATCAGGGG | | | |
| | CTGCCTGAGCCCCTCACCCTG | | | |
| | AGATGGGAGCCTCCTCCATCC | | | |
| | ACTGTCTCCAACGGTGGAGGT | | | |
| | GGGAGTGGGGGAGGAGGCAGT | | | |
| | GGGGGCGGCGGGAGTGGCGGG | | | |
| | GGGGGTTCCTTGGAGATACGG | | | |
| | GCTGCTTTTCTCCGCCAACGA | | | |
| | AACACTGCACTGCGAACCGAA | | | |
| | GTAGCAGAACTGGAACAGGAG | | | |
| | GTGCAAAGGCTCGAGAATGAG | | | |
| | GTTTCCCAGTACGAAACACGA | | | |
| | TACGGCCCTTTGGGCGGATCC | | | |
| | GGAGGGGCCAAAACCACCGCT | | | |
| | CCATCTGTCTACCCCTTGGCC | | | |
| | CCAGTGTGCGGTGGAACTACT | | | |
| | GGTAGCTCCGTGACACTGGGC | | | |
| | TGCCTGGTGAAAGGCTACTTC | | | |
| | CCTGAGCCTGTTACACTCACA | | | |
| | TGGAATTCAGGATCCCTGTCC | | | |
| | TCCGGAGTTCACACCTTCCCG | | | |
| | GCACTCCTGCAGAGCGGACTT | | | |
| | TACACACTGTCATCCTCCGTA | | | |
| | ACTGTGACAAGCAACACCTGG | | | |
| | CCTTCTCAGACCATTACTTGC | | | |
| | AACGTGGCCCATCCCGCTTCC | | | |
| | TCCACAAAAGTGGACAAAAAG | | | |
| | ATCGAACCTAGAGTCCCCATT | | | |
| | ACTCAAAATCCCTGCCCCCCG | | | |
| | CTTAAAGAGTGCCCCCCATGT | | | |
| | GCCGCCCCAGACCTGCTCGGA | | | |
| | GGGCCGAGCGTGTTTATCTTT | | | |
| | CCACCCAAGATTAAAGACGTT | | | |
| | CTGATGATTTCCCTCAGCCCT | | | |
| | ATGGTTACGTGCGTCGTTGTG | | | |
| | GATGTGTCTGAGGACGATCCC | | | |
| | GATGTTCAGATCTCCTGGTTT | | | |
| | GTAAACAATGTGGAAGTACAC | | | |
| | ACCGCTCAGACCCAGACCCAC | | | |
| | AGAGAGGACTACAACAGTACA | | | |
| | CTGCGAGTTGTAAGCGCTCTT | | | |
| | CCTATACAACATCAGGATTGG | | | |
| | ATGAGCGGTAAGGAATTTAAA | | | |
| | TGTAAAGTCAATAATAGGGCC | | | |
| | TTGCCAAGCCCAATCGAAAAG | | | |
| | ACTATTTCTAAGCCTAGGGGA | | | |
| | CCGGTCCGGGCTCCACAGGTC | | | |
| | TACGTGCTGCCACCCCCAGCC | | | |
| | GAAGAGATGACTAAGAAGGAG | | | |
| | TTCTCTCTGACGTGCATGATA | | | |
| | ACTGGCTTTCTCCCCGCAGAG | | | |
| | ATTGCCGTCGATTGGACAAGC | | | |
| | AACGGCCGGACTGAGCAGAAT | | | |
| | TACAAAAATACCGCCACAGTT | | | |
| | CTGGATTCTGACGGCTCATAC | | | |
| | TTCATGTACTCAAAGCTGCGA | | | |
| | GTCCAGAAAAGCACGTGGGAG | | | |
| | CGCGGGAGTCTGTTTGCCTGC | | | |
| | TCCGTGGTGCATGAAGGCCTG | | | |
| | CACAATCACCTGACCACTAAA | | | |
| | ACAATCAGTCGCTCTCTGGGT | | | |
| | AAGTGA | | | |
| Murine | ATGGCTCGCTCGGTGACCCTG | 47 | MARSVTLVFLVLVSLTGLYA | 48 |
| fusion | GTCTTTCTGGTGCTTGTCTCA | | SIINFEKLGCGASGGGGSGG | |
| B2M signal | CTGACCGGCCTGTATGCTAGT | | GGSIQKTPQIQVYSRHPPEN | |
| peptide- | ATCATTAATTTCGAAAAACTT | | GKPNILNCYVTQFHPPHIEI | |
| OVa₂₅₇₋₂₆₅- | GGATGTGGTGCTAGCGGTGGT | | QMLKNGKKIPKVEMSDMSFS | |
| B2M-H2Kb- | GGTGGTAGCGGAGGTGGAGGC | | KDWSFYILAHTEFTPTETDT | |
| LZL-IgG_{LC} | AGCATTCAAAAAACCCCACAG | | YACRVKHASMAEPKTVYWDR | |
| | ATCCAAGTATACTCACGCCAC | | DMGGGGSGGGGSGGGGSGGG | |
| | CCACCGGAGAATGGGAAGCCG | | GSGPHSLRYFVTAVSRPGLG | |
| | AACATACTGAACTGCTACGTA | | EPRYMEVGYVDDTEFVRFDS | |
| | ACACAGTTCCACCCGCCTCAC | | DAENPRYEPRARWMEQEGPE | |
| | ATTGAAATCCAAATGCTGAAG | | YWERETQKAKGNEQSFRVDL | |
| | AACGGGAAAAAAATTCCTAAA | | RTLLGCYNQSKGGSHTIQVI | |
| | GTAGAGATGTCAGATATGTCC | | SGCEVGSDGRLLRGYQQYAY | |
| | TTCAGCAAGGACTGGTCTTTC | | DGCDYIALNEDLKTWTAADM | |
| | TATATCCTGGCTCACACTGAA | | AALITKHKWEQAGEAERLRA | |
| | TTCACCCCCACTGAGACTGAT | | YLEGTCVEWLRRYLKNGNAT | |
| | ACATACGCCTGCAGAGTTAAG | | LLRTDSPKAHVTHHSRPEDK | |
| | CATGCCAGTATGGCCGAGCCC | | VTLRCWALGFYPADITLTWQ | |
| | AAGACCGTCTACTGGGATCGA | | LNGEELIQDMELVETRPAGD | |
| | GACATGGGCGGTGGTGGTTCC | | GTFQKWASVVVPLGKEQYYT | |
| | GGTGGAGGCGGTTCCGGAGGT | | CHVYHQGLPEPLTLRWEPPP | |
| | GGTGGATCCGGTGGTGGTGGT | | STVSNGGGGSGGGGSGGGGS | |
| | AGTGGCCCACACTCGCTGAGG | | GGGGSLEIEAAFLERENTAL | |
| | TATTTCGTCACCGCCGTGTCC | | ETRVAELRQRVQRLRNRVSQ | |
| | CGGCCCGGCCTCGGGGAGCCC | | YRTRYGPLGGSGGRRADAAP | |
| | CGGTACATGGAAGTCGGCTAC | | TVSIFPPSSEQLTSGGASVV | |
| | GTGGACGACACGGAGTTCGTG | | CFLNNFYPKDINVKWKIDGS | |
| | CGCTTCGACAGCGACGCGGAG | | ERQNGVLNSWTDQDSKDSTY | |
| | AATCCGAGATATGAGCCGCGG | | SMSSTLTLTKDEYERHNSYT | |
| | GCGCGGTGGATGGAGCAGGAG | | CEATHKTSTSPIVKSFNRNE | |
| | GGGCCCGAGTATTGGGAGCGG | | C | |
| | GAGACACAGAAAGCCAAGGGC | | | |
| | AATGAGCAGAGTTTCCGAGTG | | | |
| | GACCTGAGGACCCTGCTCGGC | | | |
| | TGTTACAACCAGAGCAAGGGC | | | |
| | GGCTCTCACACTATTCAGGTG | | | |
| | ATCTCTGGCTGTGAAGTGGGG | | | |
| | TCCGACGGGCGACTCCTCCGC | | | |
| | GGGTACCAGCAGTACGCCTAC | | | |
| | GACGGCTGCGATTACATCGCC | | | |
| | CTGAACGAAGACCTGAAAACG | | | |
| | TGGACGGCGGCGGACATGGCG | | | |
| | GCGCTGATCACCAAACACAAG | | | |
| | TGGGAGCAGGCTGGTGAAGCA | | | |
| | GAGAGACTCAGGGCCTACCTG | | | |
| | GAGGGCACGTGCGTGGAGTGG | | | |
| | CTCCGCAGATACCTGAAGAAC | | | |
| | GGGAACGCGACGCTGCTGCGC | | | |
| | ACAGATTCCCCAAAGGCCCAT | | | |
| | GTGACCCATCACAGCAGACCT | | | |
| | GAAGATAAAGTCACCCTGAGG | | | |
| | TGCTGGGCCCTGGGCTTCTAC | | | |
| | CCTGCTGACATCACCCTGACC | | | |
| | TGGCAGTTGAATGGGGAGGAG | | | |
| | CTGATCCAGGACATGGAGCTT | | | |
| | GTGGAGACCAGGCCTGCAGGG | | | |
| | GATGGAACCTTCCAGAAGTGG | | | |
| | GCATCTGTGGTGGTGCCTCTT | | | |
| | GGGAAGGAGCAGTATTACACA | | | |
| | TGCCATGTGTACCATCAGGGG | | | |
| | CTGCCTGAGCCCCTCACCCTG | | | |
| | AGATGGGAGCCTCCTCCATCC | | | |
| | ACTGTCTCCAACGGTGGAGGT | | | |
| | GGGAGTGGGGGAGGTGGATCA | | | |
| | GGCGGCGGGGGGAGCGGTGGA | | | |
| | GGGGGCAGTCTTGAGATTGAA | | | |
| | GCAGCCTTCCTGGAGAGAGAA | | | |
| | AATACAGCACTGGAGACAAGG | | | |
| | GTCGCTGAACTTAGGCAACGC | | | |
| | GTTCAACGCCTCCGGAATAGA | | | |
| | GTTAGTCAGTATAGAACACGC | | | |
| | TATGGACCTTTGGGCGGATCC | | | |
| | GGAGGGAGACGGGCTGATGCT | | | |
| | GCACCAACTGTATCCATCTTC | | | |
| | CCACCATCCAGTGAGCAGTTA | | | |
| | ACATCTGGAGGTGCCTCAGTC | | | |
| | GTGTGCTTCTTGAACAACTTC | | | |
| | TACCCCAAAGACATCAATGTC | | | |
| | AAGTGGAAGATTGATGGCAGT | | | |
| | GAACGACAAAATGGCGTCCTG | | | |
| | AACAGTTGGACTGATCAGGAC | | | |
| | AGCAAAGACAGCACCTACAGC | | | |
| | ATGAGCAGCACCCTCACGTTG | | | |
| | ACCAAGGACGAGTATGAACGA | | | |
| | CATAACAGCTATACCTGTGAG | | | |
| | GCCACTCACAAGACATCAACT | | | |
| | TCACCCATTGTCAAGAGCTTC | | | |
| | AACAGGAATGAGTGTTAA | | | |
| Furin-GSG- | CGCCGAAAACGCGGTTCTGGA | 49 | RRKRGSGHHHHHH | 50 |
| His | CACCACCATCACCATCAC | | | |
| GSG-P2A | GGAAGCGGAGCTACTAACTTC | 51 | GSGATNFSLLKQAGDVEENP | 52 |
| | AGCCTGCTGAAGCAGGCTGGA | | GP | |
| | GACGTGGAGGAGAACCCTGGA | | | |
| | CCT | | | |
| Single | ATGGACAAGATCCTGACAGCA | 53 | MDKILTASFLLLGLHLAGVN | 54 |
| Vector | TCGTTTTTACTCCTAGGCCTT | | GQQQEKRDQQQVRQSPQSLT | |
| Insert | CACCTAGCTGGGGTGAATGGC | | VWEGETAILNCSYEDSTFNY | |
| OTITCRα- | CAGCAGCAGGAGAAACGTGAC | | FPWYQQFPGEGPALLISIRS | |
| LZL- | CAGCAGCAGGTGAGACAAAGT | | VSDKKEDGRFTIFFNKREKK | |
| IgG_{HC}- | CCCCAATCTCTGACAGTCTGG | | LSLHITDSQPGDSATYFCAA | |
| furin-GSG- | GAAGGAGAGACCGCAATTCTG | | SDNYQLIWGSGTKLIIKPDI | |
| HIS-GSG- | AACTGCAGTTATGAGGACAGC | | QNPEPAVYQLKDPRSQDSTL | |
| P2A- | ACTTTTAACTACTTCCCATGG | | CLFTDFDSQINVPKTMESGT | |
| OT1TCRβ - | TACCAGCAGTTCCCTGGGGAA | | FITDKTVLDMEAMDSKSNGA | |
| LZR- | GGCCCTGCACTCCTGATATCC | | IAWSNQTSFTCQDIFKETNA | |
| mIgG_{LC} | ATACGTTCAGTGTCCGATAAA | | TYPSSDVPCGGGGSGGGGSG | |
| | AAGGAAGATGGACGATTCACA | | GGGSGGGGSLEIRAAFLRQR | |
| | ATCTTCTTCAATAAAAGGGAG | | NTALRTEVAELEQEVQRLEN | |
| | AAAAAGCTCTCCTTGCACATC | | EVSQYETRYGPLGGSGGAKT | |
| | ACAGACTCTCAGCCTGGAGAC | | TAPSVYPLAPVCGGTTGSSV | |
| | TCAGCTACCTACTTCTGTGCA | | TLGCLVKGYFPEPVTLTWNS | |
| | GCAAGTGACAACTATCAGTTG | | GSLSSGVHTFPALLQSGLYT | |
| | ATCTGGGGCTCTGGGACCAAG | | LSSSVTVTSNTWPSQTITCN | |
| | CTAATTATAAAGCCAGACATC | | VAHPASSTKVDKKIEPRVPI | |
| | CAGAACCCAGAACCTGCTGTG | | TQNPCPPLKECPPCAAPDLL | |
| | TACCAGTTAAAAGATCCTCGG | | GGPSVFIFPPKIKDVLMISL | |
| | TCTCAGGACAGCACCCTCTGC | | SPMVTCVVVDVSEDDPDVQI | |
| | CTGTTCACCGACTTTGACTCC | | SWFVNNVEVHTAQTQTHRED | |
| | CAAATCAATGTGCCGAAAACC | | YNSTLRVVSALPIQHQDWMS | |
| | ATGGAATCTGGAACGTTCATC | | GKEFKCKVNNRALPSPIEKT | |
| | ACTGACAAAACTGTGCTGGAC | | ISKPRGPVRAPQVYVLPPPA | |
| | ATGGAAGCTATGGATTCCAAG | | EEMTKKEFSLTCMITGFLPA | |
| | AGCAATGGGGCCATTGCCTGG | | EIAVDWTSNGRTEQNYKNTA | |
| | AGCAACCAGACAAGCTTCACC | | TVLDSDGSYFMYSKLRVQKS | |
| | TGCCAAGATATCTTCAAAGAG | | TWERGSLFACSVVHEGLHNH | |
| | ACCAACGCCACCTACCCCAGT | | LTTKTISRSLGKRRKRGSGH | |
| | TCAGACGTTCCCTGTGGTGGA | | HHHHHGSGATNFSLLKQAGD | |
| | GGTGGGAGTGGGGGAGGAGGC | | VEENPGPMSNTVLADSAWGI | |
| | AGTGGGGGCGGCGGGAGTGGC | | TLLSWVTVFLLGTSSADSGV | |
| | GGGGGGGGTTCCTTGGAGATA | | VQSPRHIIKEKGGRSVLTCI | |
| | CGGGCTGCTTTTCTCCGCCAA | | PISGHSNVVWYQQTLGKELK | |
| | CGAAACACTGCACTGCGAACC | | FLIQHYEKVERDKGFLPSRF | |
| | GAAGTAGCAGAACTGGAACAG | | SVQQFDDYHSEMNMSALELE | |
| | GAGGTGCAAAGGCTCGAGAAT | | DSAMYFCASSRANYEQYFGP | |
| | GAGGTTTCCCAGTACGAAACA | | GTRLTVLEDLRNVTPPKVSL | |
| | CGATACGGCCCTTTGGGCGGA | | FEPSKAEIANKQKATLVCLA | |
| | TCCGGAGGGGCCAAAACCACC | | RGFFPDHVELSWWVNGKEVH | |
| | GCTCCATCTGTCTACCCCTTG | | SGVSTDPQAYKESNYSYCLS | |
| | GCCCCAGTGTGCGGTGGAACT | | SRLRVSATFWHNPRNHFRCQ | |
| | ACTGGTAGCTCCGTGACACTG | | VQFHGLSEEDKWPEGSPKPV | |
| | GGCTGCCTGGTGAAAGGCTAC | | TQNISAEAWGRADCGGGGSG | |
| | TTCCCTGAGCCTGTTACACTC | | GGGSGGGGSGGGGSLEIEAA | |
| | ACATGGAATTCAGGATCCCTG | | FLERENTALETRVAELRQRV | |
| | TCCTCCGGAGTTCACACCTTC | | QRLRNRVSQYRTRYGPLGGS | |
| | CCGGCACTCCTGCAGAGCGGA | | GGRRADAAPTVSIFPPSSEQ | |
| | CTTTACACACTGTCATCCTCC | | LTSGGASVVCFLNNFYPKDI | |
| | GTAACTGTGACAAGCAACACC | | NVKWKIDGSERQNGVLNSWT | |
| | TGGCCTTCTCAGACCATTACT | | DQDSKDSTYSMSSTLTLTKD | |
| | TGCAACGTGGCCCATCCCGCT | | EYERHNSYTCEATHKTSTSP | |
| | TCCTCCACAAAAGTGGACAAA | | IVKSFNRNEC | |
| | AAGATCGAACCTAGAGTCCCC | | | |
| | ATTACTCAAAATCCCTGCCCC | | | |
| | CCGCTTAAAGAGTGCCCCCCA | | | |
| | TGTGCCGCCCCAGACCTGCTC | | | |
| | GGAGGGCCGAGCGTGTTTATC | | | |
| | TTTCCACCCAAGATTAAAGAC | | | |
| | GTTCTGATGATTTCCCTCAGC | | | |
| | CCTATGGTTACGTGCGTCGTT | | | |
| | GTGGATGTGTCTGAGGACGAT | | | |
| | CCCGATGTTCAGATCTCCTGG | | | |
| | TTTGTAAACAATGTGGAAGTA | | | |
| | CACACCGCTCAGACCCAGACC | | | |
| | CACAGAGAGGACTACAACAGT | | | |
| | ACACTGCGAGTTGTAAGCGCT | | | |
| | CTTCCTATACAACATCAGGAT | | | |
| | TGGATGAGCGGTAAGGAATTT | | | |
| | AAATGTAAAGTCAATAATAGG | | | |
| | GCCTTGCCAAGCCCAATCGAA | | | |
| | AAGACTATTTCTAAGCCTAGG | | | |
| | GGACCGGTCCGGGCTCCACAG | | | |
| | GTCTACGTGCTGCCACCCCCA | | | |
| | GCCGAAGAGATGACTAAGAAG | | | |
| | GAGTTCTCTCTGACGTGCATG | | | |
| | ATAACTGGCTTTCTCCCCGCA | | | |
| | GAGATTGCCGTCGATTGGACA | | | |
| | AGCAACGGCCGGACTGAGCAG | | | |
| | AATTACAAAAATACCGCCACA | | | |
| | GTTCTGGATTCTGACGGCTCA | | | |
| | TACTTCATGTACTCAAAGCTG | | | |
| | CGAGTCCAGAAAAGCACGTGG | | | |
| | GAGCGCGGGAGTCTGTTTGCC | | | |
| | TGCTCCGTGGTGCATGAAGGC | | | |
| | CTGCACAATCACCTGACCACT | | | |
| | AAAACAATCAGTCGCTCTCTG | | | |
| | GGTAAGCGCCGAAAACGCGGT | | | |
| | TCTGGACACCACCATCACCAT | | | |
| | CACGGAAGCGGAGCTACTAAC | | | |
| | TTCAGCCTGCTGAAGCAGGCT | | | |
| | GGAGACGTGGAGGAGAACCCT | | | |
| | GGACCTATGTCTAACACTGTC | | | |
| | CTCGCTGATTCTGCCTGGGGC | | | |
| | ATCACCCTGCTATCTTGGGTT | | | |
| | ACTGTCTTTCTCTTGGGAACA | | | |
| | AGTTCAGCAGATTCTGGGGTT | | | |
| | GTCCAGTCTCCAAGACACATA | | | |
| | ATCAAAGAAAAGGGAGGAAGG | | | |
| | TCCGTTCTGACGTGTATTCCC | | | |
| | ATCTCTGGACATAGCAATGTG | | | |
| | GTCTGGTACCAGCAGACTCTG | | | |
| | GGGAAGGAATTAAAGTTCCTT | | | |
| | ATTCAGCATTATGAAAAGGTG | | | |
| | GAGAGAGACAAAGGATTCCTA | | | |
| | CCCAGCAGATTCTCAGTCCAA | | | |
| | CAGTTTGATGACTATCACTCT | | | |
| | GAAATGAACATGAGTGCCTTG | | | |
| | GAACTGGAGGACTCTGCTATG | | | |
| | TACTTCTGTGCCAGCTCTCGG | | | |
| | GCCAATTATGAACAGTACTTC | | | |
| | GGTCCCGGCACCAGGCTCACG | | | |
| | GTTTTAGAGGATCTGAGAAAT | | | |
| | GTGACTCCACCCAAGGTCTCC | | | |
| | TTGTTTGAGCCATCAAAAGCA | | | |
| | GAGATTGCAAACAAACAAAAG | | | |
| | GCTACCCTCGTGTGCTTGGCC | | | |
| | AGGGGCTTCTTCCCTGACCAC | | | |
| | GTGGAGCTGAGCTGGTGGGTG | | | |
| | AATGGCAAGGAGGTCCACAGT | | | |
| | GGGGTCAGCACGGACCCTCAG | | | |
| | GCCTACAAGGAGAGCAATTAT | | | |
| | AGCTACTGCCTGAGCAGCCGC | | | |
| | CTGAGGGTCTCTGCTACCTTC | | | |
| | TGGCACAATCCTCGAAACCAC | | | |
| | TTCCGCTGCCAAGTGCAGTTC | | | |
| | CATGGGCTTTCAGAGGAGGAC | | | |
| | AAGTGGCCAGAGGGCTCACCC | | | |
| | AAACCTGTCACACAGAACATC | | | |
| | AGTGCAGAGGCCTGGGGCCGA | | | |
| | GCAGACTGTGGTGGAGGTGGG | | | |
| | AGTGGGGGAGGTGGATCAGGC | | | |
| | GGCGGGGGGAGCGGTGGAGGG | | | |
| | GGCAGTCTTGAGATTGAAGCA | | | |
| | GCCTTCCTGGAGAGAGAAAAT | | | |
| | ACAGCACTGGAGACAAGGGTC | | | |
| | GCTGAACTTAGGCAACGCGTT | | | |
| | CAACGCCTCCGGAATAGAGTT | | | |
| | AGTCAGTATAGAACACGCTAT | | | |
| | GGACCTTTGGGCGGATCCGCA | | | |
| | GGGAGACGGGCTGATGCTGCA | | | |
| | CCAACTGTATCCATCTTCCCA | | | |
| | CCATCCAGTGAGCAGTTAACA | | | |
| | TCTGGAGGTGCCTCAGTCGTG | | | |
| | TGCTTCTTGAACAACTTCTAC | | | |
| | CCCAAAGACATCAATGTCAAG | | | |
| | TGGAAGATTGATGGCAGTGAA | | | |
| | CGACAAAATGGCGTCCTGAAC | | | |
| | AGTTGGACTGATCAGGACAGC | | | |
| | AAAGACAGCACCTACAGCATG | | | |
| | AGCAGCACCCTCACGTTGACC | | | |
| | AAGGACGAGTATGAACGACAT | | | |
| | AACAGCTATACCTGTGAGGCC | | | |
| | ACTCACAAGACATCAACTTCA | | | |
| | CCCATTGTCAAGAGCTTCAAC | | | |
| | AGGAATGAGTGTTAA | | | |
| Linker | GGTGGAGGTGGGAGTGGGGGA | 55 | GGGGSGGGGSGGGGS | 56 |
| (GGGGS)3 | GGAGGCAGTGGGGGCGGCGGG | | | |
| | AGT | | | |
| Linker | GGTGGAGGTGGGAGTGGGGGA | 57 | GGGGSGGGGS | 58 |
| (GGGGS)2 | GGAGGCAGT | | | |
| HIV Gag | | | GLFKIWPSYK | 59 |
| epitope | | | | |
| Collagen- | | | MDPDLEIRAAFLRQRNTALR | 61 |
| like | | | TEVAELEQEVQRLEEVSYQE | |
| trimerization | | | TRYGPLGGGK | |
| domain | | | | |
| AZip | | | MDPDLEIEAAFLERENTALE | 62 |
| leucine | | | TRVAELRQRVQRLRNRVSQY | |
| zipper | | | RTRYGPLGGGK | |
| HER V-K | ATGCTCCTGCTGCTCGTCCCA | 71 | MLLLLVPVLEVIFTLGGTRA | 72 |
| alpha chain- | GTGCTCGAGGTGATTTTTACT | | QSVTQLDSHVSVSEGTPVLL | |
| LZL-IgG1 | CTGGGAGGAACCAGAGCCCAG | | RCNYSSSYSPSLFWYVQHPN | |
| heavy chain | TCGGTGACCCAGCTTGACAGC | | KGLQLLLKYTSAATLVKGIN | |
| | CACGTCTCTGTCTCTGAAGGA | | GFEAEFKKSETSFHLTKPSA | |
| | ACCCCGGTGCTGCTGAGGTGC | | HMSDAAEYFCVVSTLKIIFG | |
| | AACTACTCATCTTCTTATTCA | | KGTRLHILPNIQNPDPAVYQ | |
| | CCATCTCTCTTCTGGTATGTG | | LRDSKSSDKSVCLFTDFDSQ | |
| | CAACACCCCAACAAAGGACTC | | TNVSQSKDSDVYITDKTVLD | |
| | CAGCTTCTCCTGAAGTACACA | | MRSMDFKSNSAVAWSNKSDF | |
| | TCAGCGGCCACCCTGGTTAAA | | ACANAFNNSIIPEDTFFPSP | |
| | GGCATCAACGGTTTTGAGGCT | | ESSCGGGGSGGGGSGGGGSG | |
| | GAATTTAAGAAGAGTGAAACC | | GGGSLEIRAAFLRQRNTALR | |
| | TCCTTCCACCTGACGAAACCC | | TEVAELEQEVQRLENEVSQY | |
| | TCAGCCCATATGAGCGACGCG | | ETRYGPLGGSGGASTKGPSV | |
| | GCTGAGTACTTCTGTGTTGTG | | FPLAPSSKSTSGGTAALGCL | |
| | AGTACTCTCAAGATCATCTTT | | VKDYFPEPVTVSWNSGALTS | |
| | GGAAAAGGGACACGACTTCAT | | GVHTFPAVLQSSGLYSLSSV | |
| | ATTCTCCCCAATATCCAGAAC | | VTVPSSSLGTQTYICNVNHK | |
| | CCTGACCCTGCCGTGTACCAG | | PSNTKVDKKVEPKSCDKTHT | |
| | CTGAGAGACTCTAAATCCAGT | | CPPCPAPELLGGPSVFLFPP | |
| | GACAAGTCTGTCTGCCTATTC | | KPKDTLMISRTPEVTCVVVD | |
| | ACCGATTTTGATTCTCAAACA | | VSHEDPEVKFNWYVDGVEVH | |
| | AATGTGTCACAAAGTAAGGAT | | NAKTKPREEQYNSTYRVVSV | |
| | TCTGATGTGTATATCACAGAC | | LTVLHQDWLNGKEYKCKVSN | |
| | AAAACTGTGCTAGACATGAGG | | KALPAPIEKTISKAKGQPRE | |
| | TCTATGGACTTCAAGAGCAAC | | PQVYTLPPSREEMTKNQVSL | |
| | AGTGCTGTGGCCTGGAGCAAC | | TCLVKGFYPSDIAVEWESNG | |
| | AAATCTGACTTTGCATGTGCA | | QPENNYKTTPPVLDSDGSFF | |
| | AACGCCTTCAACAACAGCATT | | LYSKLTVDKSRWQQGNVFSC | |
| | ATTCCAGAAGACACCTTCTTC | | SVMHEALHNHYTQKSLSLSP | |
| | CCCAGCCCAGAAAGTTCCTGT | | GK | |
| | GGTGGAGGTGGGAGTGGGGGA | | | |
| | GGTGGATCAGGAGGCGGTGGT | | | |
| | AGTGGTGGTGGCGGTTCTTTG | | | |
| | GAGATACGGGCTGCTTTTCTC | | | |
| | CGCCAACGAAACACTGCACTG | | | |
| | CGAACCGAAGTAGCAGAACTG | | | |
| | GAACAGGAGGTGCAAAGGCTC | | | |
| | GAGAATGAGGTTTCCCAGTAC | | | |
| | GAAACACGATACGGCCCTTTG | | | |
| | GGCGGATCCGGAGGGGCTAGC | | | |
| | ACCAAGGGCCCATCGGTCTTC | | | |
| | CCCCTGGCACCCTCCTCCAAG | | | |
| | AGCACCTCTGGGGGCACAGCG | | | |
| | GCCCTGGGCTGCCTGGTCAAG | | | |
| | GACTACTTCCCCGAACCGGTG | | | |
| | ACGGTGTCGTGGAACTCAGGC | | | |
| | GCCCTGACCAGCGGCGTGCAC | | | |
| | ACCTTCCCGGCTGTCCTACAG | | | |
| | TCCTCAGGACTCTACTCCCTC | | | |
| | AGCAGCGTGGTGACCGTGCCC | | | |
| | TCCAGCAGCTTGGGCACCCAG | | | |
| | ACCTACATCTGCAACGTGAAT | | | |
| | CACAAGCCCAGCAACACCAAG | | | |
| | GTGGACAAGAAAGTTGAGCCC | | | |
| | AAATCTTGTGACAAAACTCAC | | | |
| | ACATGCCCACCGTGCCCAGCA | | | |
| | CCTGAACTCCTGGGGGGACCG | | | |
| | TCAGTCTTCCTCTTCCCCCCA | | | |
| | AAACCCAAGGACACCCTCATG | | | |
| | ATCTCCCGGACCCCTGAGGTC | | | |
| | ACATGCGTGGTGGTGGACGTG | | | |
| | AGCCACGAAGACCCTGAGGTC | | | |
| | AAGTTCAACTGGTACGTGGAC | | | |
| | GGCGTGGAGGTGCATAATGCC | | | |
| | AAGACAAAGCCGCGGGAGGAG | | | |
| | CAGTACAACAGCACGTACCGT | | | |
| | GTGGTCAGCGTCCTCACCGTC | | | |
| | CTGCACCAGGACTGGCTGAAT | | | |
| | GGCAAGGAGTACAAGTGCAAG | | | |
| | GTCTCCAACAAAGCCCTCCCA | | | |
| | GCCCCCATCGAGAAAACCATC | | | |
| | TCCAAAGCCAAAGGGCAGCCC | | | |
| | CGAGAACCACAGGTGTACACC | | | |
| | CTGCCCCCATCCCGGGAGGAG | | | |
| | ATGACCAAGAACCAGGTCAGC | | | |
| | CTGACCTGCCTGGTCAAAGGC | | | |
| | TTCTATCCCAGCGACATCGCC | | | |
| | GTGGAGTGGGAGAGCAATGGG | | | |
| | CAGCCGGAGAACAACTACAAG | | | |
| | ACCACGCCTCCCGTGCTGGAC | | | |
| | TCCGACGGCTCCTTCTTCCTC | | | |
| | TACAGCAAGCTCACCGTGGAC | | | |
| | AAGAGCAGGTGGCAGCAGGGG | | | |
| | AACGTCTTCTCATGCTCCGTG | | | |
| | ATGCATGAGGCTCTGCACAAC | | | |
| | CACTACACGCAGAAGAGCCTC | | | |
| | TCCCTGTCTCCGGGTAAATGA | | | |
| | TGA | | | |
| HERV-K | ATGGGCACCAGCCTCCTCTGC | 73 | MGTSLLCWMALCLLGADHAD | 74 |
| beta chain- | TGGATGGCCCTGTGTCTCCTG | | TGVSQDPRHKITKRGQNVTF | |
| LZR-IgG1 | GGGGCAGATCACGCAGATACT | | RCDPISEHNRLYWYRQTLGQ | |
| light chain | GGAGTCTCCCAGGACCCCAGA | | GPEFLTYFQNEAQLEKSRLL | |
| | CACAAGATCACAAAGAGGGGA | | SDRFSAERPKGSFSTLEIQR | |
| | CAGAATGTAACTTTCAGGTGT | | TEQGDSAMYLCASSIGPSEA | |
| | GATCCAATTTCTGAACACAAC | | FFGQGTRLTVVEDLNKVFPP | |
| | CGCCTTTATTGGTACCGACAG | | EVAVFEPSEAEISHTQKATL | |
| | ACCCTGGGGCAGGGCCCAGAG | | VCLATGFFPDHVELSWWVNG | |
| | TTTCTGACTTACTTCCAGAAT | | KEVHSGVSTDPQPLKEQPAL | |
| | GAAGCTCAACTAGAAAAATCA | | NDSRYCLSSRLRVSATFWQN | |
| | AGGCTGCTCAGTGATCGGTTC | | PRNHFRCQVQFYGLSENDEW | |
| | TCTGCAGAGAGGCCTAAGGGA | | TQDRAKPVTQIVSAEAWGRA | |
| | TCTTTCTCCACCTTGGAGATC | | DCGGGGSGGGGSGGGGSGGG | |
| | CAGCGCACAGAGCAGGGGGAC | | GSLEIEAAFLERENTALETR | |
| | TCGGCCATGTATCTCTGTGCC | | VAELRQRVQRLRNRVSQYRT | |
| | AGCAGCATAGGCCCGTCTGAA | | RYGPLGGSGGRTVAAPSVFI | |
| | GCTTTCTTTGGACAAGGCACC | | FPPSDEQLKSGTASVVCLLN | |
| | AGACTCACAGTTGTAGAGGAC | | NFYPREAKVQWKVDNALQSG | |
| | CTGAACAAGGTGTTCCCACCC | | NSQESVTEQDSKDSTYSLSS | |
| | GAGGTCGCTGTGTTTGAGCCA | | TLTLSKADYEKHKVYACEVT | |
| | TCAGAAGCAGAGATCTCCCAC | | HQGLSSPVTKSFNRGEC | |
| | ACCCAAAAGGCCACACTGGTG | | | |
| | TGCCTGGCCACAGGCTTCTTC | | | |
| | CCTGACCACGTGGAGCTGAGC | | | |
| | TGGTGGGTGAATGGGAAGGAG | | | |
| | GTGCACAGTGGGGTCAGCACG | | | |
| | GACCCGCAGCCCCTCAAGGAG | | | |
| | CAGCCCGCCCTCAATGACTCC | | | |
| | AGATACTGCCTGAGCAGCCGC | | | |
| | CTGAGGGTCTCGGCCACCTTC | | | |
| | TGGCAGAACCCCCGCAACCAC | | | |
| | TTCCGCTGTCAAGTCCAGTTC | | | |
| | TACGGGCTCTCGGAGAATGAC | | | |
| | GAGTGGACCCAGGATAGGGCC | | | |
| | AAACCCGTCACCCAGATCGTC | | | |
| | AGCGCCGAGGCCTGGGGTAGA | | | |
| | GCAGACTGTGGTGGAGGTGGG | | | |
| | AGTGGGGGAGGTGGATCAGGC | | | |
| | GGCGGGGGGAGCGGTGGAGGG | | | |
| | GGCAGTCTTGAGATTGAAGCA | | | |
| | GCCTTCCTGGAGAGAGAAAAT | | | |
| | ACAGCACTGGAGACAAGGGTC | | | |
| | GCTGAACTTAGGCAACGCGTT | | | |
| | CAACGCCTCCGGAATAGAGTT | | | |
| | AGTCAGTATAGAACACGCTAT | | | |
| | GGACCTTTGGGCGGATCCGGA | | | |
| | GGGCGTACGGTGGCTGCACCA | | | |
| | TCTGTCTTCATCTTCCCGCCA | | | |
| | TCTGATGAGCAGTTGAAATCT | | | |
| | GGAACTGCCTCTGTTGTGTGC | | | |
| | CTGCTGAATAACTTCTATCCC | | | |
| | AGAGAGGCCAAAGTACAGTGG | | | |
| | AAGGTGGATAACGCCCTCCAA | | | |
| | TCGGGTAACTCCCAGGAGAGT | | | |
| | GTCACAGAGCAGGACAGCAAG | | | |
| | GACAGCACCTACAGCCTCAGC | | | |
| | AGCACCCTGACGCTGAGCAAA | | | |
| | GCAGACTACGAGAAACACAAA | | | |
| | GTCTACGCCTGCGAAGTCACC | | | |
| | CATCAGGGCCTGAGCTCGCCC | | | |
| | GTCACAAAGAGCTTCAACAGG | | | |
| | GGAGAGTGTTAG | | | |
| FK10 TCR | ATGAAATCCTTGAGAGTTTTA | 75 | MKSLRVLLVILWLQLSWVWS | 76 |
| alpha chain | CTAGTGATCCTGTGGCTTCAG | | QQKEVEQNSGPLSVPEGAIA | |
| | TTGAGCTGGGTTTGGAGCCAA | | SLNCTYSDRGSQSFFWYRQY | |
| | CAGAAGGAGGTGGAGCAGAAc | | SGKSPELIMSIYSNGDKEDG | |
| | TCTGGACCCCTCAGTGTTCCA | | RFTAQLNKASQYVSLLIRDS | |
| | GAGGGAGCCATTGCCTCTCTC | | QPSDSATYLCAVETSGTYKY | |
| | AACTGCACTTACAGTGACCGA | | IFGTGTRLKVLANIQNPDPA | |
| | GGTTCCCAGTCCTTCTTCTGG | | VYQLRDSKSSDKSVCLFTDF | |
| | TACAGACAATATTCTGGGAAA | | DSQTNVSQSKDSDVYITDKT | |
| | AGCCCTGAGTTGATAATGTCC | | VLDMRSMDFKSNSAVAWSNK | |
| | ATATACTCCAATGGTGACAAA | | SDFACANAFNNSIIPEDTFF | |
| | GAAGATGGAAGGTTTACAGCA | | PSPESSC | |
| | CAGCTCAATAAAGCCAGCCAG | | | |
| | TATGTTTCTCTGCTCATCAGA | | | |
| | GACTCCCAGCCCAGTGATTCA | | | |
| | GCCACCTACCTCTGTGCCGTG | | | |
| | GAGACCTCAGGAACCTACAAA | | | |
| | TACATCTTTGGAACAGGCACC | | | |
| | AGGCTGAAGGTTTTAGCAAAT | | | |
| | ATCCAGAACCCTGACCCTGCC | | | |
| | GTGTACCAGCTGAGAGACTCT | | | |
| | AAATCCAGTGACAAGTCTGTC | | | |
| | TGCCTATTCACCGATTTTGAT | | | |
| | TCTCAAACAAATGTGTCACAA | | | |
| | AGTAAGGATTCTGATGTGTAT | | | |
| | ATCACAGACAAAACTGTGCTA | | | |
| | GACATGAGGTCTATGGACTTC | | | |
| | AAGAGCAACAGTGCTGTGGCC | | | |
| | TGGAGCAACAAATCTGACTTT | | | |
| | GCATGTGCAAACGCCTTCAAC | | | |
| | AACAGCATTATTCCAGAAGAC | | | |
| | ACCTTCTTCCCCAGCCCAGAA | | | |
| | AGTTCCTGT | | | |
| FK10 TCR | ATGGGGAGTGATCCTGATCTG | | MGSDPDLVKLPSCPDPAMGT | 78 |
| beta chain | GTAAAGCTCCCATCCTGCCCT | | RLLFWVAFCLLGADHTGAGV | |
| | GACCCTGCCATGGGCACCAGG | | SQSPSNKVTEKGKDVELRCD | |
| | CTCCTCTTCTGGGTGGCCTTC | | PISGHTALYWYRQSLGQGLE | |
| | TGTCTCCTGGGGGCAGATCAC | | FLIYFQGNSAPDKSGLPSDR | |
| | ACAGGAGCTGGAGTCTCCCAG | | FSAERTGGSVSTLTIQRTQQ | |
| | TCCCCCAGTAACAAGGTCACA | | EDSAVYLCASSFGPDGYTFG | |
| | GAGAAGGGAAAGGATGTAGAG | | SGTRLTVVEDLNKVFPPEVA | |
| | CTCAGGTGTGATCCAATTTCA | | VFEPSEAEISHTQKATLVCL | |
| | GGTCATACTGCCCTTTACTGG | | ATGFFPDHVELSWWVNGKEV | |
| | TACCGACAGAGCCTGGGGCAG | | HSGVSTDPQPLKEQPALNDS | |
| | GGCCTGGAGTTTTTAATTTAC | | RYCLSSRLRVSATFWQNPRN | |
| | TTCCAAGGCAACAGTGCACCA | | HFRCQVQFYGLSENDEWTQD | |
| | GACAAATCAGGGCTGCCCAGT | | RAKPVTQIVSAEAWGRADC | |
| | GATCGCTTCTCTGCAGAGAGG | | | |
| | ACTGGGGGcTCCGTCTCCACT | | | |
| | CTGACGATCCAGCGCACACAG | | | |
| | CAGGAGGACTCGGCCGTGTAT | | | |
| | CTCTGTGCCAGCAGCTTTGGA | | | |
| | CCAGATGGCTACACCTTCGGT | | | |
| | TCGGGGACCAGGTTAACCGTT | | | |
| | GTAGAGGACCTGAACAAGGTG | | | |
| | TTCCCACCCGAGGTCGCTGTG | | | |
| | TTTGAGCCATCAGAAGCAGAG | | | |
| | ATCTCCCACACCCAAAAGGCC | | | |
| | ACACTGGTGTGCCTGGCCACA | | | |
| | GGCTTCTTCCCTGACCACGTG | | | |
| | GAGCTGAGCTGGTGGGTGAAT | | | |
| | GGGAAGGAGGTGCACAGTGGG | | | |
| | GTCAGCACGGACCCGCAGCCC | | | |
| | CTCAAGGAGCAGCCCGCCCTC | | | |
| | AATGACTCCAGATACTGCCTG | | | |
| | AGCAGCCGCCTGAGGGTCTCG | | | |
| | GCCACCTTCTGGCAGAACCCC | | | |
| | CGCAACCACTTCCGCTGTCAA | | | |
| | GTCCAGTTCTACGGGCTCTCG | | | |
| | GAGAATGACGAGTGGACCCAG | | | |
| | GATAGGGCCAAACCCGTCACC | | | |
| | CAGATCGTCAGCGCCGAGGCC | | | |
| | TGGGGTAGAGCAGACTGT | | | |
| FK10 | ATGAAATCCTTGAGAGTTTTA | 79 | MKSLRVLLVILWLQLSWVWS | 80 |
| alpha-LZL- | CTAGTGATCCTGTGGCTTCAG | | QQKEVEQNSGPLSVPEGAIA | |
| IgG1 heavy | TTGAGCTGGGTTTGGAGCCAA | | SLNCTYSDRGSQSFFWYRQY | |
| chain | CAGAAGGAGGTGGAGCAGAAc | | SGKSPELIMSIYSNGDKEDG | |
| | TCTGGACCCCTCAGTGTTCCA | | RFTAQLNKASQYVSLLIRDS | |
| | GAGGGAGCCATTGCCTCTCTC | | QPSDSATYLCAVETSGTYKY | |
| | AACTGCACTTACAGTGACCGA | | IFGTGTRLKVLANIQNPDPA | |
| | GGTTCCCAGTCCTTCTTCTGG | | VYQLRDSKSSDKSVCLFTDF | |
| | TACAGACAATATTCTGGGAAA | | DSQTNVSQSKDSDVYITDKT | |
| | AGCCCTGAGTTGATAATGTCC | | VLDMRSMDFKSNSAVAWSNK | |
| | ATATACTCCAATGGTGACAAA | | SDFACANAFNNSIIPEDTFF | |
| | GAAGATGGAAGGTTTACAGCA | | PSPESSCGGGGSGGGGSGGG | |
| | CAGCTCAATAAAGCCAGCCAG | | GSGGGGSLEIRAAFLRQRNT | |
| | TATGTTTCTCTGCTCATCAGA | | ALRTEVAELEQEVQRLENEV | |
| | GACTCCCAGCCCAGTGATTCA | | SQYETRYGPLGGSGGASTKG | |
| | GCCACCTACCTCTGTGCCGTG | | PSVFPLAPSSKSTSGGTAAL | |
| | GAGACCTCAGGAACCTACAAA | | GCLVKDYFPEPVTVSWNSGA | |
| | TACATCTTTGGAACAGGCACC | | LTSGVHTFPAVLQSSGLYSL | |
| | AGGCTGAAGGTTTTAGCAAAT | | SSVVTVPSSSLGTQTYICNV | |
| | ATCCAGAACCCTGACCCTGCC | | NHKPSNTKVDKKVEPKSCDK | |
| | GTGTACCAGCTGAGAGACTCT | | THTCPPCPAPELLGGPSVFL | |
| | AAATCCAGTGACAAGTCTGTC | | FPPKPKDTLMISRTPEVTCV | |
| | TGCCTATTCACCGATTTTGAT | | VVDVSHEDPEVKFNWYVDGV | |
| | TCTCAAACAAATGTGTCACAA | | EVHNAKTKPREEQYNSTYRV | |
| | AGTAAGGATTCTGATGTGTAT | | VSVLTVLHQDWLNGKEYKCK | |
| | ATCACAGACAAAACTGTGCTA | | VSNKALPAPIEKTISKAKGQ | |
| | GACATGAGGTCTATGGACTTC | | PREPQVYTLPPSREEMTKNQ | |
| | AAGAGCAACAGTGCTGTGGCC | | VSLTCLVKGFYPSDIAVEWE | |
| | TGGAGCAACAAATCTGACTTT | | SNGQPENNYKTTPPVLDSDG | |
| | GCATGTGCAAACGCCTTCAAC | | SFFLYSKLTVDKSRWQQGNV | |
| | AACAGCATTATTCCAGAAGAC | | FSCSVMHEALHNHYTQKSLS | |
| | ACCTTCTTCCCCAGCCCAGAA | | LSPGK | |
| | AGTTCCTGTGGTGGAGGTGGG | | | |
| | AGTGGGGGAGGTGGATCAGGA | | | |
| | GGCGGTGGTAGTGGTGGTGGC | | | |
| | GGTTCTTTGGAGATACGGGCT | | | |
| | GCTTTTCTCCGCCAACGAAAC | | | |
| | ACTGCACTGCGAACCGAAGTA | | | |
| | GCAGAACTGGAACAGGAGGTG | | | |
| | CAAAGGCTCGAGAATGAGGTT | | | |
| | TCCCAGTACGAAACACGATAC | | | |
| | GGCCCTTTGGGCGGATCCGGA | | | |
| | GGGGCTAGCACCAAGGGCCCA | | | |
| | TCGGTCTTCCCCCTGGCACCC | | | |
| | TCCTCCAAGAGCACCTCTGGG | | | |
| | GGCACAGCGGCCCTGGGCTGC | | | |
| | CTGGTCAAGGACTACTTCCCC | | | |
| | GAACCGGTGACGGTGTCGTGG | | | |
| | AACTCAGGCGCCCTGACCAGC | | | |
| | GGCGTGCACACCTTCCCGGCT | | | |
| | GTCCTACAGTCCTCAGGACTC | | | |
| | TACTCCCTCAGCAGCGTGGTG | | | |
| | ACCGTGCCCTCCAGCAGCTTG | | | |
| | GGCACCCAGACCTACATCTGC | | | |
| | AACGTGAATCACAAGCCCAGC | | | |
| | AACACCAAGGTGGACAAGAAA | | | |
| | GTTGAGCCCAAATCTTGTGAC | | | |
| | AAAACTCACACATGCCCACCG | | | |
| | TGCCCAGCACCTGAACTCCTG | | | |
| | GGGGGACCGTCAGTCTTCCTC | | | |
| | TTCCCCCCAAAACCCAAGGAC | | | |
| | ACCCTCATGATCTCCCGGACC | | | |
| | CCTGAGGTCACATGCGTGGTG | | | |
| | GTGGACGTGAGCCACGAAGAC | | | |
| | CCTGAGGTCAAGTTCAACTGG | | | |
| | TACGTGGACGGCGTGGAGGTG | | | |
| | CATAATGCCAAGACAAAGCCG | | | |
| | CGGGAGGAGCAGTACAACAGC | | | |
| | ACGTACCGTGTGGTCAGCGTC | | | |
| | CTCACCGTCCTGCACCAGGAC | | | |
| | TGGCTGAATGGCAAGGAGTAC | | | |
| | AAGTGCAAGGTCTCCAACAAA | | | |
| | GCCCTCCCAGCCCCCATCGAG | | | |
| | AAAACCATCTCCAAAGCCAAA | | | |
| | GGGCAGCCCCGAGAACCACAG | | | |
| | GTGTACACCCTGCCCCCATCC | | | |
| | CGGGAGGAGATGACCAAGAAC | | | |
| | CAGGTCAGCCTGACCTGCCTG | | | |
| | GTCAAAGGCTTCTATCCCAGC | | | |
| | GACATCGCCGTGGAGTGGGAG | | | |
| | AGCAATGGGCAGCCGGAGAAC | | | |
| | AACTACAAGACCACGCCTCCC | | | |
| | GTGCTGGACTCCGACGGCTCC | | | |
| | TTCTTCCTCTACAGCAAGCTC | | | |
| | ACCGTGGACAAGAGCAGGTGG | | | |
| | CAGCAGGGGAACGTCTTCTCA | | | |
| | TGCTCCGTGATGCATGAGGCT | | | |
| | CTGCACAACCACTACACGCAG | | | |
| | AAGAGCCTCTCCCTGTCTCCG | | | |
| | GGTAAATGA | | | |
| FK10 beta- | ATGGGGAGTGATCCTGATCTG | 81 | MGSDPDLVKLPSCPDPAMGT | 82 |
| LZR-IgG1 | GTAAAGCTCCCATCCTGCCCT | | RLLFWVAFCLLGADHTGAGV | |
| light chain | GACCCTGCCATGGGCACCAGG | | SQSPSNKVTEKGKDVELRCD | |
| | CTCCTCTTCTGGGTGGCCTTC | | PISGHTALYWYRQSLGQGLE | |
| | TGTCTCCTGGGGGCAGATCAC | | FLIYFQGNSAPDKSGLPSDR | |
| | ACAGGAGCTGGAGTCTCCCAG | | FSAERTGGSVSTLTIQRTQQ | |
| | TCCCCCAGTAACAAGGTCACA | | EDSAVYLCASSFGPDGYTFG | |
| | GAGAAGGGAAAGGATGTAGAG | | SGTRLTVVEDLNKVFPPEVA | |
| | CTCAGGTGTGATCCAATTTCA | | VFEPSEAEISHTQKATLVCL | |
| | GGTCATACTGCCCTTTACTGG | | ATGFFPDHVELSWWVNGKEV | |
| | TACCGACAGAGCCTGGGGCAG | | HSGVSTDPQPLKEQPALNDS | |
| | GGCCTGGAGTTTTTAATTTAC | | RYCLSSRLRVSATFWQNPRN | |
| | TTCCAAGGCAACAGTGCACCA | | HFRCQVQFYGLSENDEWTQD | |
| | GACAAATCAGGGCTGCCCAGT | | RAKPVTQIVSAEAWGRADCG | |
| | GATCGCTTCTCTGCAGAGAGG | | GGGSGGGGSGGGGSGGGGSL | |
| | ACTGGGGGcTCCGTCTCCACT | | EIEAAFLERENTALETRVAE | |
| | CTGACGATCCAGCGCACACAG | | LRQRVQRLRNRVSQYRTRYG | |
| | CAGGAGGACTCGGCCGTGTAT | | PLGGSGGRTVAAPSVFIFPP | |
| | CTCTGTGCCAGCAGCTTTGGA | | SDEQLKSGTASVVCLLNNFY | |
| | CCAGATGGCTACACCTTCGGT | | PREAKVQWKVDNALQSGNSQ | |
| | TCGGGGACCAGGTTAACCGTT | | ESVTEQDSKDSTYSLSSTLT | |
| | GTAGAGGACCTGAACAAGGTG | | LSKADYEKHKVYACEVTHQG | |
| | TTCCCACCCGAGGTCGCTGTG | | LSSPVTKSFNRGEC | |
| | TTTGAGCCATCAGAAGCAGAG | | | |
| | ATCTCCCACACCCAAAAGGCC | | | |
| | ACACTGGTGTGCCTGGCCACA | | | |
| | GGCTTCTTCCCTGACCACGTG | | | |
| | GAGCTGAGCTGGTGGGTGAAT | | | |
| | GGGAAGGAGGTGCACAGTGGG | | | |
| | GTCAGCACGGACCCGCAGCCC | | | |
| | CTCAAGGAGCAGCCCGCCCTC | | | |
| | AATGACTCCAGATACTGCCTG | | | |
| | AGCAGCCGCCTGAGGGTCTCG | | | |
| | GCCACCTTCTGGCAGAACCCC | | | |
| | CGCAACCACTTCCGCTGTCAA | | | |
| | GTCCAGTTCTACGGGCTCTCG | | | |
| | GAGAATGACGAGTGGACCCAG | | | |
| | GATAGGGCCAAACCCGTCACC | | | |
| | CAGATCGTCAGCGCCGAGGCC | | | |
| | TGGGGTAGAGCAGACTGTGGT | | | |
| | GGAGGTGGGAGTGGGGGAGGT | | | |
| | GGATCAGGCGGCGGGGGGAGC | | | |
| | GGTGGAGGGGGCAGTCTTGAG | | | |
| | ATTGAAGCAGCCTTCCTGGAG | | | |
| | AGAGAAAATACAGCACTGGAG | | | |
| | ACAAGGGTCGCTGAACTTAGG | | | |
| | CAACGCGTTCAACGCCTCCGG | | | |
| | AATAGAGTTAGTCAGTATAGA | | | |
| | ACACGCTATGGACCTTTGGGC | | | |
| | GGATCCGGAGGGCGTACGGTG | | | |
| | GCTGCACCATCTGTCTTCATC | | | |
| | TTCCCGCCATCTGATGAGCAG | | | |
| | TTGAAATCTGGAACTGCCTCT | | | |
| | GTTGTGTGCCTGCTGAATAAC | | | |
| | TTCTATCCCAGAGAGGCCAAA | | | |
| | GTACAGTGGAAGGTGGATAAC | | | |
| | GCCCTCCAATCGGGTAACTCC | | | |
| | CAGGAGAGTGTCACAGAGCAG | | | |
| | GACAGCAAGGACAGCACCTAC | | | |
| | AGCCTCAGCAGCACCCTGACG | | | |
| | CTGAGCAAAGCAGACTACGAG | | | |
| | AAACACAAAGTCTACGCCTGC | | | |
| | GAAGTCACCCATCAGGGCCTG | | | |
| | AGCTCGCCCGTCACAAAGAGC | | | |
| | TTCAACAGGGGAGAGTGTTAG | | | |

1. A soluble, multimeric fusion protein that binds to a component of the MHC/TCR immune complex, comprising:
(a) a first fusion protein comprising a soluble T cell receptor (TCR) or a soluble Major Histocompatibility Complex (MHC) linked to an immunoglobulin framework by a first multimerization domain; and
(b) a second fusion protein comprising a soluble TCR or a soluble MHC linked to an immunoglobulin framework by a second multimerization domain that binds to the first multimerization domain;
wherein the first and second fusion protein form a soluble multimeric fusion protein.

2. The soluble, multimeric protein fusion of claim 1, wherein the first fusion protein comprises a soluble TCR polypeptide comprising a variable alpha (Vα) domain, and optionally a constant alpha (Cα) domain, and the second fusion protein comprises a soluble TCR polypeptide comprising a variable β domain (Vβ), and optionally a constant β domain (Cβ).

3. The soluble, multimeric protein fusion of claim 1, wherein the first fusion protein comprises a soluble TCR polypeptide comprising a Vα domain, a Vβ domain and a Cβ domain, and the second fusion protein comprises soluble TCR polypeptide comprising a Vα domain, a Vβ domain and a CP domain.

4. The soluble, multimeric protein fusion of claim 2, wherein at least one fusion protein comprises a soluble TCR polypeptide operably linked to an immunoglobulin heavy chain constant region or fragment thereof.

5. The soluble, multimeric protein fusion of claim 2, wherein at least two fusion proteins comprise a soluble TCR polypeptide operably linked to an immunoglobulin heavy chain constant region or fragment thereof.

6. The soluble, multimeric protein fusion of claim 2, wherein at least three fusion proteins comprise a soluble TCR polypeptide operably linked to an immunoglobulin heavy chain constant region or fragment thereof.

7. The soluble, multimeric protein fusion of claim 2, wherein the first fusion protein comprises a soluble TCR polypeptide operably linked to an immunoglobulin heavy chain constant region or fragment thereof, and the second fusion protein comprises a soluble TCR polypeptide operably linked to an immunoglobulin light chain constant region or fragment thereof.

8. The soluble, multimeric protein fusion of claim 2, wherein at least two first fusion proteins comprise a soluble TCR polypeptide operably linked to an immunoglobulin heavy chain constant region or fragment thereof, and at least two second fusion proteins comprise a soluble TCR polypeptide operably linked to an immunoglobulin light chain constant region or fragment thereof.

9. The soluble, multimeric protein fusion of claim 2, wherein the multimeric protein fusion is a dimer, a trimer, a tetramer or a hexamer.

10. The soluble, multimeric protein fusion of claim 9, wherein the multimeric protein fusion is a dimer.

11. The soluble, multimeric protein fusion of claim 9, wherein the multimeric protein fusion is a tetramer.

12. A soluble, multimeric TCR-immunoglobulin fusion protein comprising:
(a) a first fusion protein comprising the structure:
VαCα-X1-Ig(Fc)
wherein Vα is a TCR α variable region, Cα is TCR α constant region, X1 is a first multimerization domain, and Ig(Fc) is an immunoglobulin Fc domain or fragment thereof; and
(b) a second fusion protein comprising the structure:
VβCβ-X2-Ig(CL)
wherein Vβ is a TCR β variable region, CP is a TCR β constant region, X2 is a second multimerization domain, and Ig(CL) is an immunoglobulin light chain constant region or fragment thereof;
wherein the first and second fusion proteins form a soluble, multimeric TCR-immunoglobulin protein.

13. A soluble, multimeric TCR-immunoglobulin fusion protein comprising:
(a) a first fusion protein comprising the structure:
VαVβCβ-X1-Ig(Fc)
wherein Vα is a TCR α variable region, Vβ is a TCR β variable region, Cβ is a TCR α variable region, X1 is a first multimerization domain, and Ig(Fc) is an immunoglobulin Fc domain or fragment thereof; and
(b) a second fusion protein comprising the structure:
VαVβCβ-X2-Ig(CL)
wherein Vα is a TCR α variable region, Vβ is a TCR β variable region, Cβ is a TCR α variable region, X2 is a second multimerization domain, and Ig(CL) is an immunoglobulin light chain constant region; and
wherein the first and second fusion proteins form a soluble, multimeric TCR-immunoglobulin fusion protein.

14. The soluble, multimeric TCR-immunoglobulin fusion protein of claim 12, wherein the multimeric protein fusion is a dimer.

15. The soluble, multimeric TCR-immunoglobulin fusion protein of claim 13, wherein the multimeric protein fusion is a tetramer.

16. A soluble, multimeric TCR-immunoglobulin fusion protein comprising:
(a) a first fusion protein comprising the structure:
Vα-X1-Ig(CH)
wherein Vα is a TCR α variable region, X1 is a first multimerization domain, and Ig(CH) is an immunoglobulin heavy chain constant region or fragment thereof; and
(b) a second fusion protein comprising the structure:
Vβ-X2-Ig(CL)
wherein Vβ is a TCR β variable region, X2 is a second multimerization domain, and Ig(CL) is an immunoglobulin light chain constant region or fragment thereof;
wherein the first fusion protein and the second fusion protein form a soluble, multimeric TCR-immunoglobulin fusion protein.

17. A soluble, multimeric TCR-immunoglobulin fusion protein comprising:
(a) a first fusion protein comprising the structure:
VαCα-X1-Ig(CH)
wherein Vα is a TCR α variable region, Cα is TCR α constant region, X1 is a first multimerization domain, and Ig(CH) is an immunoglobulin heavy chain constant region or fragment thereof; and
(b) a second fusion protein comprising the structure:
VβCβ-X2-Ig(CL)
wherein Vβ is a TCR β variable region, CP is a TCR β constant region, X2 is a second multimerization domain, and Ig(CL) is an immunoglobulin light chain constant region or fragment thereof;
wherein the first fusion protein and the second fusion protein form a soluble, multimeric TCR-immunoglobulin fusion protein.

18. The soluble, multimeric TCR-immunoglobulin fusion protein of claim 16, wherein the multimeric fusion protein comprises two first fusion proteins and two second fusion proteins.

19. The soluble, multimeric TCR-immunoglobulin fusion protein of claim 18, wherein the immunoglobulin heavy chain constant region or fragment thereof of the two first fusion proteins and the immunoglobulin light chain constant region of the two second fusion proteins forms an immunoglobulin framework.

20. The soluble, multimeric TCR-immunoglobulin fusion protein of claim 16, wherein the soluble, multimeric TCR-immunoglobulin fusion protein is a dimer.

21. A soluble, multimeric TCR-immunoglobulin fusion protein comprising:
(a) a first fusion protein comprising the structure:
VαVβCβ-X1-Ig(CH)
wherein Vα is a TCR α variable region, Vβ is a TCR β variable region, Cβ is a TCR β constant region, X1 is a first multimerization domain, and Ig(CH) is an immunoglobulin heavy chain constant region or fragment thereof; and
(b) a second fusion protein comprising the structure:
VαVβCβ-X2-Ig(CL)
wherein Vα is a TCR α variable region, Vβ is a TCR β variable region, CP is a TCR β constant region, X2 is a second multimerization domain, and Ig(CL) is an immunoglobulin light chain constant region,
wherein the first fusion protein and the second fusion protein form a soluble, multimeric TCR-immunoglobulin fusion protein.

22. The soluble, multimeric TCR-immunoglobulin fusion protein of claim 21, wherein the multimeric fusion protein comprises two first fusion proteins and two second fusion proteins.

23. The soluble, multimeric TCR-immunoglobulin fusion protein of claim 22, wherein the immunoglobulin heavy chain constant region or fragment thereof of the two first fusion proteins and the immunoglobulin light chain constant region of the two second fusion proteins forms an immunoglobulin framework.

24. The soluble, multimeric TCR-immunoglobulin fusion protein ofl claim 21, wherein the soluble, multimeric TCR-immunoglobulin fusion protein is a tetramer.

25. A soluble, multimeric TCR-immunoglobulin fusion protein comprising one or more fusion proteins comprising the structure:
VαVβCβ-X-Ig(CH)
wherein Vα is a TCR α variable region, Vβ is a TCR β variable region, CP is a TCR β constant region, X is a multimerization domain, and Ig(CH) is an immunoglobulin heavy chain constant region or fragment thereof; and wherein the one or more fusion proteins form a soluble, multimeric TCR-immunoglobulin fusion protein.

26. The soluble, multimeric TCR-immunoglobulin fusion protein of claim 25, wherein the multimeric TCR-immunoglobulin fusion protein comprises two fusion proteins.

27. The soluble, multimeric TCR-immunoglobulin fusion protein of claim 26, wherein the immunoglobulin heavy chain constant region or fragment thereof of the two fusion proteins forms an immunoglobulin framework.

28. The soluble, multimeric TCR-immunoglobulin fusion protein of claim 25, wherein the soluble, multimeric TCR-immunoglobulin protein is a dimer.

29. The soluble, multimeric TCR-immunoglobulin fusion protein of claim 25, wherein the multimeric TCR-immunoglobulin fusion protein comprises three fusion proteins.

30. The soluble, multimeric TCR-immunoglobulin fusion protein of claim 29, wherein the three fusion proteins are linked through the multimerization domains, and wherein the multimeric TCR-immunoglobulin fusion protein is a trimer.

31. The soluble multimeric fusion protein of claim 2, wherein each TCR-fusion protein in the multimeric protein binds to the same peptide antigen.

32. The soluble multimeric fusion protein of claim 2, wherein at least two of the TCR-fusion protein in the multimeric protein bind to different peptide antigens.

33.-102. (canceled)

103. The soluble, multimeric protein fusion protein of claim 1, wherein the first and second multimerization domains are leucine zipper dimerization domains.

104. The soluble, multimeric protein fusion protein of claim 103, wherein the first multimerization domain and/or the second multimerization domain is LZR that comprises an amino acid sequence identified by SEQ ID NO: 8.

105. The soluble, multimeric protein fusion protein of claim 103, wherein the first multimerization domain and/or the second multimerization domain is LZL that comprises an amino acid sequence identified by SEQ ID NO: 6.

106. The soluble, multimeric protein fusion protein of claim 103, wherein the first multimerization domain is LZR that comprises an amino acid sequence identified by SEQ ID NO: 8, and the second multimerization domain is LZL that comprises an amino acid sequence identified by SEQ ID NO: 6.

107. The soluble, multimeric protein fusion protein of claim 103, wherein the first multimerization domain is LZL that comprises an amino acid sequence identified by SEQ ID NO: 6, and the second multimerization domain is LZR that comprises an amino acid sequence identified by SEQ ID NO: 8.

108. The soluble, multimeric protein fusion of claim 1, wherein the multimerization domains are self-trimerization domains.

109. The soluble, multimeric protein fusion of claim 108, wherein each self-trimerization domain comprises a collagen-like scaffold comprising (GX1X2)n, wherein G is glycine, X1 and X2 are any amino acid residues, and n is at least 5.

110. The soluble, multimeric protein fusion of claim 109, wherein X1 and X2 are proline, and
wherein the self-trimerization domain comprises (GPP)10 (SEQ ID NO: 60).

111. The soluble, multimeric protein fusion of claim 1, wherein the first or second multimerization domain comprises a leucine zipper domain operatively linked to a self-trimerization domain.

112. The soluble, multimeric protein fusion complex of claim 1, wherein at least one fusion protein comprises a peptide linker positioned between the soluble TCR polypeptide or the soluble MEW polypeptide and the multimerization domain.

113. The soluble, multimeric protein fusion of claim 112, wherein the peptide linker comprises a Gly-Ser linker.

114. The soluble, multimeric protein fusion complex of claim 113, wherein the Gly-Ser linker is selected from the group consisting of: (G4S)4 (SEQ ID NO: 9), (G4S)3 (SEQ ID NO: 56), (G4S)2 (SEQ ID NO: 58), G2SG2 (SEQ ID NO: 12), or GSG.

115. The soluble, multimeric protein fusion complex of claim 1, wherein at least one fusion protein comprises a Gly-Ser linker positioned between the soluble TCR polypeptide or the soluble MEW polypeptide and the multimerization domain, and wherein the Gly-Ser linker is selected from the group consisting of: (G4S)4 (SEQ ID NO: 9), (G4S)3 (SEQ ID NO: 56), (G4S)2 (SEQ ID NO: 58), G2SG2 (SEQ ID NO: 12), or GSG.

116. The soluble, multimeric protein fusion complex of claim 1, wherein at least one fusion protein comprises a peptide linker positioned between the multimerization domain and the immunoglobulin framework.

117. The soluble, multimeric protein fusion of claim 116, wherein the peptide linker comprises a Gly-Ser linker.

118. The soluble, multimeric protein fusion of claim 117, wherein the Gly-Ser linker comprises the amino acid sequence GGSGG (SEQ ID NO: 12).

119. The soluble, multimeric protein fusion of claim 1 comprising a signal peptide.

120. The soluble, multimeric protein fusion of claim 1, wherein the soluble TCR polypeptide in at least one fusion protein binds to an MHC peptide.

121. The soluble, multimeric protein fusion of claim 120, wherein the MHC peptide is derived from an from a cancer antigen, a viral antigen, a bacterial antigen, a parasitic antigen or an allergen.

122. The soluble, multimeric protein fusion of claim 120, wherein the MHC peptide is derived from a cancer antigen.

123. The soluble, multimeric protein fusion of claim 122, wherein the MHC peptide is derived from the human endogenous retrovirus (HERV-K) envelope protein.

124. The soluble, multimeric protein fusion of claim 120, wherein the MHC peptide is derived from a viral antigen.

125. The soluble, multimeric protein fusion of claim 124, wherein the MHC peptide is derived from the human immunodeficiency virus (HIV) group antigens (Gag) protein.

126. The soluble, multimeric protein fusion of claim 125, wherein the MHC peptide is the HLA-A02-restricted FLGKIWPSYK epitope (SEQ ID NO: 59).

127. (canceled)

128. A soluble, multimeric TCR-immunoglobulin fusion protein comprising:
(a) a first fusion protein comprising the structure:
VαCα-X1-Ig(CH)
wherein Vα is a TCR α variable region, Cα is TCR α constant region, X1 is a multimerization domain comprising a first leucine zipper domain, and Ig(CH) is an immunoglobulin heavy chain constant region or fragment thereof; and
(b) a second fusion protein comprising the structure:
VβCβ-X2-Ig(CL)
wherein Vβ is a TCR β variable region, CP is a TCR β constant region, X2 is a multimerization domain comprising a second leucine zipper domain, and Ig(CL) is an immunoglobulin light chain constant region or fragment thereof;
wherein the multimeric fusion protein comprises two first fusion proteins and two second fusion proteins, wherein the immunoglobulin heavy chain constant region or fragment thereof and the immunoglobulin light chain constant region or fragment thereof of the first and second fusion proteins forms an immunoglobulin framework, and wherein multimerization of the first and second leucine zipper domains provides a soluble, multimeric TCR-immunoglobulin protein that is a TCR dimer.

129. The soluble, multimeric TCR-immunoglobulin fusion protein of claim 128, wherein the first fusion protein comprises a TCR α chain comprising an amino acid sequence set forth by SEQ ID NO: 64 (HERV-K TCRalpha), and wherein the second fusion protein comprises a TCR β chain comprising an amino acid sequence set forth by SEQ ID NO: 66 (HERV-K TCRbeta).

130. The soluble, multimeric TCR-immunoglobulin fusion protein of claim 128, wherein the first fusion protein comprises a TCR α chain comprising an amino acid sequence set forth by SEQ ID NO: 76 (FK10 TCRalpha), and wherein the second fusion protein comprises a TCR β chain comprising an amino acid sequence set forth by SEQ ID NO: 78 (FK10 TCRbeta).

131.-168. (canceled)


SEQUENCE LISTING (83 sequences)

SEQ ID NO: 1 - DNA, 687 bp, Artificial Sequence
  Synthetic Murine  OTI TCR-alpha
atggacaagatcctgacagcatcgtttttactcctaggccttcacctagctggggtgaatggccagcagcaggagaaacgtgaccagcagcaggtgagacaaagtccccaatctctgacagtctgggaaggagagaccgcaattctgaactgcagttatgaggacagcacttttaactacttcccatggtaccagcagttccctggggaaggccctgcactcctgatatccatacgttcagtgtccgataaaaaggaagatggacgattcacaatcttcttcaataaaagggagaaaaagctctccttgcacatcacagactctcagcctggagactcagctacctacttctgtgcagcaagtgacaactatcagttgatctggggctctgggaccaagctaattataaagccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatggaagctatggattccaagagcaatggggccattgcctggagcaaccagacaagcttcacctgccaagatatcttcaaagagaccaacgccacctaccccagttcagacgttccctgt

SEQ ID NO: 2 - protein, 229 aa, Artificial Sequence
  Synthetic Murine  OTI TCR-alpha
MDKILTASFLLLGLHLAGVNGQQQEKRDQQQVRQSPQSLTVWEGETAILNCSYEDSTFNYFPWYQQFPGEGPALLISIRSVSDKKEDGRFTIFFNKREKKLSLHITDSQPGDSATYFCAASDNYQLIWGSGTKLIIKPDIQNPEPAVYQLKDPRSQDSTLCLFTDFDSQINVPKTMESGTFITDKTVLDMEAMDSKSNGAIAWSNQTSFTCQDIFKETNATYPSSDVPC

SEQ ID NO: 3 - DNA, 801 bp, Artificial Sequence
  Synthetic Murine OTI TCR-beta
atgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctacccagcagattctcagtccaacagtttgatgactatcactctgaaatgaacatgagtgccttggaactggaggactctgctatgtacttctgtgccagctctcgggccaattatgaacagtacttcggtcccggcaccaggctcacggttttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgt

SEQ ID NO: 4 - protein, 267 aa, Artificial Sequence
  Synthetic Murine OTI TCR-beta
MSNTVLADSAWGITLLSWVTVFLLGTSSADSGVVQSPRHIIKEKGGRSVLTCIPISGHSNVVWYQQTLGKELKFLIQHYEKVERDKGFLPSRFSVQQFDDYHSEMNMSALELEDSAMYFCASSRANYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADC

SEQ ID NO: 5 - DNA, 129 bp, Artificial Sequence
  Synthetic LZL
ttggagatacgggctgcttttctccgccaacgaaacactgcactgcgaaccgaagtagcagaactggaacaggaggtgcaaaggctcgagaatgaggtttcccagtacgaaacacgatacggccctttg

SEQ ID NO: 6 - protein, 43 aa, Artificial Sequence
  Synthetic LZL
LEIRAAFLRQRNTALRTEVAELEQEVQRLENEVSQYETRYGPL

SEQ ID NO: 7 - DNA, 129 bp, Artificial Sequence
  Synthetic LZR
cttgagattgaagcagccttcctggagagagaaaatacagcactggagacaagggtcgctgaacttaggcaacgcgttcaacgcctccggaatagagttagtcagtatagaacacgctatggacctttg

SEQ ID NO: 8 - protein, 43 aa, Artificial Sequence
  Synthetic LZR
LEIEAAFLERENTALETRVAELRQRVQRLRNRVSQYRTRYGPL

SEQ ID NO: 9 - DNA, 60 bp, Artificial Sequence
  Synthetic Linker (GGGGS)4
ggtggaggtgggagtgggggaggaggcagtgggggcggcgggagtggcggggggggttcc

SEQ ID NO: 10 - protein, 20 aa, Artificial Sequence
  Synthetic Linker (GGGGS)4
GGGGSGGGGSGGGGSGGGGS

SEQ ID NO: 11 - DNA, 15 bp, Artificial Sequence
  Synthetic Linker
ggcggatccggaggg

SEQ ID NO: 12 - protein, 5 aa, Artificial Sequence
  Synthetic Linker
GGSGG

SEQ ID NO: 13 - DNA, 1008 bp, Artificial Sequence
  Synthetic Murine  IgG heavy chain
gccaaaaccaccgctccatctgtctaccccttggccccagtgtgcggtggaactactggtagctccgtgacactgggctgcctggtgaaaggctacttccctgagcctgttacactcacatggaattcaggatccctgtcctccggagttcacaccttcccggcactcctgcagagcggactttacacactgtcatcctccgtaactgtgacaagcaacacctggccttctcagaccattacttgcaacgtggcccatcccgcttcctccacaaaagtggacaaaaagatcgaacctagagtccccattactcaaaatccctgccccccgcttaaagagtgccccccatgtgccgccccagacctgctcggagggccgagcgtgtttatctttccacccaagattaaagacgttctgatgatttccctcagccctatggttacgtgcgtcgttgtggatgtgtctgaggacgatcccgatgttcagatctcctggtttgtaaacaatgtggaagtacacaccgctcagacccagacccacagagaggactacaacagtacactgcgagttgtaagcgctcttcctatacaacatcaggattggatgagcggtaaggaatttaaatgtaaagtcaataatagggccttgccaagcccaatcgaaaagactatttctaagcctaggggaccggtccgggctccacaggtctacgtgctgccacccccagccgaagagatgactaagaaggagttctctctgacgtgcatgataactggctttctccccgcagagattgccgtcgattggacaagcaacggccggactgagcagaattacaaaaataccgccacagttctggattctgacggctcatacttcatgtactcaaagctgcgagtccagaaaagcacgtgggagcgcgggagtctgtttgcctgctccgtggtgcatgaaggcctgcacaatcacctgaccactaaaacaatcagtcgctctctgggtaagtga

SEQ ID NO: 14 - protein, 335 aa, Artificial Sequence
  Synthetic Murine  IgG heavy chain
AKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 15 - DNA, 327 bp, Artificial Sequence
  Synthetic Murine  IgG light chain
agacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 16 - protein, 108 aa, Artificial Sequence
  Synthetic Murine  IgG light chain
RRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - DNA, 1899 bp, Artificial Sequence
  Synthetic OTI fusion TCR-alpha-LZL-IgGHC
atggacaagatcctgacagcatcgtttttactcctaggccttcacctagctggggtgaatggccagcagcaggagaaacgtgaccagcagcaggtgagacaaagtccccaatctctgacagtctgggaaggagagaccgcaattctgaactgcagttatgaggacagcacttttaactacttcccatggtaccagcagttccctggggaaggccctgcactcctgatatccatacgttcagtgtccgataaaaaggaagatggacgattcacaatcttcttcaataaaagggagaaaaagctctccttgcacatcacagactctcagcctggagactcagctacctacttctgtgcagcaagtgacaactatcagttgatctggggctctgggaccaagctaattataaagccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatggaagctatggattccaagagcaatggggccattgcctggagcaaccagacaagcttcacctgccaagatatcttcaaagagaccaacgccacctaccccagttcagacgttccctgtggtggaggtgggagtgggggaggaggcagtgggggcggcgggagtggcggggggggttccttggagatacgggctgcttttctccgccaacgaaacactgcactgcgaaccgaagtagcagaactggaacaggaggtgcaaaggctcgagaatgaggtttcccagtacgaaacacgatacggccctttgggcggatccggaggggccaaaaccaccgctccatctgtctaccccttggccccagtgtgcggtggaactactggtagctccgtgacactgggctgcctggtgaaaggctacttccctgagcctgttacactcacatggaattcaggatccctgtcctccggagttcacaccttcccggcactcctgcagagcggactttacacactgtcatcctccgtaactgtgacaagcaacacctggccttctcagaccattacttgcaacgtggcccatcccgcttcctccacaaaagtggacaaaaagatcgaacctagagtccccattactcaaaatccctgccccccgcttaaagagtgccccccatgtgccgccccagacctgctcggagggccgagcgtgtttatctttccacccaagattaaagacgttctgatgatttccctcagccctatggttacgtgcgtcgttgtggatgtgtctgaggacgatcccgatgttcagatctcctggtttgtaaacaatgtggaagtacacaccgctcagacccagacccacagagaggactacaacagtacactgcgagttgtaagcgctcttcctatacaacatcaggattggatgagcggtaaggaatttaaatgtaaagtcaataatagggccttgccaagcccaatcgaaaagactatttctaagcctaggggaccggtccgggctccacaggtctacgtgctgccacccccagccgaagagatgactaagaaggagttctctctgacgtgcatgataactggctttctccccgcagagattgccgtcgattggacaagcaacggccggactgagcagaattacaaaaataccgccacagttctggattctgacggctcatacttcatgtactcaaagctgcgagtccagaaaagcacgtgggagcgcgggagtctgtttgcctgctccgtggtgcatgaaggcctgcacaatcacctgaccactaaaacaatcagtcgctctctgggtaagtga

SEQ ID NO: 18 - protein, 632 aa, Artificial Sequence
  Synthetic OTI fusion TCR-alpha-LZL-IgGHC
MDKILTASFLLLGLHLAGVNGQQQEKRDQQQVRQSPQSLTVWEGETAILNCSYEDSTFNYFPWYQQFPGEGPALLISIRSVSDKKEDGRFTIFFNKREKKLSLHITDSQPGDSATYFCAASDNYQLIWGSGTKLIIKPDIQNPEPAVYQLKDPRSQDSTLCLFTDFDSQINVPKTMESGTFITDKTVLDMEAMDSKSNGAIAWSNQTSFTCQDIFKETNATYPSSDVPCGGGGSGGGGSGGGGSGGGGSLEIRAAFLRQRNTALRTEVAELEQEVQRLENEVSQYETRYGPLGGSGGAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 19 - DNA, 1332 bp, Artificial Sequence
  Synthetic OTI fusion TCR-beta-LZR-IgGLC
atgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctacccagcagattctcagtccaacagtttgatgactatcactctgaaatgaacatgagtgccttggaactggaggactctgctatgtacttctgtgccagctctcgggccaattatgaacagtacttcggtcccggcaccaggctcacggttttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggtggaggtgggagtgggggaggtggatcaggcggcggggggagcggtggagggggcagtcttgagattgaagcagccttcctggagagagaaaatacagcactggagacaagggtcgctgaacttaggcaacgcgttcaacgcctccggaatagagttagtcagtatagaacacgctatggacctttgggcggatccggagggagacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 20 - protein, 443 aa, Artificial Sequence
  Synthetic OTI fusion TCR-beta-LZR-IgGLC
MSNTVLADSAWGITLLSWVTVFLLGTSSADSGVVQSPRHIIKEKGGRSVLTCIPISGHSNVVWYQQTLGKELKFLIQHYEKVERDKGFLPSRFSVQQFDDYHSEMNMSALELEDSAMYFCASSRANYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGGGGSGGGGSGGGGSGGGGSLEIEAAFLERENTALETRVAELRQRVQRLRNRVSQYRTRYGPLGGSGGRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - DNA, 666 bp, Artificial Sequence
  Synthetic Murine 2C TCR-alpha
atgctgctggctctgctgcctgtgctgggcatccacttcgtgctgagggacgcccaggcccagagcgtgacccagcctgacgccagagtgacagtgtctgagggcgccagcctgcagctgagatgcaagtacagctacagcgccaccccctacctgttttggtacgtgcagtaccccagacagggcctgcagctgctgctgaagtactacagcggcgaccctgtggtgcagggcgtgaacggcttcgaggccgagttcagcaagagcaacagcagcttccacctgagaaaggccagcgtgcattggagcgacagcgccgtgtatttttgtgccgtgagcggcttcgccagcgccctgaccttcggcagcggcacaaaagtgatcgtgctgccctacatccagaaccccgagcccgccgtgtaccagctgaaggaccccagaagccaggacagcaccctgtgcctgttcaccgacttcgacagccagatcaacgtgcccaagaccatggaaagcggcaccttcatcaccgataagtgcgtgctggacatgaaggccatggacagcaagtccaacggcgctatcgcctggtccaaccagacctcattcacatgccaggacatcttcaaagagacaaacgccacctaccccagcagcgacgtgccttgt

SEQ ID NO: 22 - protein, 222 aa, Artificial Sequence
  Synthetic Murine 2C TCR-alpha
MLLALLPVLGIHFVLRDAQAQSVTQPDARVTVSEGASLQLRCKYSYSATPYLFWYVQYPRQGLQLLLKYYSGDPVVQGVNGFEAEFSKSNSSFHLRKASVHWSDSAVYFCAVSGFASALTFGSGTKVIVLPYIQNPEPAVYQLKDPRSQDSTLCLFTDFDSQINVPKTMESGTFITDKCVLDMKAMDSKSNGAIAWSNQTSFTCQDIFKETNATYPSSDVPC

SEQ ID NO: 23 - DNA, 798 bp, Artificial Sequence
  Synthetic Murine 2C TCR-beta
atgagcaacaccgccttccccgaccctgcctggaacaccaccctgctgtcctgggtggccctgttcctgctgggcaccaagcacatggaagccgccgtgacacagagccccagaaacaaggtggccgtgaccggcggcaaagtgaccctgagctgcaaccagaccaacaaccacaacaacatgtactggtacagacaggacaccggccacggactgagactgatccactacagctacggcgctggcagcaccgagaagggcgacatccccgacggctacaaggccagcagacccagccaggaaaacttcagcctgatcctggaactggccacccctagccagaccagcgtgtacttctgcgcctctggcggcggaggaaccctgtacttcggagccggcaccagactgagcgtgctggaagatctgagaaacgtgaccccccccaaggtgtccctgttcgagcccagcaaggccgagatcgccaacaagcagaaagccaccctcgtgtgcctggccagaggcttcttccctgaccacgtggagctgtcttggtgggtgaacggcaaagaggtgcacagcggcgtctgcaccgacccccaggcctacaaagagagcaactactcctactgcctgagcagcagactgagagtgtccgccaccttctggcacaaccccagaaaccacttcagatgccaggtgcagttccatggcctgtccgaagaggacaagtggcccgagggcagccctaagcctgtgacacagaacatcagcgccgaggcctggggcagagccgactgt

SEQ ID NO: 24 - protein, 266 aa, Artificial Sequence
  Synthetic Murine 2C TCR-beta
MSNTAFPDPAWNTTLLSWVALFLLGTKHMEAAVTQSPRNKVAVTGGKVTLSCNQTNNHNNMYWYRQDTGHGLRLIHYSYGAGSTEKGDIPDGYKASRPSQENFSLILELATPSQTSVYFCASGGGGTLYFGAGTRLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADC

SEQ ID NO: 25 - DNA, 1878 bp, Artificial Sequence
  Synthetic 2C fusion TCR-alpha-LZL-IgGHC
atgctgctggctctgctgcctgtgctgggcatccacttcgtgctgagggacgcccaggcccagagcgtgacccagcctgacgccagagtgacagtgtctgagggcgccagcctgcagctgagatgcaagtacagctacagcgccaccccctacctgttttggtacgtgcagtaccccagacagggcctgcagctgctgctgaagtactacagcggcgaccctgtggtgcagggcgtgaacggcttcgaggccgagttcagcaagagcaacagcagcttccacctgagaaaggccagcgtgcattggagcgacagcgccgtgtatttttgtgccgtgagcggcttcgccagcgccctgaccttcggcagcggcacaaaagtgatcgtgctgccctacatccagaaccccgagcccgccgtgtaccagctgaaggaccccagaagccaggacagcaccctgtgcctgttcaccgacttcgacagccagatcaacgtgcccaagaccatggaaagcggcaccttcatcaccgataagtgcgtgctggacatgaaggccatggacagcaagtccaacggcgctatcgcctggtccaaccagacctcattcacatgccaggacatcttcaaagagacaaacgccacctaccccagcagcgacgtgccttgtggtggaggtgggagtgggggaggaggcagtgggggcggcgggagtggcggggggggttccttggagatacgggctgcttttctccgccaacgaaacactgcactgcgaaccgaagtagcagaactggaacaggaggtgcaaaggctcgagaatgaggtttcccagtacgaaacacgatacggccctttgggcggatccggaggggccaaaaccaccgctccatctgtctaccccttggccccagtgtgcggtggaactactggtagctccgtgacactgggctgcctggtgaaaggctacttccctgagcctgttacactcacatggaattcaggatccctgtcctccggagttcacaccttcccggcactcctgcagagcggactttacacactgtcatcctccgtaactgtgacaagcaacacctggccttctcagaccattacttgcaacgtggcccatcccgcttcctccacaaaagtggacaaaaagatcgaacctagagtccccattactcaaaatccctgccccccgcttaaagagtgccccccatgtgccgccccagacctgctcggagggccgagcgtgtttatctttccacccaagattaaagacgttctgatgatttccctcagccctatggttacgtgcgtcgttgtggatgtgtctgaggacgatcccgatgttcagatctcctggtttgtaaacaatgtggaagtacacaccgctcagacccagacccacagagaggactacaacagtacactgcgagttgtaagcgctcttcctatacaacatcaggattggatgagcggtaaggaatttaaatgtaaagtcaataatagggccttgccaagcccaatcgaaaagactatttctaagcctaggggaccggtccgggctccacaggtctacgtgctgccacccccagccgaagagatgactaagaaggagttctctctgacgtgcatgataactggctttctccccgcagagattgccgtcgattggacaagcaacggccggactgagcagaattacaaaaataccgccacagttctggattctgacggctcatacttcatgtactcaaagctgcgagtccagaaaagcacgtgggagcgcgggagtctgtttgcctgctccgtggtgcatgaaggcctgcacaatcacctgaccactaaaacaatcagtcgctctctgggtaagtga

SEQ ID NO: 26 - protein, 625 aa, Artificial Sequence
  Synthetic 2C fusion TCR-alpha-LZL-IgGHC
MLLALLPVLGIHFVLRDAQAQSVTQPDARVTVSEGASLQLRCKYSYSATPYLFWYVQYPRQGLQLLLKYYSGDPVVQGVNGFEAEFSKSNSSFHLRKASVHWSDSAVYFCAVSGFASALTFGSGTKVIVLPYIQNPEPAVYQLKDPRSQDSTLCLFTDFDSQINVPKTMESGTFITDKCVLDMKAMDSKSNGAIAWSNQTSFTCQDIFKETNATYPSSDVPCGGGGSGGGGSGGGGSGGGGSLEIRAAFLRQRNTALRTEVAELEQEVQRLENEVSQYETRYGPLGGSGGAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 27 - DNA, 1329 bp, Artificial Sequence
  Synthetic 2C fusion  TCR-beta-LZR-IgGLC
atgagcaacaccgccttccccgaccctgcctggaacaccaccctgctgtcctgggtggccctgttcctgctgggcaccaagcacatggaagccgccgtgacacagagccccagaaacaaggtggccgtgaccggcggcaaagtgaccctgagctgcaaccagaccaacaaccacaacaacatgtactggtacagacaggacaccggccacggactgagactgatccactacagctacggcgctggcagcaccgagaagggcgacatccccgacggctacaaggccagcagacccagccaggaaaacttcagcctgatcctggaactggccacccctagccagaccagcgtgtacttctgcgcctctggcggcggaggaaccctgtacttcggagccggcaccagactgagcgtgctggaagatctgagaaacgtgaccccccccaaggtgtccctgttcgagcccagcaaggccgagatcgccaacaagcagaaagccaccctcgtgtgcctggccagaggcttcttccctgaccacgtggagctgtcttggtgggtgaacggcaaagaggtgcacagcggcgtctgcaccgacccccaggcctacaaagagagcaactactcctactgcctgagcagcagactgagagtgtccgccaccttctggcacaaccccagaaaccacttcagatgccaggtgcagttccatggcctgtccgaagaggacaagtggcccgagggcagccctaagcctgtgacacagaacatcagcgccgaggcctggggcagagccgactgtggtggaggtgggagtgggggaggtggatcaggcggcggggggagcggtggagggggcagtcttgagattgaagcagccttcctggagagagaaaatacagcactggagacaagggtcgctgaacttaggcaacgcgttcaacgcctccggaatagagttagtcagtatagaacacgctatggacctttgggcggatccggagggagacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 28 - protein, 442 aa, Artificial Sequence
  Synthetic 2C fusion  TCR-beta-LZR-IgGLC
MSNTAFPDPAWNTTLLSWVALFLLGTKHMEAAVTQSPRNKVAVTGGKVTLSCNQTNNHNNMYWYRQDTGHGLRLIHYSYGAGSTEKGDIPDGYKASRPSQENFSLILELATPSQTSVYFCASGGGGTLYFGAGTRLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGGGGSGGGGSGGGGSGGGGSLEIEAAFLERENTALETRVAELRQRVQRLRNRVSQYRTRYGPLGGSGGRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 29 - DNA, 666 bp, Artificial Sequence
  Synthetic Murine  6mut 2C TCR-alpha
atgctgctggctctgctgcctgtgctgggcatccacttcgtgctgagggacgcccaggcccagagcgtgacccagcctgacgccagagtgacagtgtctgagggcgccagcctgcagctgagatgcaagtacagctacagcgccaccccctacctgttttggtacgtgcagtaccccagacagggcccccagctgctgctgaagtactacagcggcgaccctgtggtgcagggcgtgaacggcttcgaggccgagttcagcaagagcaacagcagcttccacctgagaaaggccagcgtgcataggagcgacagcgccgtgtatttttgtgccgtgagcggcttcgccagcgccctgaccttcggcagcggcacaaaagtgatcgtgctgccctacaaccagaaccccgagcccgccgtgtaccagctgaaggaccccagaagccaggacagcaccctgtgcctgttcaccgacttcgacagccagatcaacgtgcccaagaccatggaaagcggcaccttcatcaccgataagtgcgtgctggacatgaaggccatggacagcaagtccaacggcgctatcgcctggtccaaccagacctcattcacatgccaggacatcttcaaagagacaaacgccacctaccccagcagcgacgtgccttgt

SEQ ID NO: 30 - protein, 222 aa, Artificial Sequence
  Synthetic Murine  6mut 2C TCR-alpha
MLLALLPVLGIHFVLRDAQAQSVTQPDARVTVSEGASLQLRCKYSYSATPYLFWYVQYPRQGPQLLLKYYSGDPVVQGVNGFEAEFSKSNSSFHLRKASVHRSDSAVYFCAVSGFASALTFGSGTKVIVLPYNQNPEPAVYQLKDPRSQDSTLCLFTDFDSQINVPKTMESGTFITDKCVLDMKAMDSKSNGAIAWSNQTSFTCQDIFKETNATYPSSDVPC

SEQ ID NO: 31 - DNA, 798 bp, Artificial Sequence
  Synthetic Murine  6mut 2C TCR-beta
atgagcaacaccgccttccccgaccctgcctggaacaccaccctgctgtcctgggtggccctgttcctgctgggcaccaagcacatggaagccgccgtgacacagagccccagaaacaaggtggccgtgaccggcgagaaagtgaccctgagctgcaaccagaccaacaaccacaacaacatgtactggtacagacaggacaccggccacgagctgagactgatccactacagctacggcgctggcagcaccgagaagggcgacatccccgacggctacaaggccagcagacccagccaggaaaacttcagcctgatcctggaaagcgccacccctagccagaccagcgtgtacttctgcgcctctggcggcggaggaaccctgtacttcggagccggcaccagactgagcgtgctggaagatctgagaaacgtgaccccccccaaggtgtccctgttcgagcccagcaaggccgagatcgccaacaagcagaaagccaccctcgtgtgcctggccagaggcttcttccctgaccacgtggagctgtcttggtgggtgaacggcaaagaggtgcacagcggcgtctgcaccgacccccaggcctacaaagagagcaactactcctactgcctgagcagcagactgagagtgtccgccaccttctggcacaaccccagaaaccacttcagatgccaggtgcagttccatggcctgtccgaagaggacaagtggcccgagggcagccctaagcctgtgacacagaacatcagcgccgaggcctggggcagagccgactgt

SEQ ID NO: 32 - protein, 266 aa, Artificial Sequence
  Synthetic Murine  6mut 2C TCR-beta
MSNTAFPDPAWNTTLLSWVALFLLGTKHMEAAVTQSPRNKVAVTGEKVTLSCNQTNNHNNMYWYRQDTGHELRLIHYSYGAGSTEKGDIPDGYKASRPSQENFSLILESATPSQTSVYFCASGGGGTLYFGAGTRLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADC

SEQ ID NO: 33 - DNA, 1878 bp, Artificial Sequence
  Synthetic Murine 6mut 2C fusion TCR-alpha-LZL- IgGHC
atgctgctggctctgctgcctgtgctgggcatccacttcgtgctgagggacgcccaggcccagagcgtgacccagcctgacgccagagtgacagtgtctgagggcgccagcctgcagctgagatgcaagtacagctacagcgccaccccctacctgttttggtacgtgcagtaccccagacagggcccccagctgctgctgaagtactacagcggcgaccctgtggtgcagggcgtgaacggcttcgaggccgagttcagcaagagcaacagcagcttccacctgagaaaggccagcgtgcataggagcgacagcgccgtgtatttttgtgccgtgagcggcttcgccagcgccctgaccttcggcagcggcacaaaagtgatcgtgctgccctacaaccagaaccccgagcccgccgtgtaccagctgaaggaccccagaagccaggacagcaccctgtgcctgttcaccgacttcgacagccagatcaacgtgcccaagaccatggaaagcggcaccttcatcaccgataagtgcgtgctggacatgaaggccatggacagcaagtccaacggcgctatcgcctggtccaaccagacctcattcacatgccaggacatcttcaaagagacaaacgccacctaccccagcagcgacgtgccttgtggtggaggtgggagtgggggaggaggcagtgggggcggcgggagtggcggggggggttccttggagatacgggctgcttttctccgccaacgaaacactgcactgcgaaccgaagtagcagaactggaacaggaggtgcaaaggctcgagaatgaggtttcccagtacgaaacacgatacggccctttgggcggatccggaggggccaaaaccaccgctccatctgtctaccccttggccccagtgtgcggtggaactactggtagctccgtgacactgggctgcctggtgaaaggctacttccctgagcctgttacactcacatggaattcaggatccctgtcctccggagttcacaccttcccggcactcctgcagagcggactttacacactgtcatcctccgtaactgtgacaagcaacacctggccttctcagaccattacttgcaacgtggcccatcccgcttcctccacaaaagtggacaaaaagatcgaacctagagtccccattactcaaaatccctgccccccgcttaaagagtgccccccatgtgccgccccagacctgctcggagggccgagcgtgtttatctttccacccaagattaaagacgttctgatgatttccctcagccctatggttacgtgcgtcgttgtggatgtgtctgaggacgatcccgatgttcagatctcctggtttgtaaacaatgtggaagtacacaccgctcagacccagacccacagagaggactacaacagtacactgcgagttgtaagcgctcttcctatacaacatcaggattggatgagcggtaaggaatttaaatgtaaagtcaataatagggccttgccaagcccaatcgaaaagactatttctaagcctaggggaccggtccgggctccacaggtctacgtgctgccacccccagccgaagagatgactaagaaggagttctctctgacgtgcatgataactggctttctccccgcagagattgccgtcgattggacaagcaacggccggactgagcagaattacaaaaataccgccacagttctggattctgacggctcatacttcatgtactcaaagctgcgagtccagaaaagcacgtgggagcgcgggagtctgtttgcctgctccgtggtgcatgaaggcctgcacaatcacctgaccactaaaacaatcagtcgctctctgggtaagtga

SEQ ID NO: 34 - protein, 625 aa, Artificial Sequence
  Synthetic Murine 6mut 2C fusion TCR-alpha-LZL- IgGHC
MLLALLPVLGIHFVLRDAQAQSVTQPDARVTVSEGASLQLRCKYSYSATPYLFWYVQYPRQGPQLLLKYYSGDPVVQGVNGFEAEFSKSNSSFHLRKASVHRSDSAVYFCAVSGFASALTFGSGTKVIVLPYNQNPEPAVYQLKDPRSQDSTLCLFTDFDSQINVPKTMESGTFITDKCVLDMKAMDSKSNGAIAWSNQTSFTCQDIFKETNATYPSSDVPCGGGGSGGGGSGGGGSGGGGSLEIRAAFLRQRNTALRTEVAELEQEVQRLENEVSQYETRYGPLGGSGGAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 35 - DNA, 1329 bp, Artificial Sequence
  Synthetic Murine 6mut 2C fusion TCR-beta-LZR- IgGLC
atgagcaacaccgccttccccgaccctgcctggaacaccaccctgctgtcctgggtggccctgttcctgctgggcaccaagcacatggaagccgccgtgacacagagccccagaaacaaggtggccgtgaccggcgagaaagtgaccctgagctgcaaccagaccaacaaccacaacaacatgtactggtacagacaggacaccggccacgagctgagactgatccactacagctacggcgctggcagcaccgagaagggcgacatccccgacggctacaaggccagcagacccagccaggaaaacttcagcctgatcctggaaagcgccacccctagccagaccagcgtgtacttctgcgcctctggcggcggaggaaccctgtacttcggagccggcaccagactgagcgtgctggaagatctgagaaacgtgaccccccccaaggtgtccctgttcgagcccagcaaggccgagatcgccaacaagcagaaagccaccctcgtgtgcctggccagaggcttcttccctgaccacgtggagctgtcttggtgggtgaacggcaaagaggtgcacagcggcgtctgcaccgacccccaggcctacaaagagagcaactactcctactgcctgagcagcagactgagagtgtccgccaccttctggcacaaccccagaaaccacttcagatgccaggtgcagttccatggcctgtccgaagaggacaagtggcccgagggcagccctaagcctgtgacacagaacatcagcgccgaggcctggggcagagccgactgtggtggaggtgggagtgggggaggtggatcaggcggcggggggagcggtggagggggcagtcttgagattgaagcagccttcctggagagagaaaatacagcactggagacaagggtcgctgaacttaggcaacgcgttcaacgcctccggaatagagttagtcagtatagaacacgctatggacctttgggcggatccggagggagacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 36 - protein, 442 aa, Artificial Sequence
  Synthetic Murine 6mut 2C fusion TCR-beta-LZR- IgGLC
MSNTAFPDPAWNTTLLSWVALFLLGTKHMEAAVTQSPRNKVAVTGEKVTLSCNQTNNHNNMYWYRQDTGHELRLIHYSYGAGSTEKGDIPDGYKASRPSQENFSLILESATPSQTSVYFCASGGGGTLYFGAGTRLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGGGGSGGGGSGGGGSGGGGSLEIEAAFLERENTALETRVAELRQRVQRLRNRVSQYRTRYGPLGGSGGRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 37 - DNA, 60 bp, Artificial Sequence
  Synthetic Murine B2M  signal peptide
atggctcgctcggtgaccctggtctttctggtgcttgtctcactgaccggcctgtatgct

SEQ ID NO: 38 - protein, 20 aa, Artificial Sequence
  Synthetic Murine B2M  signal peptide
MARSVTLVFLVLVSLTGLYA

SEQ ID NO: 39 - DNA, 24 bp, Artificial Sequence
  Synthetic Ova257-265
agtatcattaatttcgaaaaactt

SEQ ID NO: 40 - protein, 8 aa, Artificial Sequence
  Synthetic Ova257-265
SIINFEKL

SEQ ID NO: 41 - DNA, 297 bp, Artificial Sequence
  Synthetic Murine B2M
attcaaaaaaccccacagatccaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatg

SEQ ID NO: 42 - protein, 99 aa, Artificial Sequence [sequence omitted]

SEQ ID NO: 43 - DNA, 849 bp, Artificial Sequence
  Synthetic Murine H2Kb
ggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctgttacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaac

SEQ ID NO: 44 - protein, 283 aa, Artificial Sequence
  Synthetic Murine H2Kb
GPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSN

SEQ ID NO: 45 - DNA, 2547 bp, Artificial Sequence
  Synthetic Murine fusion B2M signal peptide- Ova257-265-B2M-H2Kb-LZL-IgGHC
atggctcgctcggtgaccctggtctttctggtgcttgtctcactgaccggcctgtatgctagtatcattaatttcgaaaaacttggatgtggtgctagcggtggtggtggtagcggaggtggaggcagcattcaaaaaaccccacagatccaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggcggtggtggttccggtggaggcggttccggaggtggtggatccggtggtggtggtagtggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctgttacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacggtggaggtgggagtgggggaggaggcagtgggggcggcgggagtggcggggggggttccttggagatacgggctgcttttctccgccaacgaaacactgcactgcgaaccgaagtagcagaactggaacaggaggtgcaaaggctcgagaatgaggtttcccagtacgaaacacgatacggccctttgggcggatccggaggggccaaaaccaccgctccatctgtctaccccttggccccagtgtgcggtggaactactggtagctccgtgacactgggctgcctggtgaaaggctacttccctgagcctgttacactcacatggaattcaggatccctgtcctccggagttcacaccttcccggcactcctgcagagcggactttacacactgtcatcctccgtaactgtgacaagcaacacctggccttctcagaccattacttgcaacgtggcccatcccgcttcctccacaaaagtggacaaaaagatcgaacctagagtccccattactcaaaatccctgccccccgcttaaagagtgccccccatgtgccgccccagacctgctcggagggccgagcgtgtttatctttccacccaagattaaagacgttctgatgatttccctcagccctatggttacgtgcgtcgttgtggatgtgtctgaggacgatcccgatgttcagatctcctggtttgtaaacaatgtggaagtacacaccgctcagacccagacccacagagaggactacaacagtacactgcgagttgtaagcgctcttcctatacaacatcaggattggatgagcggtaaggaatttaaatgtaaagtcaataatagggccttgccaagcccaatcgaaaagactatttctaagcctaggggaccggtccgggctccacaggtctacgtgctgccacccccagccgaagagatgactaagaaggagttctctctgacgtgcatgataactggctttctccccgcagagattgccgtcgattggacaagcaacggccggactgagcagaattacaaaaataccgccacagttctggattctgacggctcatacttcatgtactcaaagctgcgagtccagaaaagcacgtgggagcgcgggagtctgtttgcctgctccgtggtgcatgaaggcctgcacaatcacctgaccactaaaacaatcagtcgctctctgggtaagtga

SEQ ID NO: 46 - protein, 848 aa, Artificial Sequence
  Synthetic Murine fusion B2M signal peptide- Ova257-265-B2M-H2Kb-LZL-IgGHC
MARSVTLVFLVLVSLTGLYASIINFEKLGCGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNGGGGSGGGGSGGGGSGGGGSLEIRAAFLRQRNTALRTEVAELEQEVQRLENEVSQYETRYGPLGGSGGAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 47 - DNA, 1866 bp, Artificial Sequence
  Synthetic Murine fusion B2M signal peptide- Ova257-265-B2M-H2Kb-LZL-IgGLC
atggctcgctcggtgaccctggtctttctggtgcttgtctcactgaccggcctgtatgctagtatcattaatttcgaaaaacttggatgtggtgctagcggtggtggtggtagcggaggtggaggcagcattcaaaaaaccccacagatccaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggcggtggtggttccggtggaggcggttccggaggtggtggatccggtggtggtggtagtggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctgttacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacggtggaggtgggagtgggggaggtggatcaggcggcggggggagcggtggagggggcagtcttgagattgaagcagccttcctggagagagaaaatacagcactggagacaagggtcgctgaacttaggcaacgcgttcaacgcctccggaatagagttagtcagtatagaacacgctatggacctttgggcggatccggagggagacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 48 - protein, 621 aa, Artificial Sequence
  Synthetic Murine fusion B2M signal peptide- Ova257-265-B2M-H2Kb-LZL-IgGLC
MARSVTLVFLVLVSLTGLYASIINFEKLGCGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNGGGGSGGGGSGGGGSGGGGSLEIEAAFLERENTALETRVAELRQRVQRLRNRVSQYRTRYGPLGGSGGRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 49 - DNA, 39 bp, Artificial Sequence
  Synthetic Furin-GSG-His
cgccgaaaacgcggttctggacaccaccatcaccatcac

SEQ ID NO: 50 - protein, 13 aa, Artificial Sequence
  Synthetic Furin-GSG-His
RRKRGSGHHHHHH

SEQ ID NO: 51 - DNA, 66 bp, Artificial Sequence
  Synthetic GSG-P2A
ggaagcggagctactaacttcagcctgctgaagcaggctggagacgtggaggagaaccctggacct

SEQ ID NO: 52 - protein, 22 aa, Artificial Sequence
  Synthetic GSG-P2A
GSGATNFSLLKQAGDVEENPGP

SEQ ID NO: 53 - DNA, 3333 bp, Artificial Sequence
  Synthetic Single Vector Insert OTITCR-alpha-LZL-IgGHC-furin-GSG-HIS-GSG-P2A-OT1TCR-beta-LZR-mIgG LC
atggacaagatcctgacagcatcgtttttactcctaggccttcacctagctggggtgaatggccagcagcaggagaaacgtgaccagcagcaggtgagacaaagtccccaatctctgacagtctgggaaggagagaccgcaattctgaactgcagttatgaggacagcacttttaactacttcccatggtaccagcagttccctggggaaggccctgcactcctgatatccatacgttcagtgtccgataaaaaggaagatggacgattcacaatcttcttcaataaaagggagaaaaagctctccttgcacatcacagactctcagcctggagactcagctacctacttctgtgcagcaagtgacaactatcagttgatctggggctctgggaccaagctaattataaagccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatggaagctatggattccaagagcaatggggccattgcctggagcaaccagacaagcttcacctgccaagatatcttcaaagagaccaacgccacctaccccagttcagacgttccctgtggtggaggtgggagtgggggaggaggcagtgggggcggcgggagtggcggggggggttccttggagatacgggctgcttttctccgccaacgaaacactgcactgcgaaccgaagtagcagaactggaacaggaggtgcaaaggctcgagaatgaggtttcccagtacgaaacacgatacggccctttgggcggatccggaggggccaaaaccaccgctccatctgtctaccccttggccccagtgtgcggtggaactactggtagctccgtgacactgggctgcctggtgaaaggctacttccctgagcctgttacactcacatggaattcaggatccctgtcctccggagttcacaccttcccggcactcctgcagagcggactttacacactgtcatcctccgtaactgtgacaagcaacacctggccttctcagaccattacttgcaacgtggcccatcccgcttcctccacaaaagtggacaaaaagatcgaacctagagtccccattactcaaaatccctgccccccgcttaaagagtgccccccatgtgccgccccagacctgctcggagggccgagcgtgtttatctttccacccaagattaaagacgttctgatgatttccctcagccctatggttacgtgcgtcgttgtggatgtgtctgaggacgatcccgatgttcagatctcctggtttgtaaacaatgtggaagtacacaccgctcagacccagacccacagagaggactacaacagtacactgcgagttgtaagcgctcttcctatacaacatcaggattggatgagcggtaaggaatttaaatgtaaagtcaataatagggccttgccaagcccaatcgaaaagactatttctaagcctaggggaccggtccgggctccacaggtctacgtgctgccacccccagccgaagagatgactaagaaggagttctctctgacgtgcatgataactggctttctccccgcagagattgccgtcgattggacaagcaacggccggactgagcagaattacaaaaataccgccacagttctggattctgacggctcatacttcatgtactcaaagctgcgagtccagaaaagcacgtgggagcgcgggagtctgtttgcctgctccgtggtgcatgaaggcctgcacaatcacctgaccactaaaacaatcagtcgctctctgggtaagcgccgaaaacgcggttctggacaccaccatcaccatcacggaagcggagctactaacttcagcctgctgaagcaggctggagacgtggaggagaaccctggacctatgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctacccagcagattctcagtccaacagtttgatgactatcactctgaaatgaacatgagtgccttggaactggaggactctgctatgtacttctgtgccagctctcgggccaattatgaacagtacttcggtcccggcaccaggctcacggttttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggtggaggtgggagtgggggaggtggatcaggcggcggggggagcggtggagggggcagtcttgagattgaagcagccttcctggagagagaaaatacagcactggagacaagggtcgctgaacttaggcaacgcgttcaacgcctccggaatagagttagtcagtatagaacacgctatggacctttgggcggatccggagggagacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 54 - protein, 1110 aa, Artificial Sequence
  Synthetic Single Vector Insert OTITCR-alpha-LZL-IgGHC-furin-GSG-HIS-GSG-P2A-OT1TCR-beta-LZR-mIgG LC
MDKILTASFLLLGLHLAGVNGQQQEKRDQQQVRQSPQSLTVWEGETAILNCSYEDSTFNYFPWYQQFPGEGPALLISIRSVSDKKEDGRFTIFFNKREKKLSLHITDSQPGDSATYFCAASDNYQLIWGSGTKLIIKPDIQNPEPAVYQLKDPRSQDSTLCLFTDFDSQINVPKTMESGTFITDKTVLDMEAMDSKSNGAIAWSNQTSFTCQDIFKETNATYPSSDVPCGGGGSGGGGSGGGGSGGGGSLEIRAAFLRQRNTALRTEVAELEQEVQRLENEVSQYETRYGPLGGSGGAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKRRKRGSGHHHHHHGSGATNFSLLKQAGDVEENPGPMSNTVLADSAWGITLLSWVTVFLLGTSSADSGVVQSPRHIIKEKGGRSVLTCIPISGHSNVVWYQQTLGKELKFLIQHYEKVERDKGFLPSRFSVQQFDDYHSEMNMSALELEDSAMYFCASSRANYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGGGGSGGGGSGGGGSGGGGSLEIEAAFLERENTALETRVAELRQRVQRLRNRVSQYRTRYGPLGGSGGRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 55 - DNA, 45 bp, Artificial Sequence
  Synthetic Linker (GGGGS)3
ggtggaggtgggagtgggggaggaggcagtgggggcggcgggagt

SEQ ID NO: 56 - protein, 15 aa, Artificial Sequence
  Synthetic Linker (GGGGS)3
GGGGSGGGGSGGGGS

SEQ ID NO: 57 - DNA, 30 bp, Artificial Sequence
  Synthetic Linker (GGGGS)2
ggtggaggtgggagtgggggaggaggcagt

SEQ ID NO: 58 - protein, 10 aa, Artificial Sequence
  Synthetic Linker (GGGGS)2
GGGGSGGGGS

SEQ ID NO: 59 - protein, 10 aa, Artificial Sequence
  Synthetic HIV Gag epitope
FLGKIWPSYK

SEQ ID NO: 60 - protein, 30 aa, Artificial Sequence
  Synthetic Collagen-like trimerization domain
GPPGPPGPPGPPGPPGPPGPPGPPGPPGPP

SEQ ID NO: 61 - protein, 50 aa, Artificial Sequence
  Synthetic BZip leucine zipper
MDPDLEIRAAFLRQRNTALRTEVAELEQEVQRLEEVSQYETRYGPLGGGK

SEQ ID NO: 62 - protein, 51 aa, Artificial Sequence
  Synthetic AZip leucine zipper
MDPDLEIEAAFLERENTALETRVAELRQRVQRLRNRVSQYRTRYGPLGGGK

SEQ ID NO: 63 - DNA, 672 bp, Artificial Sequence
  Synthetic HERV-K TCR alpha chain
atgctcctgctgctcgtcccagtgctcgaggtgatttttactctgggaggaaccagagcccagtcggtgacccagcttgacagccacgtctctgtctctgaaggaaccccggtgctgctgaggtgcaactactcatcttcttattcaccatctctcttctggtatgtgcaacaccccaacaaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgttgtgagtactctcaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgt

SEQ ID NO: 64 - protein, 224 aa, Artificial Sequence
  Synthetic HERV-K TCR alpha chain
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVSTLKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSC

SEQ ID NO: 65 - DNA, 786 bp, Artificial Sequence
  Synthetic HERV-K TCR beta chain
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcataggcccgtctgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgt

SEQ ID NO: 66 - protein, 262 aa, Artificial Sequence
  Synthetic HERV-K TCR beta chain
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSIGPSEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADC

SEQ ID NO: 67 - DNA, 993 bp, Artificial Sequence
  Synthetic Human IgG1 heavy chain
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 68 - protein, 330 aa, Artificial Sequence
  Synthetic Human IgG1 heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 321 bp, Artificial Sequence
  Synthetic Human IgG1 light chain
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 70 - protein, 107 aa, Artificial Sequence [sequence omitted]

SEQ ID NO: 71 - DNA, 1872 bp, Artificial Sequence
  Synthetic HERV-K alpha chain-LZL-IgG1 heavy chain
atgctcctgctgctcgtcccagtgctcgaggtgatttttactctgggaggaaccagagcccagtcggtgacccagcttgacagccacgtctctgtctctgaaggaaccccggtgctgctgaggtgcaactactcatcttcttattcaccatctctcttctggtatgtgcaacaccccaacaaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgttgtgagtactctcaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtggtggaggtgggagtgggggaggtggatcaggaggcggtggtagtggtggtggcggttctttggagatacgggctgcttttctccgccaacgaaacactgcactgcgaaccgaagtagcagaactggaacaggaggtgcaaaggctcgagaatgaggtttcccagtacgaaacacgatacggccctttgggcggatccggaggggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatga

SEQ ID NO: 72 - protein, 622 aa, Artificial Sequence
  Synthetic HERV-K alpha chain-LZL-IgG1 heavy chain
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVSTLKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGGGGSGGGGSGGGGSGGGGSLEIRAAFLRQRNTALRTEVAELEQEVQRLENEVSQYETRYGPLGGSGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 1314 bp, Artificial Sequence
  Synthetic HERV-K beta chain-LZR-IgG1 light chain
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcataggcccgtctgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggtggaggtgggagtgggggaggtggatcaggcggcggggggagcggtggagggggcagtcttgagattgaagcagccttcctggagagagaaaatacagcactggagacaagggtcgctgaacttaggcaacgcgttcaacgcctccggaatagagttagtcagtatagaacacgctatggacctttgggcggatccggagggcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 74 - protein, 437 aa, Artificial Sequence
  Synthetic HERV-K beta chain-LZR-IgG1 light chain
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSIGPSEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGGGGSGGGGSGGGGSGGGGSLEIEAAFLERENTALETRVAELRQRVQRLRNRVSQYRTRYGPLGGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 681 bp, Artificial Sequence
  Synthetic FK10 TCR alpha chain
atgaaatccttgagagttttactagtgatcctgtggcttcagttgagctgggtttggagccaacagaaggaggtggagcagaactctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtggagacctcaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgt

SEQ ID NO: 76 - protein, 227 aa, Artificial Sequence
  Synthetic FK10 TCR alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVETSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSC

SEQ ID NO: 77 - DNA, 837 bp, Artificial Sequence
  Synthetic FK10 TCR beta chain
atggggagtgatcctgatctggtaaagctcccatcctgccctgaccctgccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggctccgtctccactctgacgatccagcgcacacagcaggaggactcggccgtgtatctctgtgccagcagctttggaccagatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgt

SEQ ID NO: 78 - protein, 279 aa, Artificial Sequence
  Synthetic FK10 TCR beta chain
MGSDPDLVKLPSCPDPAMGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSFGPDGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADC

SEQ ID NO: 79 - DNA, 1878 bp, Artificial Sequence
  Synthetic FK10 alpha-LZL-IgG1 heavy chain
atgaaatccttgagagttttactagtgatcctgtggcttcagttgagctgggtttggagccaacagaaggaggtggagcagaactctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtggagacctcaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtggtggaggtgggagtgggggaggtggatcaggaggcggtggtagtggtggtggcggttctttggagatacgggctgcttttctccgccaacgaaacactgcactgcgaaccgaagtagcagaactggaacaggaggtgcaaaggctcgagaatgaggtttcccagtacgaaacacgatacggccctttgggcggatccggaggggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 80 - protein, 625 aa, Artificial Sequence
  Synthetic FK10 alpha-LZL-IgG1 heavy chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVETSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGGGGSGGGGSGGGGSGGGGSLEIRAAFLRQRNTALRTEVAELEQEVQRLENEVSQYETRYGPLGGSGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 1365 bp, Artificial Sequence
  Synthetic FK10 beta-LZR-IgG1 light chain
atggggagtgatcctgatctggtaaagctcccatcctgccctgaccctgccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggctccgtctccactctgacgatccagcgcacacagcaggaggactcggccgtgtatctctgtgccagcagctttggaccagatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggtggaggtgggagtgggggaggtggatcaggcggcggggggagcggtggagggggcagtcttgagattgaagcagccttcctggagagagaaaatacagcactggagacaagggtcgctgaacttaggcaacgcgttcaacgcctccggaatagagttagtcagtatagaacacgctatggacctttgggcggatccggagggcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 82 - protein, 454 aa, Artificial Sequence
  Synthetic FK10 beta-LZR-IgG1 light chain
MGSDPDLVKLPSCPDPAMGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSFGPDGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGGGGSGGGGSGGGGSGGGGSLEIEAAFLERENTALETRVAELRQRVQRLRNRVSQYRTRYGPLGGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 6 aa, Artificial Sequence
  Synthetic His tag
HHHHHH